(19) United States
(12) Patent Application Publication
(10) Pub. No.: US 2008/0124763 A1
(43) Pub. Date: May 29, 2008

(54) Constructs and methods for expression of recombinant proteins in methylotrophic yeast cells

(76) Inventors: Erwin Sablon, Merchtem (BE); Annie Van Broekhoven, Berchem (BE); Fons Bosman, Opwijk (BE); Erik Depla, Destelbergen (BE); Geert Deschamps, Aalter (BE)

Correspondence Address: NIXON & VANDERHYE, PC, 901 NORTH GLEBE ROAD, 11TH FLOOR, ARLINGTON, VA, 22203, US

(21) Appl. No.: 11/902,807

(22) Filed: Sep. 25, 2007

Related U.S. Application Data
(63) Continuation-in-part of application No. 10/128,587, filed on Apr. 24, 2002, now Pat. No. 7,314,925.
(60) Provisional application No. 60/305,604, filed on Jul. 17, 2001.

(30) Foreign Application Priority Data
Apr. 24, 2001 (EP) 01870088.0

(51) Int. Cl. C12P 21/00; C12N 15/52; C12N 15/74; C12N 1/19

(52) U.S. Cl. 435/69.1; 536/23.2; 435/254.2; 435/320.1

(57) ABSTRACT

The current invention relates to vectors and methods for efficient expression of proteins in methylotrophic yeast cells. More particularly said vectors comprise the coding sequence for a lysozyme signal peptide or a functional equivalent thereof joined to a protein. Said lysozyme signal peptide is efficiently removed when the protein comprising said lysozyme signal peptide joined to a protein is expressed in methylotrophic yeast cells, such as Hansenula polymorpha and Pichia pastoris.

The present application is a continuation-in part application of U.S. application Ser. No. 10/128,587, filed Apr. 24, 2002 (pending and allowed), which claims benefit of U.S. provisional application Ser. No. 60/305,604, filed Jul. 17, 2001 and EP 01870088.0, filed Apr. 24, 2001, the entire contents of each of these applications being incorporated herein in their entirety by reference.

### FIELD OF THE INVENTION

The present invention relates to the general field of recombinant protein expression. More particularly, the present invention relates to the expression of proteins in a eukaryote such as yeast, more particularly in methylotrophic yeast cells. Constructs and methods are disclosed for the expression of proteins in methylotrophic yeast cells, such as Hansenula polymorpha and Pichia pastoris.

### BACKGROUND OF THE INVENTION

Hepatitis C virus (HCV) infection is a major health problem in both developed and developing countries. It is estimated that about 1 to 5% of the world population is affected by the virus. HCV infection appears to be the most important cause of transfusion-associated hepatitis and frequently progresses to chronic liver damage. Moreover, evidence exists implicating HCV in induction of hepatocellular carcinoma. Consequently, the demand for reliable diagnostic methods and effective therapeutic agents is high. Also sensitive and specific screening methods of HCV-contaminated blood-products and improved methods to culture HCV are needed.

HCV is a positive stranded RNA virus of approximately 9,600 bases which encode a single polyprotein precursor of about 3000 amino acids. Proteolytic cleavage of the precursor coupled to co- and posttranslational modifications has been shown to result in at least three structural and six non-structural proteins. Based on sequence homology, the structural proteins have been functionally assigned as one single core protein and two envelope glycoproteins: E1 and E2. The E1 protein consists of 192 amino acids and contains 5 to 6 N-glycosylation sites, depending on the HCV genotype. The E2 protein consists of 363 to 370 amino acids and contains 9 to 11 N-glycosylation sites, depending on the HCV genotype (for reviews see: (Maertens, G. and Stuyver, L. 1997, Major, M. E. and Feinstone, S. M. 1997)). The E1 protein contains various variable domains (Maertens, G. and Stuyver, L. 1997). The E2 protein contains three hypervariable domains, of which the major domain is located at the N-terminus of the protein (Maertens, G. and Stuyver, L. 1997). The HCV glycoproteins localize predominantly in the ER where they are modified and assembled into oligomeric complexes.

In eukaryotes, sugar residues are commonly linked to four different amino acid residues. These amino acid residues are classified as O-linked (serine, threonine, and hydroxylysine) and N-linked (asparagine). The O-linked sugars are synthesized in the Golgi or rough Endoplasmic Reticulum (ER) from nucleotide sugars. The N-linked sugars are synthesized from a common precursor, and subsequently processed. It is believed that HCV envelope proteins are N-glycosylated. It is known in the art that addition of N-linked carbohydrate chains is important for stabilization of folding intermediates and thus for efficient folding, prevention of malfolding and degradation in the endoplasmic reticulum, oligomerization, biological activity, and transport of glycoproteins (see reviews by (Doms, R. W. et al. 1993a, Doms, R. W. et al. 1993b, Rose, J. K. and Doms, R. W. 1988), (Helenius, A. 1994)). The tripeptide sequences Asn-X-Ser and Asn-X-Thr (in which X can be any amino acid) on polypeptides are the consensus sites for binding N-linked oligosaccharides. After addition of the N-linked oligosaccharide to the polypeptide, the oligosaccharide is further processed into the complex type (containing N-acetylglucosamine, mannose, fucose, galactose and silica acid) or the high-mannose type (containing N-acetylglucosamine and mannose). HCV envelope proteins are believed to be of the high-mannose type. N-linked oligosaccharide biosynthesis in yeast is very different from the biosynthesis in mammalian cells. In yeast the oligosaccharide chains are elongated in the Golgi through stepwise addition of mannose, leading to elaborate high mannose structures, leading to elaborate high mannose structures, referred to as hyperglycosylation. In contrast therewith, proteins expressed in prokaryotes are never glycosylated.

To date, vaccination against disease has been proven to be the most cost effective and efficient method for controlling diseases. Despite promising results, efforts to develop an efficacious HCV vaccine, however, have been plagued with difficulties. A conditio sine qua non for vaccines is the induction of an immune response in patients. Consequently, HCV antigenic determinants should be identified, and administered to patients in a proper setting. Antigenic determinants can be divided in at least two forms, i.e. lineair and conformational epitopes. Conformational epitopes result from the folding of a molecule in a three-dimensional space, including co- and posttranslational modifications, such as glycosylation. In general, it is believed that conformational epitopes will realize the most efficacious vaccines, since they represent epitopes which resemble native-like HCV epitopes, and which may be better conserved than the actual linear amino acid sequence. Hence, the eventual degree of glycosylation of the HCV envelope proteins is of the utmost importance for generating native-like HCV antigenic determinants. However, there are seemingly insurmountable problems with culturing HCV, that result in only minute amounts of virions. In addition, there are vast problems with the expression and purification of recombinant proteins, that result in either low amounts of proteins, hyperglycosylated proteins, or proteins that are not glycosylated.

In order to obtain glycosylation of an expressed protein, said protein needs to be targeted to the endoplasmic reticulum (ER). This process requires the presence of a pre-pro- or pre-sequence, the latter also known as signal peptide or leader peptide, at the amino-terminal end of the expressed protein. Upon translocation of the protein into the lumen of the ER, the pre-sequence is removed by means of a signal peptidase complex. A large number of pre-pro- and pre-sequences is currently known in the art. These include the S. cerevisiae α-mating factor leader (pre-pro; αMF or MFα), the Carcinus maenas hyperglycemic hormone leader sequence (pre; CHH), the S. occidentalis amylase leader sequence (pre; Amyl), the S. occidentalis glucoamylase Gam1 leader sequence (pre; Gam1), the fungal phytase leader sequence (pre; Phy5), the Pichia pastoris acid phosphatase leader sequence (pre; pho1), the yeast aspartic protease 3 signal peptide (pre; YAP3), the mouse salivary amylase signal peptide (pre) and the chicken lysozyme leader sequence (pre; CL).

The CHH leader has been coupled with hirudin and G-CSF (granulocyte colony stimulating factor) and expression of the CHH-hirudin and CHH-G-CSF proteins in Hansenula polymorpha results in correct removal of the leader sequence (Weydemann, U. et al. 1995), Fischer et al. in WO00/40727). The chicken lysozyme leader sequence has been fused to human interferonα2b (IFNα2b), human serum albumin and human lysozyme or 1,4-β-N-acetylmuramidase and expressed in S. cerevisiae (Rapp in GenBank accession number AF405538, (Okabayashi, K. et al. 1991), de Baetselier et al. in EP0362183, Oberto and Davison in EP0184575). Mustilli and coworkers (Mustilli, A. C. et al. 1999d) have utilized the Kluyveromyces lactis killer toxin leader peptide for expression of HCV E2 in S. cerevisiae and K lactis.

The HCV envelope proteins have been produced by recombinant techniques in Escherichia coli, insect cells, yeast cells and mammalian cells. However, expression in higher eukaryotes has been characterised by the difficulty of obtaining large amounts of antigens for

FIG. 23. Schematic map of the vector pBSK-E2sH6 which has the sequence as defined in SEQ ID NO:45.

FIG. 24. Schematic map of the vector pYIG5HCCL-22aH6 which has the sequence as defined in SEQ ID NO:46.

FIG. 25. Schematic map of the vector pYYIGSE2H6 which has the sequence as defined in SEQ ID NO:47.

FIG. 26. Schematic map of the vector pYIG7 which has the sequence as defined in SEQ ID NO:48.

FIG. 27. Schematic map of the vector pYIG7E1 which has the sequence as defined in SEQ ID NO:49.

FIG. 28. Schematic map of the vector pSY1YIG7E1s which has the sequence as defined in SEQ ID NO:50.

FIG. 29. Schematic map of the vector pPICZalphaA which has the sequence as defined in SEQ ID NO:51.

FIG. 30. Schematic map of the vector pPICZalphaD′ which has the sequence as defined in SEQ ID NO:52.

FIG. 31. Schematic map of the vector pPICZalphaE′ which has the sequence as defined in SEQ ID NO:53.

FIG. 32. Schematic map of the vector pPICZalphaE′E1sH6 which has the sequence as defined in SEQ ID NO:58.

FIG. 33. Schematic map of the vector pPICZalphaE′E1sH6 which has the sequence as defined in SEQ ID NO:59.

eventual vaccine production. Expression in prokaryotes, such as E. coli results in HCV envelope proteins that are not glycosylated. Expression of HCV envelope proteins in yeast resulted in hyperglycosylation. As already demonstrated in WO 96/04385, the expression of HCV envelope protein E2 in Saccharomyces cerevisiae leads to proteins which are heavily glycosylated. This hyperglycosylation leads to shielding of protein epitopes. Although Mustilli and co-workers (Mustilli, A. C. et al. 1999c) claims that expression of HCV E2 in S. cerevisiae results in core-glycosylation, the analysis of the intracellularly expressed material demonstrates that part of it is at least hyperglycosylated, while the correct processing of the remainder of this material has not been shown. The need for HCV envelope proteins derived from an intracellular source is well accepted (WO 96/04385 to Maertens et al. and (Heile, J. M. et al. 2000). This need is further exemplified by the poor reactivity of the secreted yeast derived E2 with sera of chimpanzee immunized with mammalian cell culture derived E2 proteins as evidenced in FIG. 5 of Mustilli and coworkers (Mustilli, A. C. et al. 1999b). This is further documented by Rosa and colleagues (Rosa, D. et al. 1996) who show that immunization with yeast derived HCV envelope proteins fails to protect from challenge.

Consequently, there is a need for efficient expression systems resulting in large and cost-effective amounts of proteins and, in particular, such systems are needed for production of HCV envelope proteins. If a pre- or pre-pro-sequence is used to direct the protein of interest to the ER, then efficiency of the expression system is, amongst others, dependent on the efficiency and fidelity with which the pre- or pre-pro-sequences are removed from the protein of interest.

### SUMMARY OF THE INVENTION

A first aspect of the present invention relates to recombinant nucleic acids comprising a nucleotide sequence encoding a protein comprising a lysozyme leader peptide or a functional equivalent thereof joined to a protein of interest or a part thereof, wherein said protein of interest or part thereof is expressed in a methylotrophic yeast cell. More specifically said protein is characterized by the structure L-[(A1)ₐ-(PS1)_{b}-(A2)_{c}]-PROT-[(A3)_{d}-(PS2)ₑ-(A4)_{f}]

wherein:
L is a lysozyme leader peptide or a functional equivalent thereof,
A1, A2, A3 and A4 are adaptor peptides which can be different or the same,
PS1 and PS2 are processing sites which can be the different or the same,
PROT is a protein of interest or a part thereof,
a, b, c, d, e and f are 0 or 1, and
wherein, optionally, A1 and/or A2 are part of PS1 and/or wherein A3 and/or A4 are part of PS2.

The recombinant nucleic acids according to the invention may further comprise regulatory elements allowing expression of said protein in a methyltrophic yeast cell.

Another aspect of the invention relates to a recombinant nucleic acid according to the invention which are comprised in a vector. Said vector may be an expression vector and/or an autonomously replicating vector or an integrative vector.

A further aspect of the invention relates to a methylotrophic yeast cell harboring a recombinant nucleic acid according to the invention or a vector according to the invention. More particularly, said methylotrophic yeast cell is capable of expressing the protein comprising a lysozyme leader peptide or a functional equivalent thereof joined to a protein of interest or a part thereof. More specifically, said protein is characterized by the structure L-[(A1)ₐ-(PS1)_{b}-(A2)_{c}]-HCVENV-[(A3)_{d}-(PS2)ₑ-(A4)_{f}]

wherein:
L is a lysozyme leader peptide or a functional equivalent thereof,
A1, A2, A3 and A4 are adaptor peptides which can be different or the same,
PS1 and PS2 are processing sites which can be the different or the same,
PROT is a protein of interest or a part thereof,
a, b, c, d, e and f are 0 or 1, and
wherein, optionally, A1 and/or A2 are part of PS1 and/or wherein A3 and/or A4 are part of PS2.

The methylotrophic yeast cell according to the invention may be capable of removing the lysozyme leader peptide with high efficiency and fidelity and may be capable of processing the processing sites PS1 and/or PS2 in said protein translocated to the endoplasmic reticulum. Said methylotrophic yeast cell may further be capable of N-glycosylating said protein translocated to the endoplasmic reticulum or said protein translocated to the endoplasmic reticulum and processed at said sites PS1 and/or PS2.

A next aspect of the invention relates to a method for producing a protein of interest or part thereof in a methylotrophic yeast cell, said method comprising transforming said methylotrophic yeast cell with a recombinant nucleic acid according to the invention or with a vector according to the invention, and wherein said methylotrophic yeast cell is capable of expressing a protein comprising the avian lysozyme leader peptide or a functional equivalent thereof joined to a protein of interest or a part thereof. More particularly, said protein is characterized by the structure L-[(A1)ₐ-(PS1)_{b}-(A2)_{c}]-PROT-[(A3)_{d}-(PS2)ₑ-(A4)_{f}]

wherein:
L is alysozyme leader peptide or a functional equivalent thereof,
A1, A2, A3 and A4 are adaptor peptides which can be different or the same,
PS1 and PS2 are processing sites which can be the different or the same, PROT is a protein of interest or a part thereof,
a, b, c, d, e and f are 0 or 1, and
wherein, optionally, A1 and/or A2 are part of PS1 and/or wherein A3 and/or A4 are part of PS2.

The method according to the invention may further comprise cultivation of said methylotrophic yeast cells in a suitable medium to obtain expression of said protein, isolation of the expressed protein from a culture of said methylotrophic yeast cells, or from said methylotrophic yeast cells. Said isolation may include one or more of (i) lysis of said methylotrophic yeast cells in the presence of a chaotropic agent, (ii) chemical modification of the cysteine thiol-groups in the isolated proteins wherein said chemical modification may be reversible or irreversible and (iii) heparin affinity chromatography.

### FIGURE LEGENDS

FIG. 1. Schematic map of the vector pGEMT-E1sH6RB which has the sequence as defined in SEQ ID NO:6.

FIG. 2. Schematic map of the vector pCHH-Hir which has the sequence as defined in SEQ ID NO:9.

FIG. 3. Schematic map of the vector pFPMT121 which has the sequence as defined in SEQ ID NO:12.

FIG. 4. Schematic map of the vector pFPMT-CHH-E1-H6 which has the sequence as defined in SEQ ID NO:13.

FIG. 5. Schematic map of the vector pFPMT-MFα-E1-H6 which has the sequence as defined in SEQ ID NO:16.

FIG. 6. Schematic map of the vector pUC18-FMD-MFα-E1-H6 which has the sequence as defined in SEQ ID NO:17.

FIG. 7. Schematic map of the vector pUC18-FMD-CL-E1-H6 which has the sequence as defined in SEQ ID NO:20.

FIG. 8. Schematic map of the vector pFPMT-CL-E1-H6 which has the sequence as defined in SEQ ID NO:21.

FIG. 9. Schematic map of the vector pSP72E2H6 which has the sequence as defined in SEQ ID NO:22.

FIG. 10. Schematic map of the vector pMPT121 which has the sequence as defined in SEQ ID NO:23.

FIG. 11. Schematic map of the vector pFPMT-MFα-E2-H6 which has the sequence as defined in SEQ ID NO:24.

FIG. 12. Schematic map of the vector pMPT-MFα-E2-H6 which has the sequence as defined in SEQ ID NO:25.

FIG. 13. Schematic map of the vector pMF30 which has the sequence as defined in SEQ ID NO:28.

FIG. 14. Schematic map of the vector pFPMT-CL-E2-H6 which has the sequence as defined in SEQ ID NO:32.

FIG. 15. Schematic map of the vector pUC18-FMD-CL-E1 which has the sequence as defined in SEQ ID NO:35.

FIG. 16. Schematic map of the vector pFPMT-CL-E1 which has the sequence as defined in SEQ ID NO:36.

FIG. 17. Schematic map of the vector pUC18-FMD-CL-H6-E1-K-H6 which has the sequence as defined in SEQ ID NO:39.

FIG. 18. Schematic map of the vector pFPMT-CL-H6-K-E1 which has the sequence as defined in SEQ ID NO:40.

FIG. 19. Schematic map of the vector pYIG5 which has the sequence as defined in SEQ ID NO:41.

FIG. 20. Schematic map of the vector pYIG5E1H6 which has the sequence as defined in SEQ ID NO:42.

FIG. 21. Schematic map of the vector pSY1 which has the sequence as defined in SEQ ID NO:43.

FIG. 22. Schematic map of the vector pSY1aMFE1sH6a which has the sequence as defined in SEQ ID NO:44.

FIG. 34. Schematic map of the vector pPICZalphaD′E2sH6 which has the sequence as defined in SEQ ID NO:60.

FIG. 35. Schematic map of the vector pPICZalphaE′E2sH6 which has the sequence as defined in SEQ ID NO:61.

FIG. 36. Schematic map of the vector pUC18MFα which has the sequence as defined in SEQ ID NO:62.

FIG. 37. Elution profile of size exclusion chromatography of IMAC-purified E2-H6 protein expressed from the MFα-E2-H6-expressing Hansenula polymorpha (see Example 15). The X-axis indicates the elution volume (in mL). The vertical lines through the elution profile indicate the fractions collected. “P1”=pooled fractions 4 to 9, “P2”=pooled fractions 30 to 35, and “P3”=pooled fractions 37 to 44. The Y-axis indicates absorbance given in mAU (milli absorbance units). The X-axis indicates the elution volume in mL.

FIG. 38. The different pools and fractions collected after size exclusion chromatography (see FIG. 37) were analyzed by non-reducing SDS-PAGE followed by silver staining of the polyacrylamide gel. The analyzed pools (“P1”, “P2”, and “P3”) and fractions (16 to 26) are indicated on top of the picture of the silver-stained gel. At the left (lane “M”) are indicated the sizes of the molecular mass markers.

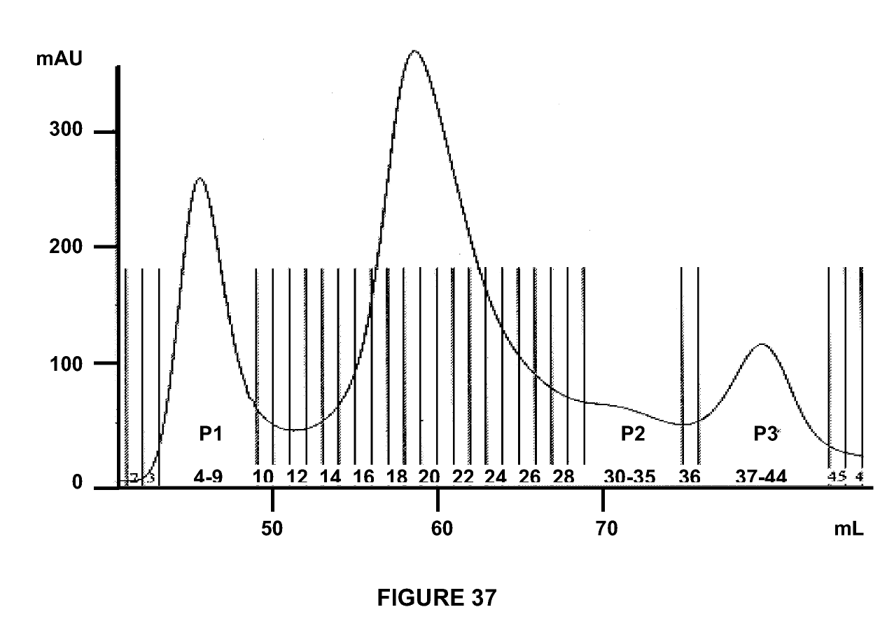

FIG. 39. Fractions 17 to 23 of the size exclusion chromatographic step as shown in FIG. 37 were pooled and alkylated. Thereafter, the protein material was subjected to Endo H treatment for deglycosylation. Untreated material and Endo H-treated material were separated on an SDS-PAGE gel and blotted to a PVDF membrane. The blot was stained with amido black.
Lane 1: Alkylated E2-H6 before Endo H-treatment
Lane 2: Alkylated E2-H6 after Endo H-treatment.

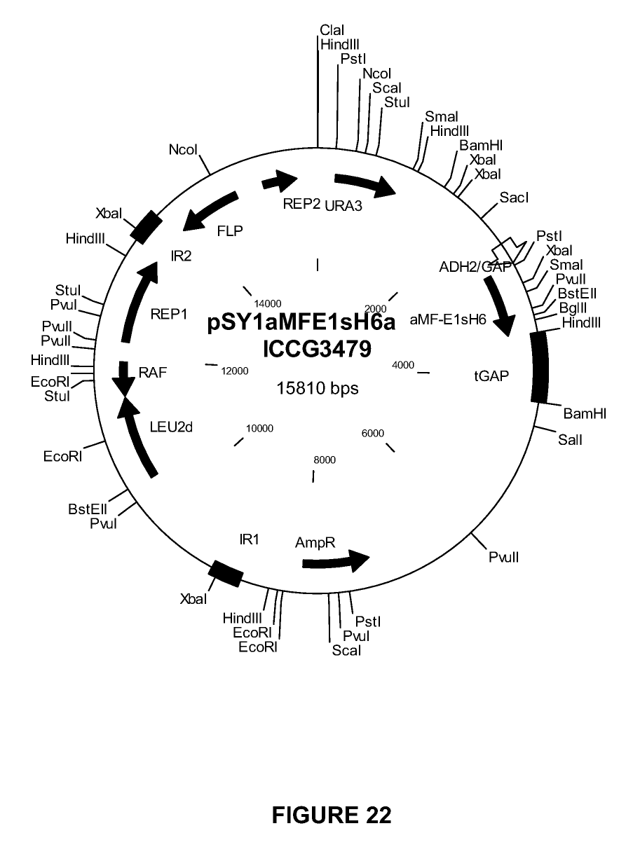

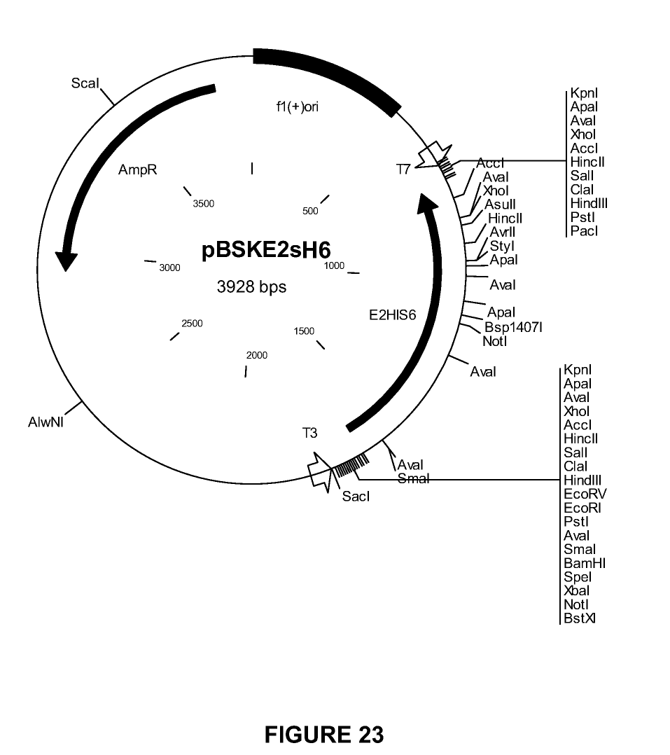

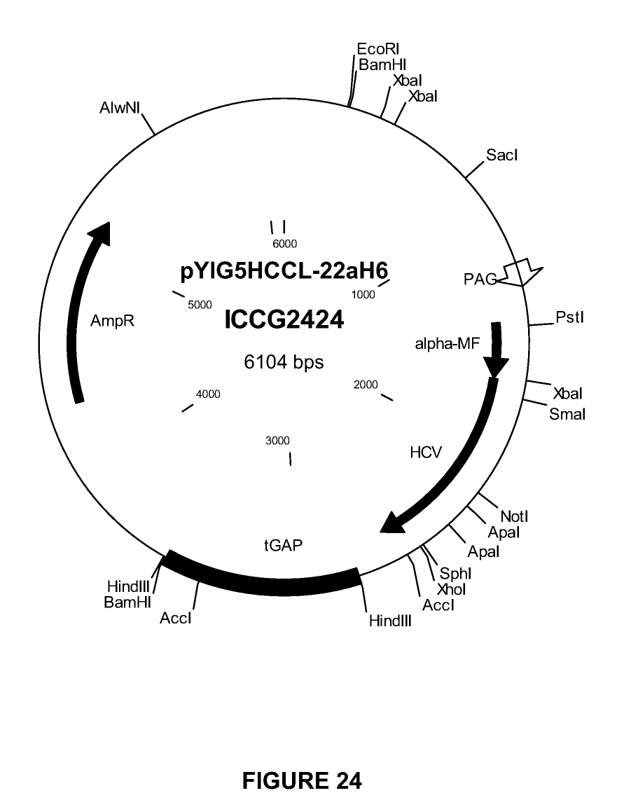

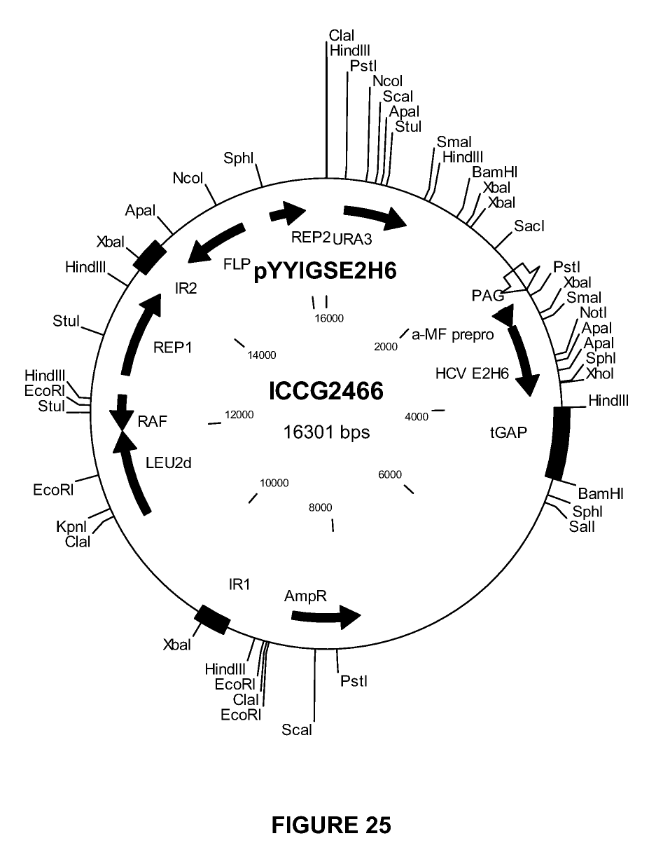

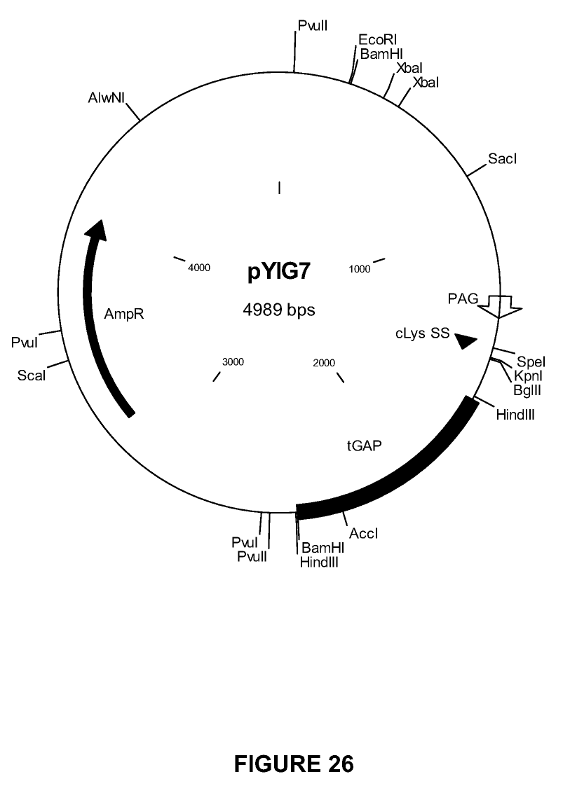

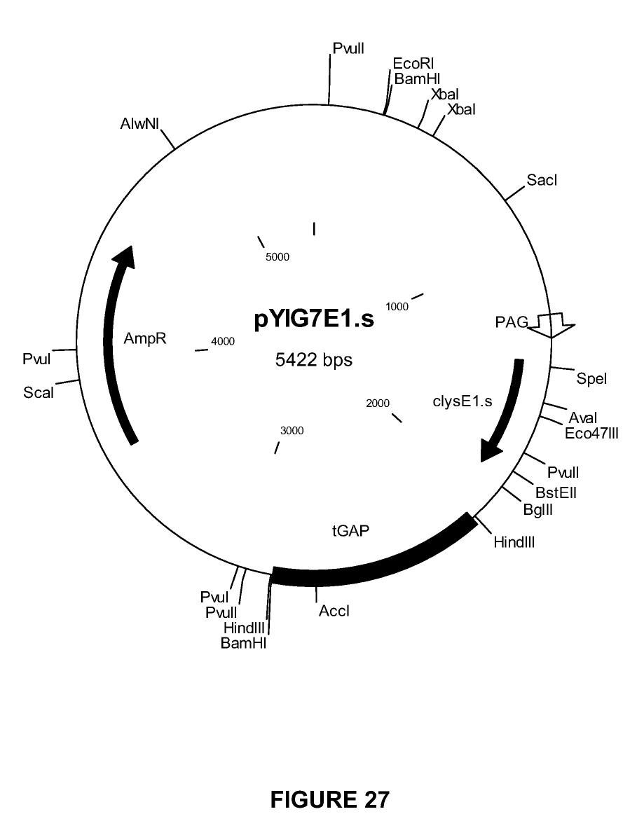

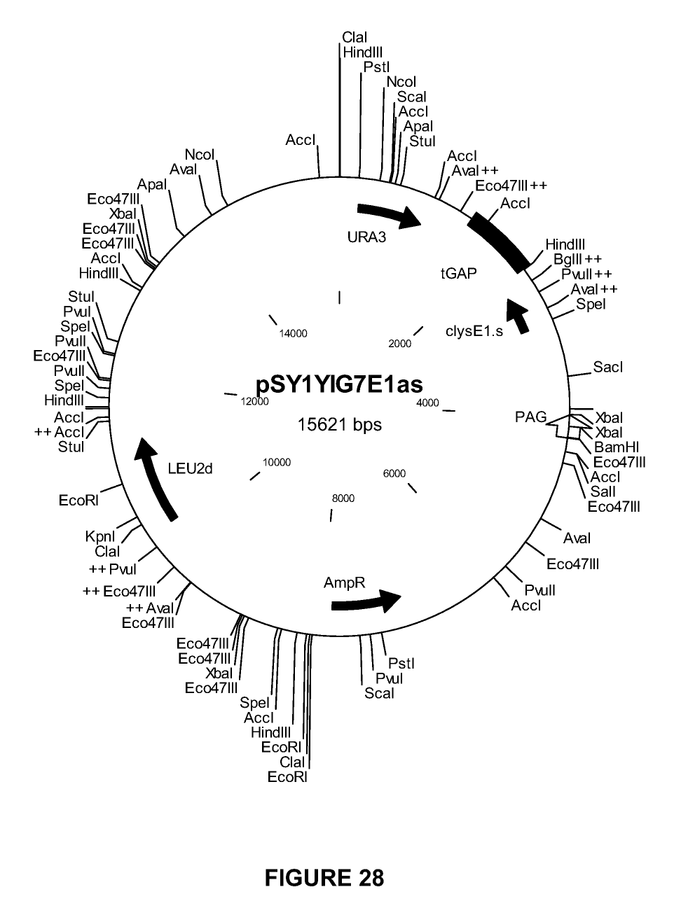

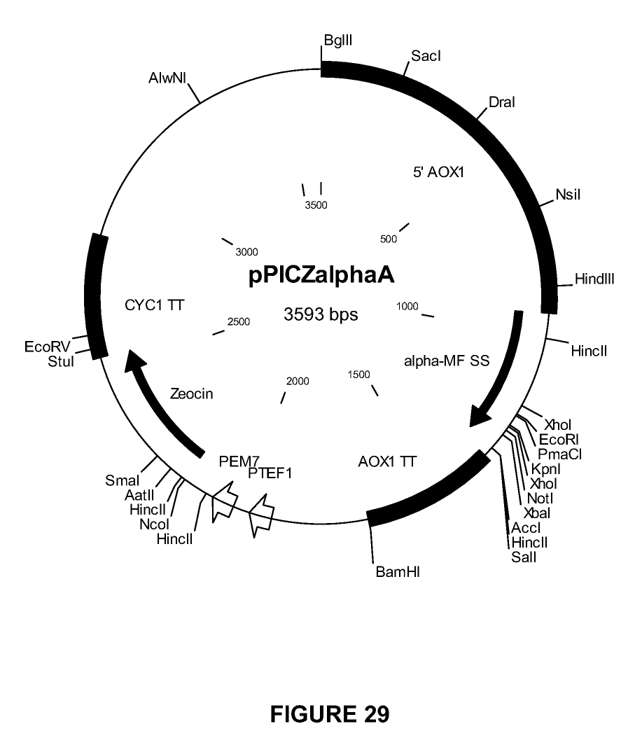

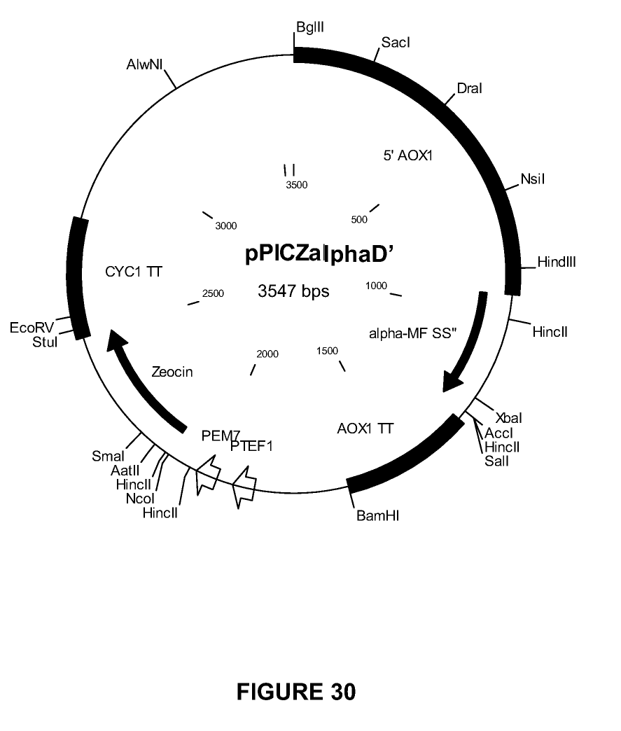

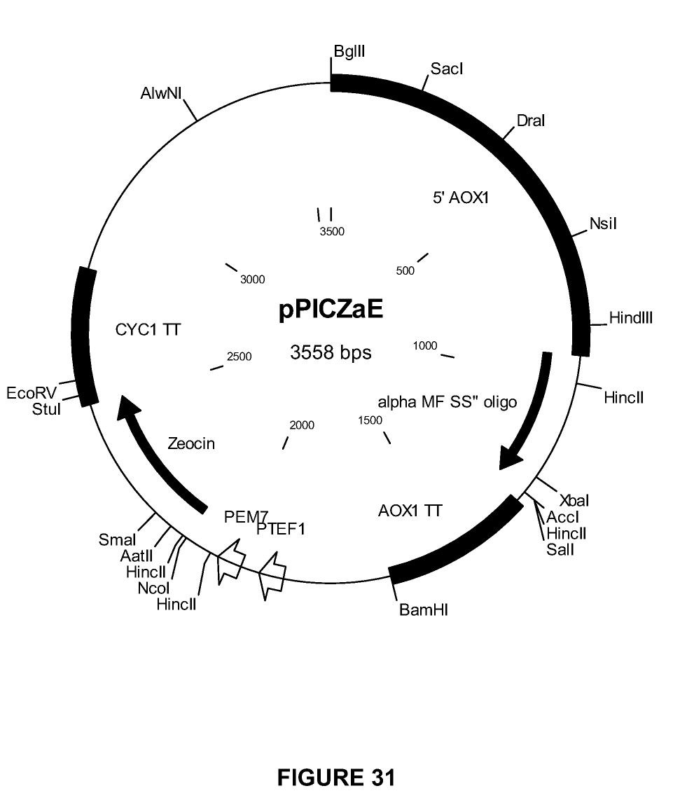

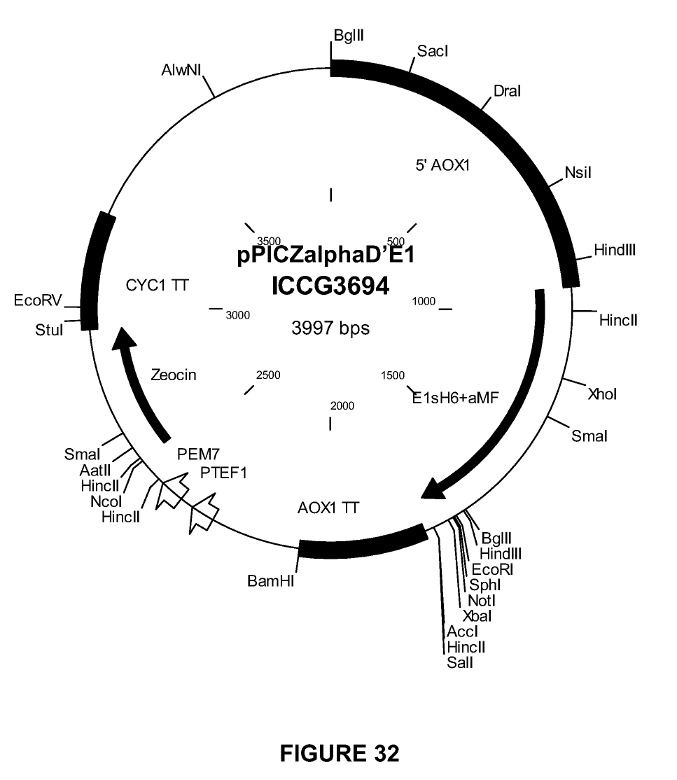

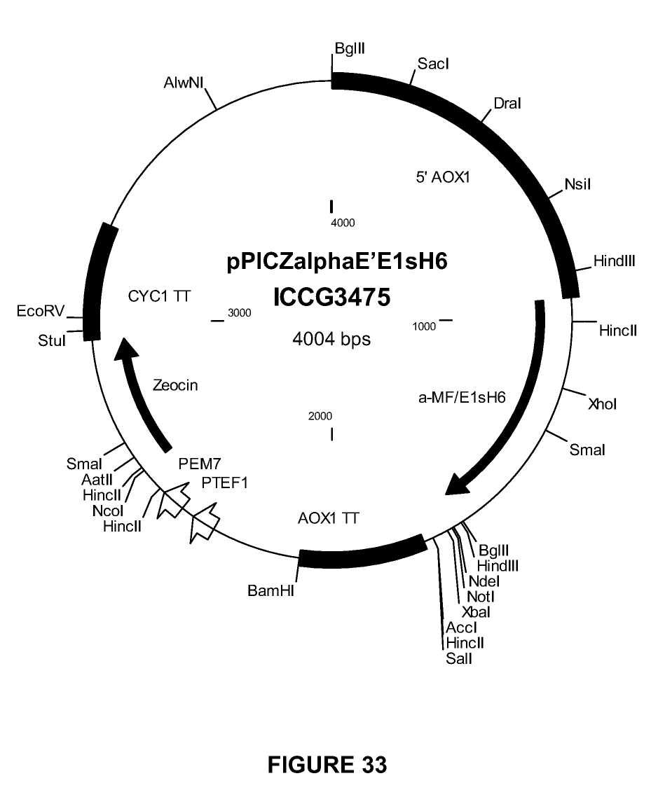

FIG. 40. Western-blot analysis of cell lysates of E1 expressed in Saccharomyces cerevisiae. The Western-blot was developed using the E1-specific monoclonal antibody IGH 201.
Lanes 1-4: expression product after 2, 3, 5 or 7 days expression, respectively, in a Saccharomyces clone transformed with pSY1YIG7E1s (SEQ ID NO:50, FIG. 28) comprising the nucleotide sequence encoding the chicken lysozyme leader peptide joined to E1-H6.
Lanes 5-7: expression product after 2, 3 or 5 days expression, respectively, in a Saccharomyces clone transformed with pSY1aMFE1sH6aYIG1 (SEQ ID NO:44, FIG. 22) comprising the nucleotide sequence encoding the α-mating factor leader peptide joined to E1-H6.
Lane 8: molecular weight markers with sizes as indicated.
Lane 9: purified E1s produced by HCV-recombinant vaccinia virus-infected mammalian cells.

FIG. 41. Analysis of the immobilized metal ion affinity chromatography (IMAC)-purified E2-H6 protein expressed by and processed from CL-E2-H6 to E2-H6 by H. polymorpha (see Example 17). Proteins in different wash fractions (lanes 2 to 4) and elution fractions (lanes 5 to 7) were analyzed by reducing SDS-PAGE followed by silver staining of the gel (A, top picture) or by western blot using a specific monoclonal antibody directed against E2 (B, bottom picture). The sizes of the molecular mass markers are indicated at the left.

FIG. 42. Elution profile of the first IMAC chromatography step on a Ni-IDA column (Chelating Sepharose FF loaded with Ni²⁺, Pharmacia) for the purification of the sulfonated H6-K-E1 protein produced by H. polymorpha (see Example 18). The column was equilibrated with buffer A (50 mM phosphate, 6 M GuHCl, 1% Empigen BB (v/v), pH 7.2) supplemented with 20 mM imidazole. After sample application, the column was washed sequentially with buffer A containing 20 mM and 50 mM imidazole, respectively (as indicated on chromatogram). A further washing and elution step of the His-tagged products was performed by the sequential application of buffer B (PBS, 1% empigen BB, pH 7.2) supplemented with 50 mM imidazole and 200 mM imidazole respectively (as indicated on chromatogram). Following fractions were pooled: the wash pool 1 (fractions 8 to 11, wash with 50 mM imidazole). The eluted material was collected as separate fractions 63 to 72 or an elution pool (fractions 63 to 69) was made. The Y-axis indicates absorbance given in mAU (milli absorbance units). The X-axis indicates the elution volume in mL

FIG. 43. Analysis of the IMAC-purified H6-K-E1 protein (see FIG. 42) expressed by and processed from CL-H6-K-E1 to H6-K-E1 by H. polymorpha. Proteins in the wash pool 1 (lane 12) and elution fractions 63 to 72 (lanes 2 to 11) were analyzed by reducing SDS-PAGE followed by silver staining of the gel (A, top picture). Proteins present in the sample before IMAC (lane 2), in the flow-through pool (lane 4), in wash pool 1 (lane 5) and in the elution pool (lane 6) were analyzed by western blot using a specific monoclonal antibody directed against E1 (IGH201) (B, bottom picture; no sample was loaded in lane 3). The sizes of the molecular mass markers (lanes M) are indicated at the left.

FIG. 44. Elution profile of the second IMAC chromatography step on a Ni-IDA column (Chelating Sepharose FF loaded with Ni²⁺, Pharmacia) for the purification of E1 resulting from the in vitro processing of H6-K-E1 (purification: see FIG. 42) with Endo Lys-C. The flow through was collected in different fractions (1 to 40) that were screened for the presence of E1s-products. The fractions (7 to 28), containing intact E1 processed from H6-K-E1 were pooled. The Y-axis indicates absorbance given in mAU (milli absorbance units). The X-axis indicates the elution volume in 1 mL

FIG. 45. Western-blot analysis indicating specific E1s proteins bands reacting with biotinylated heparin (see also Example 19). E1s preparations purified from HCV-recombinant vaccinia virus-infected mammalian cell culture or expressed by H. polymorpha were analyzed. The panel right from the vertical line shows a Western-blot developed with the biotinylated E1 specific monoclonal IGH 200. The panel left from the vertical line shows a Western-blot developed with biotinylated heparin. From these results it is concluded that mainly the lower-glycosylated E1s has high affinity for heparin.
Lanes M: molecular weight marker (molecular weights indicated at the left).
Lanes 1: E1s from mammalian cells and alkylated during isolation.
Lanes 2: E1s-H6 expressed by H. polymorpha and sulphonated during isolation.
Lanes 3: E1s-H6 expressed by H. polymorpha and alkylated during isolation.
Lanes 4: same material as loaded in lane 2 but treated with dithiotreitol to convert the sulphonated Cys-thiol groups to Cys-thiol.

FIG. 46. Size exclusion chromatography (SEC) profile of the purified H. polymorpha-expressed E2-H6 in its sulphonated form, submitted to a run in PBS, 3% betain to force virus-like particle formation by exchange of Empigen BB for betain. The pooled fractions containing the VLPs used for further study are indicated by [symbol] . The Y-axis indicates absorbance given in mAU (milli absorbance units). The X-axis indicates the elution volume in mL. See also Example 20.

FIG. 47. Size exclusion chromatography (SEC) profile of the purified H. polymorpha-expressed E2-H6 in its alkylated form, submitted to a run in PBS, 3% betain to force virus-like particle formation by exchange of Empigen BB for betain. The pooled fractions containing the VLPs are indicated by [symbol] . The Y-axis indicates absorbance given in mAU (milli absorbance units). The X-axis indicates the elution volume in mL. See also Example 20.

FIG. 48. Size exclusion chromatography (SEC) profile of the purified H. polymorpha-expressed E1 in its sulphonated form, submitted to a run in PBS, 3% betain to force virus-like particle formation by exchange of Empigen BB for betain. The pooled fractions containing the VLPs are indicated by [symbol] . The Y-axis indicates absorbance given in mAU (milli absorbance units). The X-axis indicates the elution volume in mL. See also Example 20.

FIG. 49. Size exclusion chromatography (SEC) profile of the purified H. polymorpha-expressed E1 in its alkylated form, submitted to a run in PBS, 3% betain to force virus-like particle formation by exchange of Empigen BB for betain. The pooled fractions containing the VLPs are indicated by [symbol] . The Y-axis indicates absorbance given in mAU (milli absorbance units). The X-axis indicates the elution volume in mL. See also Example 20.

FIG. 50. SDS-PAGE (under reducing conditions) and western blot analysis of VLPs as isolated after size exclusion chromatography (SEC) as described in FIGS. 48 and 49. Left panel: silver-stained SDS-PAGE gel. Right panel: western blot using a specific monoclonal antibody directed against E1 (IGH201). Lanes 1: molecular weight markers (molecular weights indicated at the left); lanes 2: pool of VLPs containing sulphonated E1 (cfr. FIG. 48); lanes 3: pool of VLPs containing alkylated E1 (cfr. FIG. 49). See also Example 20.

FIG. 51. E1 produced in mammalian cells (“M”) or Hansenula-produced E1 (“H”) were coated on a ELISA solid support to determine the end point titer of antibodies present in sera after vaccination of mice with E1 produced in mammalian cells (top panel), or after vaccination of mice with Hansenula-produced E1 (bottom panel). The horizontal bar represents the mean antibody titer. The end-point titers (fold-dilution) are indicated on the Y-axis. See also Example 22.

FIG. 52. Hansenula-produced E1 was alkylated (“A”) or sulphonated (“S”) and coated on a ELISA solid support to determine the end point titer of antibodies present in sera after vaccination of mice with Hansenula-produced E1 that was alkylated (top panel), or after vaccination of mice with Hansenula-produced E1 that was sulphonated (bottom panel). The horizontal bar represents the mean antibody titer. The end-point titers (fold-dilution) are indicated on the Y-axis. See also Example 23.

FIG. 53. HCV E1 produced by HCV-recombinant vaccinia virus-infected mammalian cells and HCV E1 produced by H. polymorpha were coated directly to ELISA plates. End point titers of antibodies were determined in sera of chimpanzees vaccinated with E1 produced by mammalian cells (top panel) and of murine monoclonal antibodies raised against E1 produced by mammalian cells (bottom panel). Chimpanzees Yoran and Marti were prophylactically vaccinated. Chimpanzees Ton, Phil, Marcel, Peggy and Femma were therapeutically vaccinated. Black filled bars: ELISA plate coated with E1 produced by mammalian cells. Open bars: ELISA plate coated with E1 produced by Hansenula. The end-point titers (fold-dilution) are indicated on the Y-axis. See also Example 24.

FIG. 54. Fluorophore-assisted carbohydrate gelelectrophoresis of oligosaccharides released from E1 produced by recombinant vaccinia virus-infected mammalian cells and from E1-H6 protein produced by Hansenula.
Lane 1: Glucose ladder standard with indication at the left of the number of monosaccharides (3 to 10, indicated by G3 to G10).
Lane 2: 25 μg N-linked oligosaccharides released from (alkylated) E1 produced by mammalian cells.
Lane 3: 25 μg N-linked oligosaccharides released from (alkylated) E1-H6 produced by Hansenula.
Lane 4: 100 pmoles maltotetraose.

### See also Example 25.

FIG. 55. Schematic map of the vector pUC18-FMD-M-E1-H6 which has the sequence as defined in SEQ ID NO: 104.

FIG. 56. Schematic map of the vector pUC18-FMD-HL-E1-H6 which has the sequence as defined in SEQ ID NO:105.

FIG. 57. Schematic map of the vector pFPMT121 which has the sequence as defined in SEQ ID NO:106.

FIG. 58. Schematic map of the vector pFPMT-HL-E1-H6 which has the sequence as defined in SEQ ID NO:107.

FIG. 59. Direct comparison by Western Blot of intracellular soluble fractions of H. polymorpha containing HL- or CL-E1-H6 after 16 h incubation at 37° C. with or without PNGaseF. MAB 11B7D8 was used as the primary antibody in combination with the Western Breeze kit (mouse; Novex).

### M: molecular weight marker with sizes indicated

Lane 1: HL-E1-H6 expressed by H. polymorpha, without PNGaseF digestion
Lane 2: HL-E1-H6 expressed by H. polymorpha, with PNGaseF digestion
Lane 3: CL-E1-H6 expressed by H. polymorpha, without PNGaseF digestion
Lane 4: CL-E1-H6 expressed by H. polymorpha, with PNGaseF digestion
Lane 5: pFMPT121/RB11: negative control, untreated

FIG. 60. Schematic map of the vector pFPMT-MFα-IL-4 which has the sequence as defined in SEQ ID NO:109.

FIG. 61. Schematic map of the vector pUC18-MFα-IL-4 which has the sequence as defined in SEQ ID NO:110.

FIG. 62. Schematic map of the vector pUC18-CL-IL-4 which has the sequence as defined in SEQ ID NO:113.

FIG. 63. Schematic map of the vector pUC18-CL-IL-4-H6 which has the sequence as defined in SEQ ID NO:114.

FIG. 64. Schematic map of the vector pFPMT-CL-IL-4-H6 which has the sequence as defined in SEQ ID NO:117.

FIG. 65. Qualitative screening of intracellular soluble fractions of Hansenula polymorpha harbouring pFMPT-CL-IL-4-H6 for presence of IL-4. Strains were been subjected to MeOH-induction. Intracellular soluble fractions were prepared and analyzed by Western Blot. MAB21E1B4 (H6) was used as the primary antibody in combination with the Western Breeze kit (mouse; Novex).

### M: molecular weight marker with sizes indicated

Lane 1-12: CL-IL-4-H6 expressed by H. polymorpha
Lane 13: pFMPT121/RB11: negative control

FIG. 66. PNGaseF assay of intracellular soluble fractions of Hansenula polymorpha strains expressing CL-IL-4-H6 from plasmid pFMPT-CL-IL-4-H6. The indicated strains were subjected to a repeated MeOH induction. The intracellular soluble fractions were prepared and incubated for 16 h with or without PNGaseF. For Western Blot analysis, MAB21E1B4 (H6) was used as the primary antibody in combination with the Western Breeze kit (mouse; Novex).

### M: molecular weight marker with sizes indicated

Lane 1: CL-IL-4-H6/#3 expressed by H. polymorpha, with PNGaseF digestion
Lane 2: CL-IL-4-H6/#3 expressed by H. polymorpha, without PNGaseF digestion
Lane 3: CL-IL-4-H6/#5 expressed by H. polymorpha, with PNGaseF digestion
Lane 4: CL-IL-4-H6/#5 expressed by H. polymorpha, without PNGaseF digestion
Lane 5: CL-IL-4-H6/#8 expressed by H. polymorpha, with PNGaseF digestion
Lane 6: CL-IL-4-H6/#8 expressed by H. polymorpha, without PNGaseF digestion
Lane 7: human IL-4 standard
Lane 8: pFMPT121/RB11: negative control, untreated

### DETAILED DESCRIPTION OF THE INVENTION

In work leading to the present invention, it was observed that expression of HCV envelope proteins as αMF-HCVENV (αmating factor-HCV envelope protein) pre-proproteins in Saccharomyces cerevisiae, Pichia pastoris and Hansenula polymorpha was possible but that the extent of removal of the pre-pro- or pre-sequences was unacceptably low and that removal of pre-pro- or pre-sequences is very often not occurring with high fidelity. As a result, many different HCV envelope proteins are produced in these yeasts which do not have a natural amino-terminus (see Example 15). The majority of the HCV envelope proteins expressed in these yeast species were glycosylated (see Examples 6, 10, 13 and 25). More specifically the S. cerevisiae (glycosylation deficient mutant)- and H. polymorpha-expressed HCV envelope proteins were glycosylated in a manner resembling core-glycosylation. The HCV envelope proteins expressed in Pichia pastoris were hyperglycosylated despite earlier reports that proteins expressed in this yeast are normally not hyperglycosylated (Gellissen, G. 2000b, Sugrue, R. J. et al. 1997a).

Constructs were made for expression of the HCV envelope proteins as pre-pro- or pre-proteins wherein these pre-pro- or pre-sequences were either the Carcinus maenas hyperglycemic hormone leader sequence (pre; CHH), the S. occidentalis amylase leader sequence (pre; Amyl), the S. occidentalis glucoamylase Gam1 leader sequence (pre; Gam1), the fungal phytase leader sequence (pre; Phy5), the Pichia pastoris acid phosphatase leader sequence (pre; pho1), the yeast aspartic protease 3 signal peptide (pre; YAP3), the mouse salivary amylase signal peptide (pre) and the chicken lysozyme leader sequence (pre; CL). Only for one of these pre-pro-HCVENV or pre-HCVENV proteins, removal of the pre-pro- or pre-sequence with high frequency and high fidelity was observed. This was surprisingly found for the chicken lysozyme leader sequence (CL) and was confirmed both in S. cerevisiae and H. polymorpha (see Example 16). The CL signal peptide is thus performing very well for expression of glycosylated HCV envelope proteins in eukaryotic cells. This unexpected finding is reflected in the different aspects and embodiments of the present invention as presented below.

Based on this surprising finding, additional constructs were made for expression of target proteins using lysozyme leader sequences in a methylotrophic yeast expression system, such as Hansenula polymorpha or Pichia pastoris and the like. For example, it was demonstrated that the human lysozyme leader peptide (pre; HL) provides for an HCV envelope protein-translocation comparable to that observed with the CL-pre-sequence (see Example 28). Further, it was demonstrated that the promising properties of the CL-pre-sequence in combination with the methylotrophic yeast expression system can be transferred to other, non-HCV target proteins also, e.g. the interleukin-4 (IL4) which is a human cytokine involved in at least several B-cell activation processes (see Example 31). These results indicate the general suitability of lysozyme pre-sequences for target protein-targeting to the ER of methylotrophic yeast cells.

A first aspect of the current invention relates to a recombinant nucleic acid comprising a nucleotide sequence encoding a protein comprising an avian lysozyme leader peptide or a functional equivalent thereof joined to an HCV envelope protein or a part thereof.

In one embodiment thereto, the recombinant nucleic acid comprising nucleotide sequence encodes characterized by the structure

CL-[(A1)ₐ-(PS1)_{b}-(A2)_{c}]-HCVENV-[(A3)_{d}-(PS2)ₑ-(A4)_{f}]

wherein:
CL is an avian lysozyme leader peptide or a functional equivalent thereof,
A1, A2, A3 and A4 are adaptor peptides which can be different or the same,
PS1 and PS2 are processing sites which can be the different or the same,
HCVENV is a HCV envelope protein or a part thereof,
a, b, c, d, e and f are 0 or 1, and
wherein, optionally, A1 and/or A2 are part of PS1 and/or wherein A3 and/or A4 are part of PS2.

In a further embodiment, the recombinant nucleic acids according to the invention further comprise regulatory elements allowing expression in a eukaryotic host cell of said protein comprising an avian lysozyme leader peptide or a functional equivalent thereof joined to an HCV envelope protein or a part thereof, or of said protein characterized by the structure CL-[(A1)ₐ-(PS1)_{b}-(A2)_{c}]-HCVENV-[(A3)_{d}-(PS2)ₑ-(A4)_{f}].

The terms “polynucleotide”, “polynucleic acid”, “nucleic acid sequence”, “nucleotide sequence”, “nucleic acid molecule”, “oligonucleotide”, “probe” or “primer”, when used herein refer to nucleotides, either ribonucleotides, deoxyribonucleotides, peptide nucleotides or locked nucleotides, or a combination thereof, in a polymeric form of any length or any shape (e.g. branched DNA). Said terms furthermore include double-stranded (ds) and single-stranded (ss) polynucleotides as well as triple-stranded polynucleotides. Said terms also include known nucleotide modifications such as methylation, cyclization and ‘caps’ and substitution of one or more of the naturally occurring nucleotides with an analog such as inosine or with non-amplifiable monomers such as HEG (hexethylene glycol). Ribonucleotides are denoted as NTPs, deoxyribonucleotides as dNTPs and dideoxyribonucleotides as ddNTPs.

Nucleotides can generally be labeled radioactively, chemiluminescently, fluorescently, phosphorescently or with infrared dyes or with a surface-enhanced Raman label or plasmon resonant particle (PRP).

Said terms “polynucleotide”, “polynucleic acid”, “nucleic acid sequence”, “nucleotide sequence”, “nucleic acid molecule”, “oligonucleotide”, “probe” or “primer” also encompass peptide nucleic acids (PNAs), a DNA analogue in which the backbone is a pseudopeptide consisting of N-(2-aminoethyl)-glycine units rather than a sugar. PNAs mimic the behavior of DNA and bind complementary nucleic acid strands. The neutral backbone of PNA results in stronger binding and greater specificity than normally achieved. In addition, the unique chemical, physical and biological properties of PNA have been exploited to produce powerful biomolecular tools, antisense and antigene agents, molecular probes and biosensors. PNA probes can generally be shorter than DNA probes and are generally from 6 to 20 bases in length and more optimally from 12 to 18 bases in length (Nielsen, P. E. 2001). Said terms further encompass locked nucleic acids (LNAs) which are RNA derivatives in which the ribose ring is constrained by a methylene linkage between the 2′-oxygen and the 4′-carbon. LNAs display unprecedented binding affinity towards DNA or RNA target sequences. LNA nucleotides can be oligomerized and can be incorporated in chimeric or mix-meric LNA/DNA or LNA/RNA molecules. LNAs seem to be nontoxic for cultured cells (Orum, H. and Wengel, J. 2001, Wahlestedt, C. et al. 2000). In general, chimeras or mix-mers of any of DNA, RNA, PNA and LNA are considered as well as any of these wherein thymine is replaced by uracil.

The term “protein” refers to a polymer of amino acids and does not refer to a specific length of the product; thus, peptides, oligopeptides, and polypeptides are included within the definition of protein. This term also does not refer to or exclude post-expression modifications of the protein, for example, glycosylations, acetylations, phosphorylations and the like. Included within the definition are, for example, polypeptides containing one or more analogues of an amino acid (including, for example, unnatural amino acids, PNA, etc.), polypeptides with substituted linkages, as well as other modifications known in the art, both naturally occurring and non-naturally occurring.

With “pre-pro-protein” or “pre-protein” is, when used herein, meant a protein comprising a pre-pro-sequence joined to a protein of interest or a protein comprising a pro-sequence joined to a protein of interest, respectively. As alternatives for “pre-sequence”, the terms “signal sequence”, “signal peptide”, “leader peptide”, or “leader sequence” are used; all refer to an amino acid sequence that targets a pre-protein to the rough endoplasmic reticulum (ER) which is a prerequisite for (N-)glycosylation. The “signal sequence”, “signal peptide”, “leader peptide”, or “leader sequence” is cleaved off, i.e. “removed” from the protein comprising the signal sequence joined to a protein of interest, at the on the luminal side of this ER by host specific proteases referred to as signal peptidases. Likewise, a pre-pro-protein is converted to a pro-protein upon translocation to the lumen of the ER. Depending on the nature of the “pro” amino acid sequence, it can or can not be removed by the host cell expressing the pre-pro-protein. A well known pre-pro-amino acid sequence is the a mating factor pre-pro-sequence of the S. cerevisiae a mating factor.

With “recombinant nucleic acid” is intended a nucleic acid of natural or synthetic origin which has been subjected to at least one recombinant DNA technical manipulation such as restriction enzyme digestion, PCR, ligation, dephosphorylation, phosphorylation, mutagenesis, adaptation of codons for expression in a heterologous cell etc. In general, a recombinant nucleic acid is a fragment of a naturally occurring nucleic acid or comprises at least two nucleic acid fragments not naturally associated or is a fully synthetic nucleic acid.

With “an avian leader peptide or a functional equivalent thereof joined to a HCV envelope protein or a part thereof” is meant that the C-terminal amino acid of said leader peptide is covalently linked via a peptide bond to the N-terminal amino acid of said HCV envelope protein or part thereof. Alternatively, the C-terminal amino acid of said leader peptide is separated from the N-terminal amino acid of said HCV envelope protein or part thereof by a peptide or protein. Said peptide or protein may have the structure -[(A1)ₐ-(PS1)_{b}-(A2)_{c}] as defined above.

The derivation of the HCV envelope protein of interest from the protein comprising an avian lysozyme leader peptide or a functional equivalent thereof joined to an HCV envelope protein or a part thereof or of the protein characterized by the structure CL-[(A1)ₐ-(PS1)_{b}-(A2)_{c}]-HCVENV-[(A3)_{d}-(PS2)ₑ-(A4)_{f}] can be performed in vivo by the proteolytic machinery of the cells in which the pre-protein protein is expressed. More specifically, the step consisting of removal of the avian leader peptide is preferably performed in vivo by the proteolytic machinery of the cells in which the pre-protein is expressed. Derivation may, however, also be performed solely in vitro after and/or during isolation and/or purification of the pre-protein and/or protein from the cells expressing the pre-protein and/or from the culture fluid in which the cells expressing the pre-protein are grown. Alternatively, said in vivo derivation is performed in combination with said in vitro derivation. Derivation of the HCV protein of interest from a recombinantly expressed pre-protein can further comprise the use of (an) proteolytic enzyme(s) in a polishing step wherein all or most of the contaminating proteins co-present with the protein of interest are degraded and wherein the protein of interest is resistant to the polishing proteolytic enzyme(s). Derivation and polishing are not mutually exclusive processes and may be obtained by using the same single proteolytic enzyme. As an example is given here the HCV E1s protein of HCV genotype 1b (SEQ ID NO:2) which is devoid of Lys-residues. By digesting of a protein extract containing said HCV E1 proteins with the Endoproteinase Lys-C (endo-lys C), the E1 proteins will not be degraded whereas contaminating proteins containing one or more Lys-residues are degraded. Such a process may significantly simplify or enhance isolation and/or purification of the HCV E1 proteins. Furthermore, by including in a pre-protein an additional Lys-residue, e.g. between a leader peptide and a HCV E1 protein, the additional advantageous possibility of correct in vitro separation of the leader peptide from the HCV E1 pre-protein is obtainable. Other HCV E1 proteins may comprise a Lys-residue at either one or more of the positions 4, 40, 42, 44, 61, 65 or 179 (wherein position 1 is the first, N-terminal natural amino acid of the E1 protein, i.e. position 192 in the HCV polyprotein). In order to enable the use of endo-lys C as described above, said Lys-residues may be mutated into another amino acid residue, preferably into an Arg-residue.

With a “correctly removed” leader peptide is meant that said leader peptide is removed from the protein comprising the signal sequence joined to a protein of interest with high efficiency, i.e. a large number of pre-(pro-)proteins is converted to pro-proteins or proteins, and with high fidelity, i.e. only the pre-amino acid sequence is removed and not any amino acids of the protein of interest joined to said pre-amino acid sequence. With “removal of a leader peptide with high efficiency” is meant that at least about 40%, but more preferentially about 50%, 55%, 60%, 65%, 70%, 75%, 80%, 85%, 90%, 95%, 96%, 97%, 98% or even 99% of the pre-proteins is converted to the protein from which the pre-sequence is removed. Alternatively, if a substantial part of the expressed pre-proteins is not converted to the protein from which the pre-sequence is removed, these pre-proteins may still be purified.

With “functional equivalent of the avian lysozyme (CL) leader peptide” is meant a CL leader peptide wherein one or more amino acids have been substituted for another amino acid and whereby said substitution is a conservative amino acid substitution. With “conservative amino acid substitution” is meant a substitution of an amino acid belonging to a group of conserved amino acids with another amino acid belonging to the same group of conserved amino acids. As groups of conserved amino acids are considered: the group consisting of Met, Ile, Leu and Val; the group consisting of Arg, Lys and H is; the group consisting of Phe, Trp and Tyr; the group consisting of Asp and Glu; the group consisting of Asn and Gln; the group consisting of Cys, Ser and Thr; and the group consisting of Ala and Gly. An exemplary conservative amino acid substitution in the CL leader peptide is the naturally variation at position 6, the amino acid at this position being either Val or Ile; another variation occurs at position 17, the amino acid at this position being, amongst others, Leu or Pro (see SEQ ID NO:1). The resulting CL leader peptides are thus to be considered as functional equivalents. Other functional equivalents of the CL leader peptides include those leader peptides reproducing the same technical aspects as the CL leader peptides as described throughout the current invention, including deletion variants and insertion variants. Examples of avian lysozyme leader peptides are given in SEQ ID NO: 1 and SEQ ID NO: 99 (see also Example section).

With “A” or “adaptor peptide” is meant a peptide (e.g. 1 to 30 amino acids) or a protein which may serve as a linker between e.g. a leader peptide and a processing site (PS), a leader peptide and a protein of interest, a PS and a protein of interest, and/or a protein of interest and a PS; and/or may serve as a linker N- or C-terminal of e.g. a leader peptide, a PS or a protein of interest. The adaptor peptide “A” may have a certain three-dimensional structure, e.g. an α-helical or β-sheet structure or a combination thereof. Alternatively the three-dimensional structure of A is not well defined, e.g. a coiled-coil structure. The adaptor A may be part of e.g. a pre-sequence, a pro-sequence, a protein of interest sequence or a processing site. The adaptor A may serve as a tag enhancing or enabling detection and/or purification and/or processing of the protein of which A is a part. One examples of an A peptide is the his-tag peptide (HHHHHH; SEQ ID NO:63) Hₙ wherein n usually is six, but may be 7, 8, 9, 10, 11, or 12. Other examples of A-peptides include the peptides EEGEPK (Kjeldsen et al. in WO98/28429; SEQ ID NO:64) or EEAEPK (Kjeldsen et al. in WO97/22706; SEQ ID NO:65) which, when present at the N-terminal of the a protein of interest, were reported to increase fermentation yield but also to protect the N-terminus of the protein of interest against processing by dipeptidyl aminopeptidase and thus resulting in a homogenous N-terminus of the polypeptide. At the same time, in vitro maturation of the protein of interest, i.e. removal of said peptides EEGEPK (SEQ ID NO:64) and EEAEPK (SEQ ID NO:65) from the protein of interest can be achieved by using e.g. endo-lys C which cleaves C-terminal of the Lys-residue in said peptides. Said peptides thus serve the function of adaptor peptide (A) as well as processing site (PS), (see below). Adaptor peptides are given in SEQ ID NOs:63-65, 70-72 and 74-82. Another example of an adaptor peptide is the G4S immunosilent linker. Other examples of adaptor peptides or adaptor proteins are listed in Table 2 of Stevens (Stevens et al. 2000).

With “PS” or “processing site” is meant a specific protein processing or processable site. Said processing may occur enzymatically or chemically. Examples of processing sites prone to specific enzymatic processing include IEGR↓X (SEQ ID NO:66), IDGR↓X (SEQ ID NO:67), AEGR↓X (SEQ ID NO:68), all recognized by and cleaved between the Arg and Xaa (any amino acid) residues as indicated by the “↓” by the bovine factor Xa protease (Nagai, K. and Thogersen, H. C. 1984). Another example of a PS site is a dibasic site, e.g. Arg-Arg, Lys-Lys, Arg-Lys or Lys-Arg, which is cleavable by the yeast Kex2 protease (Julius, D. et al. 1984). The PS site may also be a monobasic Lys-site. Said monobasic Lys-PS-site may also be included at the C-terminus of an A peptide. Examples of A adaptor peptides comprising a C-terminal monobasic Lys-PS-site are given by SEQ ID NOs:64-65 and 74-76. Exoproteolytic removal of a His-tag (HHHHHH; SEQ ID NO:63) is possible by using the dipeptidyl aminopeptidase I (DAPase) alone or in combination with glutamine cyclotransferase (Qcyclase) and pyroglutamic aminopeptidase (pGAPase) (Pedersen, J. et al. 1999). Said exopeptidases comprising a recombinant His-tag (allowing removal of the peptidase from the reaction mixture by immobilize metal-affinity chromatography, IMAC) are commercially available, e.g. as the TAGZyme System of Unizyme Laboratories (Hørsholm, DK). With “processing” is thus generally meant any method or procedure whereby a protein is specifically cleaved or cleavable at least one processing site when said processing site is present in said protein. A PS may be prone to endoproteolytic cleavage or may be prone to exproteolytic cleavage, in any case the cleavage is specific, i.e. does not extend to sites other than the sites recognized by the processing proteolytic enzyme. A number of PS sites are given in SEQ ID NOs:66-68 and 83-84.

The versatility of the [(A1/3)_{a/d}-(PS1/2)_{b/e}-(A2/4)_{c/f}] structure as outlined above is demonstrated by means of some examples. In a first example, said structure is present at the C-terminal end of a protein of interest comprised in a pre-protein and wherein A3 is the “VIEGR” peptide (SEQ ID NO:69) which is overlapping with the factor Xa “IEGRX” PS site (SEQ ID NO:66) and wherein X=A4 is the histidine-tag (SEQ ID NO:63) (d, e and f thus are all 1 in this case). The HCV protein of interest can (optionally) be purified by IMAC. After processing with factor Xa, the (optionally purified) HCV protein of interest will carry at its C-terminus a processed PS site which is “IEGR” (SEQ ID NO:70). Variant processed factor Xa processing site, can be IDGR (SEQ ID NO:71) or AEGR (SEQ ID NO:72). In a further example, the [(A1/3)_{a/d}-(PS1/2)_{b/e}-(A2/4)_{c/f}] structure is present at the N-terminus of the HCV protein of interest. Furthermore, A1 is the histidine-tag (SEQ ID NO:63), PS is the factor Xa recognition site (any of SEQ ID NOs:66-68) wherein X is the protein of interest, and wherein a=b=1 and c=0. Upon correct removal of a leader peptide, e.g. by the host cell, the resulting HCV protein of interest can be purified by IMAC (optional). After processing with factor Xa, the protein of interest will be devoid of the [(A1)ₐ-(PS1)_{b}-(A2)_{c}] structure.

It will furthermore be clear that any of A1, A2, A3, A4, PS1 and PS2, when present, may be present in a repeat structure. Such a repeat structure, when present, is in this context still counted as 1, i.e. a, b, c, d, e, or f are 1 even if e.g. A1 is occurring as e.g. 2 repeats (A1-A1).

With “HCV envelope protein” is meant a HCV E1 or HCV E2 envelope protein or a part thereof whereby said proteins may be derived from a HCV strain of any genotype. More specifically, HCVENV is chosen from the group of amino acid sequences consisting of SEQ ID NOs:85 to 98, amino acid sequences which are at least 90% identical to SEQ ID NOs:85 to 98, and fragments of any thereof. As “identical” amino acids are considered the groups of conserved amino acids as described above, i.e. the group consisting of Met, Ile, Leu and Val; the group consisting of Arg, Lys and H is; the group consisting of Phe, Trp and Tyr; the group consisting of Asp and Glu; the group consisting of Asn and Gln; the group consisting of Cys, Ser and Thr; and the group consisting of Ala and Gly.

More specifically, the term “HCV envelope proteins” relates to a polypeptide or an analogue thereof (e.g. mimotopes) comprising an amino acid sequence (and/or amino acid analogues) defining at least one HCV epitope of either the E1 or the E2 region, in addition to a glycosylation site. These envelope proteins may be both monomeric, hetero-oligomeric or homo-oligomeric forms of recombinantly expressed envelope proteins. Typically, the sequences defining the epitope correspond to the amino acid sequences of either the E1 or the E2 region of HCV (either identically or via substitutions of analogues of the native amino acid residue that do not destroy the epitope).

It will be understood that the HCV epitope may co-locate with the glycosylation site. In general, the epitope-defining sequence will be 3 or 4 amino acids in length, more typically, 5, 6, or 7 amino acids in length, more typically 8 or 9 amino acids in length, and even more typically 10 or more amino acids in length. With respect to conformational epitopes, the length of the epitope-defining sequence can be subject to wide variations, since it is believed that these epitopes are formed by the three-dimensional shape of the antigen (e.g. folding). Thus, the amino acids defining the epitope can be relatively few in number, but widely dispersed along the length of the molecule being brought into the correct epitope conformation via folding. The portions of the antigen between the residues defining the epitope may not be critical to the conformational structure of the epitope. For example, deletion or substitution of these intervening sequences may not affect the conformational epitope provided sequences critical to epitope conformation are maintained (e.g. cysteines involved in disulfide bonding, glycosylation sites, etc.). A conformational epitope may also be formed by 2 or more essential regions of subunits of a homo-oligomer or hetero-oligomer.

As used herein, an epitope of a designated polypeptide denotes epitopes with the same amino acid sequence as the epitope in the designated polypeptide, and immunologic equivalents thereof. Such equivalents also include strain, subtype (=genotype), or type(group)-specific variants, e.g. of the currently known sequences or strains belonging to genotypes 1a, 1b, 1c, 1d, 1e, 1f, 2a, 2b, 2c, 2d, 2e, 2f, 2g, 2h, 2i, 3a, 3b, 3c, 3d, 3e, 3f, 3g, 4a, 4b, 4c, 4d, 4e, 4f, 4g, 4h, 4i, 4j, 4k, 4i, 5a, 5b, 6a, 6b, 6c, 7a, 7b, 7c, 8a, 8b, 9a, 9b, 10a, 11 (and subtypes thereof), 12 (and subtypes thereof) or 13 (and subtypes thereof) or any other newly defined HCV (sub)type. It is to be understood that the amino acids constituting the epitope need not be part of a linear sequence, but may be interspersed by any number of amino acids, thus forming a conformational epitope.

The HCV antigens of the present invention comprise conformational epitopes from the E1 and/or E2 (envelope) domains of HCV. The E1 domain, which is believed to correspond to the viral envelope protein, is currently estimated to span amino acids 192-383 of the HCV polyprotein (Hijikata, M. et al. 1991). Upon expression in a mammalian system (glycosylated), it is believed to have an approximate molecular weight of 35 kDa as determined via SDS-PAGE. The E2 protein, previously called NS1, is believed to span amino acids 384-809 or 384-746 (Grakoui, A. et al. 1993) of the HCV polyprotein and also to be an envelope protein. Upon expression in a vaccinia system (glycosylated), it is believed to have an apparent gel molecular weight of about 72 kDa. It is understood that these protein endpoints are approximations (e.g. the carboxy terminal end of E2 could lie somewhere in the 730-820 amino acid region, e.g. ending at amino acid 730, 735, 740, 742, 744, 745, preferably 746, 747, 748, 750, 760, 770, 780, 790, 800, 809, 810, 820). The E2 protein may also be expressed together with E1, and/or core (aa 1-191), and/or P7 (aa 747-809), and/or NS2 (aa 810-1026), and/or NS3 (aa 1027-1657), and/or NS4A (aa 1658-1711) and/or NS4B (aa 1712-1972) and/or NS5A (aa 1973-2420), and/or NS5B (aa 2421-3011), and/or any part of any of these HCV proteins different from E2. Likewise, the E1 protein may also be expressed together with the E2, and/or core (aa 1-191), and/or P7 (aa 747-809), and/or NS2 (aa 810-1026), and/or NS3 (aa 1027-1657), and/or NS4A (aa 1658-1711) and/or NS4B (aa 1712-1972), and/or NS5A (aa 1973-2420), and/or NS5B (aa 2421-3011), and/or any part of any of these HCV proteins different from E1. Expression together with these other HCV proteins may be important for obtaining the correct protein folding.

The term “E1” as used herein also includes analogs and truncated forms that are immunologically cross-reactive with natural E1, and includes E1 proteins of genotypes 1, 2, 3, 4, 5, 6, 7, 8, 9, 10, 11, 12 or 13 or any other newly identified HCV type or subtype. The term ‘E2’ as used herein also includes analogs and truncated forms that are immunologically cross-reactive with natural E2, and includes E2 proteins of genotypes 1, 2, 3, 4, 5, 6, 7, 8, 9, 10, 11, 12 or 13 or any other newly identified HCV type or subtype. For example, insertions of multiple codons between codon 383 and 384, as well as deletions of amino acids 384-387 have been reported (Kato, N. et al. 1992). It is thus also understood that the isolates used in the examples section of the present invention were not intended to limit the scope of the invention and that any HCV isolate from type 1, 2, 3, 4, 5, 6, 7, 8, 9, 10, 11, 12 or 13 or any other new genotype of HCV is a suitable source of E1 and/or E2 sequence for the practice of the present invention. Similarly, as described above, the HCV proteins that are co-expressed with the HCV envelope proteins of the present invention, can be derived from any HCV type, thus also from the same type as the HCV envelope proteins of the present invention.

“E1/E2” as used herein refers to an oligomeric form of envelope proteins containing at least one E1 component and at least one E2 component.

The term “specific oligomeric” E1 and/or E2 and/or E1/E2 envelope proteins refers to all possible oligomeric forms of recombinantly expressed E1 and/or E2 envelope proteins which are not aggregates. E1 and/or E2 specific oligomeric envelope proteins are also referred to as homo-oligomeric E1 or E2 envelope proteins (see below). The term ‘single or specific oligomeric’ E1 and/or E2 and/or E1/E2 envelope proteins refers to single monomeric E1 or E2 proteins (single in the strict sense of the word) as well as specific oligomeric E1 and/or E2 and/or E1/E2 recombinantly expressed proteins. These single or specific oligomeric envelope proteins according to the present invention can be further defined by the following formula (E1)ₓ(E2)_{y} wherein x can be a number between 0 and 100, and y can be a number between 0 and 100, provided that x and y are not both 0. With x=1 and y=0 said envelope proteins include monomeric E1.

The term “homo-oligomer” as used herein refers to a complex of E1 or E2 containing more than one E1 or E2 monomer, e.g. E1/E1 dimers, E1/E1/E1 trimers or E1/E1/E1/E1 tetramers and E2/E2 dimers, E2/E2/E2 trimers or E2/E2/E2/E2 tetramers, E1 pentamers and hexamers, E2 pentamers and hexamers or any higher-order homo-oligomers of E1 or E2 are all ‘homo-oligomers’ within the scope of this definition. The oligomers may contain one, two, or several different monomers of E1 or E2 obtained from different types or subtypes of hepatitis C virus including for example those described by Maertens et al. in WO 94/25601 and WO 96/13590 both by the present applicants. Such mixed oligomers are still homo-oligomers within the scope of this invention, and may allow more universal diagnosis, prophylaxis or treatment of HCV.

The E1 and E2 antigens used in the present invention may be full-length viral proteins, substantially full-length versions thereof, or functional fragments thereof (e.g. fragments comprising at least one epitope and/or glycosylation site). Furthermore, the HCV antigens of the present invention can also include other sequences that do not block or prevent the formation of the conformational epitope of interest. The presence or absence of a conformational epitope can be readily determined through screening the antigen of interest with an antibody (polyclonal serum or monoclonal to the conformational epitope) and comparing its reactivity to that of a denatured version of the antigen which retains only linear epitopes (if any). In such screening using polyclonal antibodies, it may be advantageous to adsorb the polyclonal serum first with the denatured antigen and see if it retains antibodies to the antigen of interest.

The HCV proteins of the present invention may be glycosylated. Glycosylated proteins intend proteins that contain one or more carbohydrate groups, in particular sugar groups. In general, all eukaryotic cells are able to glycosylate proteins. After alignment of the different envelope protein sequences of HCV genotypes, it may be inferred that not all 6 glycosylation sites on the HCV E1 protein are required for proper folding and reactivity. For instance, HCV subtype 1b E1 protein contains 6 glycosylation sites, but some of these glycosylation sites are absent in certain other (sub)types. The fourth carbohydrate motif (on Asn250), present in types 1b, 6a, 7, 8, and 9, is absent in all other types know today. This sugar-addition motif may be mutated to yield a type 1b E1 protein with improved reactivity. Also, the type 2b sequences show an extra glycosylation site in the V5 region (on Asn299). The isolate S83, belonging to genotype 2c, even lacks the first carbohydrate motif in the VI region (on Asn), while it is present on all other isolates (Stuyver, L. et al. 1994). However, even among the completely conserved sugar-addition motifs, the presence of the carbohydrate may not be required for folding, but may have a role in evasion of immune surveillance. Thus, the identification of the role of glycosylation can be further tested by mutagenesis of the glycosylation motifs. Mutagenesis of a glycosylation motif (NXS or NXT sequences) can be achieved by either mutating the codons for N, S, or T, in such a way that these codons encode amino acids different from N in the case of N, and/or amino acids different from S or T in the case of S and in the case of T. Alternatively, the X position may be mutated into P, since it is known that NPS or NPT are not frequently modified with carbohydrates. After establishing which carbohydrate-addition motifs are required for folding and/or reactivity and which are not, combinations of such mutations may be made. Such experiments have been described extensively by Maertens et al. in WO 96/04385 (Example 8), which is included herein specifically by reference. The term glycosylation as used in the present invention refers to N-glycosylation unless otherwise specified.

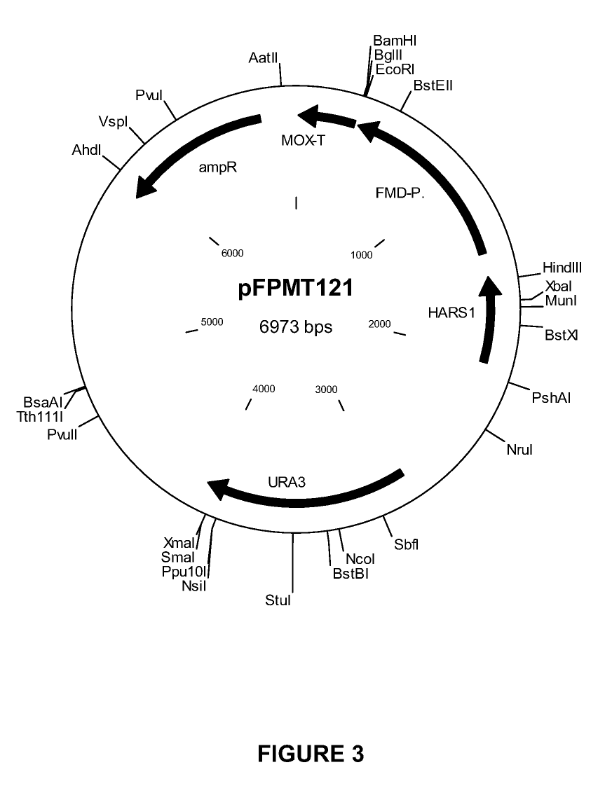

In particular, the present invention relates to HCV envelope proteins, or parts thereof that are core-glycosylated. In this respect, the term “core-glycosylation” refers to a structure “similar” to the structure as depicted in the boxed structure in FIG. 3 of Herscovics and Orlean (Herscovics, A. and Orlean, P. 1993). Thus, the carbohydrate structure referred to contains 10 or 11 mono-saccharides. Notably, said disclosure is herein incorporated by reference. The term “similar” intends that not more than about 4 additional mono-saccharides have been added to the structure or that not more than about 3 mono-saccharides have been removed from the structure. Consequently, a carbohydrate structure consists most preferentially of 10 mono-saccharides, but minimally of 7, and more preferentially of 8 or 9 mono-sacchariden, and maximally of 15 mono-saccharides, and more preferentially of 14, 13, 12, or 11 mono-saccharides. The mono-saccharides connoted are preferentially glucose, mannose or N-acetyl glucosamine.

Another aspect of the present invention covers vectors comprising a polynucleic acid, or a part thereof, of the invention. Such vectors comprise universal cloning vectors such as the pUC-series or pEMBL-series vectors and furthermore include other cloning vectors such as cloning vectors requiring a DNA topoisomerase reaction for cloning, TA-cloning vectors and recombination-based cloning vectors such as those used in the Gateway system (InVitrogen). Vectors comprise plasmids, phagemids, cosmids, bacmids (baculovirus vectors) or may be viral or retroviral vectors. A vector can merely function as a cloning tool and/or -vehicle or may additionally comprise regulatory sequences such as promoters, enhancers and terminators or polyadenylation signals. Said regulatory sequences may enable expression of the information contained within the DNA fragment of interest cloned into a vector comprising said regulatory sequences. Expression may be the production of RNA molecules or mRNA molecules and, optionally, the production of protein molecules thereof. Expression may be the production of an RNA molecule by means of a viral polymerase promoter (e.g. SP6, T7 or T3 promoter) introduced to the 5′- or 3′-end of the DNA of interest. Expression may furthermore be transient expression or stable expression or, alternatively, controllable expression. Controllable expression comprises inducible expression, e.g. using a tetracyclin-regulatable promoter, a stress-inducible (e.g. human hsp70 gene promoter), a methallothionine promoter, a glucocorticoid promoter or a progesterone promoter. Expression vectors are known in the art that mediate expression in bacteria (e.g. Escherichia coli, Streptomyces species), insect cells (Spodoptera frugiperda cells, Sf9 cells), plant cells (e.g. potato virus X-based expression vectors, see e.g. Vance et al. 1998 in WO98/44097) and mammalian cells (e.g. CHO or COS cells, Vero cells, cells from the HeLa cell line).

This aspect of the invention thus specifically relates to a vector comprising the recombinant nucleic acids according to the invention encoding a protein comprising an avian lysozyme leader peptide or a functional equivalent thereof joined to an HCV envelope protein or a part thereof, or a protein characterized by the structure CL-[(A1)ₐ-(PS1)_{b}-(A2)_{c}]-HCVENV-[(A3)_{d}-(PS2)ₑ-(A4)_{f}].

Embodied in the present invention are also said vectors further comprising regulatory sequences allowing expression of said protein.

In a specific embodiment, said vector according to the invention is an expression vector.

In another specific embodiment, said vector according to the invention is an autonomously replicating vector or an integrative vector.

In yet another specific embodiment, said vector according to the invention is chosen from any of SEQ ID NOs: 20, 21, 32, 35, 36, 39, 40.

Suitable vectors or expression vectors of the invention are yeast vectors. A yeast vector may comprise a DNA sequence enabling the vector to replicate autonomously. Examples of such sequences are the yeast plasmid 2μ replication genes REP 1-3 and origin of replication. Other vectors are integrating partially or completely in the yeast genome. Such integrative vectors are either targeted to specific genomic loci or integrate randomly. In P. pastoris, foreign DNA is targeted to the AOX1 and the HIS4 genes (Cregg, J. M. 1999), in P. methanolica to the AUG1 gene (Raymond, C. K. 1999). In most recombinant H. polymorpha strains, foreign DNA can be randomly integrated using HARS-sequence-harboring circular plasmids for transformation (Hollenberg, C. P. and Gellissen, G. 1997c). Targeted integration can be achieved by homologous recombination using the MOX/TRP3 locus for disruption/integration (Agaphonov, M. O. et al. 1995, Sohn, J. H. et al. 1999), the LEU2 gene (Agaphonov, M. O. et al. 1999) or the rDNA cluster (Cox, H. et al. 2000). Transformations in H. polymorpha typically result in a variety of individual, mitotically stable strains containing single to multiple copies of the expression cassette in a head-to-tail arrangement. Strains with up to 100 copies have been identified (Hollenberg, C. P. and Gellissen, G. 1997b). Random multiple-copy integration can be forced in the uracil-auxotroph H. polymorpha strain RB11 by a sequence of passages under selective conditions if a H. polymorpha or S. cerevisiae-derived URA3 gene is present. A HARS sequence can be excluded (Gatzke, R. et al. 1995) or can be present (Hollenberg, C. P. and Gellissen, G. 1997a). This passaging furthermore leads to mitotically stable strains. The vector may also comprise a selectable marker, e.g. the Schizosaccharomyces pombe TPI gene as described by Russell (Russell, P. R. 1985), or the yeast URA3 gene. Other marker genes so far used for transformation of Saccharomyces, for example TRP5, LEU2, ADE1, ADE2, HIS3, HIS4, LYS2, may be obtained from e.g. Hansenula, Pichia or Schwanniomyces.

“Regulatory elements (or sequences) allowing expression of a protein in a eukaryotic host” are to be understood to comprise at least a genetic element displaying promoter activity and a genetic element displaying terminator activity whereby said regulatory elements are operably linked to the open reading frame encoding the protein to be expressed. The term “promoter” is a nucleotide sequence which is comprised of consensus sequences which allow the binding of RNA polymerase to the DNA template in a manner such that mRNA production initiates at the normal transcription initiation site for the adjacent structural gene.

The term “operably linked” refers to a juxtaposition wherein the components so described are in a relationship permitting them to function in their intended manner. A control sequence “operably linked” to a coding sequence is ligated in such a way that expression of the coding sequence is achieved under conditions compatible with the control sequences.

An “open reading frame” (ORF) is a region of a polynucleotide sequence which encodes a polypeptide and does not contain stop codons; this region may represent a portion of a coding sequence or a total coding sequence.

A “coding sequence” is a polynucleotide sequence which is transcribed into mRNA and/or translated into a polypeptide when placed under the control of appropriate regulatory sequences. The boundaries of the coding sequence are determined by a translation start codon at the 5′-terminus and a translation stop codon at the 3′-terminus. A coding sequence can include but is not limited to mRNA, DNA (including cDNA), and recombinant polynucleotide sequences.

Many regulatory elements are known in the art. Examples of suitable yeast promoters are the Saccharomyces cerevisiae MFα1, TPI, ADH I, ADH II or PGK promoters, or corresponding promoters from other yeast species, e.g. Schizosaccharomyces pombe. Examples of suitable promoters are described by, for instance, (Alber, T. and Kawasaki, G. 1982, Ammerer, G. 1983, Ballou, L. et al. 1991c, Hitzeman, R. A. et al. 1980, Kawasaki, G. and Fraenkel, D. G. 1982, Russell, D. W. et al. 1983, Russell, P. R. 1983, Russell, P. R. and Hall, B. D. 1983). A suitable yeast terminator is, e.g. the TPI terminator (Alber, T. and Kawasaki, G. 1982), or the yeast CYC1 terminator. For methylotrophic or facultative methylotrophic yeast species, the strong and regulatable promoters of the enzymes involved in the methanol utilization pathway are good candidate promoters and include the promoters of the alcohol oxidase genes (AOX1 of Pichia pastoris, AUG1 of P. methanolica, AOD1 of Candida boidinii, and MOX of Hansenula polymorpha), the formaldehyde dehydrogenase promoter (FLD1 of P. pastoris), the dihydroxyacetone synthase promoter (DAS1 of C. boidinii) and the formate dehydrogenase promoter (FMD of H. polymorpha). Other promoters include the GAP1 promoter of P. pastoris or H. polymorpha and the PMA1 and TPS1 promoter of H. polymorpha (Gellissen, G. 2000a), and references cited therein). The terminator element derived from any of these genes are examples of suitable terminator elements, more specifically suitable terminator elements include the AOD1, AOX1 and MOX terminator elements.

A further aspect of the current invention covers host cells comprising a recombinant nucleic acid or a vector according to the invention.

In a specific embodiment thereto, said host cells comprising a recombinant nucleic acid or a vector according to the invention are capable of expressing the protein according to the invention comprising the avian leader lysozyme leader peptide or a functional variant thereof joined to an HCV envelope protein or a part thereof.

In an alternative embodiment, said host cells are capable of expressing the protein characterized by the structure CL-[(A1)ₐ-(PS1)_{b}-(A2)_{c}]-HCVENV-[(A3)_{d}-(PS2)ₑ-(A4)_{f}]

wherein:
CL is an avian lysozyme leader peptide or a functional equivalent thereof,
A1, A2, A3 and A4 are adaptor peptides which can be different or the same,
PS1 and PS2 are processing sites which can be the different or the same,
HCVENV is a HCV envelope protein or a part thereof,
a, b, c, d, e and f are 0 or 1, and
wherein, optionally, A1 and/or A2 are part of PS1 and/or wherein A3 and/or A4 are part of PS2.

In a further specific embodiment thereto, said host cells comprising a recombinant nucleic acid or a vector according to the invention are capable of translocating the protein comprising the avian lysozyme leader peptide or a functional equivalent thereof joined to an HCV envelope protein or a part thereof to the endoplasmic reticulum upon removal of the avian lysozyme leader peptide.

In a further specific embodiment thereto, said host cells comprising a recombinant nucleic acid or a vector according to the invention are capable of translocating the protein [(A1)ₓ-(PS1)_{y}-(A2)_{z}]-HCVENV-[(A3)ₙ-(PS2)_{y}-(A4)_{z}] to the endoplasmic reticulum upon removal of the CL peptide wherein said protein and said CL peptide are derived from the protein characterized by the structure CL-[(A1)ₐ-(PS1)_{b}-(A2)_{c}]-HCVENV-[(A3)_{d}-(PS2)ₑ-(A4)_{f}]

wherein:
CL is an avian lysozyme leader peptide or a functional equivalent thereof,
A1, A2, A3 and A4 are adaptor peptides which can be different or the same,
PS1 and PS2 are processing sites which can be the different or the same,
HCVENV is a HCV envelope protein or a part thereof,
a, b, c, d, e and f are 0 or 1, and
wherein, optionally, A1 and/or A2 are part of PS1 and/or wherein A3 and/or A4 are part of PS2.

Also embodied are host cells comprising a recombinant nucleic acid or a vector according to the invention which are capable of processing the processing sites PS1 and/or PS2 in said protein translocated to the endoplasmic reticulum.

Also embodied are host cells comprising a recombinant nucleic acid or a vector according to the invention which are capable of N-glycosylating said protein translocated to the endoplasmic reticulum.

Also embodied are host cells comprising a recombinant nucleic acid or a vector according to the invention which are capable of N-glycosylating said protein translocated to the endoplasmic reticulum and processed at said sites PS1 and/or PS2.

More specifically, the host cells comprising a recombinant nucleic acid or a vector according to the invention are eukaryotic cells and, more particularly, yeast cells such as cells of strains of Saccharomyces, such as Saccharomyces cerevisiae, Saccharomyces kluyveri, or Saccharomyces uvarum, Schizosaccharomyces, such as Schizosaccharomyces pombe, Kluyveromyces, such as Kluyveromyces lactis, Yarrowia, such as Yarrowia lipolytica, Hansenula, such as Hansenula polymorpha, Pichia, such as Pichia pastoris, Aspergillus species, Neurospora, such as Neurospora crassa, or Schwanniomyces, such as Schwanniomyces occidentalis, or mutant cells derived from any thereof.

The term “eukaryotic cells” includes lower eukaryotic cells as well as higher eukaryotic cells. Lower eukaryotic cells are cells such as yeast cells, fungal cells and the like. Particularly suited host cells in the context of the present invention are yeast cells or mutant cells derived from any thereof as described above. Mutant cells include yeast glycosylation minus strains, such as Saccharomyces glycosylation minus strains as used in the present invention. Glycosylation minus strains are defined as strains carrying a mutation, in which the nature of the mutation is not necessarily known, but resulting in a glycosylation of glycoproteins comparable to the core-glycosylation In particular, it is contemplated that Saccharomyces glycosylation minus strains carry a mutation resulting in a significant shift in mobility on PAGE of the invertase protein. Invertase is a protein which is normally present in Saccharomyces in a hyperglycosylated form only (Ballou, L. et al. 1991b). Glycosylation minus strains include mnn2, and/or och1 and/or mnn9 deficient strains. The mutant host cells of the invention do not include cells which, due to the mutation, have lost their capability to remove the avian lysozyme leader peptide from a protein comprising said leader peptide joined to a protein of interest.

Higher eukaryotic cells include host cells derived from higher animals, such as mammals, reptiles, insects, and the like. Presently preferred higher eukaryote host cells are derived from Chinese hamster (e.g. CHO), monkey (e.g. COS and Vero cells), baby hamster kidney (BHK), pig kidney (PK15), rabbit kidney 13 cells (RK13), the human osteosarcoma cell line 143 B, the human cell line HeLa and human hepatoma cell lines like Hep G2, and insect cell lines (e.g. Spodoptera frugiperda). The host cells may be provided in suspension or flask cultures, tissue cultures, organ cultures and the like. Alternatively the host cells may also be transgenic animals or transgenic plants.

Introduction of a vector, or an expression vector, into a host cell may be effectuated by any available transformation or transfection technique applicable to said host cell as known in the art. Such transformation or transfection techniques comprise heat-shock mediated transformation (e.g. of E. coli), conjugative DNA transfer, electroporation, PEG-mediated DNA uptake, liposome-mediated DNA uptake, lipofection, calcium-phosphate DNA coprecipitation, DEAE-dextran mediated transfection, direct introduction by e.g. microinjection or particle bombardment, or introduction by means of a virus, virion or viral particle.

Yet another aspect of the invention relates to methods for producing a HCV envelope protein or part thereof in a host cell, said method comprising transforming said host cell with the recombinant nucleic acid according to the invention or with the vector according to the invention, and wherein said host cell is capable of expressing a protein comprising the avian lysozyme leader peptide or a functional equivalent thereof joined to a HCV envelope protein or a part thereof.

In a specific embodiment thereto, said method for producing a HCV envelope protein or part thereof in a host cell is comprising the step of transforming said host cell with the recombinant nucleic acid according to the invention or with the vector according to the invention, and wherein said host cell is capable of expressing the protein characterized by the structure CL-[(A1)ₐ-(PS1)_{b}-(A2)_{c}]-HCVENV-[(A3)_{d}-(PS2)ₑ-(A4)_{f}]

wherein:
CL is an avian lysozyme leader peptide or a functional equivalent thereof,
A1, A2, A3 and A4 are adaptor peptides which can be different or the same,
PS1 and PS2 are processing sites which can be the different or the same,
HCVENV is a HCV envelope protein or a part thereof,
a, b, c, d, e and f are 0 or 1, and
wherein, optionally, A1 and/or A2 are part of PS1 and/or wherein A3 and/or A4 are part of PS2.

In another specific embodiment thereto, the host cell in said method is capable of translocating the protein CL-[(A1)ₐ-(PS1)_{b}-(A2)_{c}]-HCVENV-[(A3)_{d}-(PS2)ₑ-(A4)_{f}] to the endoplasmic reticulum upon removal of the CL peptide wherein said protein and said CL peptide are derived from the protein characterized by the structure CL-[(A1)ₐ-(PS1)_{b}-(A2)_{c}]-HCVENV-[(A3)_{d}-(PS2)ₑ-(A4)_{f}]

wherein:
CL is an avian lysozyme leader peptide or a functional equivalent thereof,
A1, A2, A3 and A4 are adaptor peptides which can be different or the same,
PS1 and PS2 are processing sites which can be the different or the same,
HCVENV is a HCV envelope protein or a part thereof,
a, b, c, d, e and f are 0 or 1, and
wherein, optionally, A1 and/or A2 are part of PS1 and/or wherein A3 and/or A4 are part of PS2.

Also embodied is the method for producing a HCV envelope protein or part thereof wherein said host cell is capable of N-glycosylating said protein translocated to the endoplasmic reticulum.

Further embodied is the method for producing a HCV envelope protein or part thereof wherein said host cell is capable of N-glycosylating said protein translocated to the endoplasmic reticulum and processed at said sites PS1 and/or PS2.

More specifically, the host cell in any of said methods for producing a HCV envelope protein or part thereof is an eukaryotic cell and, more particularly, a yeast cell such as a cell of strains of Saccharomyces, such as Saccharomyces cerevisiae, Saccharomyces kluyveri, or Saccharomyces uvarum, Schizosaccharomyces, such as Schizosaccharomyces pombe, Kluyveromyces, such as Kluyveromyces lactis, Yarrowia, such as Yarrowia lipolytica, Hansenula, such as Hansenula polymorpha, Pichia, such as Pichia pastoris, Aspergillus species, Neurospora, such as Neurospora crassa, or Schwanniomyces, such as Schwanniomyces occidentalis, or mutant cells derived from any thereof.

Any of the methods according to the invention for producing a HCV envelope protein or part thereof may further comprise cultivation of the host cells comprising a recombinant nucleic acid or a vector according to the invention in a suitable medium to obtain expression of said protein.

A further embodiment thereto comprises isolation of the produced HCV envelope protein or part thereof from a culture of said host cells, or, alternatively, from said host cells. Said isolation step may include one or more of (i) lysis of said host cells in the presence of chaotropic agent, (ii) chemical and/or enzymatic modification of the cysteine thiol-groups in the isolated proteins wherein said modification may be reversible or irreversible, and producing a HCV envelope protein or part thereof (iii) heparin affinity chromatography.

Exemplary “chaotropic agents” are guanidinium chloride and urea. In general, a chaotropic agent is a chemical that can disrupt the hydrogen bonding structure of water. In concentrated solutions they can denature proteins because they reduce the hydrophobic effect

In the HCV envelope proteins or parts thereof as described herein comprising at least one cysteine residue, but preferably 2 or more cysteine residues, the cysteine thiol-groups can be irreversibly protected by chemical or enzymatic means. In particular, “irreversible protection” or “irreversible blocking” by chemical means refers to alkylation, preferably alkylation of the HCV envelope proteins by means of alkylating agents, such as, for example, active halogens, ethylenimine or N-(iodoethyl)trifluoro-acetamide. In this respect, it is to be understood that alkylation of cysteine thiol-groups refers to the replacement of the thiol-hydrogen by (CH₂)ₙR, in which n is 0, 1, 2, 3 or 4 and R═H, COOH, NH₂, CONH₂, phenyl, or any derivative thereof. Alkylation can be performed by any method known in the art, such as, for example, active halogens X(CH₂)ₙR in which X is a halogen such as I, Br, Cl or F. Examples of active halogens are methyliodide, iodoacetic acid, iodoacetamide, and 2-bromoethylamine. Other methods of alkylation include the use of NEM (N-ethylmaleimide) or Biotin-NEM, a mixture thereof, or ethylenimine or N-(iodoethyl)trifluoroacetamide both resulting in substitution of —H by —CH₂—CH₂—NH₂ (Hermanson, G. T. 1996). The term “alkylating agents” as used herein refers to compounds which are able to perform alkylation as described herein. Such alkylations finally result in a modified cysteine, which can mimic other aminoacids. Alkylation by an ethylenimine results in a structure resembling lysine, in such a way that new cleavage sites for trypsine are introduced (Hermanson, G. T. 1996). Similarly, the usage of methyliodide results in an amino acid resembling methionine, while the usage of iodoacetate and iodoacetamide results in amino acids resembling glutamic acid and glutamine, respectively. In analogy, these amino acids are preferably used in direct mutation of cysteine. Therefore, the present invention pertains to HCV envelope proteins as described herein, wherein at least one cysteine residue of the HCV envelope protein as described herein is mutated to a natural amino acid, preferentially to methionine, glutamic acid, glutamine or lysine. The term “mutated” refers to site-directed mutagenesis of nucleic acids encoding these amino acids, ie to the well known methods in the art, such as, for example, site-directed mutagenesis by means of PCR or via oligonucleotide-mediated mutagenesis as described in (Sambrook, J. et al. 1989). It should be understood that for the Examples section of the present invention, alkylation refers to the use of iodo-acetamide as an alkylating agent unless otherwise specified.

It is further understood that in the purification procedure, the cysteine thiol-groups of the HCV proteins or the parts thereof of the present invention can be reversibly protected. The purpose of reversible protection is to stabilize the HCV protein or part thereof. Especially, after reversible protection the sulfur-containing functional group (eg thiols and disulfides) is retained in a non-reactive condition. The sulfur-containing functional group is thus unable to react with other compounds, e.g. have lost their tendency of forming or exchanging disulfide bonds, such as, for example

R₁—SH+R₂—SH - - - X - - - >R₁—S—S—R₂;

R₁—S—S—R₂+R₃—SH - - - X - - - >R₁—S—S—R₃+R₂—SH;

R₁—S—S—R₂+R₃—S—S—R₄- - - X - - - >R₁—S—S—R₃+R₂—S—S—R₄.

The described reactions between thiols and/or disulphide residues are not limited to intermolecular processes, but may also occur intramolecularly.

The term “reversible protection” or “reversible blocking” as used herein contemplates covalently binding of modification agents to the cysteine thiol-groups, as well as manipulating the environment of the HCV protein such, that the redox state of the cysteine thiol-groups remains unaffected throughout subsequent steps of the purification procedure (shielding). Reversible protection of the cysteine thiol-groups can be carried out chemically or enzymatically.

The term “reversible protection by enzymatical means” as used herein contemplates reversible protection mediated by enzymes, such as for example acyl-transferases, e.g. acyl-transferases that are involved in catalysing thio-esterification, such as palmitoyl acyltransferase (see below).

The term “reversible protection by chemical means” as used herein contemplates reversible protection:
1. by modification agents that reversibly modify cysteinyls such as for example by sulphonation and thio-esterification;
   Sulphonation is a reaction where thiol or cysteines involved in disulfide bridges are modified to S-sulfonate: RSH→RS—SO₃⁻ (Darbre, A. 1986) or RS—SR→2 RS—SO₃⁻ (sulfitolysis; (Kumar, N. et al. 1986)). Reagents for sulfonation are e.g. Na₂SO₃, or sodium tetrathionate. The latter reagents for sulfonation are used in a concentration of 10-200 mM, and more preferentially in a concentration of 50-200 mM. Optionally sulfonation can be performed in the presence of a catalysator such as, for example Cu²⁺ (100 μM-1 mM) or cysteine (1-10 mM).
   The reaction can be performed under protein denaturing as well as native conditions (Kumar, N. et al. 1985, Kumar, N. et al. 1986).
   Thioester bond formation, or thio-esterification is characterised by:

RSH+R′COX→RS—COR′
in which X is preferentially a halogenide in the compound R′CO—X.
2. by modification agents that reversibly modify the cysteinyls of the present invention such as, for example, by heavy metals, in particular Zn²⁺, Cd²⁺, mono-, dithio- and disulfide-compounds (e.g. aryl- and alkylmethanethiosulfonate, dithiopyridine, dithiomorpholine, dihydrolipoamide, Ellmann reagent, Aldrothiol™ (Aldrich) (Rein, A. et al. 1996), dithiocarbamates), or thiolation agents (e.g. gluthathion, N-Acetyl cysteine, cysteineamine). Dithiocarbamate comprise a broad class of molecules possessing an R₁R₂NC(S)SR₃ functional group, which gives them the ability to react with sulphydryl groups. Thiol containing compounds are preferentially used in a concentration of 0.1-50 mM, more preferentially in a concentration of 1-50 mM, and even more preferentially in a concentration of 10-50 mM;
3. by the presence of modification agents that preserve the thiol status (stabilise), in particular antioxidantia, such as for example DTT, dihydroascorbate, vitamins and derivates, mannitol, amino acids, peptides and derivates (e.g. histidine, ergothioneine, carnosine, methionine), gallates, hydroxyanisole, hydroxytoluene, hydroquinon, hydroxymethylphenol and their derivates in concentration range of 10 μM-10 mM, more preferentially in a concentration of 1-10 mM;
4. by thiol stabilising conditions such as, for example, (i) cofactors as metal ions (Zn Mg²⁺), ATP, (ii) pH control (e.g. for proteins in most cases pH ˜5 or pH is preferentially thiol pKₐ-2; e.g. for peptides purified by Reversed Phase Chromatography at pH ˜2).

Combinations of reversible protection as described in (1), (2), (3) and (4) may result in similarly pure and refolded HCV proteins. In effect, combination compounds can be used, such as, for example Z103 (Zn carnosine), preferentially in a concentration of 1-10 mM. It should be clear that reversible protection also refers to, besides the modification groups or shielding described above, any cysteinyl protection method which may be reversed enzymatically or chemically, without disrupting the peptide backbone. In this respect, the present invention specifically refers to peptides prepared by classical chemical synthesis (see above), in which, for example, thioester bounds are cleaved by thioesterase, basic buffer conditions (Beekman, N. J. et al. 1997) or by hydroxylamine treatment (Vingerhoeds, M. H. et al. 1996).

Thiol containing HCV proteins can be purified, for example, on affinity chromatography resins which contain (1) a cleavable connector arm containing a disulfide bond (e.g. immobilised 5,5′ dithiobis(2-nitrobenzoic acid) (Jayabaskaran, C. et al. 1987) and covalent chromatography on activated thiol-Sepharose 4B (Pharmacia)) or (2) a aminohexanoyl-4-aminophenylarsine as immobilised ligand. The latter affinity matrix has been used for the purification of proteins, which are subject to redox regulation and dithiol proteins that are targets for oxidative stress (Kalef, E. et al. 1993).

Reversible protection may also be used to increase the solubilisation and extraction of peptides (Pomroy, N. C. and Deber, C. M. 1998).

The reversible protection and thiol stabilizing compounds may be presented under a monomeric, polymeric or liposomic form.

The removal of the reversibly protection state of the cysteine residues can chemically or enzymatically accomplished by e.g.:
a reductant, in particular DTT, DTE, 2-mercaptoethanol, dithionite, SnCl₂, sodium borohydride, hydroxylamine, TCEP, in particular in a concentration of 1-200 mM, more preferentially in a concentration of 50-200 mM;
removal of the thiol stabilising conditions or agents by e.g. pH increase;
enzymes, in particular thioesterases, glutaredoxine, thioredoxine, in particular in a concentration of 0.01-5 μM, even more particular in a concentration range of 0.1-5 μM.;
combinations of the above described chemical and/or enzymatical conditions.

The removal of the reversibly protection state of the cysteine residues can be carried out in vitro or in vivo, e.g. in a cell or in an individual.

It will be appreciated that in the purification procedure, the cysteine residues may or may not be irreversibly blocked, or replaced by any reversible modification agent, as listed above.

A reductant according to the present invention is any agent which achieves reduction of the sulfur in cysteine residues, e.g. “S—S” disulfide bridges, desulphonation of the cysteine residue (RS—SO₃⁻→RSH). An antioxidant is any reagent which preserves the thiol status or minimises “S—S” formation and/or exchanges. Reduction of the “S—S” disulfide bridges is a chemical reaction whereby the disulfides are reduced to thiol (—SH). The disulfide bridge breaking agents and methods disclosed by Maertens et al. in WO 96/04385 are hereby incorporated by reference in the present description. “S—S” Reduction can be obtained by (1) enzymatic cascade pathways or by (2) reducing compounds. Enzymes like thioredoxin, glutaredoxin are known to be involved in the in vivo reduction of disulfides and have also been shown to be effective in reducing “S—S” bridges in vitro. Disulfide bonds are rapidly cleaved by reduced thioredoxin at pH 7.0, with an apparent second order rate that is around 10⁴ times larger than the corresponding rate constant for the reaction with DTT. The reduction kinetic can be dramatically increased by preincubation the protein solution with 1 mM DTT or dihydrolipoamide (Holmgren, A. 1979). Thiol compounds able to reduce protein disulfide bridges are for instance Dithiothreitol (DTT), Dithioerythritol (DTE), β-mercaptoethanol, thiocarbamates, bis(2-mercaptoethyl) sulfone and N,N′-bis(mercaptoacetyl)hydrazine, and sodium-dithionite. Reducing agents without thiol groups like ascorbate or stannous chloride (SnCl₂), which have been shown to be very useful in the reduction of disulfide bridges in monoclonal antibodies (Thakur, M. L. et al. 1991), may also be used for the reduction of HCV proteins. In addition, changes in pH values may influence the redox status of HCV proteins. Sodium borohydride treatment has been shown to be effective for the reduction of disulfide bridges in peptides (Gailit, J. 1993). Tris (2-carboxyethyl)phosphine (TCEP) is able to reduce disulfides at low pH (Burns, J. et al. 1991). Selenol catalyses the reduction of disulfide to thiols when DTT or sodium borohydride is used as reductant. Selenocysteamine, a commercially available diselenide, was used as precursor of the catalyst (Singh, R. and Kats, L. 1995).

Heparin is known to bind to several viruses and consequently binding to the HCV envelope has already been suggested (Garson, J. A. et al. 1999a). In this respect, in order to analyze potential binding of HCV envelope proteins to heparin, heparin can be biotinylated and subsequently the interaction of heparin with HCV envelope proteins can be analyzed, e.g. on microtiterplates coated with HCV envelope proteins. In this way different expression systems can be scrutinized. For example, a strong binding is observed with part of the HCV E1 expressed in Hansenula, while binding with HCV E1 from mammalian cell culture is absent. In this respect, the term “heparin affinity chromatography” relates to an immobilized heparin, which is able to specifically bind to HCV envelope proteins. Proteins of the high-mannose type bind agglutinins such as Lens culinaris, Galanthus nivalis, Narcissus pseudonarcissus Pisum sativum or Allium ursinum. Moreover, N-acetylglucosamine can be bound by lectins, such as WGA (wheat germ agglutinin) and its equivalents. Therefore, one may employ lectins bound to a solid phase to separate the HCV envelope proteins of the present invention from cell culture supernatants, cell lysates and other fluids, e.g. for purification during the production of antigens for vaccine or immunoassay use.

With “HCV-recombinant vaccinia virus” is meant a vaccinia virus comprising a nucleic acid sequence encoding a HCV protein or part thereof.

A further aspect of the invention relates to an isolated HCV envelope protein or part thereof resulting from the method of production as described herein. In particular, the invention relates to an isolated HCV envelope protein or part thereof resulting from the expression in an eukaryotic cell of a recombinant nucleic acid comprising a nucleotide sequence encoding a protein comprising an avian lysozyme leader peptide or a functional equivalent thereof joined to said HCV envelope protein or a part thereof. More specifically, said recombinant nucleic acid is encoding a protein which is characterized by the structure CL-[(A1)ₐ-(PS1)_{b}-(A2)_{c}]-HCVENV-[(A3)_{d}-(PS2)ₑ-(A4)_{f}]

wherein:
CL is an avian lysozyme leader peptide or a functional equivalent thereof,
A1, A2, A3 and A4 are adaptor peptides which can be different or the same,
PS1 and PS2 are processing sites which can be the different or the same,
HCVENV is a HCV envelope protein or a part thereof,
a, b, c, d, e and f are 0 or 1, and
wherein, optionally, A1 and/or A2 are part of PS1 and/or wherein A3 and/or A4 are part of PS2.

In a specific embodiment, the isolated HCV envelope protein or part thereof is derived from said protein comprising an avian lysozyme leader peptide or a functional equivalent thereof joined to said HCV envelope protein or a part thereof. In another specific embodiment, the isolated HCV envelope protein or part thereof is derived from said protein which is characterized by the structure CL-[(A1)ₐ-(PS1)_{b}-(A2)_{c}]-HCVENV-[(A3)_{d}-(PS2)ₑ-(A4)_{f}]

wherein:
CL is an avian lysozyme leader peptide or a functional equivalent thereof,
A1, A2, A3 and A4 are adaptor peptides which can be different or the same,
PS1 and PS2 are processing sites which can be the different or the same,
HCVENV is a HCV envelope protein or a part thereof,
a, b, c, d, e and f are 0 or 1, and
wherein, optionally, A1 and/or A2 are part of PS1 and/or wherein A3 and/or A4 are part of PS2.

Another aspect of the current invention relates to the use of the avian lysozyme leader peptide to direct a recombinantly expressed protein to the endoplasmic reticulum of Hansenula polymorpha or any mutant thereof.

Thus, all aspects and embodiments of the current invention as described above and relating to a HCV envelope protein can, specific for H. polymorpha or any mutant thereof as host cell, be read as relating to a protein instead of relating to a HCV envelope protein.

More specifically, the current invention also relates to a recombinant nucleic acid comprising a nucleotide sequence encoding a protein comprising an avian lysozyme leader peptide or a functional equivalent thereof joined to a protein of interest or a part thereof.

In one embodiment thereto, the recombinant nucleic acid comprising nucleotide sequence encodes characterized by the structure

CL-[(A1)ₐ-(PS1)_{b}-(A2)_{c}]-PROT-[(A3)_{d}-(PS2)ₑ-(A4)_{f}]

wherein:
CL is an avian lysozyme leader peptide or a functional equivalent thereof,
A1, A2, A3 and A4 are adaptor peptides which can be different or the same,
PS1 and PS2 are processing sites which can be the different or the same,
PROT is a protein of interest or a part thereof,
a, b, c, d, e and f are 0 or 1, and
wherein, optionally, A1 and/or A2 are part of PS1 and/or wherein A3 and/or A4 are part of PS2.

In a further embodiment, the recombinant nucleic acids according to the invention further comprise regulatory elements allowing expression in a H. polymorpha cell or any mutant thereof of said protein comprising an avian lysozyme leader peptide or a functional equivalent thereof joined to a protein of interest or a part thereof, or of said protein characterized by the structure CL-[(A1)ₙ-(PS1)_{y}-(A2)_{z}]-PROT-[(A3)_{d}-(PS2)_{y}-(A4)_{f}]. Further included are vectors comprising said recombinant nucleic acids, host cells comprising said recombinant nucleic acids or said vectors, said host cells expressing the protein comprising an avian lysozyme leader peptide or a functional variant thereof joined to a protein of interest and methods for producing said protein of interest in said host cells.

A further aspect of the invention relates to an isolated protein of interest or part thereof resulting from the expression in a Hansenula cell of a recombinant nucleic acid comprising a nucleotide sequence encoding a protein comprising an avian lysozyme leader peptide or a functional equivalent thereof joined to said protein of interest or a part thereof. More specifically, said recombinant nucleic acid is encoding a protein which is characterized by the structure CL-[(A1)ₐ-(PS1)_{b}-(A2)_{c}]-PROT-[(A3)_{d}-(PS2)ₑ-(A4)_{f}]

wherein:
CL is an avian lysozyme leader peptide or a functional equivalent thereof,
A1, A2, A3 and A4 are adaptor peptides which can be different or the same,
PS1 and PS2 are processing sites which can be the different or the same,
PROT is a protein of interest or a part thereof,
a, b, c, d, e and f are 0 or 1, and
wherein, optionally, A1 and/or A2 are part of PS1 and/or wherein A3 and/or A4 are part of PS2.

In a specific embodiment, the isolated protein of interest or part thereof is derived from said protein comprising an avian lysozyme leader peptide or a functional equivalent thereof joined to said protein of interest or a part thereof. In another specific embodiment, the isolated protein of interest or part thereof is derived from said protein which is characterized by the structure CL-[(A1)ₐ-(PS1)_{b}-(A2)_{c}]-PROT-[(A3)_{d}-(PS2)ₑ-(A4)_{f}]

wherein:
CL is an avian lysozyme leader peptide or a functional equivalent thereof,
A1, A2, A3 and A4 are adaptor peptides which can be different or the same,
PS1 and PS2 are processing sites which can be the different or the same,
PROT is a protein of interest or a part thereof,
a, b, c, d, e and f are 0 or 1, and
wherein, optionally, A1 and/or A2 are part of PS1 and/or wherein A3 and/or A4 are part of PS2.

In a specific embodiment of the invention, said protein of interest or fragment thereof can e.g. be a viral envelope protein or a fragment thereof such as a HCV envelope protein or HBV (hepatitis B) envelope protein, or fragments thereto. In general, said protein of interest or fragment thereof can be any protein needing the N-glycosylation characteristics of the current invention. Other examplary viral envelope proteins include the HIV (human immunodeficiency virus) envelope protein gp120 and viral envelope proteins of a virus belonging to the Flavirideae.

Another aspect of the current invention relates to the use of a lysozyme leader peptide (L) to direct a recombinantly expressed protein to the endoplasmic reticulum of a methylotrophic yeast cell, such as Hansenula polymorpha or Pichia pastoris, or any mutant thereof.

Thus, all aspects and embodiments of the current invention as described above and relating to an avian lysozyme leader sequence and a HCV envelope protein can, specific for a methylotrophic yeast cell, such as Hansenula polymorpha or Pichia pastoris, or any mutant thereof as host cell, be read as relating to a lysozyme leader sequence instead of an avian lysozyme leader sequence and a protein instead of relating to a HCV envelope protein.

More specifically, the current invention relates to a recombinant nucleic acid comprising a nucleotide sequence encoding a protein comprising a lysozyme leader peptide or a functional equivalent thereof joined to a protein of interest or a part thereof, wherein said protein of interest is expressed in a methylotrophic yeast cell.

In one embodiment thereto, the recombinant nucleic acid comprising nucleotide sequence encodes characterized by the structure

L-[(A1)ₐ-(PS1)_{b}-(A2)_{c}]-PROT-[(A3)_{d}-(PS2)ₑ-(A4)_{f}]

wherein:
L is a lysozyme leader peptide or a functional equivalent thereof,
A1, A2, A3 and A4 are adaptor peptides which can be different or the same,
PS1 and PS2 are processing sites which can be the different or the same,
PROT is a protein of interest or a part thereof,
a, b, c, d, e and f are 0 or 1, and
wherein, optionally, A1 and/or A2 are part of PS1 and/or wherein A3 and/or A4 are part of PS2.

In a further embodiment, the recombinant nucleic acids according to the invention further comprise regulatory elements allowing expression in a methylotrophic yeast cell or any mutant thereof of said protein comprising a lysozyme leader peptide or a functional equivalent thereof joined to a protein of interest or a part thereof, or of said protein characterized by the structure L-[(A1)ₙ-(PS1)_{y}— (A2)_{z}]-PROT-[(A3)ₙ-(PS2)_{y}— (A4)_{z}]. Also included are vectors comprising said recombinant nucleic acids. Said vectors according to the invention can be chosen from any of SEQ ID NOs: 21, 32, 36, 40, 107 and 117. Further included are methylothropic yeast cells comprising said recombinant nucleic acids or said vectors, said methylothropic yeast cells expressing the protein comprising a lysozyme leader peptide or a functional variant thereof joined to a protein of interest and methods for producing said protein of interest in said methylothropic yeast cells.

A further aspect of the invention relates to an isolated protein of interest or part thereof resulting from the expression in a methylotrophic yeast cell of a recombinant nucleic acid comprising a nucleotide sequence encoding a protein comprising a lysozyme leader peptide or a functional equivalent thereof joined to said protein of interest or a part thereof. More specifically, said recombinant nucleic acid is encoding a protein which is characterized by the structure L-[(A1)ₐ-(PS1)_{b}-(A2)_{c}]-PROT-[(A3)_{d}-(PS2)ₑ-(A4)_{f}]

wherein:
L is a lysozyme leader peptide or a functional equivalent thereof,
A1, A2, A3 and A4 are adaptor peptides which can be different or the same,
PS1 and PS2 are processing sites which can be different or the same,
PROT is a protein of interest or a part thereof,
a, b, c, d, e and f are 0 or 1, and
wherein, optionally, A1 and/or A2 are part of PS1 and/or wherein A3 and/or A4 are part of PS2.

In a specific embodiment, the isolated protein of interest or part thereof is derived from said protein comprising a lysozyme leader peptide or a functional equivalent thereof joined to said protein of interest or a part thereof. In another specific embodiment, the isolated protein of interest or part thereof is derived from said protein which is characterized by the structure

L-[(A1)ₐ-(PS1)_{b}-(A2)_{c}]-PROT-[(A3)_{d}-(PS2)ₑ-(A4)_{f}]

wherein:
L is a lysozyme leader peptide or a functional equivalent thereof,
A1, A2, A3 and A4 are adaptor peptides which can be different or the same,
PS1 and PS2 are processing sites which can be the different or the same,
PROT is a protein of interest or a part thereof,
a, b, c, d, e and f are 0 or 1, and
wherein, optionally, A1 and/or A2 are part of PS1 and/or wherein A3 and/or A4 are part of PS2.

In a specific embodiment of the invention, said protein of interest or fragment thereof can e.g. be a viral envelope protein or a fragment thereof such as a HCV envelope protein or HBV (hepatitis B) envelope protein, or fragments thereto. In general, said protein of interest or fragment thereof can be any protein needing the N-glycosylation characteristics of the current invention. Other examplary viral envelope proteins include the HIV (human immunodeficiency virus) envelope protein gp120 and viral envelope proteins of a virus belonging to the Flavirideae. Said protein of interest or part thereof can also be a non-viral target protein, such as a target protein derived from a eukaryote or a prokaryote.

In another specific embodiment, said lysozyme leader sequences (or lysozyme pre-sequences) have amino acid sequences as defined in SEQ ID NO:1 or SEQ ID NO:99 (avian lysozyme leader sequences; CL) or SEQ ID NO:101 (human lysozyme leader sequence; HL) (see also Example section).

Methylotrophic yeast cells include the four known genera Hansenula, Pichia, Candida, and Torulopsis. They share a common metabolic metabolic pathway that enables them to use methanol as a sole carbon source. In a transcriptionally regulated response to methanol induction, several of the enzymes are rapidly synthesized at high levels. The hyperglycosylation as observed in Saccharomyces yeast cells is a very rare occurrence in methylotrophic yeast cells.

In another specific embodiment, the invention relates to a recombinant nucleic acid comprising a nucleotide sequence encoding a protein comprising a lysozyme leader peptide or a functional equivalent thereof joined to a protein of interest or a part thereof, wherein said protein of interest or part thereof is expressed in a methylotrophic yeast cell, and wherein said lysozyme leader sequence is not an avian lysozyme leader sequence and wherein said protein of interest or fragment thereof is not an HCV envelope protein or fragment thereof.

The terms “HCV virus-like particle formed of a HCV envelope protein” “oligomeric particles formed of HCV envelope proteins” are herein defined as structures of a specific nature and shape containing several basic units of the HCV E1 and/or E2 envelope proteins, which on their own are thought to consist of one or two E1 and/or E2 monomers, respectively. It should be clear that the particles of the present invention are defined to be devoid of infectious HCV RNA genomes. The particles of the present invention can be higher-order particles of spherical nature which can be empty, consisting of a shell of envelope proteins in which lipids, detergents, the HCV core protein, or adjuvant molecules can be incorporated. The latter particles can also be encapsulated by liposomes or apolipoproteins, such as, for example, apolipoprotein B or low density lipoproteins, or by any other means of targeting said particles to a specific organ or tissue. In this case, such empty spherical particles are often referred to as “virus-like particles” or VLPs. Alternatively, the higher-order particles can be solid spherical structures, in which the complete sphere consists of HCV E1 or E2 envelope protein oligomers, in which lipids, detergents, the HCV core protein, or adjuvant molecules can be additionally incorporated, or which in turn may be themselves encapsulated by liposomes or apolipoproteins, such as, for example, apolipoprotein B, low density lipoproteins, or by any other means of targeting said particles to a specific organ or tissue, e.g. asialoglycoproteins. The particles can also consist of smaller structures (compared to the empty or solid spherical structures indicated above) which are usually round (see further)-shaped and which usually do not contain more than a single layer of HCV envelope proteins. A typical example of such smaller particles are rosette-like structures which consist of a lower number of HCV envelope proteins, usually between 4 and 16. A specific example of the latter includes the smaller particles obtained with E1s in 0.2% CHAPS as exemplified herein which apparently contain 8-10 monomers of E1s. Such rosette-like structures are usually organized in a plane and are round-shaped, e.g. in the form of a wheel. Again lipids, detergents, the HCV core protein, or adjuvant molecules can be additionally incorporated, or the smaller particles may be encapsulated by liposomes or apolipoproteins, such as, for example, apolipoprotein B or low density lipoproteins, or by any other means of targeting said particles to a specific organ or tissue. Smaller particles may also form small spherical or globular structures consisting of a similar smaller number of HCV E1 or E2 envelope proteins in which lipids, detergents, the HCV core protein, or adjuvant molecules could be additionally incorporated, or which in turn may be encapsulated by liposomes or apolipoproteins, such as, for example, apolipoprotein B or low density lipoproteins, or by any other means of targeting said particles to a specific organ or tissue. The size (i.e. the diameter) of the above-defined particles, as measured by the well-known-in-the-art dynamic light scattering techniques (see further in examples section), is usually between 1 to 100 nm, more preferentially between 2 to 70 nm, even more preferentially between 2 and 40 nm, between 3 to 20 nm, between 5 to 16 nm, between 7 to 14 nm or between 8 to 12 nm.

In particular, the present invention relates to a method for purifying hepatitis C virus (HCV) envelope proteins, or any part thereof, suitable for use in an immunoassay or vaccine, which method comprising:
(i) growing Hansenula or Saccharomyces glycosylation minus strains transformed with an envelope gene encoding an HCV E1 and/or HCV E2 protein, or any part thereof, in a suitable culture medium;
(ii) causing expression of said HCV E1 and/or HCV E2 gene, or any part thereof; and
(iii) purifying said HCV E1 and/or HCV E2 protein, or any part thereof, from said cell culture.

The invention further pertains to a method for purifying hepatitis C virus (HCV) envelope proteins, or any part thereof, suitable for use in an immunoassay or vaccine, which method comprising:
(i) growing Hansenula or Saccharomyces glycosylation minus strains transformed with an envelope gene encoding an HCV E1 and/or HCV E2 protein, or any part thereof, in a suitable culture medium;
(ii) causing expression of said HCV E1 and/or HCV E2 gene, or any part thereof; and
(iii) purifying said intracellularly expressed HCV E1 and/or HCV E2 protein, or any part thereof, upon lysing the transformed host cell.

The invention further pertains to a method for purifying hepatitis C virus (HCV) envelope proteins, or any part thereof, suitable for use in an immunoassay or vaccine, which method comprising:
(i) growing Hansenula or Saccharomyces glycosylation minus strains transformed with an envelope gene encoding an HCV E1 and/or HCV E2 protein, or any part thereof, in a suitable culture medium, in which said HCV E1 and/or HCV E2 protein, or any part thereof, comprises at least two Cys-amino acids;
(ii) causing expression of said HCV E1 and/or HCV E2 gene, or any part thereof; and
(iii) purifying said HCV E1 and/or HCV E2 protein, or any part thereof, in which said Cys-amino acids are reversibly protected by chemical and/or enzymatic means, from said culture.

The invention further pertains to a method for purifying hepatitis C virus (HCV) envelope proteins, or any part thereof, suitable for use in an immunoassay or vaccine, which method comprising:
(i) growing Hansenula or Saccharomyces glycosylation minus strains transformed with an envelope gene encoding an HCV E1 and/or HCV E2 protein, or any part thereof, in a suitable culture medium, in which said HCV E1 and/or HCV E2 protein, or any part thereof, comprises at least two Cys-amino acids;
(ii) causing expression of said HCV E1 and/or HCV E2 gene, or any part thereof; and,
(iii) purifying said intracellulary expressed HCV E1 and/or HCV E2 protein, or any part thereof, upon lysing the transformed host cell, in which said Cys-amino acids are reversibly protected by chemical and/or enzymatic means.

The present invention specifically relates to a method for purifying recombinant HCV yeast proteins, or any part thereof, as described herein, in which said purification includes heparin affinity chromatography.

Hence, the present invention also relates to a method for purifying recombinant HCV yeast proteins, or any part thereof, as described above, in which said chemical means is sulfonation.

Hence, the present invention also relates to a method for purifying recombinant HCV yeast proteins, or any part thereof, as described above, in which said reversibly protection of Cys-amino acids is exchanged for an irreversible protection by chemical and/or enzymatic means.

Hence, the present invention also relates to a method for purifying recombinant HCV yeast proteins, or any part thereof, as described above, in which said irreversible protection by chemical means is iodo-acetamide.

Hence, the present invention also relates to a method for purifying recombinant HCV yeast proteins, or any part thereof, as described above, in which said irreversible protection by chemical means is NEM or Biotin-NEM or a mixture thereof.

The present invention also relates to a composition as defined above which also comprises HCV core, E1, E2, P7, NS2, NS3, NS4A, NS4B, NS5A and/or NS5B protein, or parts thereof. The core-glycosylated proteins E1, E2, and/or E1/E2 of the present invention may, for example, be combined with other HCV antigens, such as, for example, core, P7, NS3, NS4A, NS4B, NS5A and/or NS5B. The purification of these NS3 proteins will preferentially include a reversible modification of the cysteine residues, and even more preferentially sulfonation of cysteines. Methods to obtain such a reversible modification, including sulfonation have been described for NS3 proteins in Maertens et al. (PCT/EP99/02547). It should be stressed that the whole content, including all the definitions, of the latter document is incorporated by reference in the present application.

Also, the present invention relates to the use of a envelope protein as described herein for inducing immunity against HCV, characterized in that said HCV envelope protein is used as part of a series of time and compounds. In this regard, it is to be understood that the term “a series of time and compounds” refers to administering with time intervals to an individual the compounds used for eliciting an immune response. The latter compounds may comprise any of the following components: a HCV envelope protein according to the invention, HCV DNA vaccine composition, HCV polypeptides.

In this respect, a series comprises administering, either:
(i) an HCV antigen, such as, for example, a HCV envelope protein according to the invention, with time intervals, or
(ii) an HCV antigen, such as, for example, a HCV envelope protein according to the invention in combination with a HCV DNA vaccine composition, in which said envelope protein and said HCV DNA vaccine composition, can be administered simultaneously, or at different time intervals, including at alternating time intervals, or
(iii) either (i) or (ii), possibly in combination with other HCV peptides, with time intervals.

In this regard, it should be clear that a HCV DNA vaccine composition comprises nucleic acids encoding HCV envelope peptide, including E1-, E2-, E1/E2-peptides, NS3 peptide, other HCV peptides, or parts of said peptides. Moreover, it is to be understood that said HCV peptides comprises HCV envelope peptides, including E1-, E2-, E1/E2-peptides, other HCV peptides, or parts thereof. The term “other HCV peptides” refers to any HCV peptide or fragment thereof. In item (ii) of the above scheme, the HCV DNA vaccine composition comprises preferentially nucleic acids encoding HCV envelope peptides. In item (ii) of the above scheme, the HCV DNA vaccine composition consists even more preferentially of nucleic acids encoding HCV envelope peptides, possibly in combination with a HCV-NS3 DNA vaccine composition. In this regard, it should be clear that an HCV DNA vaccine composition comprises a plasmid vector comprising a polynucleotide sequence encoding an HCV peptide as described above, operably linked to transcription regulatory elements. As used herein, a “plasmid vector” refers to a nucleic acid molecule capable of transporting another nucleic acid to which it has been linked. Preferred vectors are those capable of autonomous replication and/or expression of nucleic acids to which they have been linked. In general, but not limited to those, plasmid vectors are circular double stranded DNA loops which, in their vector form, are not bound to the chromosome. As used herein, a “polynucleotide sequence” refers to polynucleotides such as deoxyribonucleic acid (DNA), and, where appropriate, ribonucleic acid (RNA). The term should also be understood to include, as equivalents, analogs of either RNA or DNA made from nucleotide analogs, and single (sense or antisense) and double-stranded polynucleotides. As used herein, the term “transcription regulatory elements” refers to a nucleotide sequence which contains essential regulatory elements, such that upon introduction into a living vertebrate cell it is able to direct the cellular machinery to produce translation products encoded by the polynucleotide. The term “operably linked” refers to a juxtaposition wherein the components are configured so as to perform their usual function. Thus, transcription regulatory elements operably linked to a nucleotide sequence are capable of effecting the expression of said nucleotide sequence. Those skilled in the art can appreciate that different transcriptional promoters, terminators, carrier vectors or specific gene sequences may be used successfully.

Alternatively, the DNA vaccine may be delivered through a live vector such as adenovirus, canary pox virus, MVA, and the like.

The HCV envelope proteins of the present invention, or the parts thereof, are particularly suited for incorporation into an immunoassay for the detection of anti-HCV antibodies, and/or genotyping of HCV, for prognosing/monitoring of HCV disease, or as a therapeutic agent.

A further aspect of the invention relates to a diagnostic kit for the detection of the presence of anti-HCV antibodies in a sample suspected to comprise anti-HCV antibodies, said kit comprising a HCV envelope protein or part thereof according to the invention. In a specific embodiment thereto, said HCV envelope protein or part thereof is attached to a solid support. In a further embodiment, said sample suspected to comprise anti-HCV antibodies is a biological sample.

The term “biological sample” as used herein, refers to a sample of tissue or fluid isolated from an individual, including but not limited to, for example, serum, plasma, lymph fluid, the external sections of the skin, respiratory-, intestinal- or genito-urinary tracts, oocytes, tears, saliva, milk, blood cells, tumors, organs, gastric secretions, mucus, spinal cord fluid, external secretions such as, for example, excrement, urine, sperm, and the like.

Another aspect of the invention refers to a composition comprising an isolated HCV envelope protein or fragment thereof according to the invention. Said composition may further comprise a pharmaceutically acceptable carrier and can be a medicament or a vaccine.

A further aspect of the invention covers a medicament or a vaccine comprising a HCV envelope protein or part thereof according to the invention.

Yet another aspect of the invention comprises a pharmaceutical composition for inducing a HCV-specific immune response in a mammal, said composition comprising an effective amount of a HCV envelope protein or part thereof according to the invention and, optionally, a pharmaceutically acceptable adjuvant. Said pharmaceutical composition comprising an effective amount of a HCV envelope protein or part thereof according to the invention may also be capable of inducing HCV-specific antibodies in a mammal, or capable of inducing a T-cell function in a mammal. Said pharmaceutical composition comprising an effective amount of a HCV envelope protein or part thereof according to the invention may be prophylactic composition or a therapeutic composition. In a specific embodiment said mammal is a human.

A “mammal” is to be understood as any member of the higher vertebrate class Mammalia, including humans; characterized by live birth, body hair, and mammary glands in the female that secrete milk for feeding the young. Mammals thus also include non-human primates and trimera mice (Zauberman et al. 1999).

A “vaccine” or “medicament” is a composition capable of eliciting protection against a disease, whether partial or complete, whether against acute or chronic disease; in this case the vaccine or medicament is a prophylactic vaccine or medicament. A vaccine or medicament may also be useful for treatment of an already ill individual, in which case it is called a therapeutic vaccine or medicament. Likewise, a pharmaceutical composition can be used for either prophylactic and/or therapeutic purposes in which cases it is a prophylactic and/or therapeutic composition, respectively.

The HCV envelope proteins of the present invention can be used as such, in a biotinylated form (as explained in WO 93/18054) and/or complexed to Neutralite Avidin (Molecular Probes Inc., Eugene, Oreg., USA), avidin or streptavidin. It should also be noted that “a vaccine” or “a medicament” may comprise, in addition to an active substance, a “pharmaceutically acceptable carrier” or “pharmaceutically acceptable adjuvant” which may be a suitable excipient, diluent, carrier and/or adjuvant which, by themselves, do not induce the production of antibodies harmful to the individual receiving the composition nor do they elicit protection. Suitable carriers are typically large slowly metabolized macromolecules such as proteins, polysaccharides, polylactic acids, polyglycolic acids, polymeric amino acids, amino acid copolymers and inactive virus particles. Such carriers are well known to those skilled in the art. Preferred adjuvants to enhance effectiveness of the composition include, but are not limited to: aluminium hydroxide, aluminium in combination with 3-O-deacylated monophosphoryl lipid A as described in WO 93/19780, aluminium phosphate as described in WO 93/24148, N-acetyl-muramyl-L-threonyl-D-isoglutamine as described in U.S. Pat. No. 4,606,918, N-acetyl-normuramyl-L-alanyl-D-isoglutamine, N-acetylmuramyl-L-alanyl-D-isoglutamyl-L-alanine-2-(1′2′dipalmitoyl-sn-glycero-3-hydroxyphosphoryloxy) ethylamine, RIBI (ImmunoChem Research Inc., Hamilton, Mont., USA) which contains monophosphoryl lipid A, detoxified endotoxin, trehalose-6,6-dimycolate, and cell wall skeleton (MPL+TDM+CWS) in a 2% squalene/Tween 80 emulsion. Any of the three components MPL, TDM or CWS may also be used alone or combined 2 by 2. The MPL may also be replaced by its synthetic analogue referred to as RC-529. Additionally, adjuvants such as Stimulon (Cambridge Bioscience, Worcester, Mass., USA), SAF-1 (Syntex) or bacterial DNA-based adjuvants such as ISS (Dynavax) or CpG (Coley Pharmaceuticals) may be used, as well as adjuvants such as combinations between QS21 and 3-de-O-acetylated monophosphoryl lipid A (WO94/00153), or MF-59 (Chiron), or poly[di(carboxylatophenoxy) phosphazene] based adjuvants (Virus Research Institute), or blockcopolymer based adjuvants such as Optivax (Vaxcel, Cythx) or inulin-based adjuvants, such as Algammulin and GammaInulin (Anutech), Incomplete Freund's Adjuvant (IFA) or Gerbu preparations (Gerbu Biotechnik). It is to be understood that Complete Freund's Adjuvant (CFA) may be used for non-human applications and research purposes as well. “A vaccine composition” may further contain excipients and diluents, which are inherently non-toxic and non-therapeutic, such as water, saline, glycerol, ethanol, wetting or emulsifying agents, pH buffering substances, preservatives, and the like. Typically, a vaccine composition is prepared as an injectable, either as a liquid solution or suspension. Injection may be subcutaneous, intramuscular, intravenous, intraperitoneal, intrathecal, intradermal. Other types of administration comprise implantation, suppositories, oral ingestion, enteric application, inhalation, aerosolization or nasal spray or drops. Solid forms, suitable for solution on, or suspension in, liquid vehicles prior to injection may also be prepared. The preparation may also be emulsified or encapsulated in liposomes for enhancing adjuvant effect. The polypeptides may also be incorporated into Immune Stimulating Complexes together with saponins, for example Quil A (ISCOMS). Vaccine compositions comprise an effective amount of an active substance, as well as any other of the above-mentioned components. “Effective amount” of an active substance means that the administration of that amount to an individual, either in a single dose or as part of a series, is effective for prevention or treatment of a disease or for inducing a desired effect. This amount varies depending upon the health and physical condition of the individual to be treated, the taxonomic group of the individual to be treated (e.g. human, non-human primate, primate, etc.), the capacity of the individual's immune system to mount an effective immune response, the degree of protection desired, the formulation of the vaccine, the treating doctor's assessment, the strain of the infecting pathogen and other relevant factors. It is expected that the amount will fall in a relatively broad range that can be determined through routine trials. Usually, the amount will vary from 0.01 to 1000 μg/dose, more particularly from 0.1 to 100 g/dose. Dosage treatment may be a single dose schedule or a multiple dose schedule. The vaccine may be administered in conjunction with other immunoregulatory agents.

The present invention is illustrated by the Examples as set forth below. These Examples are merely illustrative and are not construed to restrict or limit the invention in any way.

### EXAMPLES

### Example 1

### Construction of pFPMT-MFα-E1-H6 Shuttle Vector

Plasmids for Hansenula polymorpha transformation were constructed as follows. The pFPMT-MFα-E1-H6 shuttle vector has been constructed in a multi-step procedure. Initially the nucleic acid sequence encoding the HCV E1s protein (SEQ ID NO:2) was cloned after a CHH leader sequence (CHH=Carcinus maenas hyperglycemic hormone) which was subsequently changed for a MFα leader sequence (MFα=Saccharomyces cerevisiae α-mating factor).

At first a pUC18 derivative has been constructed harboring the CHH-E1-H6 unit as a EcoRI/BamHI fragment by the seamless cloning method (Padgett, K. A. and Sorge, J. A. 1996a). Thereto, the E1s-H6-encoding DNA fragment and the pCHH-Hir-derived acceptor plasmid were generated by PCR as described below.

### Generation of E1s-H6-Encoding DNA Fragment

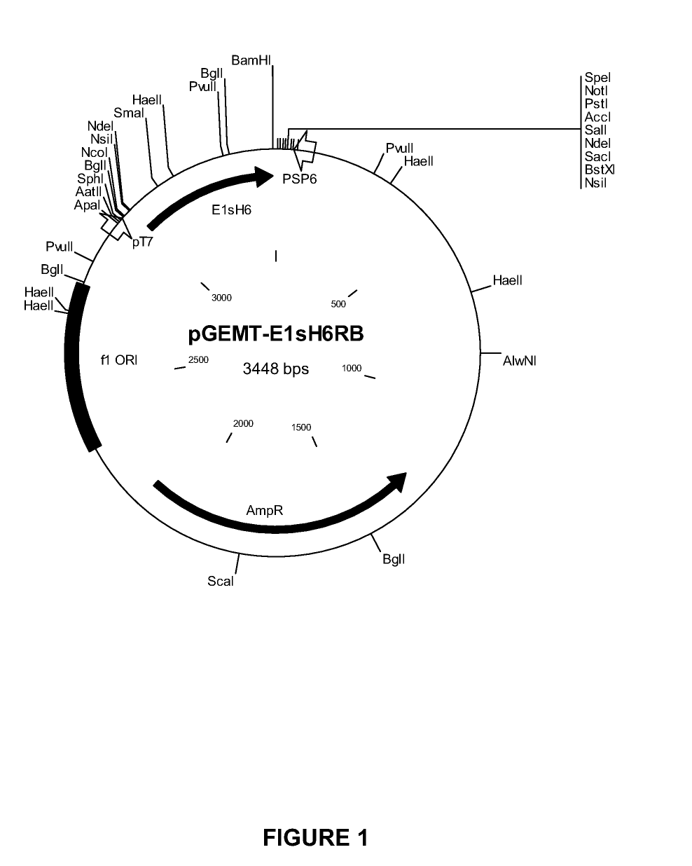

The E1-H6 DNA fragment (coding for HCV type 1b E1s protein consisting of the amino acids 192 to 326 of E1s elongated with 6 His-residues; SEQ ID NO:5) was isolated by PCR from the plasmid pGEMTE1sH6 (SEQ ID NO:6; FIG. 1). The following primers were used thereto:

CHHE1-F:

(SEQ ID NO:7)

5′-agttactcttca.aggtatgaggtgcgcaacgtgtccg-3′;

The Eam1104I site is underlined, the dot marks the cleavage site. The bold printed bases are complementary to those of primer CHH-links. The non-marked bases anneal within the start region of E1 (192-326) in sense direction; and

CHHE1-R:

(SEQ ID NO:8)

5′-agttactcttca.cagggatcctccttaatggtgatggtggtggtgc
c-3′;

The Eam1104I site is underlined, the dot marks the cleavage site. The bold printed bases are complementary to those of primer MF30-rechts. The bases forming the BamHI site useful for later cloning procedures are printed in italics. The non-marked bases anneal in antisense direction within the end of the E1-H6 unit, including the stop codon and three additional bases between the stop codon and the BamHI site.

The reaction mixture was constituted as follows: total volume of 50 μL containing 20 ng of Eco311-linearized pGEMTE1sH6, each 0.2 μM of primers CHHE1-F and CHHE1-R, dNTP's (each at 0.2 μM), 1× buffer 2 (Expand Long Template PCR System; Boehringer; Cat No 1681 834), 2.5 U polymerase mix (Expand Long Template PCR System; Boehringer; Cat No 1681 834).

Program 1 was used, said program consisting of the following steps:
1. denaturation: 5 min 95° C.;
2. 10 cycles of 30 sec denaturation at 95° C., 30 sec annealing at 65° C., and 130 sec elongation at 68° C.
3. termination at 4° C.

Then 5 μL 10× buffer 2 (Expand Long Template PCR System; Boeringer; Cat No 1681 834), 40 μL H₂O, and 5 μL of [dATP, dGTP, and dTTP (2 mM each); 10 mM 5-methyl-dCTP] were added to the sample derived from program 1, and further amplification was performed following program 2 consisting of the following steps:
1. denataruation: 5 min at 95° C.
2. 5 cycles of 45 sec denaturation at 95° C., 30 sec annealing at 65° C., and 130 sec at 68° C.
3. termination at 4° C.

### Generation of pCHH-Hir-Derived Acceptor Plasmid

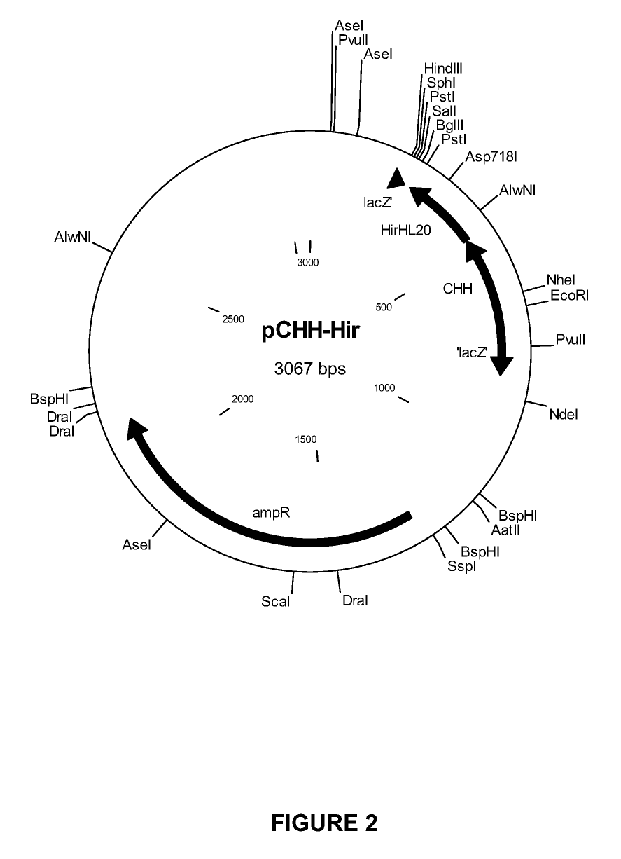

The acceptor fragment was made by PCR from the pCHH-Hir plasmid (SEQ ID NO:9; FIG. 2) and consists of almost the complete pCHH-Hir plasmid, except that the Hir-coding sequence is not present in the PCR product. Following primers were used for this PCR:
1.

CHH-links:

(SEQ ID NO:10)

5′-agttactcttca.cctcttttccaacgggtgtgtag-3′;

The Eam1104I site is underlined, the dot marks the cleavage site. The bold printed bases are complementary to those of primer CHHE1-F. The non-marked bases anneal within the end of the CHH sequence in antisense direction; and
2.

MF30-rechts:

(SEQ ID NO:11)

5′-agtcactcttca.ctgcaggcatgcaagcttggcg-3′;

The Eam1104I site is underlined, the dot marks the cleavage site. The bold printed bases are complementary to those of primer CHHE1-R. The non-marked bases anneal within the pUC18 sequences behind the cloned CHH-Hirudin HL20 of pCHH-Hir, pointing away from the insert.

The reaction mixture was constituted as follows: total volume of 50 μL containing 20 ng of Asp718I-linearized pCHH-Hir, each 0.2 μM of primers CHH-links and MF30-rechts, dNTP's (each at 0.2 μM), 1× buffer 2 (Expand Long Template PCR System; Boeringer; Cat No 1681 834), 2.5 U polymerase mix (Expand Long Template PCR System; Boeringer; Cat No 1681 834).

Program 1 was as described above was used.

Then 5 μL 10× buffer 2 (Expand Long Template PCR System; Boeringer; Cat No 1681 834), 40 μL H₂O, and 5 μL of [dATP, dGTP, and dTTP (2 mM each); 10 mM 5-methyl-dCTP] were added to the sample derived from program 1, and further amplification was performed following program 2 as described above.

### Generation of Vector pCHHE1

The E1s-H6-encoding DNA fragment and the pCHH-Hir-derived acceptor plasmid generated by PCR as described above were purified using the PCR product purification kit (Qiagen) according to the supplier's specifications. Subsequently the purified fragments were digested separately with Eam1104I. Subsequently, the E1s-H6 DNA fragment was ligated into the pCHH-Hir-derived acceptor plasmid using T4 ligase (Boehringer) following the specifications of the supplier. E. coli XL-Gold cells were transformed with the ligation mixture and the plasmid DNA of several ampicillin-resistant colonies were analyzed by digestion with EcoRI and BamHI. A positive clone was selected and denominated as pCHHE1.

### Generation of Vector pFPMT-CHH-E1H6

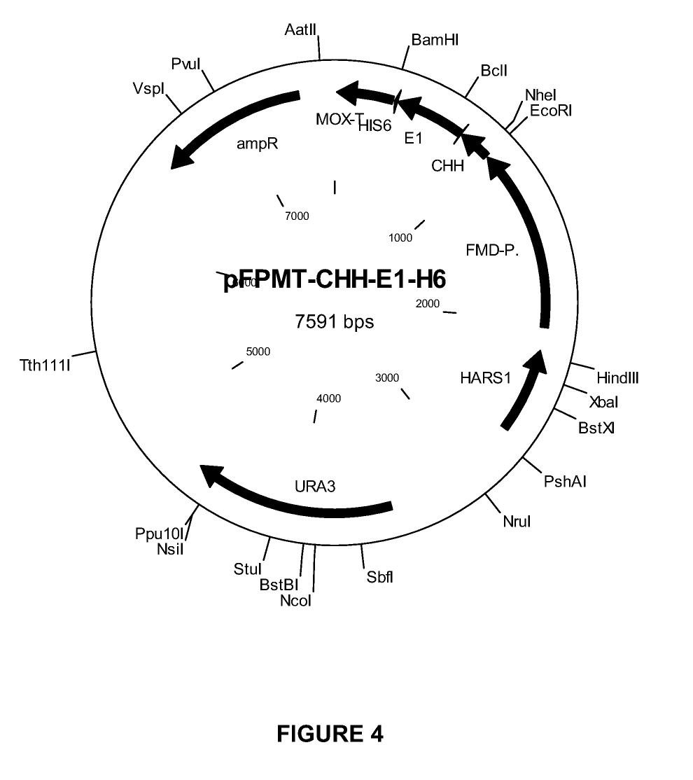

The EcoRI/BamHI fragment of pCHHE1 was ligated with the EcoRI/BamHI digested vector pFPMT121 (SEQ ID NO:12; FIG. 3). T4 ligase (Boehringer) was used according to the supplier's instructions. The ligation mixture was used to transform E. coli DH5αF′ cells. Several transformants were analyzed on restriction pattern of the plasmid DNA and a positive clone was withheld which was denominated pFPMT-CHH-E1H6 (SEQ ID NO:13; FIG. 4).

### Generation of pFPMT-MFα-E1-H6

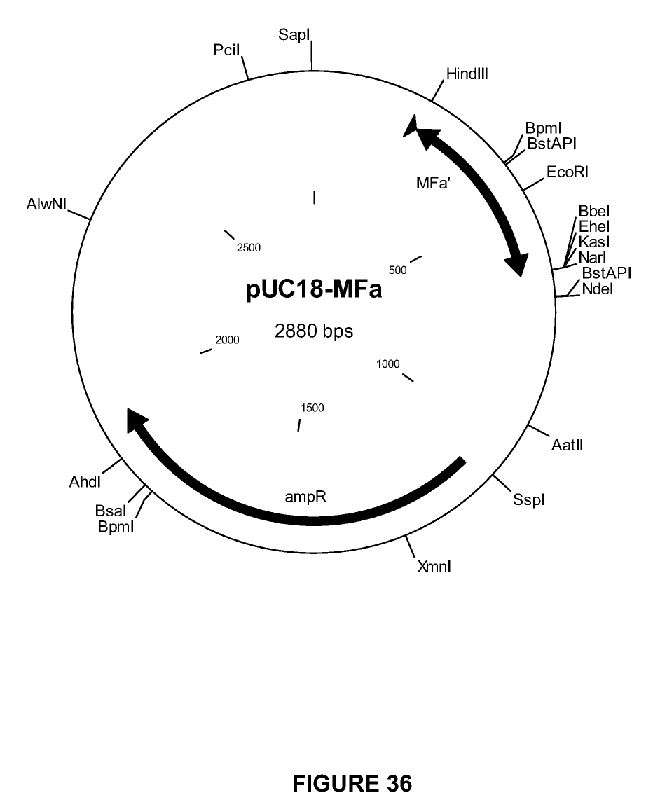

Finally the shuttle vector pFPMT-MFα-E1-H6 was generated by ligation of three fragments, said fragments being:
1. the 6.961 kb EcoRI/BamHI digested pFPMT121 (SEQ ID NO:12; FIG. 3),
2. the 0.245 EcoRI/HindIII fragment of pUC18-MFα (SEQ ID NO:62; FIG. 36), and
3. the 0.442 kb HindIII/BamHI fragment of a 0.454 kb PCR product derived from pFPMT-CHH-E1H6.

The 0.454 kb PCR product giving rise to fragment No. 3 was obtained by PCR using the following primers:

primer MFa-E1 f-Hi:

(SEQ ID NO:14)

5′-aggggtaagcttggataaaaggtatgaggtgcgcaacgtgtccggga
tgt-3′;
and
primer E1 back-Bam:

(SEQ ID NO:15)

5′-agttacggatccttaatggtgatggtggtggtgccagttcat-3′.

The reaction mixture was constituted as follows: Reaction mixture volume 50 μL, pFPMT-CHH-E1-H6 (EcoRI-linearized; 15 ng/μL), 0.5 μL; primer MFα-E1f-Hi (50 μM), 0.25 μL; primer E1 back-Bam (50 μM), 0.25 μL; dNTP's (all at 2 mM), 5 μL; DMSO, 5 μL; H₂O, 33.5 μL; Expand Long Template PCR System (Boeringer Mannheim; Cat No 1681 834) Buffer 2 (10× concentrated), 5 μL; Expand Long Template PCR System Polymerase mixture (1 U/μL), 0.5 μL.

The PCR program consisting of the following steps was used:
1. denaturation: 5 min at 95° C.
2. 29 cycles of 45 sec denaturation at 95° C., 45 sec annealing at 55° C., and 40 sec elongation at 68° C.
3. termination at 4° C.

Based on the primers used, the resulting 0.454 kb PCR product contained the codons of E1(192-326) followed by six histidine codons and a “taa” stop codon, upstream flanked by the 22 3′-terminal base pairs of the MFα prepro sequence (including the cloning relevant HindIII site plus a six base pairs overhang) and downstream flanked by a (cloning relevant) BamHI site and a six base pairs overhang.

For the ligation reaction, T4 DNA ligase (Boehringer Mannheim) has been used according to the supplier's conditions (sample volume 20 μL).

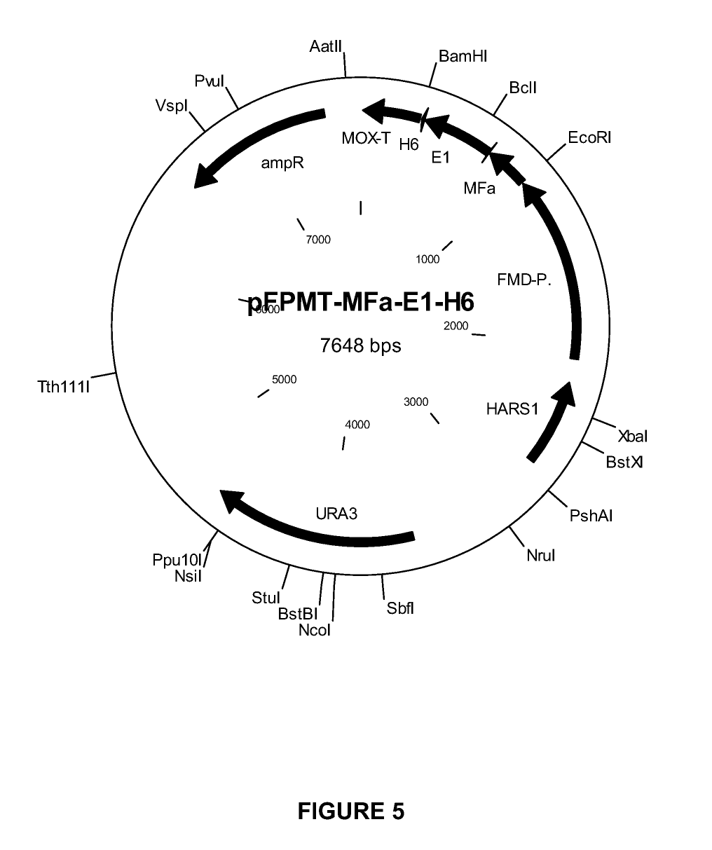

E. coli HB101 cells were transformed with the ligation mixture and positive clones withheld after restriction analysis of the plasmids isolated from several transformants. A positive plasmid was selected and denominated as pFPMT-MFα-E1-H6 (SEQ ID NO:16; FIG. 5).

### Example 2

### Construction OF pFPMT-CL-E1-H6 Shuttle Vector

Plasmids for Hansenula polymorpha transformation were constructed as follows. The pFPMT-CL-E1-H6 shuttle vector was constructed in three steps starting from pFPMT-MFα-E1-H6 (SEQ ID NO:16, FIG. 5).

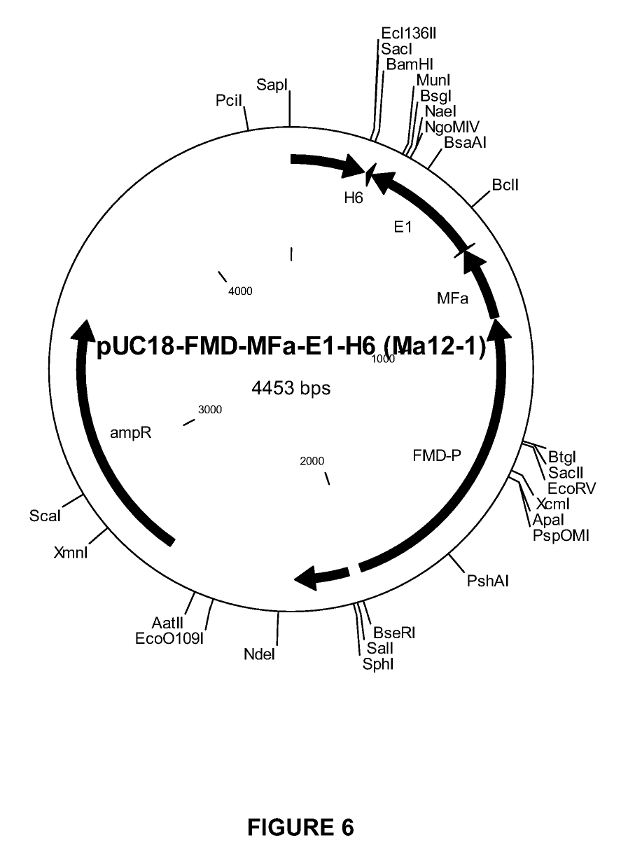

In a first step, the MFα-E1-H6 reading frame of pFPMT-MFα-E1-H6 was subcloned into the pUC18 vector. Therefore a 1.798 kb SalI/BamHI fragment of pFPMT-MFα-E1-H6 (containing the FMD promotor plus MFα-E1-H6) was ligated to the SalI/BamHI vector fragment of pUC18 with T4 ligase (Boehringer) according to the supplier's conditions. This resulted in plasmid that is depicted in FIG. 6 (SEQ ID NO:17), and further denominated as pMa12-1 (pUC18-FMD-MFα-E1-H6). The ligation mixture was used to transform E. coli DH5αF′ cells. Several ampicillin-resistant colonies were picked and analyzed by restriction enzyme digestion of plasmid DNA isolated from the picked clones. A positive clone was further analyzed by determining the DNA sequence of the MFα-E1-H6 coding sequence. A correct clone was used for PCR directed mutagenesis to replace the MFα pre-pro-sequence with the codons of the avian lysozyme pre-sequence (“CL”; corresponding to amino acids 1 to 18 of avian lysozyme; SEQ ID NO:1). The principle of the applied PCR-directed mutagenesis method is based on the amplification of an entire plasmid with the desired alterations located at the 5′-ends of the primers. In downstream steps, the ends of the linear PCR product are modified prior to self-ligation resulting in the desired altered plasmid.

The following primers were used for the PCR reaction:

primer CL hin:

(SEQ ID NO:18)

5′-tgcttcctaccactagcagcactaggatatgaggtgcgcaacgtgtc
cggg-3′;
primer CL her neu:

(SEQ ID NO:19)

5′-tagtactagtattagtaggcttcgcatgaattcccgatgaaggcaga
gagcg-3′.

The underlined 5′ regions of the primers contain the codons of about half of the avian lysozyme pre-sequence. Primer CL her neu includes a SpeI restriction site (italic). The non-underlined regions of the primers anneal with the codons for amino acid residues 192 to 199 of E1 (CL hin) or the with the “atg” start codon over the EcoRI site up to position −19 (counted from the EcoRI site) of FMD promoter. The primers are designed to amplify the complete pMa12-1 thereby replacing the codons of the MFα pre-pro-sequence with the codons of the avian lysozyme pre sequence.

The reaction mixture was constituted as follows: pUC18-FMD-Mfα-E1-H6 (pMa12-1; 1.3 ng/μL), 1 μL; primer CL hin (100 μM), 2 μL; primer CL her neu (100 μM), 2 μL; dNTP's (all at 2.5 mM), 8 μL; H₂O, 76 μL; Expand Long Template PCR System (Boeringer; Cat No 1681 834) Buffer 2 (10× concentrated), 10 μL; Expand Long Template PCR System Polymerase mixture (1 U/μL), 0.75 μL.

The PCR program consisting of the following steps was applied:
1. denaturation: 15 min at 95° C.
2. 35 cycles of 30 sec denaturation at 95° C., 1 min annealing at 60° C., and 1 min elongation at 72° C.
3. termination at 4° C.

The resulting PCR product was checked by agarose gel electrophoresis for its correct size (3.5 kb). Thereafter the 3′-A overhangs form the PCR product were removed by a T4 polymerase reaction resulting in blunt ends with 3′- and 5′-OH-groups. Therefore, the PCR product was treated with T4 polymerase (Boehringer; 1 U/μL): to the remaining 95 μL of PCR reaction mix were added 1 μL T4 polymerase and 4 μL dNTP's (all at 2.5 mM). The sample was incubated for 20 min at 37° C. Subsequently, the DNA was precipitated with ethanol and taken up in 16 μL H₂O.

Subsequently 5′-phosphates were added to the blunt-ended PCR product by a kinase reaction. Therefore, to the 16 μL blunt-ended PCR product were added 1 μL T4 polynucleotide kinase (Boehringer; 1 U/μL), 2 μL 10-fold concentrated T4 polynucleotide kinase reaction buffer (Boehringer), and 1 μL ATP (10 mM). The sample was incubated for 30 min at 37° C. Subsequently the DNA was applied onto a 1% agarose gel and the correct product band was isolated by means of the gel extraction kit (Qiagen) according to the supplier's conditions. Fifty (50) ng of the purified product was then self-ligated by use of T4 ligase (Boehringer) according to the supplier's conditions. After 72 h incubation at 16° C., the DNA in the ligation mix was precipitated with ethanol and dissolved in 20 μL water.

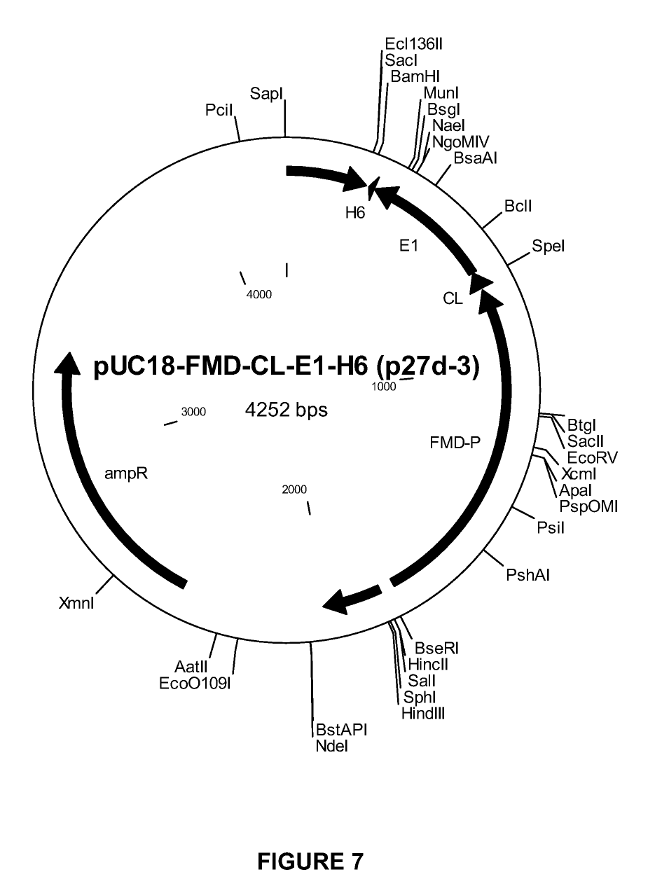

E. coli DH5α-F′ cells were subsequently transformed with 10 μL of the ligation sample. The plasmid DNA of several ampicillin-resistant clones was checked by means of restriction enzyme digestion. A positive clone was withheld and denominated p27d-3 (pUC18-FMD-CL-E1-H6, SEQ ID NO:20, FIG. 7). Subsequently the CL-E1-H6 reading frame was verified by DNA sequencing.

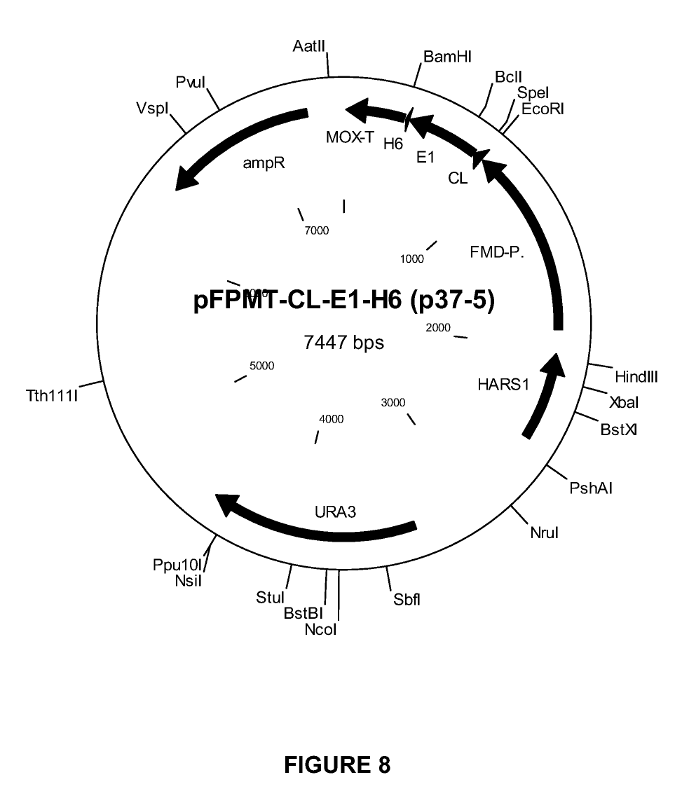

In a last step the pFPMT-CL-E1-H6 shuttle vector was constructed as described below. The 0.486 kb EcoRI/BamHI fragment of p27d-3 (harboring CL-E1(192-326)-H6) was ligated with EcoRI/BamHI-digested pFPMT121 (SEQ ID NO:12, FIG. 3). For the reaction, T4 ligase (Boehringer) has been used according to the supplier's recommendations. The DNA in the ligation sample was precipitated with ethanol and dissolved in 10 μL H₂O. E. coli DH5α F′ cells were transformed with 10 μL of the ligation sample, and the plasmid DNA of several ampicillin-resistant colonies were analyzed by digestion with EcoRI and BamHI. Plasmid clone p37-5 (pFPMT-CL-E1-H6; SEQ ID NO:21, FIG. 8) showed the desired fragment sizes of 0.486 kb and 6.961 kb. The correct sequence of CL-E1-H6 of p37-5 was verified by sequencing.

### Example 3

### Construction of pFPMT-MFα-E2-H6 and pMPT-MFα-E2-H6 Shuttle Vectors

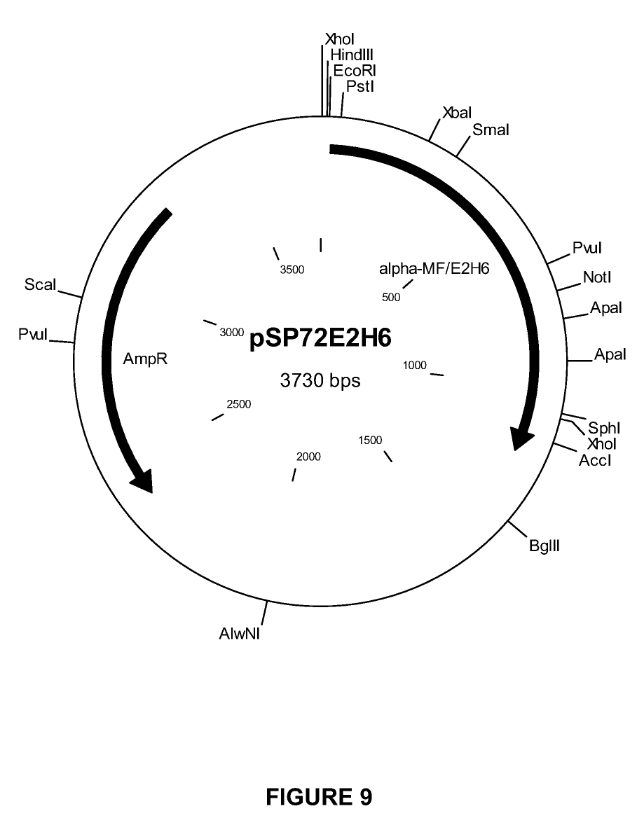

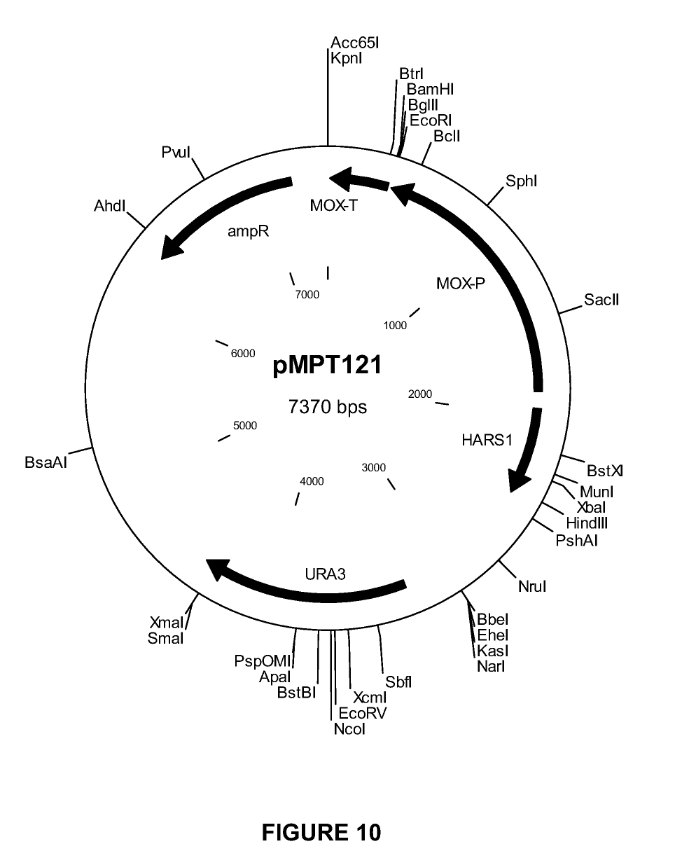

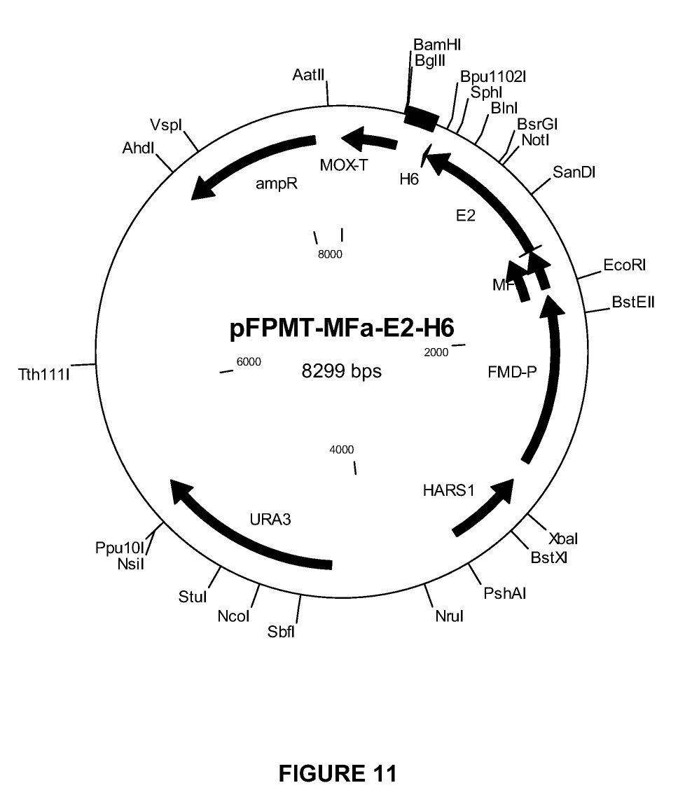

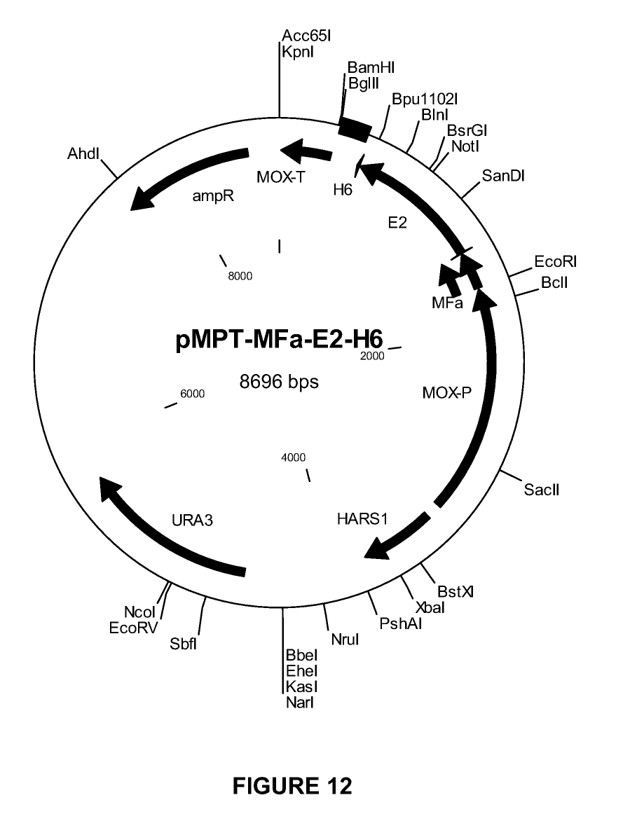

Plasmids for Hansenula polymorpha transformation were constructed as follows. The DNA sequence encoding the MFα-E2s (amino acids 384-673 of HCV E2)-VIEGR-His6 (SEQ ID NO:5) was isolated as a 1.331 kb EcoRI/BglII fragment from plasmid pSP72E2H6 (SEQ ID NO:22, FIG. 9). This fragment was ligated with either the EcoRI/BglII-digested vectors pFPMT121 (SEQ ID NO:12, Figure C+2) or pMPT121 (SEQ ID NO:23, FIG. 10) using T4 DNA ligase (Boehringer Mannheim) according to the supplier's recommendations. After transformation of E. coli and checking of plasmid DNA isolated from different transformants by restriction enzyme digestion, positive clones were withheld and the resulting shuttle vectors are denominated pFPMT-MFα-E2-H6 (SEQ ID NO:22, FIG. 11) and pMPT-MFα-E2-H6 (SEQ ID NO:23, FIG. 12), respectively.

### Example 4

### Construction of pFPMT-CL-E2-H6 Shuttle Vector

The shuttle vector pFPMT-CL-E2-H6 was assembled in a three-step procedure. An intermediate construct was prepared in which the E2 coding sequence was cloned behind the signal sequence of α-amylase of Schwanniomyces accidentalis. This was done by the seamless cloning method (Padgett, K. A. and Sorge, J. A. 1996b).

### Generation of E2s-H6 Encoding DNA Fragment

At first the DNA sequence encoding E2-H6 (amino acids 384 to 673 of HCV E2 extended with the linker peptide “VIEGR” and with 6 His residues, SEQ ID NO:5) was amplified from the pSP72E2H6 plasmid (SEQ ID NO:24, FIG. 11) by PCR. The used primers were denoted MF30E2/F and MF30E2/R and have the following sequences:
primer MF30E2/F: 5′-agtcactcttca. aggcatacccgcgtgtcaggaggg-3′ (SEQ ID NO:26; the Eam1104I site is underlined, the dot marks the enzyme's cleavage site; the last codon of the S. occidentalis signal sequence is printed in bold; the non-marked bases anneal with the codons of E2 (amino acids 384-390 of HCV E2);
primer MF30E2/R: 5′-agtcactcttca.cagggatccttagtgatggtggtgatg-3′ (SEQ ID NO:27; the Eam1104I site is underlined, the dot marks the enzyme's cleavage site; the bold printed bases are complementary to the bold printed bases of primer MF30-Rechts (see below); a BamHI site to be introduced into the construct is printed in italic; the non-marked sequence anneals with the stop codon and the six terminal H is codons of E2 (384-673)-VIEGR-H6 (SEQ ID NO:5).

The reaction mixture was constituted as follows: total volume of 50 μL containing 20 ng of the 1.33 kb EcoRI/BglII fragment of pSP72E2H6, each 0.2 mM of primers MF30E2/F and MF30E2/R, dNTP's (each 0.2 μM), 1× buffer 2 (Expand Long Template PCR System; Boeringer; Cat No 1681 834), 2.5 U polymerase mix (Expand Long Template PCR System; Boeringer; Cat No 1681 834).

The PCR program 3 consisting of the following steps was used:
1. denaturation: 5 min at 95° C.
2. 10 cycles of 30 sec denaturation at 95° C., 30 sec annealing at 65° C., and 1 min elongation at 68° C.
3. termination at 4° C.

Then 10 μL 10× buffer 2 (Expand Long Template PCR System; Boeringer; Cat No 1681 834), 40 μL H₂O, and 5 μL of [dATP, dGTP, and dTTP (2 mM each); 10 mM 5-methyl-dCTP] have been added to the sample derived from PCR program 3, and it has been continued with PCR program 4 consisting of the following steps:
1. denaturation: 5 min at 95° C.
2. 5 cycles of 45 sec denaturation at 95° C., 30 sec annealing at 65° C., and 1 min elongation at 68° C.
3. termination at 4° C.

### Generation of pMF30-Derived Acceptor Plasmid

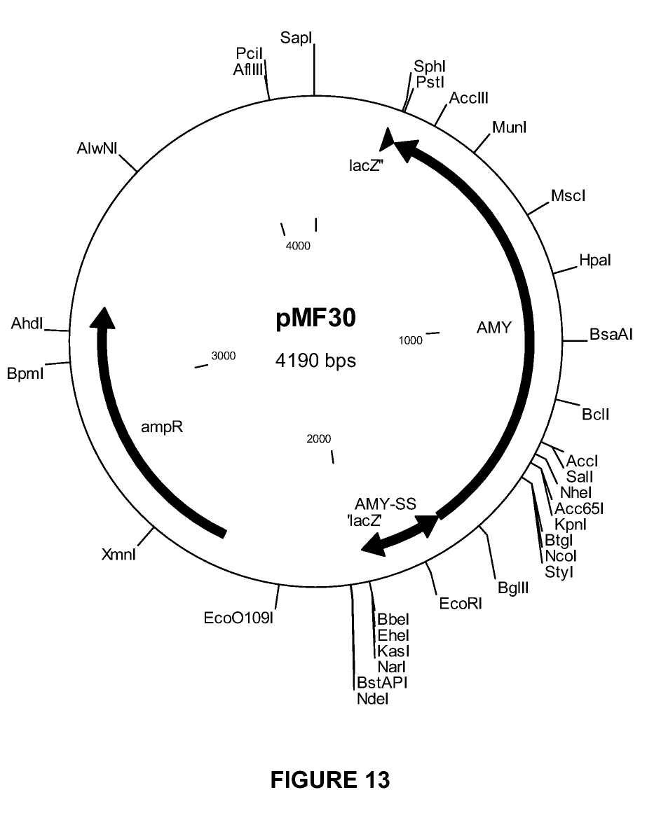

The second fragment originated from the plasmid pMF30 (SEQ ID NO:28, FIG. 13), the amplicon was almost the complete pMF30 plasmid excluding the codons of the mature α-amylase of S. occidentalis, modifications relevant for cloning were introduced by primer design. The following set of primers was used:
primer MF30-Links: 5′-agtcactcttca.cctcttgtcaaaaataatcggttgag-3′ (SEQ ID NO:29; the Eam1104I site is underlined, the dot marks the enzyme's cleavage site; the bold printed “cct” is complementary to the bold printed “agg” of primer MF30E2/F (see above); the non-marked and the bold printed bases anneal with the 26 terminal bases of the codons of the α-Amylase of S. occidentalis in pMF30);
primer MF30-Rechts: 5′-agtcactcttca. ctgcaggcatgcaagcttggcg-3′ (SEQ ID NO:11; the Eam1104I site is underlined, the dot marks the enzyme's cleavage site; the bold printed “ctg” is complementary to the bold printed “cag” of primer MF30E2/R (see above); the non-marked bases anneal with pUC18 sequences downstream of the stop codon of the α-Amylase of S. occidentalis in pMF30).

The reaction mixture was constituted as follows: total volume of 50 μL containing 20 ng of the BglII-linearized pMF30, each 0.2 μM of primers MF30-Links and MF30-Rechts, dNTP's (each 0.2 μM), 1× buffer 1 (Expand Long Template PCR System; Boeringer; Cat No 1681 834), 2.5 U polymerase mix (Expand Long Template PCR System; Boeringer; Cat No 1681 834). The same PCR programs (programs 3 and 4) as described above were used, except for the elongation times which were extended from 1 minute to 4 minutes in both programs.

### Generation of Vector pAMY-E2

The E2s-H6 encoding DNA fragment and pMF30-derived acceptor plasmid obtained by PCR were controlled on their respective size by gel electrophoresis on a 1% agarose gel. The PCR products were purified with a PCR product purification kit (Qiagen) according to the supplier's instructions. Subsequently the purified fragments were digested separately with Eam11004I. Ligation of the E2s-H6 fragment with the pMF30-derived acceptor plasmid was performed by using T4 ligase (Boehringer) according to the supplier's recommendations. The ligation mixture was used to transform E. coli DH5αF′ cells and the plasmid DNA of several clones was analyzed by EcoRI/BamHI digestion. A positive clone was selected, its plasmid further denominated as pAMY-E2, and utilized for further modifications as described below.

### Generation of Vector pUC18-CL-E2-H6

The pAMY-E2 was subjected to PCR-directed mutagenesis in order to replace the codons of the α-amylase signal sequence with the codons of the avian lysozyme pre sequence. This is further denominated as “CL”, corresponding to the first 18 amino acids of avian lysozyme ORF (SEQ ID NO:1). For this mutagenesis following primers were used:

primer CL2 hin:

(SEQ ID NO:30)

5′-tgcttcctaccactagcagcactaggacatacccgcgtgtcaggagg
ggcag-3′;
and
primer CL2 her:

(SEQ ID NO:31)

5′-tagtactagtattagtaggcttcgcatggaattcactggccgtcgt
ttta-caacgtc-3′.

The underlined 5′-regions of the primers contain the DNA sequence of about half of the avian lysozyme pre sequence. Primer CL2 her includes SpeI (italic) and EcoRI (italic, double underlined) restriction sites. The non-underlined regions of the primers anneal with the codons of amino acid residues 384 to 392 of E2 (CL2 hin) or the with the “atg” start codon over the EcoRI site up to position −19 (counted from the EcoRI site) of FMD promoter. The primers are designed to amplify the complete pAMY-E2 vector thereby replacing the codons of the α-amylase signal sequence with the codons of the avian lysozyme pre-sequence.

The PCR reaction was performed according to the following program:
1. denaturation: 15 min at 95° C.
2. 35 cycles of 30 sec denaturation at 95° C., 1 min annealing at 60° C., and 1 min elongation at 72° C.
3. termination at 4° C.

The following reaction mixture was used: pAMY-E2 (1 ng/μL), 1 μL; primer CL2 hin (100 μM), 2 μL; primer CL2 her (100 μM), 2 μL; dNTP's (2.5 mM each), 8 μL; H₂O, 76 μL; Expand Long Template PCR System (Boeringer; Cat No 1681 834) Buffer 2 (10× concentrated), 10 μL; Expand Long Template PCR System Polymerase mixture (1 U/μL), 0.75 μL.

The resulting PCR product was checked by gel electrophoresis on a 1% agarose gel. Prior to ligation the PCR fragment was modified as follows. The 3′-A overhangs were removed by T4 polymerase resulting in blunt ends with 3′- and 5′-OH-groups. Thereto 1 μL T4 polymerase (Boehringer, 1 U/μL) was added to the residual 95 μL PCR reaction mixture along with 4 μL dNTP's (2.5 mM each). The sample was incubated for 20 min at 37° C. Subsequently the DNA was precipitated with ethanol and dissolved in 16 μL deionized water. This was followed by a kinase treatment to add 5′-phosphates to the blunt-ended PCR product. To the 16 μL dissolved blunt-ended PCR product were added 1 μL T4 polynucleotide kinase (Boehringer, 1 U/μL), 2 μL 10-fold concentrated T4 polynucleotide kinase reaction buffer (Boehringer) and 1 μL ATP (10 mM). The sample was incubated for 30 min at 37° C.

The kinase treated sample was subsequently separated on a 1% agarose gel. The product band was isolated. The DNA was extracted from the agarose slice by means of the Gel Extraction kit (Qiagen) according to the supplier's recommendations. Fifty (50) ng of the purified product was then self-ligated by use of T4 ligase (Boehringer) according to the supplier's conditions. After 16 h incubation at 16° C., the DNA in the ligation mix was precipitated with ethanol and dissolved in 20 μL H₂O (ligation sample).

E. coli DH5αF′ cells were transformed with 10 μL of the ligation sample. Several ampicillin-resistant clones were further characterized via restriction analysis of the isolated plasmid DNA. A positive clone was denominated as pUC18-CL-E2-H6 and was used for further modifications as described below.

### Generation of Shuttle Vector pFPMT-CL-E2-H6

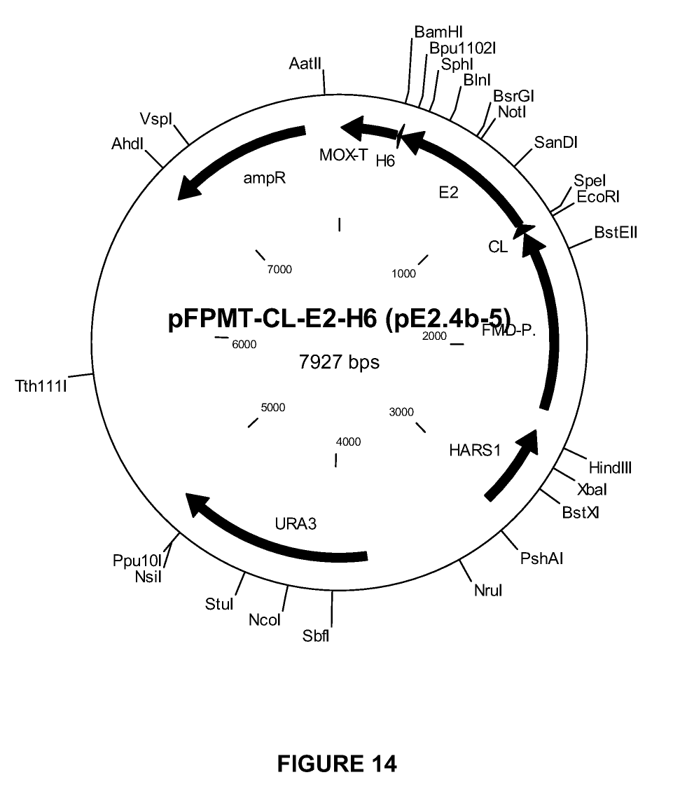

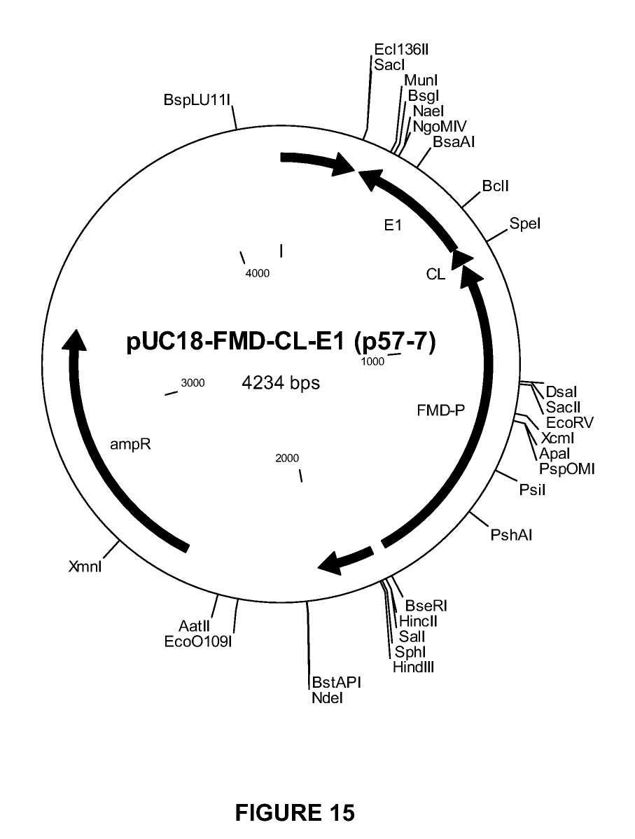

A 0.966 kb EcoRI/BamHI fragment was isolated from pUC18-CL-E2-H6 (harboring CL-E2(384-673)-VIEGR-H6) and was ligated into the EcoRI/BamHI-digested pFPMT121 (SEQ ID NO:12, FIG. 3). For the reaction, T4 ligase (Boehringer) was used according to the supplier's conditions. The ligation sample was precipitated with ethanol and dissolved in 10 μL water. This was used to transform E. coli DH5αF′ cells, a positive clone was withheld after restriction analysis and the respective plasmid is denominated pFPMT-CL-E2-H6 (SEQ ID NO:32, FIG. 14).

### Example 5

### Construction OF pFPMT-CL-K-H6-E1 Shuttle Vector

The construction of the shuttle vector was comprised of two steps.

In a first step the pUC18-FMD-CL-H6-K-E1-H6 construct was constructed by site-directed mutagenesis. The pUC18-FMD-CL-E1-H6 was used as template (SEQ ID NO:20; FIG. 7).

The following primers were used:

Primer H6K hin neu:

(SEQ ID NO:37)

5′-catcacaaatatgaggtgcgcaacgtgtccgggatgtac-3′.
Primer H6KRK her neu:

(SEQ ID NO:38)

5′-gtgatggtggtgtcctagtgctgctagtggtaggaagcatag-3′.

(The Bases Providing Additional Codons are Underlined.)

The PCR reaction mixture was constituted as follows: pUC18-FMD-CL-E1-H6 (2 ng/μL), 1 μL; primer H6K hin neu (100 μM), 2 μL; primer H6KRK her neu (100 μM), 2 μL; dNTP's (2.5 mM each), 8 μL; H₂O, 76 μL; Expand Long Template PCR System (Boeringer; Cat No 1681 834) Buffer 2 (10× concentrated), 10 μL; Expand Long Template PCR System Polymerase mixture (1 U/μL), 0.75 μL.

The PCR program used consisted of the following steps:
denaturation step: 15 min at 95° C.
35 cycles of 30 sec denaturation at 95° C., 1 min annealing at 60° C., and 5 min elongation at 72° C.
termination at 4° C.

An aliquot of the PCR sample was analyzed on a 1% agarose gel to check its size, which was correct (˜4.2 kb).

Thereafter the 3′-A overhangs from the PCR product were removed by a T4 polymerase reaction resulting in blunt ends with 3′- and 5′-OH groups. Therefore, to the remaining 95 μL of the PCR reaction were added 1 μL T4 polymerase (Boehringer; 1 U/μL) and 4 μL dNTP's (2.5 mM each). The sample was incubated for 20 min at 37° C. Subsequently, the DNA in the sample was precipitated with ethanol and dissolved in 16 μL H₂O, Subsequently 5′-phosphates were added to the blunt-ended PCR product by a kinase reaction. Therefore, to the 16 μL dissolved blunt-ended PCR product were added 1 μL T4 polynucleotide kinase (Boehringer; 1 U/μL), 2 μL 10-fold concentrated T4 polynucleotide kinase reaction buffer (Boehringer), and 1 μL ATP (10 mM). The sample was incubated for 30 min at 37° C.

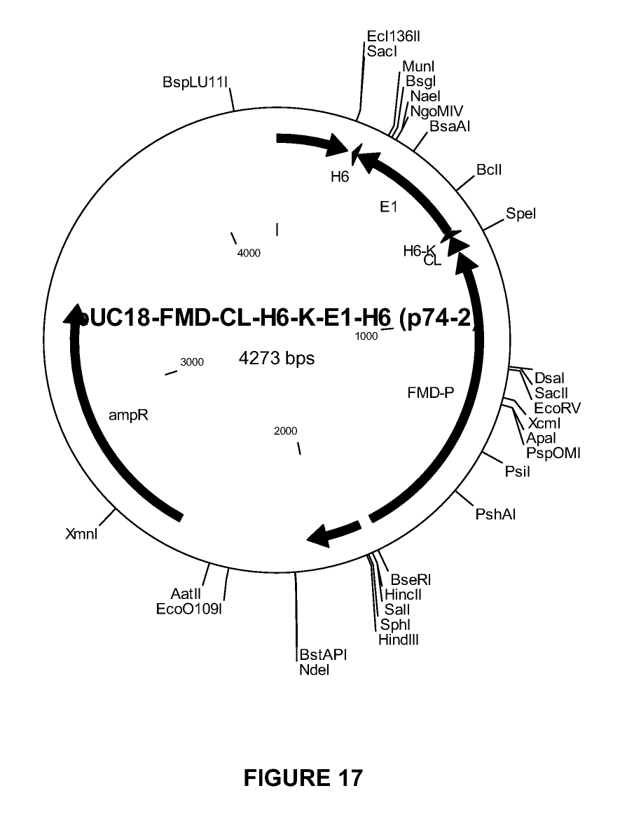

Subsequently the sample was applied onto a 1% agarose gel and the correct product band was isolated, by means of the gel extraction kit (Qiagen) according to the supplier's conditions. Fifty (50) ng of the purified product has then been self-ligated by use of T4 ligase (Boehringer) according to the supplier's recommendations. After 72 h incubation at 16° C. the DNA in the ligation sample was precipitated with ethanol and dissolved in 10 μL water. E. coli DH5αF′ cells were transformed with 5 μL of the ligation sample. The plasmid DNA of several ampicillin-resistant colonies was analyzed by restriction enzyme digestion, a positive clone was withheld and the corresponding plasmid denominated: pUC18-FMD-CL-H6-E1-K-H6 (SEQ ID NO:39, FIG. 17).

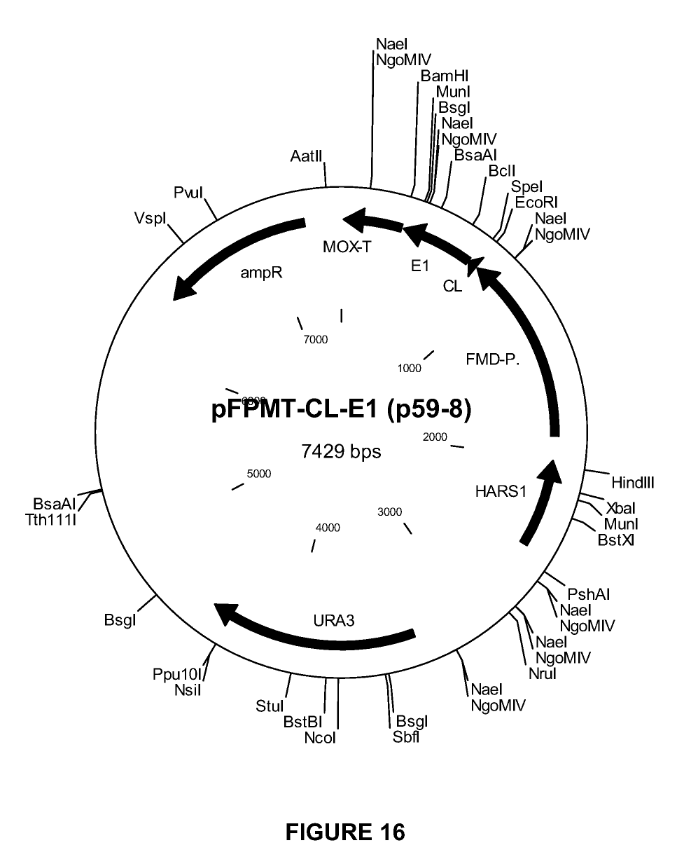

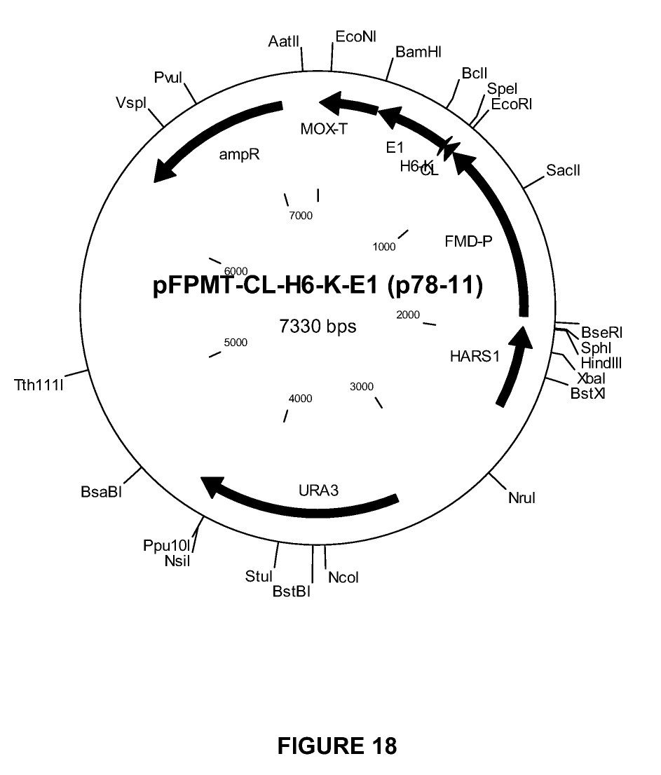

In a second step the transfer vector was constructed by a two-fragment ligation. In the following construction fragments with BclI cohesive ends were involved. Since BclI can cleave its site only on unmethylated DNA, an E. coli dam⁻ strain was transformed with the involved plasmids pUC18-FMD-CL-H6-K-E1-H6 (SEQ ID NO:39, FIG. 17) and pFPMT-CL-E1 (SEQ ID NO:36, FIG. 16). From each transformation, an ampicillin-resistant colony was picked, grown in a liquid culture and the unmethylated plasmid DNAs were prepared for the further use. The 1.273 kb BclI/HindIII fragment of the unmethylated plasmid pUC18-FMD-CL-H6-K-E1-H6 (harbouring the FMD promoter, the codons of the CL-H6-K unit, and the start of E1) and the 6.057 kb BclI/HindIII fragment of plasmid pFPMT-CL-E1 (harbouring the missing part of the E1 reading frame starting from the BclI site, without C-terminal His tag, as well as the pFPMT121-located elements except for the FMD promoter) were prepared and ligated together for 72 h at 16° C. by use of T4 ligase (Boehringer) in a total volume of 20 μL according to the supplier's specifications. Subsequently, the ligation mixture was placed on a piece of nitrocellulose membrane floating on sterile deionized water in order to desalt the ligation mixture (incubation for 30 min at room temperature). E. coli TOP10 cells were transformed by electroporation with 5 μL of the desalted sample. The plasmid DNA of several resulting ampicillin-resistant colonies was analyzed by restriction enzyme digestion. A positive clone was withheld and denominated pFPMT-CL-H6-K-E1 (SEQ ID NO:40, FIG. 18).

### Example 6

### Transformation of Hansenula Polymorpha and Selection of Transformants

H. polymorpha strain RB11 was been transformed (PEG-mediated DNA uptake protocol essentially as described by (Klebe, R. J. et al. 1983c) with the modification of (Roggenkamp, R. et al. 1986) with the different parental shuttle vectors as described in Examples 1 to 5. For each transformation, 72 uracil-prototrophic colonies were selected and used for strain generation by the following procedure. For each colony, a 2 mL liquid culture was inoculated and grown in test tubes for 48 h (37° C.; 160 rpm; angle 45°) in selective medium (YNB/glucose, Difco). This step is defined as the first passaging step. A 150 μL aliquot of the cultures of the first passaging step were used to inoculate 2 mL fresh YNB/glucose medium. Again, the cultures have been incubated as described above (second passaging step). Together, eight of such passaging steps were carried out. Aliquots of the cultures after the third and the eighth passaging steps were used to inoculate 2 mL of non-selective YPD medium (Difco). After 48 h of incubation at 37° C. (160 rpm; angle 45°; the so-called first stabilization step), 150 μL aliquots of these YPD cultures have been used to inoculate fresh 2 mL YPD cultures which were incubated as described above (second stabilization step). Aliquots of the cultures of the second stabilization step were then streaked on plates containing selective YNB/agar. These plates were incubated for four days until macroscopic colonies became visible. A well-defined single colony of each separation was defined as strain and used for further expression analysis.

Expression analysis was performed on small-scale shake flask cultures. A colony was picked from the above mentioned YNB/agar plate and inoculated in 2 mL YPD and incubated for 48 h as mentioned above. This 2 mL-aliquot was used as seed culture for 20 mL shake flask culture. YPGlycerol (1%) was used as medium and the shake flask was incubated on a rotary shaker (200 rpm, 37° C.). After 48 h of growth 1% MeOH was added to the culture for induction of the expression cassette. At different time intervals cell pellets of 1 mL aliquots were collected and stored at −20° C. until further analysis. Specific protein expression was analyzed by SDS-PAGE/Western blotting. Therefore cell pellets were solubilized in sample-buffer (Tris HCl-SDS) and incubated for >15 minutes at 95° C. Proteins were separated on a 15% polyacryl-amide gel and blotted (wet-blot; bicarbonate buffer) onto nitrocellulose membranes. Blots were developed using a specific murine anti-E1 (IGH 201) or murine anti-E2 (IGH 216, described by Maertens et al. in WO96/04385) as first antibody, Rabbit-Anti-Mouse-AP was used as second antibody. Staining was performed with NBT-BCIP.

Positive strains were withheld for further investigation.

Five of these positive clones were used in a shake flask expression experiment. A colony of the respective strain was picked from YNB plate and used to inoculate 2 mL YPD. These cultures were incubated as described above. This cell suspension was used to inoculate a second seed culture of 100 mL YPD medium in a 500 mL shake flask. This shake flask was incubated on a rotary shaker for 48 h at 37° C. and 200 rpm. A 25 mL aliquot of this seed culture was used to inoculate 250 mL YPGlycerol (1%) medium and was incubated in a baffled 2-1 shake flask under the above described conditions. 48 h after inoculation 1% MeOH (promotor induction) was added and the shake flasks were further incubated under the above described conditions. 24 h post induction, the experiment was stopped and cell pellets collected by centrifugation. The expression level of the five different clones was analyzed by SDS-PAGE/Western blotting (conditions as above). A titration series of each clone was loaded onto the gel and the most productive strain was selected for further fermentation and purification trials.

Surprisingly, H. polymorpha, a yeast strain closely related to Pichia pastoris (Gellissen, G. 2000c), is able to express HCV proteins essentially without hyperglycosylation and thus with sugar moieties comparable in size to the HCV envelope proteins expressed by HCV-recombinant vaccinia virus-infected mammalian cells.

The Hansenula polymorpha strain RB11 was deposited on Apr. 19, 2002 under the conditions of the Budapest Treaty at the Mycothéque de l'UCL (MUCL), Universite Catholique de Louvain, Laboratoire de mycologie, Place Croix du Sud 3 bte 6, B-1348 Louvain-la-Neuve, Belgium and has the MUCL accession number MUCL43805.

### Example 7

### Construction of pSY1aMFE1sH6a Vector

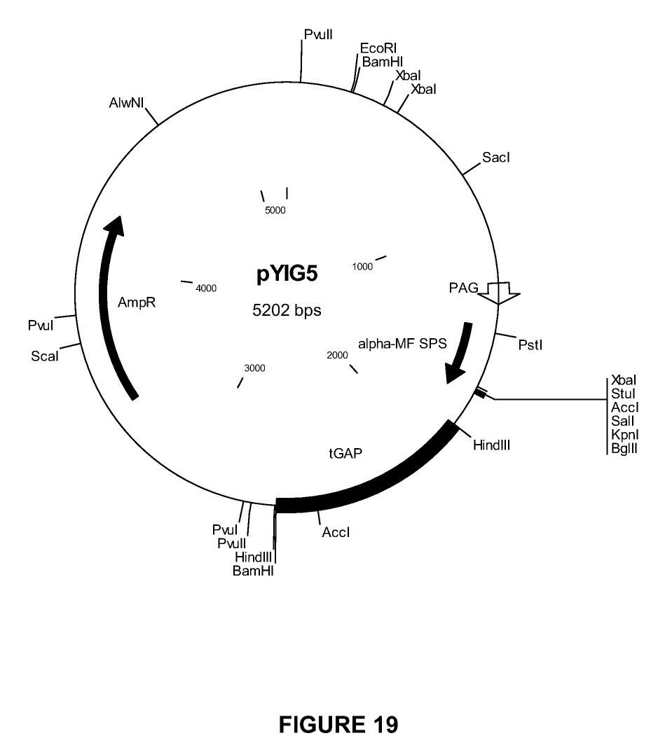

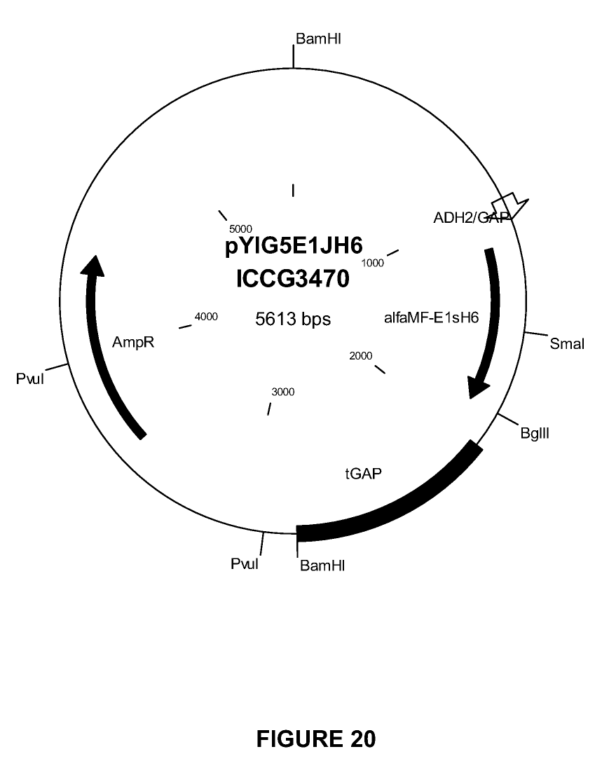

The S. cerevisiae expression plasmid was constructed as follows. An E1-coding sequence was isolated as a NsI1/Eco52I fragment from pGEMT-E1sH6 (SEQ ID NO:6, FIG. 1) which was made blunt-ended (using T4 DNA polymerase) and cloned in the pYIG5 vector (SEQ ID NO:41, FIG. 19) using T4 DNA ligase (Boehringer) according to the supplier's specifications. The cloning was such that the E1s-H6 encoding fragment was joined directly and in frame to the αMF-coding sequence. The ligation mixture was transformed in E. coli DH5αF′ cells. Subsequently, the plasmid DNA of several ampicillin resistant clones was analyzed by restriction digestion and a positive clone was withheld and denominated as pYIG5E1H6 (ICCG3470; SEQ ID NO:42, FIG. 20).

The expression cassette (containing the αMF-sequence and the E1s-coding region with a His-tag) was transferred as a BamHI fragment (2790 bp) of pYIG5E1H6 into the BamHI-digested E coli/S. cerevisiae pSY1 shuttle vector (SEQ ID NO:21, FIG. 43). The ligation was performed with T4 DNA ligase (Boehringer) according to supplier's conditions. The ligation mix was transformed to E coli DH5αF′ cells, and the plasmid DNA of several ampicillin resistant colonies was analyzed by restriction enzyme digestion. A positive clone was withheld and denominated pSY1aMFE1sH6a (ICCG3479; SEQ ID NO:44, FIG. 22).

### Example 8

### Construction of pSYYIGSE2H6 Vector

The S. cerevisiae expression plasmid pSYYIGSE2H6 was constructed as follows. An E2 coding sequence was isolated as a SalI/KpnI fragment from pBSK-E2sH6 (SEQ ID NO:45, FIG. 23) which was made blunt-ended (using T4 DNA polymerase) and subsequently cloned in the pYIG5 vector (SEQ ID NO:41, FIG. 19) using T4 DNA ligase (Boehringer) according to the supplier's specifications. The cloning was such that the E2-H6 encoding fragment was joined directly and in frame to the αMF-coding sequence. The ligation mixture was then transformed to E. coli DH5αF′ cells, the plasmid DNA of several ampicillin resistant clones was analyzed by restriction digestion and a positive clone withheld and denominated as pYIG5HCCL-22aH6 (ICCG2424; SEQ ID NO:46, FIG. 24).

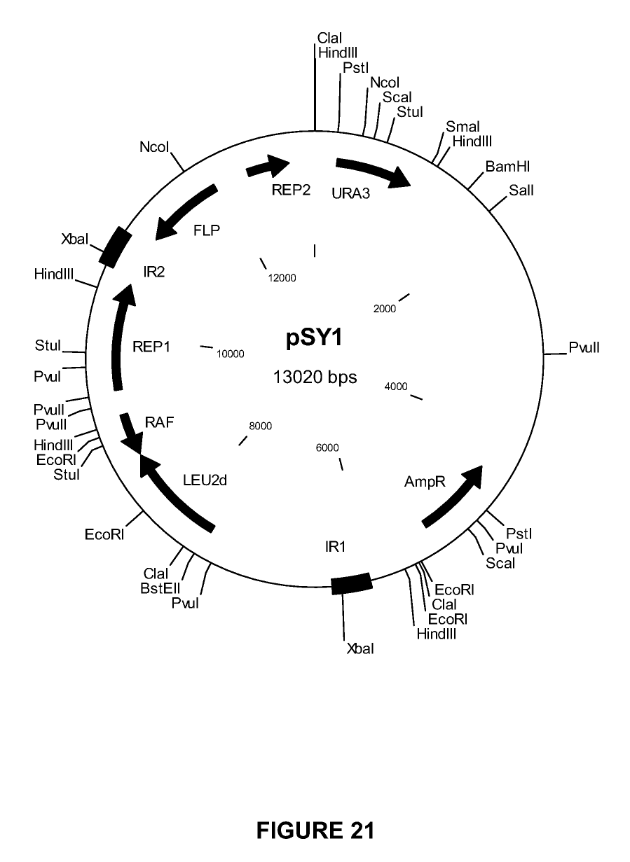

The expression cassette (containing the αMF-sequence and the E2 (384-673) coding region with a His-tag) was transferred as a BamHI fragment (3281 bp) of pYIG5HCCL-22aH6 into the BamHI opened E. coli/S. cerevisiae pSY1 shuttle vector (SEQ ID NO:43, FIG. 21). The ligation was performed with T4 DNA ligase (Boehringer) according to supplier's conditions. The ligation mix was transformed to E. coli DH5αF′ cells and the plasmid DNA of several ampicillin resistant colonies was analyzed by restriction enzyme digestion. A restriction positive clone was withheld and denominated pSYYIGSE2H6 (ICCG2466; SEQ ID NO:47, FIG. 25).

### Example 9

### Construction of pSY1YIG7E1s Vector

The S. cerevisiae expression plasmid pSY1YIG7E1s was constructed as follows. An E1 coding sequence was isolated as a NsI1/Eco52I fragment from pGEMT-E1s (SEQ ID NO:6, FIG. 1) which was made blunt-ended and cloned into the pYIG7 vector (SEQ ID NO:48, FIG. 26) using T4 DNA ligase (Boehringer) according to the supplier's specifications. The cloning was such that the E1-encoding fragment was joined directly and in frame to the αMF-coding sequence. The ligation mixture was transformed to E. coli DH5αF′ cells, the plasmid DNA of several ampicillin resistant clones analyzed by restriction digestion and a positive clone withheld and denominated as pYIG7E1 (SEQ ID NO:49, FIG. 27). The expression cassette (containing the CL leader sequence and the E1 (192-326) coding region) was transferred as a BamHI fragment (2790 bp) of pYIG7E1 into the BamHI-digested E. coli/S. cerevisiae pSY1 shuttle vector (SEQ ID NO:43, FIG. 21). The ligation was performed with T4 DNA ligase (Boehringer) according to supplier's conditions. The ligation mix was transformed to E. coli DH5αF′ cells and the plasmid DNA of several ampicillin resistant colonies was analyzed by restriction enzyme digestion. A positive clone was withheld and denominated pSY1YIG7E1s (SEQ ID NO:50, FIG. 28).

### Example 10

### Transformation of Saccharomyces Cerevisiae and Selection of Transformants

In order to overcome hyper-glycosylation problems, often reported for proteins over-expressed in Saccharomyces cerevisiae, a mutant screening was set-up. This screening was based on the method of Ballou (Ballou, L. et al. 1991a), whereby spontaneous recessive orthovanadate-resistant mutants were selected. Initial strain selection was performed based on the glycosylation pattern of invertase, as observed after native gel electrophoresis. A strain, reduced in glycosylation capabilities, was withheld for further recombinant protein expression experiments and denominated strain IYCC155. The nature of mutation has not been further studied.

Said glycosylation-deficient strain IYCC155 was transformed with the plasmids as described in Examples 7 to 9 essentially by to the lithium acetate method as described by Elble (Elble, R. 1992). Several Ura complemented strains were picked from a selective YNB+2% agar plate (Difco) and used to inoculate 2 ml YNB+2% glucose. These cultures were incubated for 72 h, 37° C., 200 rpm on orbital shaker, and the culture supernatant and intracellular fractions were analysed for expression of E1 by western blot developed with a E1 specific murine monoclonal antibody (IGH 201). A high producing clone was withheld for further experiments.

The expression of proteins in the S. cerevisiae glycosylation deficient mutant used here is hampered by the suboptimal growth characteristics of such strains which leads to a lower biomass yield and thus a lower yield of the desired proteins compared to wild-type S. cerevisiae strains. The yield of the desired proteins was still substantially higher than in mammalian cells.

### Example 11

### Construction of pPICZalphaD′E1sH6 and pPICZalphaE′E1sH6 Vectors

The shuttle vector pPICZalphaE′E1sH6 was constructed starting from the pPICZalphaA vector (Invitrogen; SEQ ID NO:51, FIG. 29). In a first step said vector was adapted in order to enable cloning of the E1 coding sequence directly behind the cleavage site of the KEX2 or STE13 processing proteases, respectively. Therefore pPICZalphaA was digested with XhoI and NotI. The digest was separated on a 1% agarose gel and the 3519 kb fragment (major part of vector) was isolated and purified by means of a gel extraction kit (Qiagen). This fragment was then ligated using T4 polymerase (Boehringer) according to the supplier's conditions in presence of specific oligonucleotides yielding pPICZalphaD′ (SEQ ID NO:52, FIG. 30) or pPICZalphaE′ (SEQ ID NO:53, FIG. 31).

The following oligonucleotides were used:

for constructing pPICZalphaD′:

(SEQ ID NO:54)

| | |
|---|---|
| 8822: | 5′-TCGAGAAAAGGGGCCCGAATTCGCATGC-3′; |
| and | |

(SEQ ID NO:55)

| | |
|---|---|
| 8823: | 5′-GGCCGCATGCGAATTCGGGCCCCTTTTC-3′ |

which yield, after annealing, the linker oligonucleotide:

(SEQ ID NO:54)

TCGAGAAAAGGGGCCCGAATTCGCATGC

(SEQ ID NO:55)

CTTTTCCCCGGGCTTAAGCGTACGCCGG
for constructing pPICZalphaE′

(SEQ ID NO:56)

| | |
|---|---|
| 8649: | 5′-TCGAGAAAAGAGAGGCTGAAGCCTGCAGCATATGC-3′ |

(SEQ ID NO:57)

| | |
|---|---|
| 8650: | 5′-GGCCGCATATGCTGCAGGCTTCAGCCTCTCTTTTC-3′ |

which yield, after annealing, the linker oligonucleotide:

(SEQ ID NO:56)

TCGAGAAAAGAGAGGCTGAAGCCTGCAGCATATGC

(SEQ ID NO:57)

CTTTTCTCTCCGACTTCGGACGTCGTATACGCCGG

These shuttle vectors pPICZalphaD′ and pPICZalphaE′ have newly introduced cloning sites directly behind the cleavage site of the respective processing proteases, KEX2 and STE13. The E1-H6 coding sequence was isolated as a NsI1/Eco52I fragment from pGEMT-E1sH6 (SEQ ID NO:6, FIG. 1). The fragment was purified using a gel extraction kit (Qiagen) after separation of the digest on a 1% agarose gel. The resulting fragment was made blunt-ended (using T4 DNA polymerase) and ligated into either pPICZalphaD′ or pPICZalphaE′ directly behind the respective processing protease cleavage site.

The ligation mixtures were transformed to E. coli TOP10F′ cells and plasmid DNA of several zeocin resistant colonies analyzed by restriction enzyme digestion. Positive clones were withheld and denominated pPICZalphaD′E1sH6 (ICCG3694; SEQ ID NO:58, FIG. 32) and pPICZalphaE′E1sH6 (ICCG3475; SEQ ID NO:59, FIG. 33), respectively.

### Example 12

### Construction of pPICZalphaD′E2sH6 and pPICZalphaE′E2sH6 Vectors

The shuttle vectors pPICZalphaD′ and pPICZalphaE′ were constructed as described in Example 11.

The E2-H6 coding sequence was isolated as a SalI/KpnI fragment from pBSK-E2sH6 (SEQ ID NO:45, FIG. 23). The fragment was purified with a gel extraction kit (Qiagen) after separation of the digest on a 1% agarose gel. The resulting fragment was made blunt-ended (using T4 DNA polymerase) and ligated into either pPICZalphaD′ or pPICZalphaE′ directly behind the respective processing protease cleavage site.

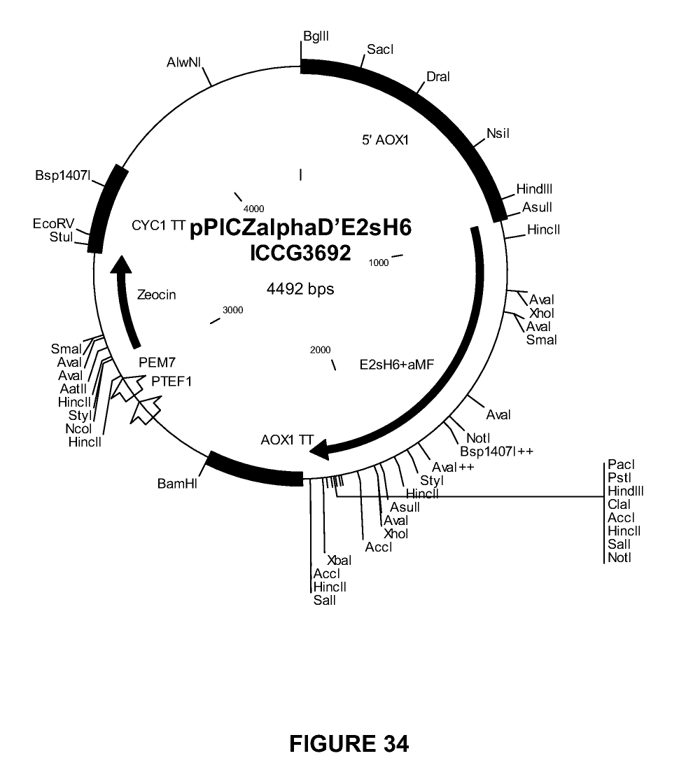

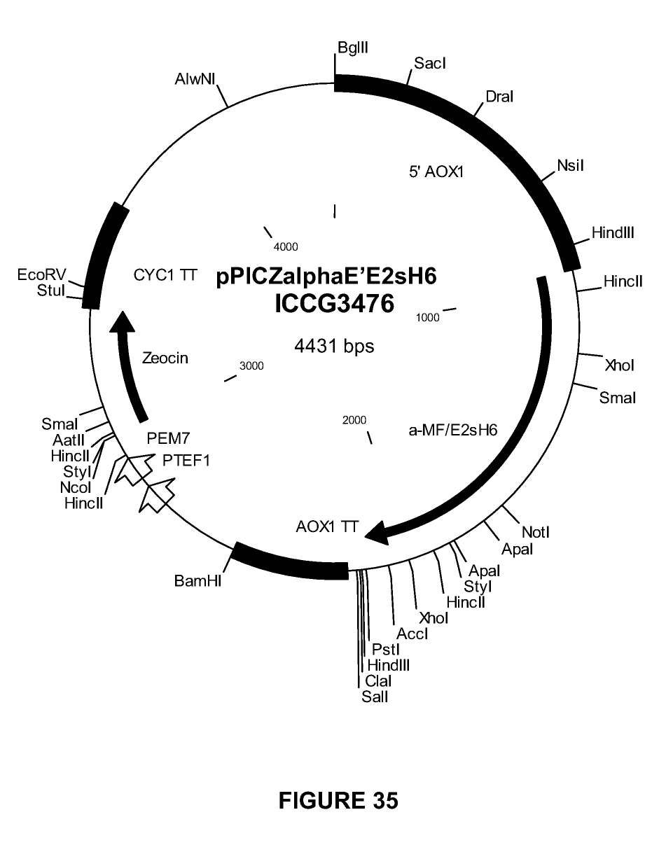

The ligation mixture was transformed to E. coli TOP10F′ cells and the plasmid DNA of several zeocin resistant colonies was analyzed by restriction enzyme digestion. Positive clone were withheld and denominated pPICZalphaD′E2sH6 (ICCG3692; SEQ ID NO:60, FIG. 34) and pPICZalphaE′E2sH6 (ICGG3476; SEQ ID NO:61, FIG. 35), respectively.

### Example 13

### Transformation of Pichia Pastoris and Selection of Transformants

The P. pastoris shuttle plasmids as described in Examples 11 and 12 were transformed to P. pastoris cells according to the supplier's conditions (Invitrogen). An E1- and an E2-producing strain were withheld for further characterization.

The HCV envelope proteins were expressed in P. pastoris, a yeast strain well known for the fact that hyperglycosylation is normally absent (Gellissen, G. 2000d) and previously used to express dengue virus E protein as GST fusion (Sugrue, R. J. et al. 1997b). Remarkably, the resulting P. pastoris-expressed HCV envelope proteins displayed a comparable glycosylation as is observed in wild-type Saccharomyces strains. More specifically, the HCV envelope proteins produced by P. pastoris are hyperglycosylated (based on the molecular weight of the expression products detected in western-blots of proteins isolated from transformed P. pastoris cells).

### Example 14

### Culture Conditions for Saccharomyces Cerevisiae, Hansenula Polymorpha and Pichia Pastoris

### Saccharomyces cerevisiae

### Cell Banking

Of the selected recombinant clone a master cell bank and working cell bank were prepared. Cryo-vials were prepared from a mid-exponentially grown shake flask culture (incubation conditions as for fermentation seed cultures, see below). Glycerol was added (50% final conc.) as a cryoprotectant.

### Fermentation

Seed cultures were started from a cryo-preserved working cell bank vial and grown in 500 mL medium (YNB supplemented with 2% sucrose, Difco) in a 2 L Erlenmeyer shake flasks at 37° C., 200 rpm for 48 h.

Fermentations were typically performed in Biostat C fermentors with a working volume of 15 L (B. Braun Int., Melsungen, Germany). The fermentation medium contained 1% Yeast Extract, 2% Peptone and 2% sucrose as carbon source. Poly-ethylene glycol was used as anti-foam agent.

Temperature, pH and dissolved oxygen were typically controlled during the fermentation, applicable set-points are summarised in Table 1. Dissolved oxygen was cascade controlled by agitation/aeration. pH was controlled by addition of NaOH (0.5 M) or H₃PO₄ solution (8.5

TABLE 1
Typical parameter settings for S. cerevisiae fermentations

| Parameter | set-point | |
|---|---|---|
| Temperature | 33-37° | C. |
| pH | 4.2-5.0 | |
| DO (growth phase) | 10-40% | air saturation |
| DO (induction) | 0-5% | |
| aeration | 0.5-1.8 | vvm* |
| agitation | 150-900 | rpm |

*volume replacement per minute

The fermentation was started by the addition of 10% seed-culture. During the growth phase the sucrose concentration was monitored off-line by HPLC analysis (Polysphere Column OAKC Merck).

During the growth phase the dissolved oxygen was controlled by cascade control (agitation/aeration). After complete metabolisation of sucrose the heterologous protein production was driven by the endogenous produced ethanol supplemented with stepwise addition of EtOH in order to maintain the concentration at approximately 0.5% (off-line HPLC analysis, polyspher OAKC column) During this induction phase the dissolved oxygen was controlled below 5% air-saturation, by manual adjustment of airflow rate and agitator speed.

Typically the fermentation was harvested 48 to 72 h post induction by concentration via tangential flow filtration followed by centrifugation of the concentrated cell suspension to obtain cell pellets. If not analyzed immediately, cell pellets were stored at −70° C.

### Hansenula polymorpha

### Cell Banking

Of the selected recombinant clone a master cell bank and working cell bank were prepared.

Cryo-vials were prepared from a mid-exponentially grown shake flask culture (incubation conditions as for fermentation seed cultures, see below). Glycerol was added (50% final conc.) as a cryoprotectant.

### Fermentation

Seed cultures were started from a cryo-preserved (−70° C.) working cell bank vial and grown in 500 mL medium (YPD, Difco) in a 2 L Erlenmeyer shake flasks at 37° C., 200 rpm for 48 h. Fermentations were typically performed in Biostat C fermentors with a working volume of 15 L (B. Braun Int., Melsungen, Germany). The fermentation medium contained 1% Yeast Extract, 2% Peptone and 1% glycerol as carbon source. Poly-ethylene glycol was used as anti-foam agent.

Temperature, pH, air-in and dissolved oxygen were typically controlled during the fermentation, applicable set-points are summarised in Table 2. Dissolved oxygen was controlled by agitation. pH was controlled by addition of NaOH (0.5 M) or H₃PO₄ solution (8.5%).

TABLE 2
Typical parameter settings for H. polymorpha fermentations

| Parameter | set-point | |
|---|---|---|
| Temperature | 30-40° | C. |
| pH | 4.2-5.0 | |
| DO | 10-40% | air saturation |
| aeration | 0.5-1.8 | vvm* |
| agitation | 150-900 | rpm |

*volume replacement per minute

The fermentation was started by the addition of 10% seed-culture. During the growth phase the glycerol concentration was monitored off-line (Polysphere Column OAKC Merck) and 24 h after complete glycerol consumption 1% methanol was added in order to induce the heterologous protein expression. The fermentation was harvested 24 h post induction by concentration via tangential flow filtration followed by centrifugation of the concentrated cell suspension to obtain cell pellets. If not analyzed immediately, cell pellets were stored at −70° C.

### Pichia pastoris

Small scale protein production experiments with recombinant Pichia pastoris were set up in shake flask cultures. Seed cultures were grown overnight in YPD medium (Difco). Initial medium pH was corrected to 4.5. Shake flasks were incubated on a rotary shaker at 200-250 rpm, 37° C.

The small scale production was typically performed at 500 mL scale in 2 L shake flasks and were started with a 10% inoculation in expression medium, containing 1% Yeast extract, 2% Peptone (both Difco), and 2% glycerol as carbon source. Incubation conditions were as for the seed culture. Induction was started by addition of 1% MeOH approximately 72 h after inoculation. The cells were collected 24 h post induction by centrifugation. If not analyzed immediately, cell pellets were stored at −70° C.

### Example 15

### Leader Peptide Removal from MFα-E1-H6 and MFα-E2-H6 Proteins Expressed in Selected Yeast Cells

The expression products in Hansenula polymorpha and a Saccharomyces cerevisiae glycosylation minus strain of the HCV E1 and E2 protein constructs with the α-mating factor (αMF) leader sequence of S. cerevisiae were further analyzed. Since both genotype 1b HCV E1s (aa 192-326) and HCV E2s (aa 383-673 extended by the VIEGR (SEQ ID NO:69)-sequence) were expressed as C-terminal his-tagged (H6, HHHHHH, SEQ ID NO:63; said HCV proteins are furtheron in this Example denoted as αMF-E1-H6 and αMF-E2-H6) proteins, a rapid and efficient purification of the expressed products after guanidinium chloride (GuHCl)-solubilization of the yeast cells was performed on Ni-IDA (Ni-iminodiacetic acid). In brief, cell pellets were resuspended in 50 mM phosphate, 6M GuHCl, pH 7.4 (9 vol/g cells). Proteins were sulfonated overnight at room temperature (RT) in the presence of 320 mM (4% w/v) sodium sulfite and 65 mM (2% w/v) sodium tetrathionate. The lysate was cleared after a freeze-thaw cycle by centrifugation (10.000 g, 30 min, 4° C.) and Empigen (Albright & Wilson, UK) and imidazole were added to the supernatant to final concentrations of 1% (w/v) and 20 mM, respectively. The sample was filtrated (0.22 μM) and loaded on a Ni-IDA Sepharose FF column, which was equilibrated with 50 mM phosphate, 6M GuHCl, 1% Empigen (buffer A) supplemented with 20 mM imidazole. The column was washed sequentially with buffer A containing 20 mM and 50 mM imidazole, respectively, till absorbance at 280 nm reached baseline level. The his-tagged products were eluted by applying buffer D, 50 mM phosphate, 6M GuHCl, 0.2% (for E1) or 1% (for E2) Empigen, 200 mM imidazole. The eluted materials were analyzed by SDS-PAGE and western-blot using a specific monoclonal antibodies directed against E1 (IGH201), or E2 (IGH212). The E1-products were immediately analyzed by Edman degradation.

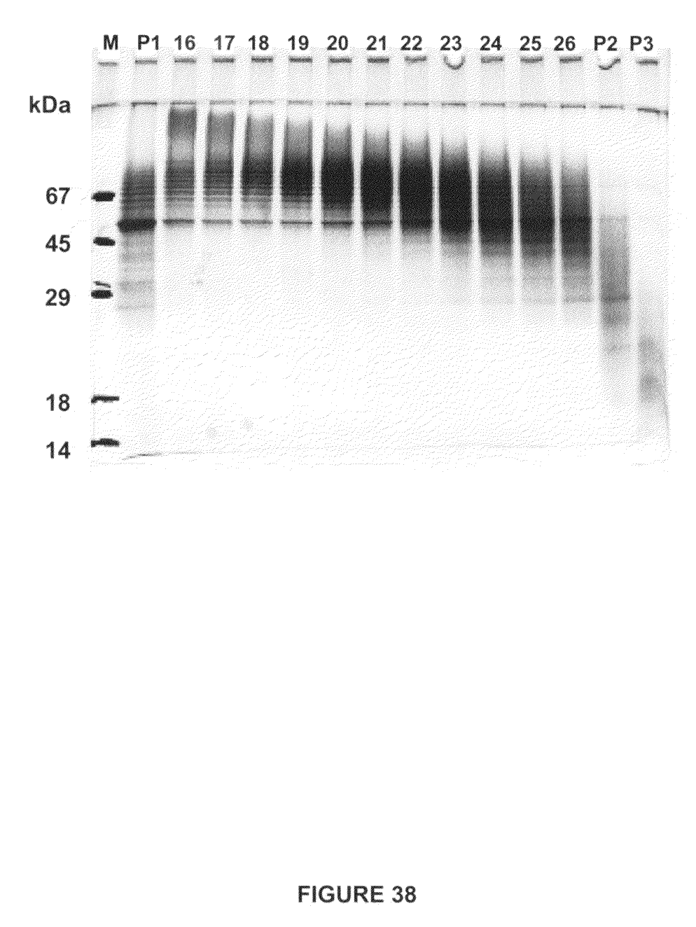

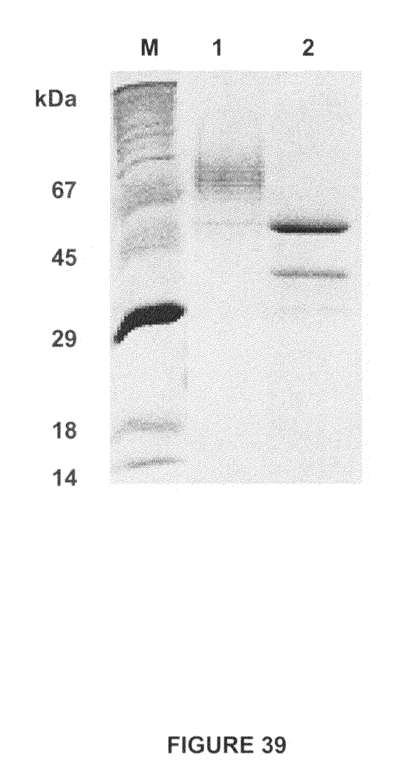

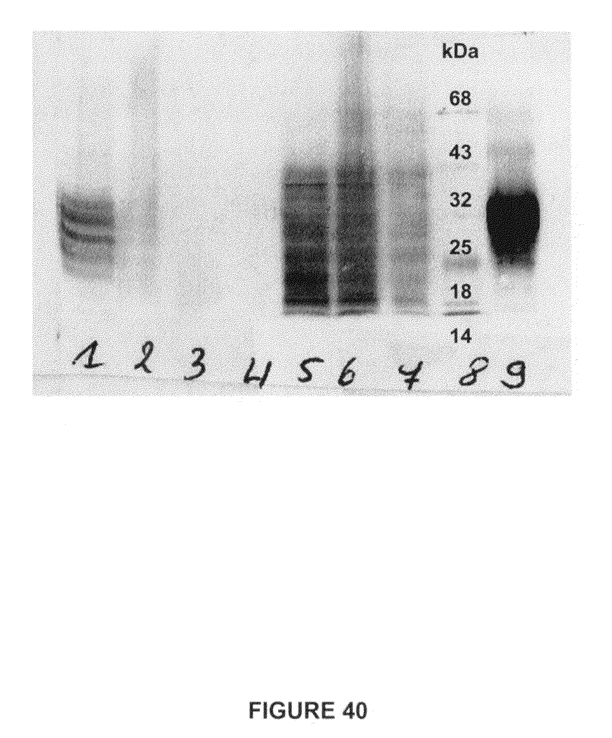

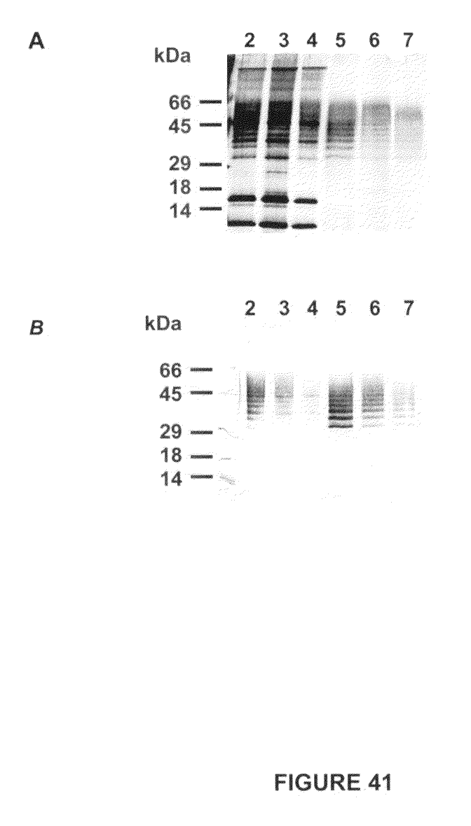

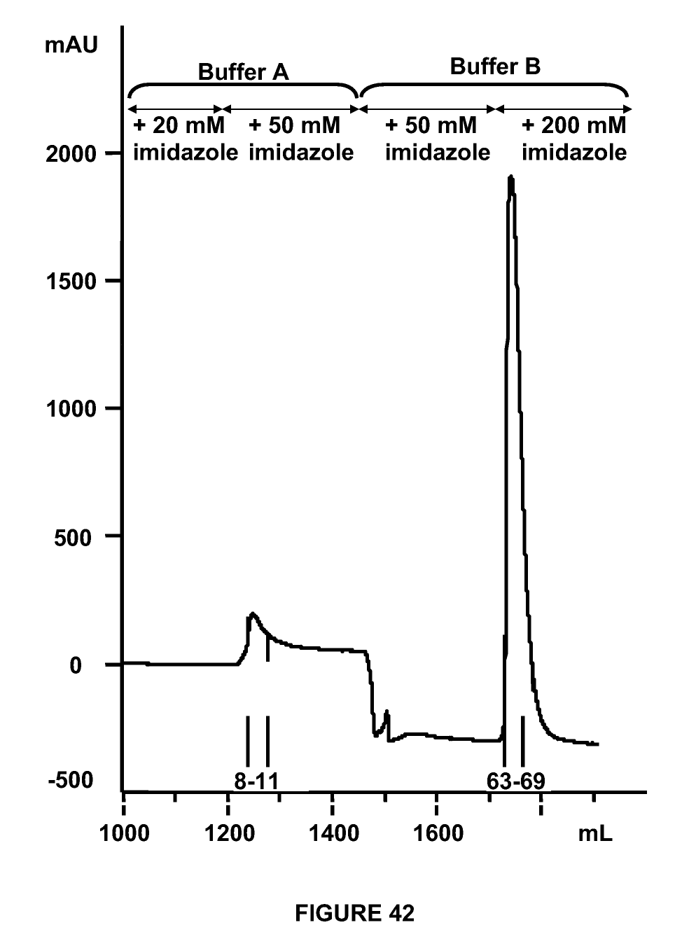

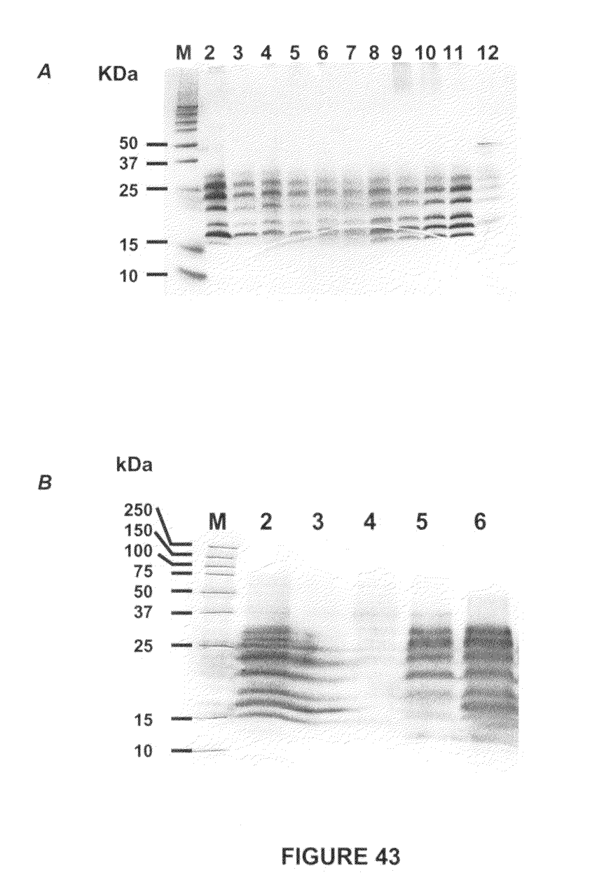

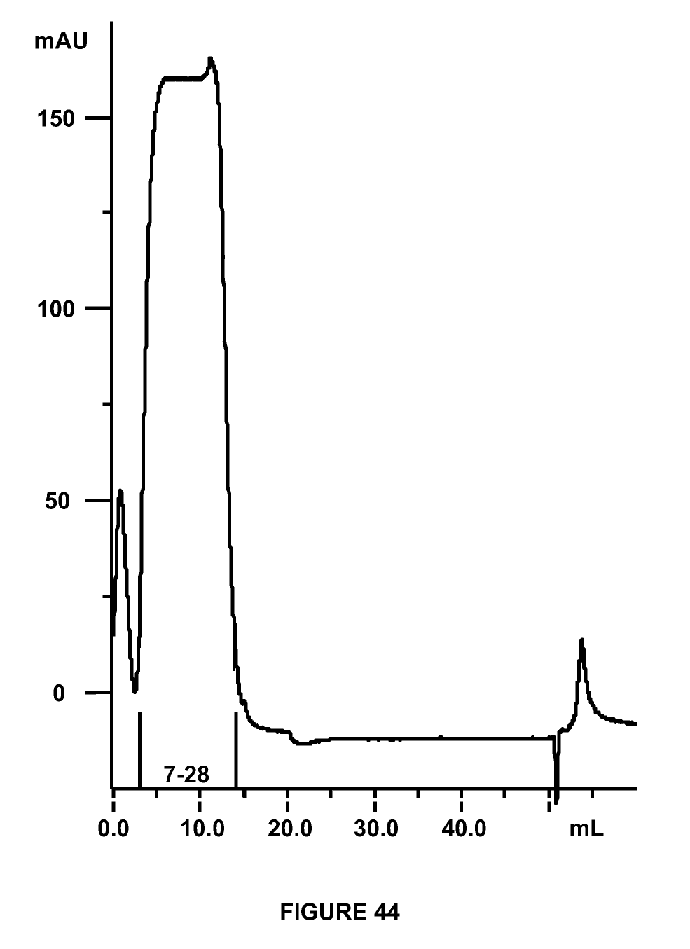

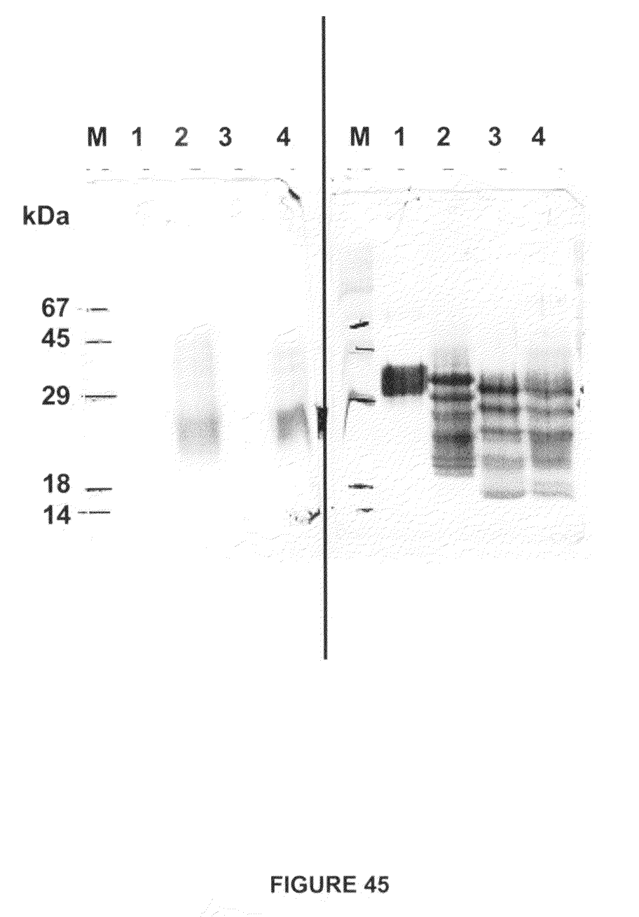

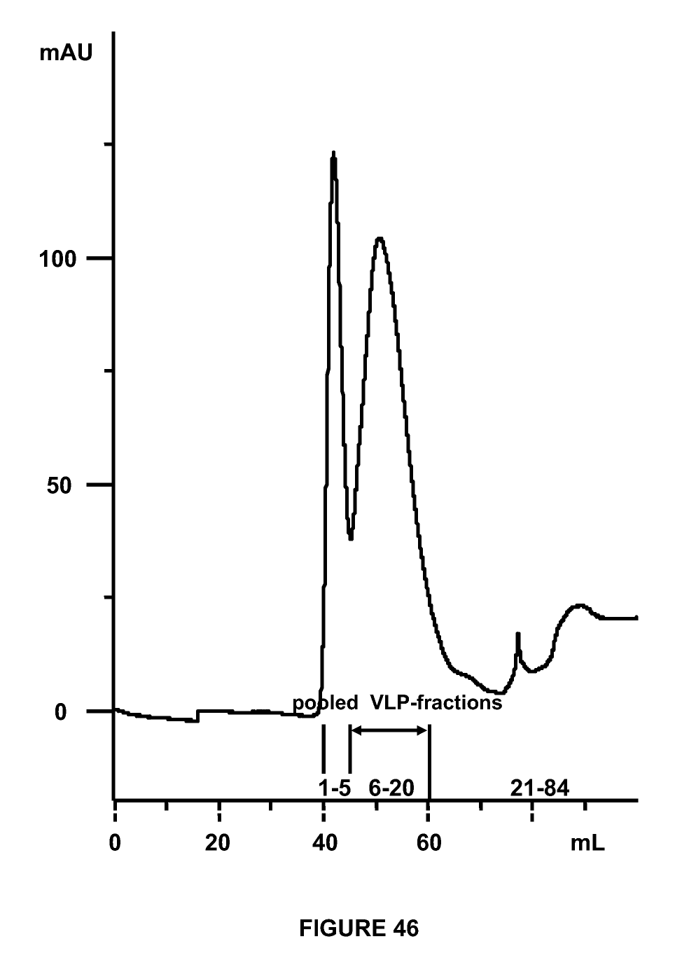

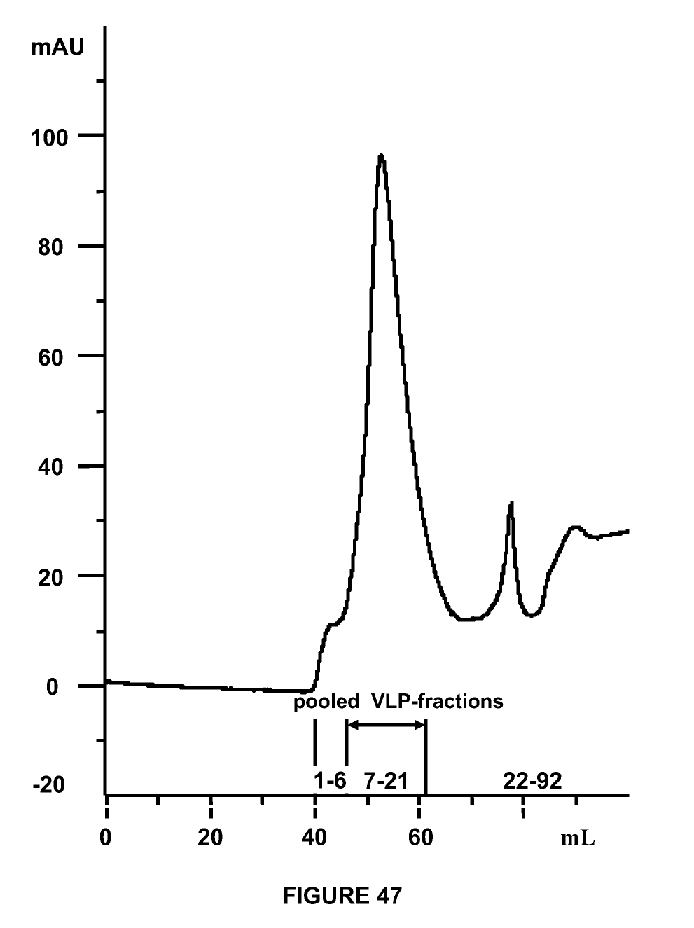

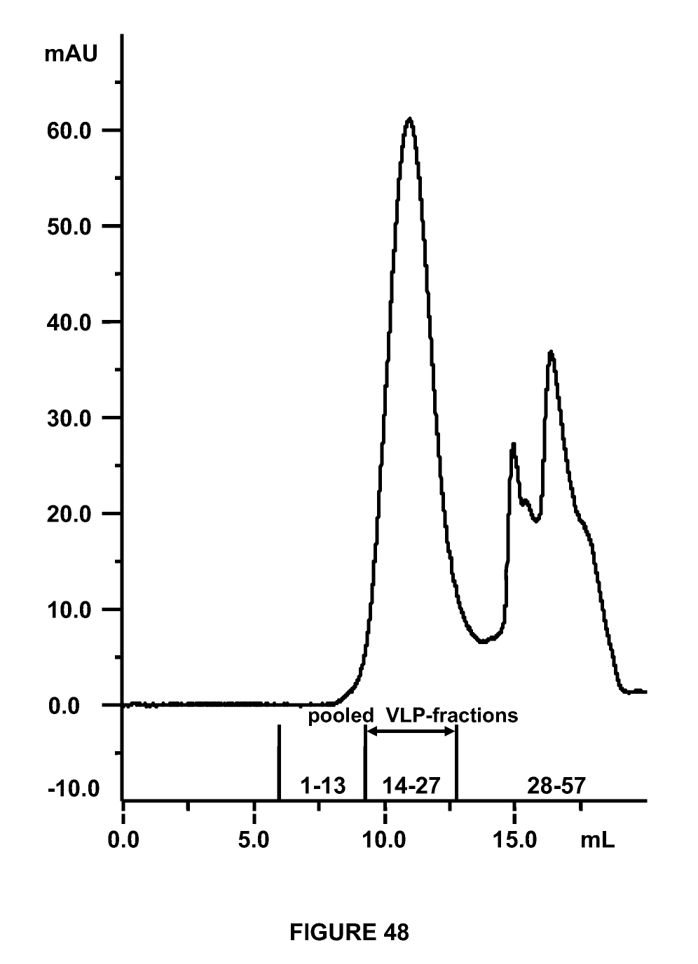

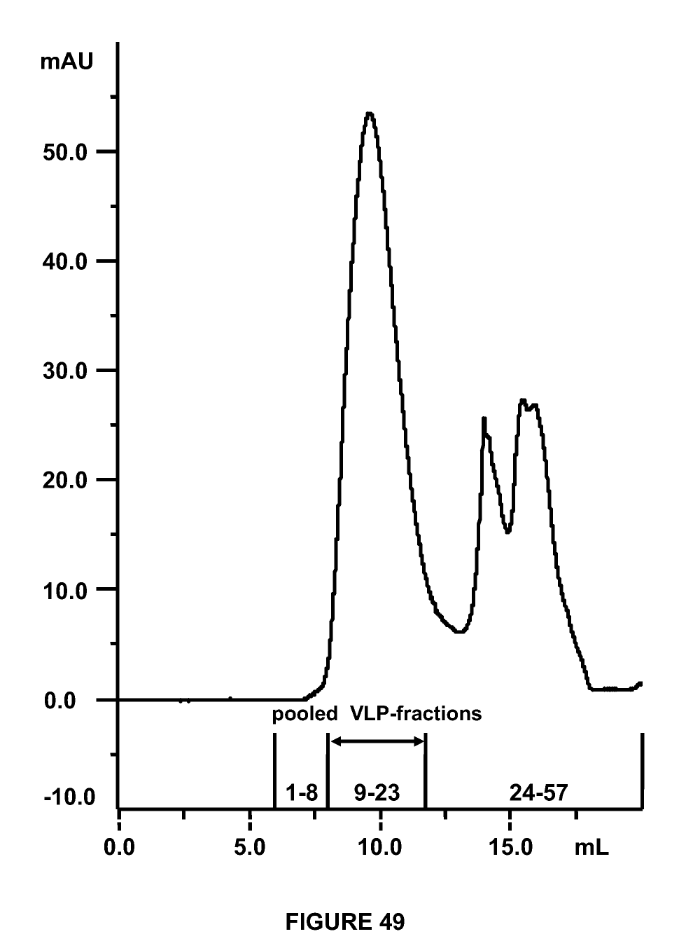

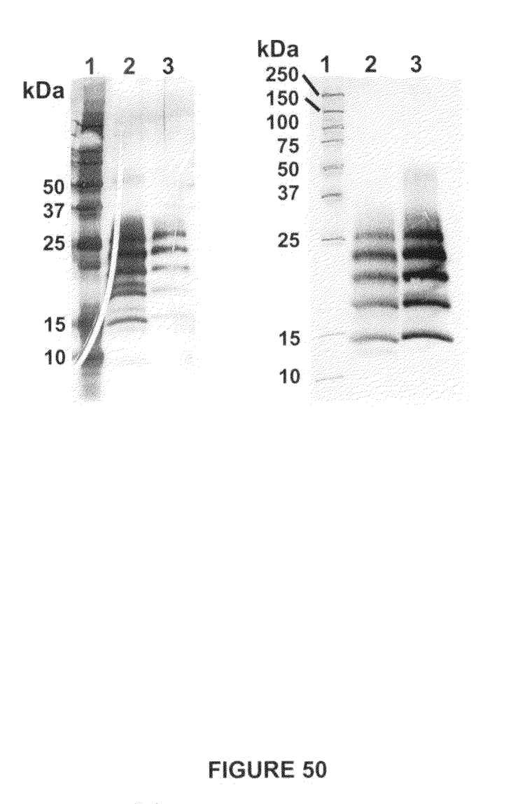

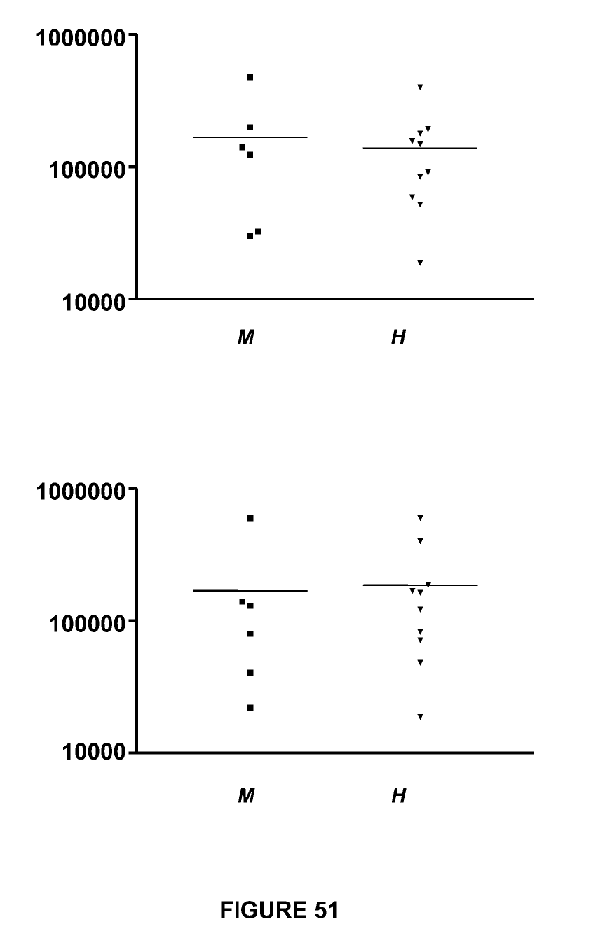

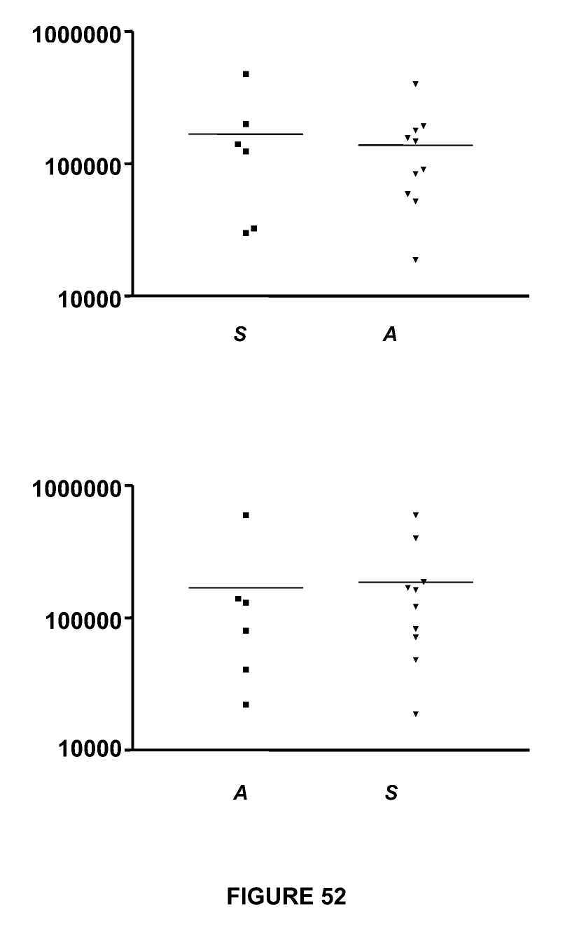

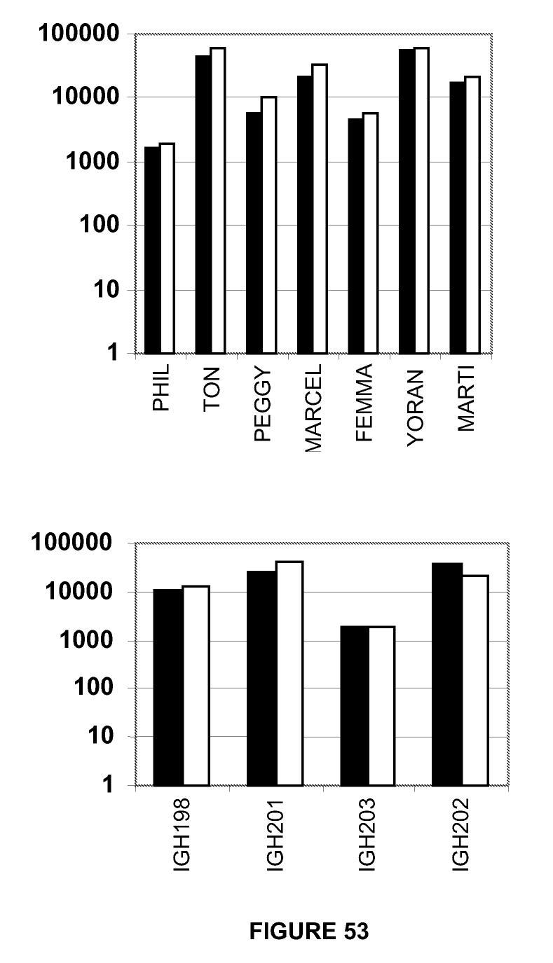

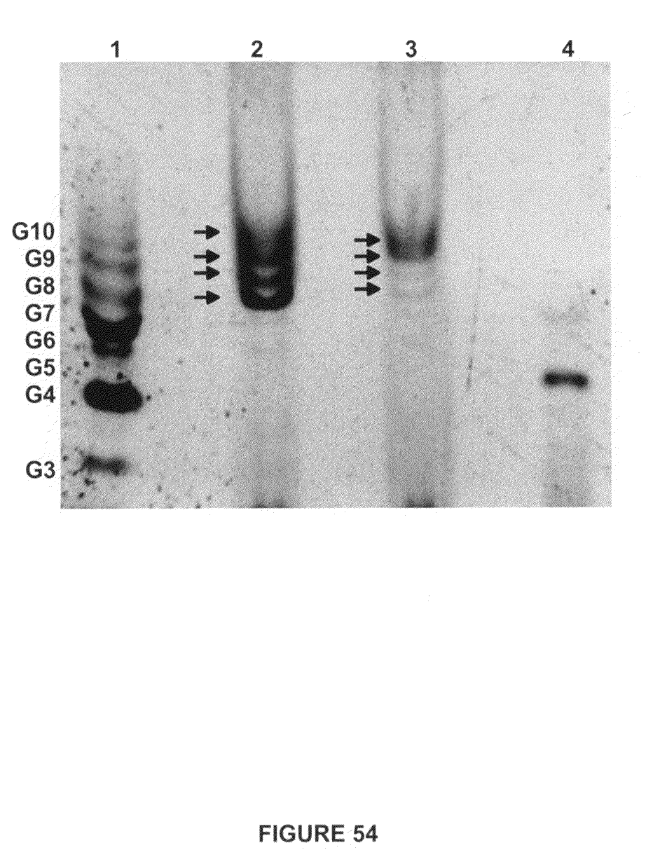

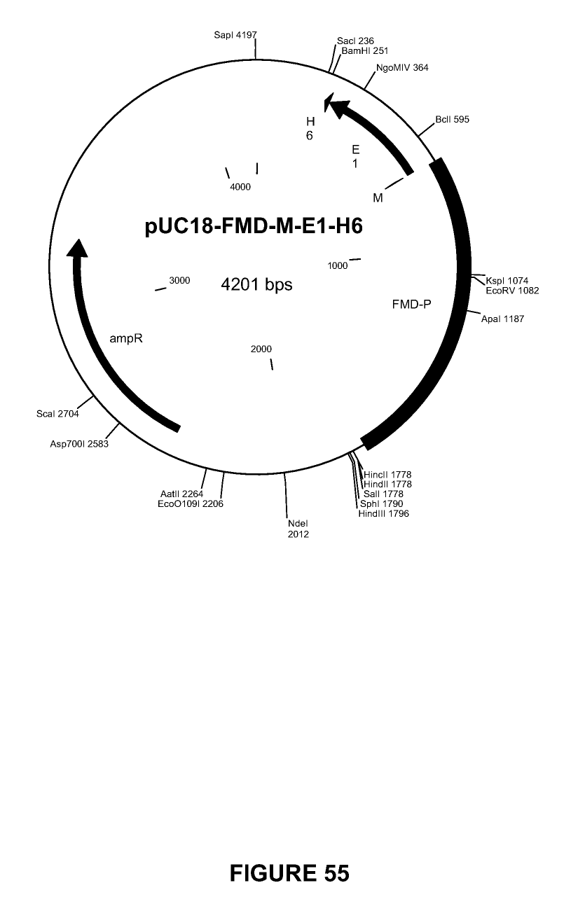

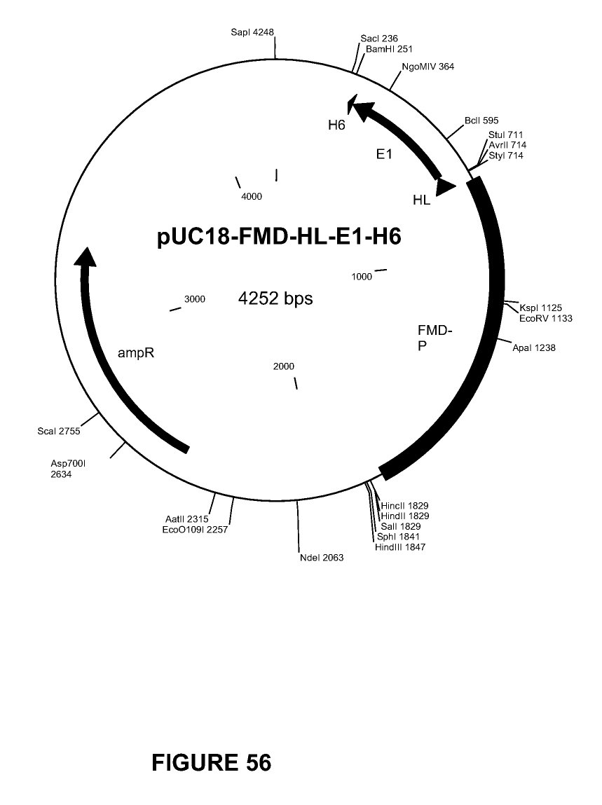

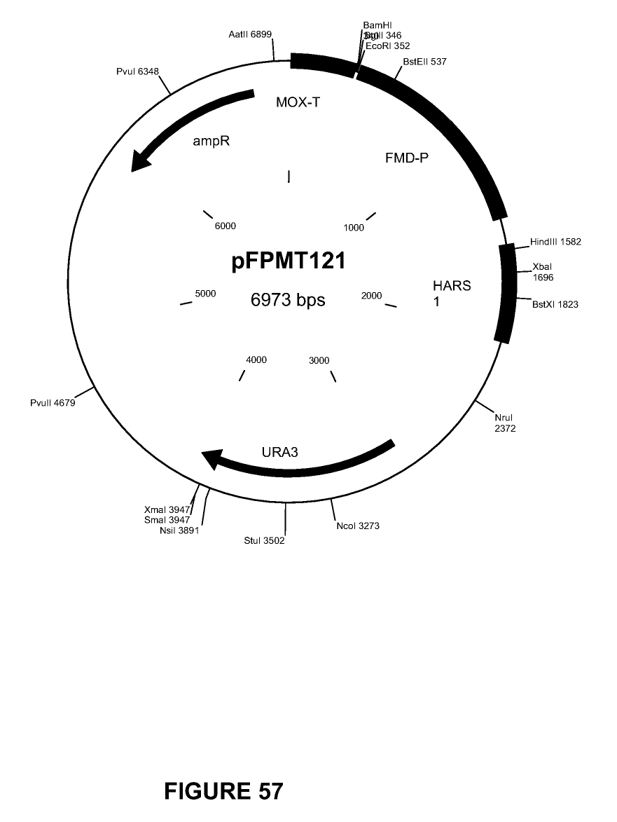

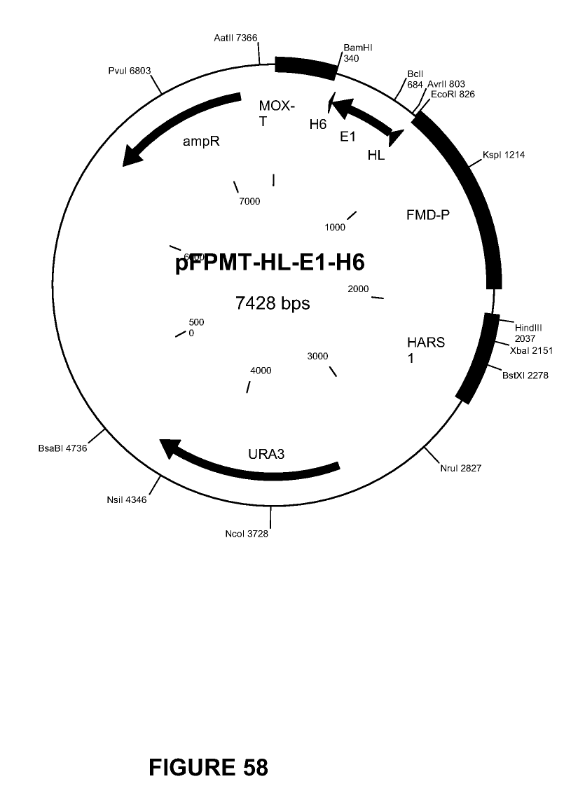

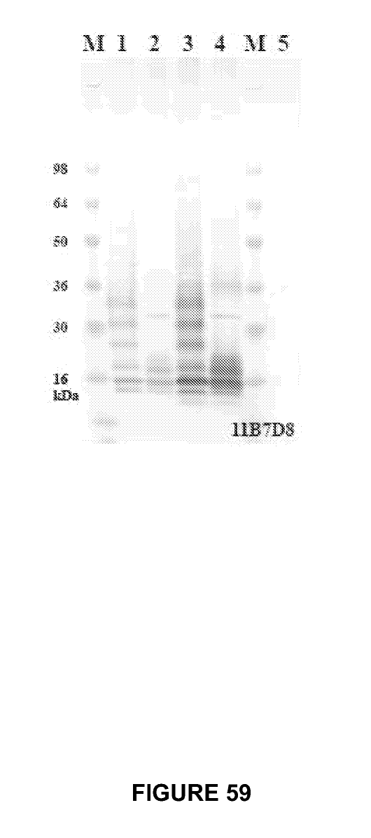

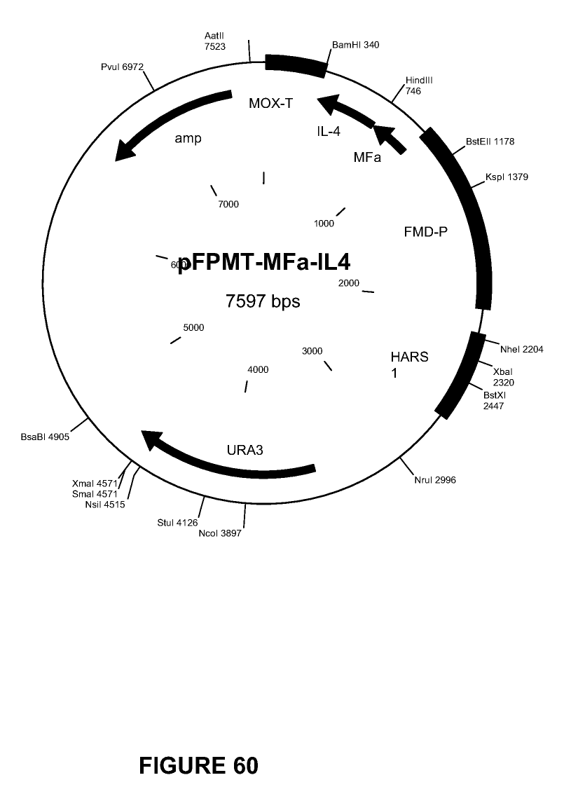

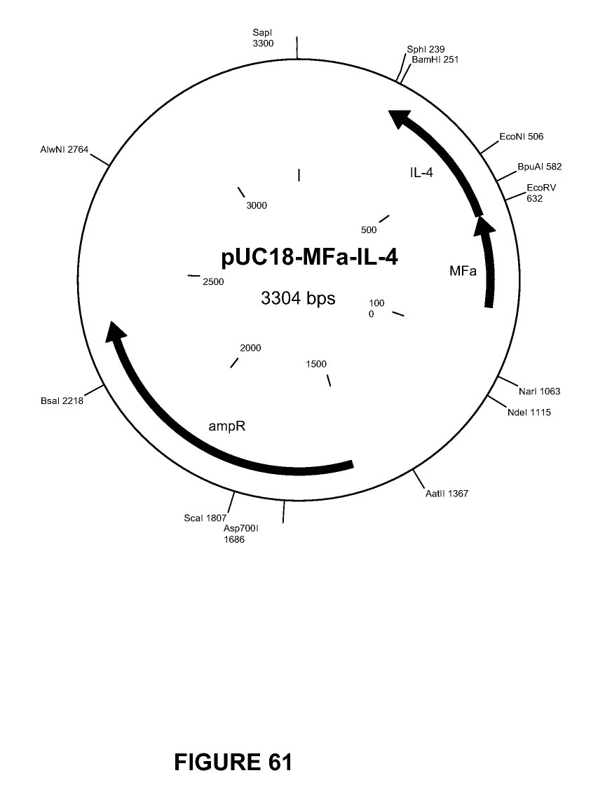

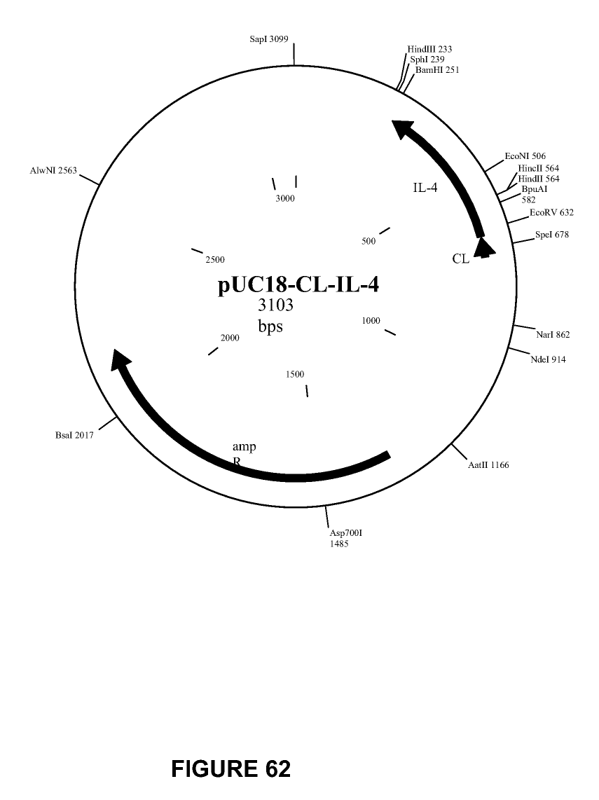

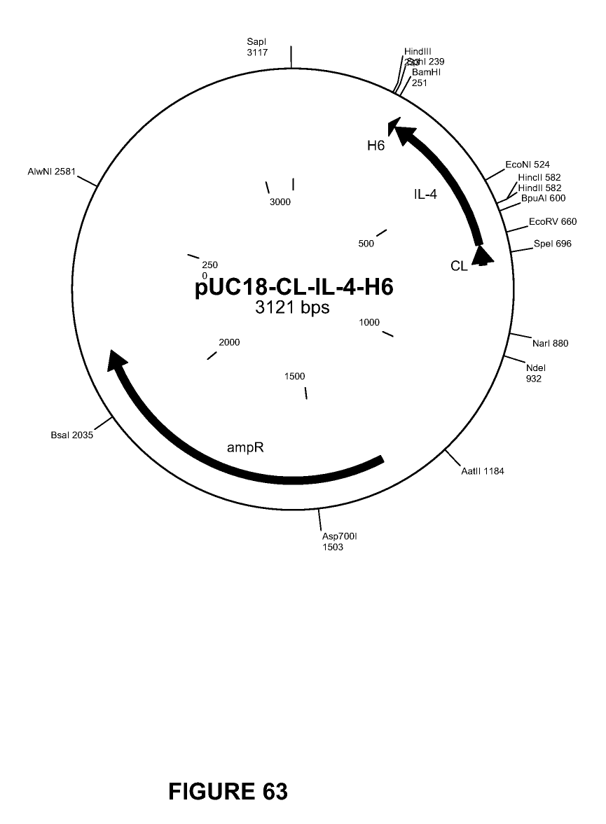

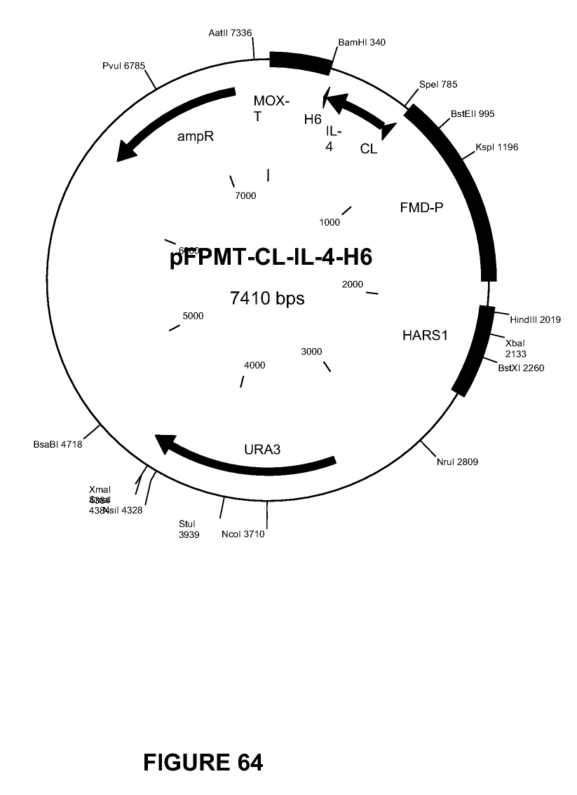

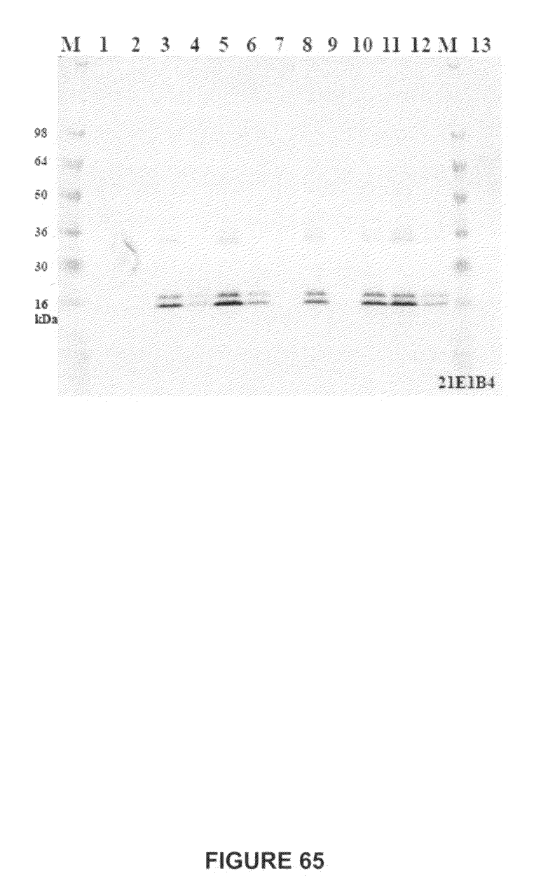

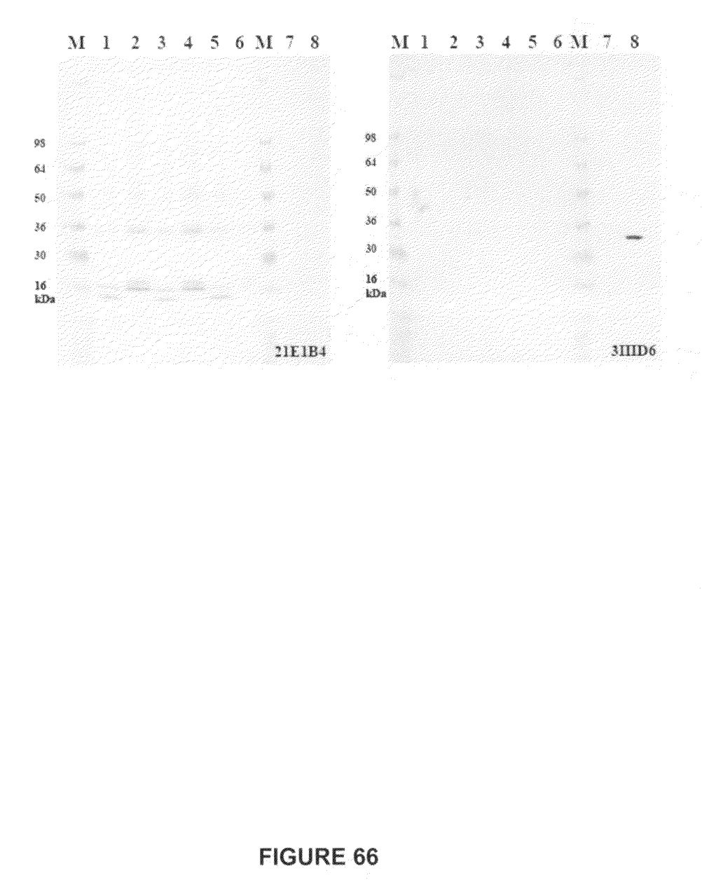

Since at this stage, SDS-PAGE revealed already a very complex picture of protein bands for HCV E2, a further fractionation by size exclusion chromatography was performed. The Ni-IDA eluate was concentrated by ultrafiltration (MWCO 10 kDa, centriplus, Amicon, Millipore) and loaded on Superdex G200 (10/30 or 16/60; Pharmacia) in PBS, 1% Empigen or PBS, 3% Empigen. Elution fractions, containing E2 products, with a Mr between ˜80 kDa and ˜45 kDa, i.e. fractions 17-23 of the elution profile in FIG. 37 based on the migration on SDS-PAGE (FIG. 38), were pooled and alkylated (incubation with 10 mM DTT 3h at RT followed by incubation with 30 mM iodo-acetamide for 3 hours at RT). Samples for amino-terminal sequencing were treated with Endo H (Roche Biochemicals) or left untreated. The glycosylated and deglycosylated E2 products were blotted on PVDF-membranes for amino-terminal sequencing. An amido-black stained blot of glycosylated and deglycosylated E2 is shown in FIG. 39.

The sequencing of both E1 and E2 purified products lead to the disappointing observation that removal of the signal sequence from the HCV envelope proteins is occurring only partially (see Table 3). In addition, the majority of the side products (degradation products and products still containing the leader sequence or part thereof) are glycosylated. This glycosylation resides even in part on the non-cleaved fragment of the signal sequence which contains also an N-glycosylation site. These sites can be mutated in order to result in less glycosylated side products. However, even more problematic is the finding that some alternatively cleaved products have only 1 to 4 amino acids difference compared to the desired intact envelope protein. Consequently, purification of the correctly processed product is virtually impossible due to the lack of sufficiently discriminating biochemical characteristics between the different expression products. Several of the degradation products may be a result of a Kex-2 like cleavage (e.g. the cleavage observed after aa 196 of E1 which is a cleavage after an arginine), which is also required for the cleavage of the α-mating factor leader and which can thus not be blocked without disturbing this essential process.

A high E1 producing clone derived from transformation of S. cerevisiae IYCC155 with pSY1YIG7E1s (SEQ ID NO:50; FIG. 28) was compared with a high producing clone derived from transformation of S. cerevisiae IYCC155 with pSY1aMFE1sH6aYIG1E1s (SEQ ID NO:44; FIG. 22). The intracellular expression of the E1 protein was evaluated after 2 up to 7 days after induction, and this by means of Western-blot using the E1 specific monoclonal antibody (IGH 201). As can be judged from FIG. 40, maximal expression was observed after 2 days for both strains but the expression patterns for both strains are completely different. Expression with the α-mating factor leader results in a very complex pattern of bands, which is a consequence from the fact that the processing of the leader is not efficient. This leads to several expression products with a different amino-terminus and of which some are modified by 1 to 5 N-glycosylations. However, for the E1 expressed with the CL leader a limited number of distinct bands is visible which reflects the high level of correct CL leader removal and the fact that only this correctly processed material may be modified by N-glycosylation (1 to 5 chains), as observed for Hansenula-derived E1 expressed with the same CL leader (see Example 16).

The hybridoma cell line producting the monoclonal antibody directed against E1 (IGH201) was deposited on Mar. 12, 1998 under the conditions of the Budapest Treaty at the European Collection of Cell Cultures, Centre for Applied Microbiology & Research, Salisbury, Wiltshire SP4 0JG, UK, and has the accession number ECACC 98031216. The monoclonal antibody directed against E2 (IGH212) has been described as antibody 12D11F2 in Example 7.4 by Maertens et al. in WO96/04385.

TABLE 3
Identification of N-termini of αMF-E1-H6 and αMF-E2-H6 proteins expressed in
S. cerevisiae or H. polymorpha. Based on the N-terminal sequencing the amount
of N-termini of the mature E1-H6 and E2-H6 proteins could be estimated (“mature”
indicating correct removal of the αMF signal sequence). The total amount of
protein products was calculated as pmol of protein based on the intensity of the
peaks recovered by Edman degradation. Subsequently, for each specific protein
(i.e. for each ‘detected N-terminus’) the mol % versus the total was estimated.

| Yeast | αMF-E1-H6 | αMF-E2-VIEGR-H6 |
|---|---|---|
| S. cerevisiae | Experiment 1: | / |
| | 16% of proteins still containing | |
| | αMF sequences | |
| | 18% of proteins cleaved | |
| | between aa 195 and 196 of E1 | |
| | 66% of proteins with correctly | |
| | removed αMF | |
| | Experiment 2 | / |
| | 18% of proteins still containing | |
| | αMF sequences | |
| | 33% of proteins cleaved | |
| | between aa 195 and 196 of E1 | |
| | 8% of other proteins other E1 | |
| | cleavage products | |
| | 44% of proteins with correctly | |
| | removed αMF | |
| H. polymorpha | 64% of proteins still containing | 75% of proteins still |
| | αMF sequences | containing αMF sequences |
| | 6% of proteins cleaved between | 25% of proteins with |
| | aa 192 and 193 of E1 | correctly removed αMF |
| | 30% of proteins with correctly | |
| | removed αMF | |

### Example 16

### Expression of an E1 Construct in Yeast Suitable for Large Scale Production and Purification

Several other leader sequences were used to replace the S. cerevisiae αMF leader peptide including CHH (leader sequence of Carcinus maenas hyperglycemic hormone), Amy1 (leader sequence of amylase from S. occidentalis), Gam1 (leader sequence of glucoamylase from S. occidentalis), Phy5 (leader sequence from fungal phytase), pho1 (leader sequence from acid phosphatase from Pichia pastoris) and CL (leader of avian lysozyme C, 1,4-beta-N-acetylmuramidase C) and linked to E1-H6 (i.e. E1 with C-terminal his-tag). All constructs were expressed in Hansenula polymorpha and each of the resulting cell lysates was subjected to western blot analysis. This allowed already to conclude that the extent of removal of the leader or signal sequence or peptide was extremely low, except for the construct wherein CL is used as leader peptide. This was confirmed for the CHH-E1-H6 construct by Edman-degradation of Ni-IDA purified material: no correctly cleaved product could be detected although several different sequences were recovered (see Table 4).

TABLE 4
Identification of N-termini of CHH-E1-H6 proteins expressed
in H. polymorpha, based on N-terminal amino acid sequencing of
different protein bands after separation by SDS-PAGE and
blotting to a PVDF membrane.

| Molecular | |
|---|---|
| size | Identified N-termini |
| 45 kD | starts at amino acid 27 of CHH leader = only pre-sequence |
| | cleaved, pro-sequence still attached |
| 26 kD | partially starts at amino acid 1 of CHH leader = no |
| | removal of pre-pro-sequence |
| | partially starts at amino acid 9 of CHH leader = product |
| | of alternative translation starting at second AUG codon |
| 24 kD | partially starts at amino acid 1 of CHH leader = no |
| | removal of pre-pro-sequence |
| | partially starts at amino acid 9 of CHH leader = product |
| | of alternative translation starting at second AUG codon |

As mentioned already, the western-blots of the cell lysates revealed a pattern of E1 specific protein bands, indicative for a higher degree of correct removal of the CL leader peptide. This is surprising since this leader is not derived from a yeast. Amino acid sequencing by Edman degradation of GuHCl solubilized and Ni-IDA purified material indeed confirmed that 84% of the E1 proteins is correctly cleaved and the material is essentially free of degradation products. Still 16% of non-processed material is present but since this material is non-glycosylated it can be easily removed from the mixture allowing specific enrichment of correctly cleaved and glycosylated E1. Such a method for enrichment may be an affinity chromatography on lectins, other alternatives are also given in Example 19. Alternatively, the higher hydrophobic character of the non-glycosylated material may be used to select and optimize other enrichment procedures. The correct removal of the CL leader peptide from the CL-E1-H6 protein was further confirmed by mass spectrometry which also confirmed that up to 4 out of the 5 N-glycosylation sites of genotype 1b E1s can be occupied, whereby the sequence NNSS (amino acids 233 to 236; SEQ ID NO:73) are considered to be a single N-glycosylation site.

### Example 17

### Purification and Biochemical Characterization of the HCV E2 Protein Expressed in Hansenula Polymorpha from the CL-E2-H6 Encoding Construct

The efficiency of removal of the CL leader peptide from CL-E2-VIEGR-H6 (furtheron in this Example denoted as “CL-E2-H6”) protein expressed in Hansenula polymorpha was analyzed. Since the HCV E2s (aa 383-673) was expressed as a his-tagged protein, a rapid and efficient purification of the expressed protein after GuHCl-solubilization of collected cells was performed on Ni-IDA. In brief, cell pellets were resuspended in 30 mM phosphate, 6 M GuHCl, pH 7.2 (9 mL buffer/g cells). The protein was sulfonated overnight at room temperature in the presence of 320 mM (4% w/v) sodium sulfite and 65 mM (2% w/v) sodium tetrathionate. The lysate was cleared after a freeze-thaw cycle by centrifugation (10.000 g, 30 min, 4° C.). Empigen BB (Albright & Wilson) and imidazole were added to a final concentration of 1% (w/v) and 20 mM, respectively. All further chromatographic steps were executed on an Åkta FPLC workstation (Pharmacia). The sample was filtrated through a 0.22 μm pore size membrane (cellulose acetate) and loaded on a Ni-IDA column (Chelating Sepharose FF loaded with Ni²⁺, Pharmacia), which was equilibrated with 50 mM phosphate, 6 M GuHCl, 1% Empigen BB, pH 7.2 (buffer A) supplemented with 20 mM imidazole. The column was washed sequentially with buffer A containing 20 mM and 50 mM imidazole, respectively, till the absorbance at 280 nm reached the baseline level. The his-tagged products were eluted by applying buffer D, 50 mM phosphate, 6 M GuHCl, 0.2% Empigen BB (pH 7.2), 200 mM imidazole. The purified materials were analysed by SDS-PAGE and western-blot using a specific monoclonal antibody directed against E2 (IGH212) (FIG. 41). The IMAC-purified E2-H6 protein was also subjected to N-terminal sequencing by Edman degradation. Thereto proteins were treated with N-glycosidase F (Roche) (0.2 U/μg E2, 1 h incubation at 37° C. in PBS/3% empigen BB) or left untreated. The glycosylated and deglycosylated E2-H6 proteins were subjected to SDS-PAGE and blotted on a PVDF-membrane for amino acid sequencing (analysis was performed on a PROCISE™ 492 protein sequencer, Applied Biosystems). Since at this stage, SDS-PAGE revealed some degradation products, a further fractionation by size exclusion chromatography was performed. Hereto, the Ni-IDA eluate was concentrated by ultrafiltration (MWCO 10 kDa, centriplus, Amicon, Millipore) and loaded on a Superdex G200 (Pharmacia) in PBS, 1% Empigen BB. Elution fractions, containing mainly intact E2s related products with a Mr between ˜30 kDa and ˜70 kDa based on the migration on SDS-PAGE, were pooled and eventually alkylated (incubation with 5 mM DTT for 30 minutes at 37° C., followed by incubation with 20 mM iodoacetamide for 30 minutes at 37° C.). The possible presence of degradation products after IMAC purification can thus be overcome by a further fractionation of the intact product by means of size exclusion chromatography. An unexpectedly good result was obtained. Based on the N-terminal sequencing the amount of E2 product from which the CL leader peptide is removed could be estimated. The total amount of protein products is calculated as pmol of protein based on the intensity of the peaks recovered by Edman degradation. Subsequently, for each specific protein (i.e. for each ‘detected N-terminus’) the mol % versus the total is estimated. In the current experiment, only the correct N-terminus of E2-H6 was detected and other variants of E2-H6 lacking amino acid of the E2 protein or containing N-terminal amino acids not comprised in the E2 protein were absent. In conclusion, the E2-H6 protein expressed by H. polymorpha as CL-E2-H6 protein was isolated without any further in vitro processing as a >95% correctly cleaved protein. This is in sharp contrast with the fidelity of leader peptide removal by H. polymorpha of the αMF-E2-H6 protein to the E2-H6 protein, which was estimated to occur in 25% of the isolated proteins (see Table 3).

### Example 18

### Purification and Biochemical Characterization of the HCV E1 Protein Expressed in Hansenula Polymorpha from the CL-H6-K-E1

### Encoding Construct and In Vitro Processing of H6-Containing Proteins

The efficiency of removal of the CL leader peptide from the CL-H6-K-E1 protein expressed in H. polymorpha was analyzed, as well as the efficiency of subsequent in vitro processing in order to remove the H6 (his-tag)-adaptor peptide and the Endo Lys-C processing site. Since the HCV E1s (aa 192-326) was expressed as a N-terminal His-K-tagged protein CL-H6-K-E1, a rapid and efficient purification could be performed as described in Example 17. The elution profile of the IMAC-chromatographic purification of H6-K-E1 (and possibly residual CL-H6-K-E1) proteins is shown in FIG. 42. After SDS-PAGE and silver staining of the gel and western-blot analysis using a specific monoclonal antibody directed against E1 (IGH201) (FIG. 43), the elution fractions (63-69) containing the recombinant E1s products were pooled (‘IMAC pool’) and subjected to an overnight Endoproteinase Lys-C (Roche) treatment (enzyme/substrate ratio of 1/50 (w/w), 37° C.) in order to remove the H6-K-fusion tail. Removal of non-processed fusion product was performed by a negative IMAC chromatography step on a Ni-IDA column whereby Endo-Lys-C-processed proteins are collected in the flow-through fraction. Hereto the Endoproteinase Lys-C digested protein sample was applied on a Ni-IDA column after a 10-fold dilution with 10 mM NaH₂PO₄.3H₂O, 1% (v/v) Empigen B, pH 7.2 (buffer B) followed by washing with buffer B till the absorbance at 280 nm reached the baseline level. The flow through was collected in different fractions (1-40) that were screened for the presence of E1s-products (FIG. 44). The fractions (7-28), containing intact E1 from which the N-terminal H6-K (and possibly residual CL-H6-K) tail is removed (with a Mr between ˜15 kDa and ˜30 kDa based on the migration on SDS-PAGE followed by silver staining or western blot analysis using a specific monoclonal antibody directed against E1 (IGH201), were pooled and alkylated (incubation with 5 mM DTT for 30 minutes at 37° C., followed by incubation with 20 mM iodoacetamide for 30 minutes at 37° C.).

This material was subjected to N-terminal sequencing (Edman degradation). Hereto, protein samples were treated with N-glycosidase F (Roche) (0.2 U/μg E1, 1 h incubation at 37° C. in PBS/3% empigen BB) or left untreated. The glycosylated and deglycosylated E1 proteins were then separated by SDS-PAGE and blotted on a PVDF-membrane for further analysis by Edman degradation (analysis was performed on a PROCISE 492 protein sequencer, Applied Biosystems). Based on the N-terminal sequencing the amount of correctly processed E1 product could be estimated (processing includes correct cleavage of the H6-K-sequence).

The total amount of protein products is calculated as pmol of protein based on the intensity of the peaks recovered by Edman degradation. Subsequently, for each specific protein (i.e. for each ‘detected N-terminus’) the mol % versus the total is estimated. In the current experiment, only the correct N-terminus of E1 was detected and not the N-termini of other processing variants of H6-K-E1. Based thereon, in vitro processing by Endo Lys-C of the H6-K-E1 E1 (and possibly residual CL-H6-K-E1) protein to the E1 protein was estimated to occur with a fidelity of more than 95%.

### Example 19

### Specific Removal of Low-Glycosylated Forms of HCV E1 by Heparin

In order to find specific purification steps for HCV envelope proteins from yeast cells binding with heparin was evaluated. Heparin is known to bind to several viruses and consequently binding to the HCV envelope has already been suggested (Garson, J. A. et al. 1999b). In order to analyze this potential binding, heparin was biotinylated and interaction with HCV E1 analyzed in microtiterplates coated with either sulfonated HCV E1 from H. polymorpha, alkylated HCV E1 from H. polymorpha (both produced as described in Example 16) and alkylated HCV E1 from a culture of mammalian cells transfected with a vaccinia expression vector. Surprisingly, a strong binding could only be observed with sulfonated HCV E1 from H. polymorpha, while binding with HCV E1 from mammalian cell culture was completely absent. By means of western-blot we could show that this binding was specific for the lower molecular weight bands of the HCV E1 protein mixture (FIG. 45), corresponding to low-glycosylated mature HCV E1s. FIG. 45 also reveals that sulfonation is not essential for heparin binding since upon removal of this sulfonation binding is still observed for the low molecular weight E1 (lane 4). Alternatively, alkylation is reducing this binding substantially, however, this may be caused by the specific alkylation agent (iodo-acetamide) used in this example. This finding further demonstrated the industrial applicability of the CL-HCV-envelope expression cassettes for yeast since we specifically can enrich HCV E1 preparations towards a preparation with HCV E1 proteins with a higher degree of glycosylation (i.e. more glycosylation sites occupied).

### Example 20

### Formation and Analysis of Virus-Like Particles (VLPS)

Conversion of the HCV E1 and E2 envelope proteins expressed in H. polymorpha (Examples 16 to 18) to VLPs was done essentially as described by Depla et al. in WO99/67285 and by Bosman et al. in WO01/30815. Briefly, after cultivation of the transformed H. polymorpha cells during which the HCV envelope proteins were expressed, cells were harvested, lysed in GuHCl and sulphonated as described in Example 17. His-tagged proteins were subsequently purified by IMAC and concentrated by ultrafiltration as described in Example 17.

### VLP-Formation of HCV Envelope Proteins with Sulphonated Cys-Thiol Groups

The concentrated HCV envelope proteins sulphonated during the isolation procedure were not subjected to a reducing treatment and loaded on a size-exclusion chromatography column (Superdex G200, Pharmacia) equilibrated with PBS, 1% (v/v) Empigen. The eluted fractions were analyzed by SDS-PAGE and western blotting. The fractions with a relative Mr ˜29-˜15 kD (based on SDS-PAGE migration) were pooled, concentrated and loaded on Superdex G200, equilibrated with PBS, 3% (w/v) betain, to enforce virus like particle formation (VLP). The fractions were pooled, concentrated and desalted to PBS, 0.5% (w/v) betain.

### VLP-Formation of HCV Envelope Proteins with Irreversibly Modified Cys-Thiol Groups

The concentrated HCV envelope proteins sulphonated during the isolation procedure were subjected to a reducing treatment (incubation in the presence of 5 mM DTT in PBS) to convert the sulphonated Cys-thiol groups to free Cys-thiol groups. Irreversible Cys-thiol modification was performed by (i) incubation for 30 min in the presence of 20 mM iodoacetamide, or by (ii) incubation for 30 min in the presence of 5 mM N-ethylmaleimide (NEM) and 15 mM biotin-N-ethylmaleimide. The proteins were subsequently loaded on a size-exclusion chromatography column (Superdex G200, Pharmacia) equilibrated with PBS, 1% (v/v) Empigen in case of iodoacetamide-blocking, or with PBS, 0.2% CHAPS in case of blocking with NEM and biotin-NEM. The eluted fractions were analyzed by SDS-PAGE and Western blotting. The fractions with a relative Mr ˜29-˜15 kD (based on SDS-PAGE migration) were pooled, concentrated and, to force virus-like particle formation, loaded on a Superdex G200 column equilibrated with PBS, 3% (w/v) betain. The fractions were pooled, concentrated and desalted to PBS, 0.5% (w/v) betain in case of iodoacetamide-blocking, or with PBS, 0.05% CHAPS in case of blocking with NEM and biotin-NEM.

### VLP-Formation of HCV Envelope Proteins with Reversibly Modified Cys-Thiol Groups

The concentrated HCV envelope proteins sulphonated during the isolation procedure were subjected to a reducing treatment (incubation in the presence of 5 mM DTT in PBS) to convert the sulphonated Cys-thiol groups to free Cys-thiol groups. Reversible Cys-thiol modification was performed by incubation for 30 min in the presence of dithiodipyridine (DTDP), dithiocarbamate (DTC) or cysteine. The proteins were subsequently loaded on a size-exclusion chromatography column (Superdex G200, Pharmacia) equilibrated with PBS, 1% (v/v) Empigen. The eluted fractions were analyzed by SDS-PAGE and Western blotting. The fractions with a relative Mr ˜29-˜15 kD (based on SDS-PAGE migration) were pooled, concentrated and loaded on Superdex G200, equilibrated with PBS, 3% (w/v) betain, to enforce virus like particle formation (VLP). The fractions were pooled, concentrated and desalted to PBS, 0.5% (w/v) betain.

The elution profiles of size-exclusion chromatography in PBS, 3% (w/v) betain to obtain VLPs of H. polymorpha-expressed E2-H6 are shown in FIG. 46 (sulphonated) and FIG. 47 (alkylated with iodoacetamide).

The elution profiles of size-exclusion chromatography in PBS, 3% (w/v) betain to obtain VLPs of H. polymorpha-expressed E1 are shown in FIG. 48 (sulphonated) and FIG. 49 (alkylated with iodoacetamide). The resulting VLPs were analyzed by SDS-PAGE and western blotting as shown in FIG. 50.

### Size-Analysis of VLPS Formed by H. polymorpha-Expressed HCV Envelope Proteins

The VLP particle size was determined by Dynamic Light Scattering. For the light-scattering experiments, a particle-size analyzer (Model Zetasizer 1000 HS, Malvern Instruments Ltd., Malvern, Worcester UK) was used which was controlled by photon correlation spectroscopy (PCS) software. Photon correlation spectroscopy or dynamic light scattering (DLS) is an optical method that measures brownian motion and relates this to the size of particles. Light from a continuous, visible laser beam is directed through an ensemble of macromolecules or particles in suspension and moving under brownian motion. Some of the laser light is scattered by the particles and this scattered light is measured by a photomultiplier. Fluctuations in the intensity of scattered light are converted into electrical pulses which are fed into a correlator. This generates the autocorrelation function which is passed to a computer where the appropriate data analysis is performed. The laser used was a 10 mW monochromatic coherent He—Ne laser with a fixed wavelength of 633 nm. For each sample, three to six consecutive measurements were taken.

The results of these experiments are summarized in Table 5.

TABLE 5
Results of dynamic light scattering analysis on the
indicated VLP-compositions of HCV envelope proteins
expressed by H. polymorpha. The VLP particle sizes
are given as mean diameter of the particles.

| | | | |
|---|---|---|---|
| Cys-thiol | | | |
| modification | E1-H6 | E2-VIEGR-H6 | E1 |

| | | | | |
|---|---|---|---|---|
| sulphonation | 25-45 nm | 20 | nm | 20-26 nm |
| alkylation | 23-56 nm | 20-56 | nm | 21-25 nm |
| (iodoacetamide) | | | | |

The observation that sulphonated HCV E1 derived from H. polymorpha still forms particles with a size in the same range as alkylated HCV E1 from Hansenula is surprising. Such an effect was not expected since the high (up to 8 Cys-thiol groups can be modified on HCV E1) net increase of negative charges as a consequence of sulphonation should induce an ionic repulsion between the subunits. The other reversible cysteine modifying agents tested also allowed particle formation, the HCV E1 produced in this way, however, proved to be less stable than the sulphonated material, resulting in disulfide-based aggregation of the HCV E1. In order to use these other reversible blockers, further optimization of the conditions is required.

### Example 21

### Antigenic Equivalence of Hansenula-Produced HCV E1-H6 and HCV E1 Produced by Vaccinia-Infected Mammalian Cells

The reactivity of Hansenula-produced HCV E1-H6 with sera from HCV chronic carriers was compared to the reactivity of HCV E1 produced by HCV-recombinant vaccinia virus-infected mammalian cells as described by Depla et al. in WO 99/67285. Both HCV-E1 preparations tested consisted of VLP's wherein the HCV E1 proteins were alkylated with NEM and biotin-NEM.

The reactivities of both HCV E1 VLP-preparations with sera from HCV chronic carriers was determined by ELISA. The results are summarized in Table 6. As can be derived from Table 6, no differences in reactivity were noted between HCV E1 expressed in HCV-recombinant vaccinia virus-infected mammalian cells and HCV E1 expressed in H. polymorpha.

TABLE 6
Antigenicity of E1 produced in a mammalian cell culture or
produced in H. polymorpha were evaluated on a panel of sera
from human HCV chronic carriers. For this purpose biotinylated
E1 was bound to streptavidin coated ELISA plates. Thereafter
human sera were added at a 1/20 dilution and bound
immunoglobulins from the sera bound to E1 were detected with
a rabbit-anti-human IgG-Fc specific secondary antibody labeled
with peroxidase. Results are expressed as OD-values. The
average values are the averages of the OD-values
of all serum samples tested.

| Serum | Hansenula | mammalian |
|---|---|---|
| 17766 | 1.218 | 1.159 |
| 17767 | 1.513 | 1.363 |
| 17777 | 0.806 | 0.626 |
| 17784 | 1.592 | 1.527 |
| 17785 | 1.508 | 1.439 |
| 17794 | 1.724 | 1.597 |
| 17798 | 1.132 | 0.989 |
| 17801 | 1.636 | 1.504 |
| 17805 | 1.053 | 0.944 |
| 17810 | 1.134 | 0.999 |
| 17819 | 1.404 | 1.24 |
| 17820 | 1.308 | 1.4 |
| 17826 | 1.163 | 1.009 |
| 17827 | 1.668 | 1.652 |
| 17849 | 1.595 | 1.317 |
| 55333 | 1.217 | 1.168 |
| 55337 | 1.591 | 1.416 |
| 55348 | 1.392 | 1.261 |
| 55340 | 1.202 | 0.959 |
| 55342 | 1.599 | 1.477 |
| 55345 | 1.266 | 1.428 |
| 55349 | 1.329 | 1.137 |
| 55350 | 1.486 | 1.422 |
| 55352 | 0.722 | 1.329 |
| 55353 | 1.065 | 1.157 |
| 55354 | 1.118 | 1.092 |
| 55355 | 0.754 | 0.677 |
| 55362 | 1.43 | 1.349 |
| 55365 | 1.612 | 1.608 |
| 55368 | 0.972 | 0.959 |
| 55369 | 1.506 | 1.377 |
| average | 1.313 | 1.245 |

### Example 22

### Immunogenic Equivalence of Hansenula-Produced HCV E1-H6 and HCV E1 Produced by Vaccinia-Infected Mammalian Cells

The immunogenecity of Hansenula-produced HCV E1-H6 was compared to the immunogenecity of HCV E1 produced by HCV-recombinant vaccinia virus-infected mammalian cells as described by Depla et al. in WO99/67285. Both HCV-E1 preparations tested consisted of VLP's wherein the HCV E1 proteins were alkylated with iodoacetamide. Both VLP preparations were formulated with alum and injected in Balb/c mice (3 intramuscular/subcutaneous injections with a three week interval between each and each consisting of 5 μg E1 in 125 μl containing 0.13% Alhydrogel, Superfos, Denmark). Mice were bled ten days after the third immunization.

Results of this experiment are shown in FIG. 51. For the top part of FIG. 51, antibodies raised following immunization with VLPs of E1 produced in mammalian cells were determined. Antibody titers were determined by ELISA (see Example 21) wherein either E1 produced in mammalian cells (“M”) or Hansenula-produced E1 (“H”) were coated directly on the ELISA solid support whereafter the ELISA plates were blocked with casein. For the bottom part of FIG. 51, antibodies raised following immunization with VLPs of Hansenula-produced E1 were determined. Antibody titers were determined by ELISA (see Example 21) wherein either E1 produced in mammalian cells (“M”) or Hansenula-produced E1 (“H”) were coated directly on the ELISA solid support whereafter the ELISA plates were blocked with casein.

The antibody titers determined were end point titers. The end point titer is determined as the dilution of serum resulting in an OD (as determined by ELISA) equal to two times the mean of the background of the assay.

FIG. 51 shows that no significant differences were observed between the immunogenic properties of both E1-compositions and that the determined antibody titers are independent of the antigen used in the ELISA to perform the end point titration.

The yeast-derived HCV E1 induced upon vaccination a protective response similar to the protective response obtained upon vaccination with alkylated HCV E1 derived from mammalian cell culture. The latter response was able to prevent chronic evolution of HCV after an acute infection.

### Example 23

### Antigenic and Immunogenic Profile of Hansenula-Produced HCV E1-H6 which is Sulphonated

The reactivity of Hansenula-produced HCV E1-H6 with sera from HCV chronic carriers was compared to the reactivity of HCV E1 produced by HCV-recombinant vaccinia virus-infected mammalian cells as described by Depla et al. in WO99/67285. Both HCV-E1 preparations tested consisted of VLP's wherein the Hansenula-produced HCV E1 proteins were sulphonated and the HCV E1 produced by mammalian cells was alkylated. The results are given in Table 7. Although the overall (average) reactivity was identical, some major differences were noted for individual sera. This implies that the sulphonated material presents at least some of its epitopes in a way different from alkylated HCV E1.

The immunogenecity of Hansenula-produced HCV E1-H6 which was sulphonated was compared to the immunogenecity of Hansenula-produced HCV E1-H6 which was alkylated. Both HCV-E1 preparations tested consisted of VLP's. Both VLP preparations were formulated with alum and injected in Balb/c mice (3 intramuscular/subcutaneous injections with a three week interval between each and each consisting of 5 μg E1 in 125 μl containing 0.13% Alhydrogel, Superfos, Denmark). Mice were bled ten days after the third immunization.

Antibody titers were determined similarly as described in Example 22. Surprisingly, immunization with sulphonated material resulted in higher antibody titers, regardless of the antigen used in ELISA to assess these titers (FIG. 51; top panel: titration of antibodies raised against alkylated E1; bottom panel: titration of antibodies raised against sulphonated E1; “A”: alkylated E1 coated on ELISA plate; “S”: sulphonated E1 coated on ELISA plate). However, in this experiment individual titers are different dependent on the antigen used for analysis which confirms the observation noted with sera from HCV patients. Consequently, HCV E1 wherein the cysteine thiol-groups are modified in a reversible way may be more immunogenic and thus have an increased potency as a vaccine protecting against HCV (chronic infection). In addition thereto, induction of a response to neo-epitopes induced by irreversible blocking is less likely to occur.

TABLE 7
Antigenicity of alkylated E1 (produced in mammalian cell
culture) or sulphonated E1-H6 (produced in H. polymorpha) was
evaluated on a panel of sera from human HCV chronic carriers
(“patient sera”) and a panel of control sera (“blood donor sera”).
To this purpose E1 was bound to ELISA plates, after which the
plates were further saturated with casein. Human sera were added
at a 1/20 dilution and bound immunoglobulins were detected with
a rabbit-anti-human IgG-Fc specific secondary antibody labeled
with peroxidase. Results are expressed as OD-values. The average
values are the averages of the OD-values of all serum samples tested.

| | | |
|---|---|---|
| sernr | Hansenula | mammalian |

patient sera

| | | |
|---|---|---|
| 17766 | 0.646 | 0.333 |
| 17777 | 0.46 | 0.447 |
| 17785 | 0.74 | 0.417 |
| 17794 | 1.446 | 1.487 |
| 17801 | 0.71 | 0.902 |
| 17819 | 0.312 | 0.539 |
| 17827 | 1.596 | 1.576 |
| 17849 | 0.586 | 0.964 |
| 55333 | 0.69 | 0.534 |
| 55338 | 0.461 | 0.233 |
| 55340 | 0.106 | 0.084 |
| 55345 | 1.474 | 1.258 |
| 55352 | 1.008 | 0.668 |
| 55355 | 0.453 | 0.444 |
| 55362 | 0.362 | 0.717 |
| 55369 | 0.24 | 0.452 |
| average | 0.706 | 0.691 |

blood donor sera

| | | |
|---|---|---|
| F500 | 0.055 | 0.054 |
| F504 | 0.05 | 0.05 |
| F508 | 0.05 | 0.054 |
| F510 | 0.05 | 0.058 |
| F511 | 0.05 | 0.051 |
| F512 | 0.051 | 0.057 |
| F513 | 0.051 | 0.052 |
| F527 | 0.057 | 0.054 |
| average | 0.052 | 0.054 |

### Example 24

### Identical Antigenic Reactivity of Hansenula-Produced HCV E1-H6 and HCV E1 Produced by Vaccinia-Infected Mammalian Cells with SERA from Vaccinated Chimpanzees

The reactivities of the E1 produced by HCV-recombinant vaccinia virus-infected mammalian cells and the E1-H6 produced by Hansenula (both alkylated) with sera from vaccinated chimpanzees and with monoclonal antibodies were compared. Thereto, said E1 proteins were coated directly to ELISA plates followed by saturation of the plates with casein. The end point titers of antibodies binding the E1 proteins coated to the ELISA plates was determined for chimpanzee sera and for specific murine monoclonal antibodies, all obtained from animals immunized with E1 produced by mammalian cells. End point titer determination was done as described in Example 22. The murine monoclonal antibodies used were IGH201 (see Example 15), IGH198 (IGH198=23C12 in Maertens et al. in WO96/04385), IGH203 (IGH203=15G6 in Maertens et al. in WO96/04385) and IGH202 (IGH202=3F3 in Maertens et al. in WO99/50301).

As can be derived from FIG. 53, the reactivities of 7 different chimpanzee are identical when tested with E1 protein produced by either Hansenula or mammalian cells. The reactivities of the monoclonal antibodies against HCV E1 are also almost equal. Two of the chimpanzees (Yoran and Marti) were involved in a prophylactic vaccine study and were able to clear an acute infection upon challenge while a control animal did not clear the infection. The five other chimpanzees (Ton, Phil, Marcel, Peggy, Femma) were involved in therapeutic vaccination studies and showed a reduction in liver damage, as measured by ALT in serum and/or histological activity index on liver biopsy, upon the HCV E1 immunizations.

The results obtained in this experiment are clearly different from the findings of Mustilli and coworkers (Mustilli, A. C. et al. 1999a) who expressed the HCV E2 protein both in Saccharomyces cerevisiae and Kluyveromyces lactis. The purified yeast-produced E2 was, however, different from the HCV E2 produced by mammalian (CHO) cells in that a lower reactivity was observed with sera from chimpanzees immunized with HCV E2 produced by mammalian cells while reactivity with monoclonal antibodies was higher for the yeast-produced HCV E2.

### Example 25

### Glycoprofiling of HCV E1 by Fluorophore-Assisted Carbohydrate Electrophoresis (FACE)

The glycosylation profiles were compared of Hansenula-produced HCV E1 and HCV E1 produced by HCV-recombinant vaccinia virus-infected mammalian cells as described by Depla et al. in WO99/67285. This was done by means of fluorophore-assisted carbohydrate electrophoresis (FACE). Thereto, oligosaccharides were released from E1s produced by mammalian cells or Hansenula by peptide-N-glycosidase (PNGase F) and labelled with ANTS (the E1 proteins were alkylated with iodoacetamide prior to PNGase F digestion). ANTS-labeled oligosaccharides were separated by PAGE on a 21% polyacrylamide gel at a current of 15 mA at 4° C. for 2-3 h. From FIG. 54, it was concluded that the oligosaccharides on E1 produced by mammalian cells and E1-H6 produced by Hansenula migrate like oligomaltose with a degree of polymerization between 7 and 11 monosaccharides. This indicates that the Hansenula expression system surprisingly leads to an E1 protein which is not hyperglycosylated and which has sugar chains with a length similar to the sugar chains added to E1 proteins produced in mammalian cells.

### Example 26

### Construction OF pFPMT-HL-E1-H6 Shuttle Vector

The pFPMT-HL-E1-H6 shuttle vector for transformation of Hansenula polymorpha has been constructed in a multi-step procedure.

### Generation of pUC18-FMD-HL-E1-H6

The HL-E1-H6 DNA fragment gene (SEQ ID NO:100; coding for HCV type 1b E1s protein consisting of the amino acids 192 to 326 of E1s elongated with 6 His residues and upstream flanked by the human lysozyme signal peptide [HL]) was constructed by using the method of PCR-directed mutagenesis. The principle of PCR-directed mutagenesis is described in Example 2 and is based on the amplification of an entire template plasmid with the desired alterations located at the 5′ ends of the primers. In downstream steps, the ends of the linear PCR product are modified to self-ligation resulting in the desired altered plasmid.

As a template DNA, the plasmid pUC18-FMD-M-E1-H6 was used (SEQ ID NO:104; FIG. 55). Primers were designed to amplify the complete pUC18-FMD-M-E1-H6 thereby replacing the codons of the M sequence with the codons of the human lysozyme pre sequence. Two primers, containing about the half of the codons of the HL sequence (SEQ ID NO: 101) at its 5′-ends, were used thereto. The first primer (SEQ ID NO: 102) anneals within the initial codons of the mature E1 thereby pointing towards the E1 C-terminus. The 3′-region of the second primer (SEQ ID NO:103) was designed to anneal between the M start codon and the proximal parts of the FMD promoter thereby pointing towards the FMD promoter.

The PCR reaction, T4 polymerase reaction, kinase reaction, ligation reaction and subsequent transformation of E. coli cells were carried out as described in Example 2.

From the PCR-directed mutagenesis as described above, a pUC18-FMD-HL-E1-H6 with a correct insert sequence was obtained (SEQ ID NO105: FIG. 56).

### Generation of pFPMT-HL-E1-H6

Finally, the shuttle vector pFPMT-HL-E1-H6 was generated by transferring the HL-E1-H6 fusion gene as an EcoRI/BamHI fragment from pUC18-FMD-HL-E1-H6 to pFPMT121 (SEQ ID NO: 106; FIG. 57) to generate the corresponding expression plasmid.

For the ligation reaction, T4 DNA ligase (Boehringer Mannheim) has been used according to the supplier's conditions (sample volume 20 μL).

E. coli HB101 cells were transformed with the ligation mixture and positive clones withheld after restriction analysis of the plasmids isolated from several transformants. A positive plasmid was selected and denominated as pFPMT-HL-E1-H6 (SEQ ID NO:107; FIG. 58). The presence of the correct insert was verified and confirmed by sequence analysis.

### Example 27

### Transformation of Hansenula Polymorpha and Selection of Transformants

H. polymorpha strain RB11 was transformed (PEG-mediated DNA uptake protocol essentially as described by (Klebe, R. J. et al. 1983b) with the modification of (Roggenkamp, R. et al. 1986) with the parental shuttle vector as described in Example 26. A set of 36 uracil-prototrophic colonies were selected and used for strain generation by the procedure as described in Example 6.

Expression analysis of the E1 products was performed in 20 of these strains by Western Blot analysis of intracellular soluble fractions, generated after MeOH-induction. For obtaining the intracellular soluble fraction, 3 ml cell culture was harvested and cells were resuspended in ⅙ culture volume (here 500 μl) of a buffer containing 50 mM Tris-HCl pH 7.5, 150 mM NaCl, 0.1% Triton X-100 and 2 mM PMSF. Cells were disrupted with glass beads (here 250 μl). The supernatant was transferred to a new tube and centrifugated for 15 minutes at 13500 rpm at 4° C. The clear supernatant was defined as the intracellular soluble fraction.

Twelve out of the twenty tested strains produced E1. Except for one strain which contained only moderate amounts of E1 products, all other positive strains were strong E1 producer strains.

### Example 28

### Comparative Analysis of the HCV E1 Protein Expressed in Hansenula Polymorpha Derived from the CL-E1-H6 Versus HL-E1-H6 Construct

To define the differences between the HL- and CL-E1-H6-derived expression products, MeOH-inductions were performed of the H. polymorpha strains carrying the CL-E1-H6 and the HL-E1-H6 construct. Intracellular soluble fractions prepared from these inductions were subjected to PNGaseF-digestion and then directly compared by protein gel electrophoresis/Western Blot (FIG. 59). To each 32 μl of intracellular soluble fraction of the strains (see Example 27), 4 μl 1% SDS, and, if appropriate, 4u PNGaseF (Roche) were added and the solution was incubated for 16 h at 37° C. in a total volume of 44 μl. After adding 5 μl of 4×SAB, samples were denaturated for 5′ at 95° C., and sample volumes of 2 μl were loaded on a protein gel and separated via SDS PAGE. Subsequently, Western Blotting was carried out with the mouse derived MAB 11B7D8 (E1-specific) as the primary antibody in combination with the respective Western Breeze Kit (Novex) according to the supplier's instructions. The consistencies of the HL- or CL-E1-H6-derived soluble products were apparently indistinguishable. Both provided the “16 kDa-band” (see FIG. 59), and both displayed a staple of slower migrating bands which in case of the CL-construct have been shown to correspond to core-glycosylated processed molecules (see WO02/086101). The band-staple between 18 and 34 kDa responded to PNGaseF and thus indeed should correspond to core-glycosylated molecule species.

In summary, the HL-pre-sequence provides an E1-translocation comparable to that observed with the CL-pre-sequence. These results indicate the general suitability of lysozyme pre-sequences for protein-targeting to the ER of H. polymorpha.

### Example 29

### Construction of pFPMT-CL-IL4-H6 Shuttle Vector

So far the CL-pre-sequence was fused exclusively to HCV protein targets. It was demonstrated that the promising properties of the CL-pre-sequence in combination with the H. polymorpha expression system can be transferred to other, non-HCV target proteins also, e.g. the interleukin-4 (IL4) which is a human cytokine involved in at least several B-cell activation processes.

The construction of pFPMT-CL-IL-4-H6 for Hansenula polymorpha transformation required four steps, starting from pFPMT-MFα-IL-4 (SEQ ID NO:109; FIG. 60).

### Generation of pUC18-MFα-IL-4

The MFα-IL-4 DNA fragment of the expression plasmid pFPMT-MFα-IL-4 was cloned as a 0.652 kb EcoRI/BamHI fragment (partial EcoRI-digestion) into pUC18. For the ligation reaction, T4 DNA ligase (Boehringer Mannheim) has been used according to the supplier's conditions. E. coli HB101 cells were transformed with the ligation mixture and positive clones withheld after restriction analysis of the plasmids isolated from several transformants. A positive plasmid was selected and denominated as pUC18-MFα-IL-4 (SEQ ID NO:110; FIG. 61).

### Generation of pUC18-CL-IL-4-H6

In the second step, the codons of the MFα prepro-sequence of pUC18-MFα-IL-4 were replaced with those of the CL pre-sequence, by PCR site directed mutagenesis. The principle of PCR-directed mutagenesis is described in Example 2 and is based on the amplification of an entire template plasmid with the desired alterations located at the 5′ ends of the primers. In downstream steps, the ends of the linear PCR product are modified to self-ligation resulting in the desired altered plasmid.

As a template DNA, the plasmid pUC18-MFα-IL-4 was used (SEQ ID NO:110; FIG. 61). Primers were designed to amplify the complete pUC18-MFα-IL-4 thereby replacing the codons of the MFα prepro-sequence with the codons of the CL pre-sequence. Two primers, containing about the half of the codons of the CL sequence (SEQ ID NO: 99) at its 5′-ends, were used thereto. The two primers used were defined by SEQ ID NO:111 and SEQ ID NO:112.

The PCR reaction, T4 polymerase reaction, kinase reaction, ligation reaction and subsequent transformation of E. coli cells were carried out as described in Example 2. From the PCR-directed mutagenesis as described above, a pUC18-CL-IL-4 with a correct insert sequence was obtained (SEQ ID NO:113; FIG. 62). Similarly, using pUC18-CL-IL-4 as a template, the codons of a C-terminal His tag were added, again by PCR-directed mutagenesis, generating pUC18-CL-IL-4-H6 (SEQ ID NO:114; FIG. 63). The two primers used were defined by SEQ ID NO:115 and SEQ ID NO:116).

### Generation of pFPMT-CL-IL-4-H6

In the fourth step, the CL-IL-4-H6 ORF of pUC18-CL-IL-4-H6 was transferred to pFPMT121 (SEQ ID NO:106; FIG. 57). For the ligation reaction, T4 DNA ligase (Boehringer Mannheim) has been used according to the supplier's conditions (sample volume 20 μL). E. coli HB101 cells were transformed with the ligation mixture and positive clones withheld after restriction analysis of the plasmids isolated from several transformants. A positive plasmid was selected and denominated as pFPMT-CL-IL-4-H6 (SEQ ID NO:117; FIG. 64). The presence of the correct insert was verified and confirmed by sequence analysis.

### Example 30

### Transformation of Hansenula Polymorpha and Selection of Transformants

H. polymorpha strain RB11 was transformed (PEG-mediated DNA uptake protocol essentially as described by (Klebe, R. J. et al. 1983a) with the modification of (Roggenkamp, R. et al. 1986) with the parental shuttle vector pFPMT-CL-IL-4-H6 as described in Example 29. Thirty-six strains were generated and essentially evaluated as described in Example 6. Positive clones were withheld for further investigation.

### Example 31

### Analysis of the Human IL-4 Protein Expressed in Hansenula Polymorpha Derived from the CL-IL-4-H6Construct

The intracellular soluble fractions of twelve of these strains were prepared (see Example 27) and analyzed by Western Blot, using the (His)₆-specific mouse derived MAB 21E1B4. Specific bands absent in the control strain material were observed in the case of eight of the twelve strains tested (FIG. 65).

To get more insight in the nature of the bands derived from expression of CL-IL-4-H6 in H. polymorpha, three IL-4-positive strains were selected for further evaluation. The intracellular soluble fractions of these strains were subjected to a PNGaseF digestion and analysed by Western Blot (FIG. 66). As shown at three examples (strains #s 3, 5, and 8) the 16 kDa band did not respond to PNGaseF digestion while the 18 kDa band did, resulting in a 14 kDa band. This result indicates that the 16 kDa band probably corresponds to unprocessed CL-IL-4-H6 while the 18 kDa band corresponds to processed core-N-glycosylated molecules. The 14 kDa band appearing upon PNGaseF digestion reflects the de-N-glycosylated form of the 18 kDa product. Presence of this core-N-glycosylated IL-4-H6 population proves achievement of proper targeting to and further processing in the ER.

To summarize the results, the CL-pre-sequence is functional in a non-HCV protein which is targeted to the ER if fused to the CL pre-sequence.

### REFERENCE LIST

Agaphonov, M. O., Beburov, M. Y., Ter Avanesyan, M. D., and Smirnov, V. N. (1995) A disruption-replacement approach for the targeted integration of foreign genes in Hansenula polymorpha. Yeast 11: 1241-1247.
Agaphonov, M. O., Trushkina, P. M., Sohn, H., Choi, E. S., Rhee, S. K., and Ter Avanesyan, M. D. (1999) Vectors for rapid selection of integrants with different plasmid copy numbers in the yeast Hansenula polymorpha DL1. Yeast 15:541-551.
Alber, T. and Kawasaki, G. (1982) Nucleotide sequence of the triose phosphate isomerase gene of Saccharomyces cerevisiae. J. Mol. Appl. Genet. 1:419-434.
Ammerer, G. (1983) Expression of genes in yeast using the ADCI promoter. Methods Enzymol. 101:192-201.
Ballou, L., Hitzeman, R. A., Lewis, M. S., and Ballou, C. E. (1991a) Vanadate-resistant yeast mutants are defective in protein glycosylation. Proc. Natl. Acad. Sci. U.S.A 88:3209-3212.
Ballou, L., Hitzeman, R. A., Lewis, M. S., and Ballou, C. E. (1991b) Vanadate-resistant yeast mutants are defective in protein glycosylation. Proc. Natl. Acad. Sci. U.S.A 88:3209-3212.
Ballou, L., Hitzeman, R. A., Lewis, M. S., and Ballou, C. E. (1991c) Vanadate-resistant yeast mutants are defective in protein glycosylation. Proc. Natl. Acad. Sci. U.S.A 88:3209-3212.
Beekman, N. J., Schaaper, W. M., Tesser, G. I., Dalsgaard, K., Kamstrup, S., Langeveld, J. P., Boshuizen, R. S., and Meloen, R. H. (1997) Synthetic peptide vaccines: palmitoylation of peptide antigens by a thioester bond increases immunogenicity. J. Pept. Res. 50:357-364.
Burns, J., Butler, J., and Whitesides, G. (1991) Selective reduction of disulfides by Tris(2-carboxyethyl)phosphine. J. Org. Chem. 56:2648-2650.
Cox, H., Mead, D., Sudbery, P., Eland, R. M., Mannazzu, I., and Evans, L. (2000) Constitutive expression of recombinant proteins in the methylotrophic yeast Hansenula polymorpha using the PMA1 promoter. Yeast 16:1191-1203.
Cregg, J. M. (1999) Expression in the methylotophic yeast Pichia pastoris. In Gene expression systems: using nature for the art of expression, J. M. Fernandez and J. P. Hoeffler, eds (San Diego: Academic Press), pp. 157-191.
Darbre, A. (1986) Practical protein chemistry: a handbook. Whiley & Sons Ltd.
Doms, R. W., Lamb, R. A., Rose, J. K., and Helenius, A. (1993a) Folding and assembly of viral membrane proteins. Virology 193:545-562.
Doms, R. W., Lamb, R. A., Rose, J. K., and Helenius, A. (1993b) Folding and assembly of viral membrane proteins. Virology 193:545-562.
Elble, R. (1992) A simple and efficient procedure for transformation of yeasts. Biotechniques 13:18-20.
Gailit, J. (1993) Restoring free sulfhydryl groups in synthetic peptides. Anal. Biochem. 214:334-335.
Garson, J. A., Lubach, D., Passas, J., Whitby, K., and Grant, P. R. (1999b) Suramin blocks hepatitis C binding to human hepatoma cells in vitro. J. Med. Virol. 57:238-242.
Garson, J. A., Lubach, D., Passas, J., Whitby, K., and Grant, P. R. (1999a) Suramin blocks hepatitis C binding to human hepatoma cells in vitro. J. Med. Virol. 57:238-242.
Gatzke, R., Weydemann, U., Janowicz, Z. A., and Hollenberg, C. P. (1995) Stable multicopy integration of vector sequences in Hansenula polymorpha. Appl. Microbiol. Biotechnol. 43:844-849.
Gellissen, G. (2000a) Heterologous protein production in methylotrophic yeasts. Appl. Microbiol. Biotechnol. 54:741-750.
Gellissen, G. (2000c) Heterologous protein production in methylotrophic yeasts. Appl. Microbiol. Biotechnol. 54:741-750.
Gellissen, G. (2000b) Heterologous protein production in methylotrophic yeasts. Appl. Microbiol. Biotechnol. 54:741-750.
Gellissen, G. (2000d) Heterologous protein production in methylotrophic yeasts. Appl. Microbiol. Biotechnol. 54:741-750.
Grakoui, A., Wychowski, C., Lin, C., Feinstone, S. M., and Rice, C. M. (1993) Expression and identification of hepatitis C virus polyprotein cleavage products. J. Virol. 67:1385-1395.
Heile, J. M., Fong, Y. L., Rosa, D., Berger, K., Saletti, G., Campagnoli, S., Bensi, G., Capo, S., Coates, S., Crawford, K., Dong, C., Wininger, M., Baker, G., Cousens, L., Chien, D., Ng, P., Archangel, P., Grandi, G., Houghton, M., and Abrignani, S. (2000) Evaluation of hepatitis C virus glycoprotein E2 for vaccine design: an endoplasmic reticulum-retained recombinant protein is superior to secreted recombinant protein and DNA-based vaccine candidates. J. Virol. 74:6885-6892.
Helenius, A. (1994) How N-linked oligosaccharides affect glycoprotein folding in the endoplasmic reticulum. Mol. Biol. Cell 5:253-265.
Hermanson, G. T. (1996) Bioconjugate techniques. San Diego: Academic Press.
Herscovics, A. and Orlean, P. (1993) Glycoprotein biosynthesis in yeast. FASEB J. 7:540-550.
Huikata, M., Kato, N., Ootsuyama, Y., Nakagawa, M., and Shimotohno, K. (1991) Gene mapping of the putative structural region of the hepatitis C virus genome by in vitro processing analysis. Proc. Natl. Acad. Sci. U.S.A 88:5547-5551.
Hitzeman, R. A., Clarke, L., and Carbon, J. (1980) Isolation and characterization of the yeast 3-phosphoglycerokinase gene (PGK) by an immunological screening technique. J. Biol. Chem. 255:12073-12080.
Hollenberg, C. P. and Gellissen, G. (1997a) Production of recombinant proteins by methylotrophic yeasts. Curr. Opin. Biotechnol. 8:554-560.
Hollenberg, C. P. and Gellissen, G. (1997b) Production of recombinant proteins by methylotrophic yeasts. Curr. Opin. Biotechnol. 8:554-560.
Hollenberg, C. P. and Gellissen, G. (1997c) Production of recombinant proteins by methylotrophic yeasts. Curr. Opin. Biotechnol. 8:554-560.
Holmgren, A. (1979) Thioredoxin catalyzes the reduction of insulin disulfides by dithiothreitol and dihydrolipoamide. J. Biol. Chem. 254:9627-9632.
Jayabaskaran, C., Davison, P. F., and Paulus, H. (1987) Facile preparation and some applications of an affinity matrix with a cleavable connector arm containing a disulfide bond. Prep. Biochem. 17:121-141.
Julius, D., Brake, A., Blair, L., Kunisawa, R., and Thorner, J. (1984) Isolation of the putative structural gene for the lysine-arginine-cleaving endopeptidase required for processing of yeast prepro-alpha-factor. Cell 37:1075-1089.
Kalef, E., Walfish, P. G., and Gitler, C. (1993) Arsenical-based affinity chromatography of vicinal dithiol-containing proteins: purification of L1210 leukemia cytoplasmic proteins and the recombinant rat c-erb A beta 1 T3 receptor. Anal. Biochem. 212:325-334.
Kato, N., Ootsuyama, Y., Tanaka, T., Nakagawa, M., Nakazawa, T., Muraiso, K., Ohkoshi, S., Hijikata, M., and Shimotohno, K. (1992) Marked sequence diversity in the putative envelope proteins of hepatitis C viruses. Virus Res. 22:107-123.
Kawasaki, G. and Fraenkel, D. G. (1982) Cloning of yeast glycolysis genes by complementation. Biochem. Biophys. Res. Commun. 108:1107-1122.
Klebe, R. J., Harriss, J. V., Sharp, Z. D., and Douglas, M. G. (1983a) A general method for polyethylene-glycol-induced genetic transformation of bacteria and yeast. Gene 25:333-341.
Klebe, R. J., Harriss, J. V., Sharp, Z. D., and Douglas, M. G. (1983b) A general method for polyethylene-glycol-induced genetic transformation of bacteria and yeast. Gene 25:333-341.
Klebe, R. J., Harriss, J. V., Sharp, Z. D., and Douglas, M. G. (1983c) A general method for polyethylene-glycol-induced genetic transformation of bacteria and yeast. Gene 25:333-341.
Kumar, N., Kella, D., and Kinsella, J. E. (1985) A method for the controlled cleavage of disulfide bonds in proteins in the absence of denaturants. J. Biochem. Biophys. Methods 11:251-263.
Kumar, N., Kella, D., and Kinsella, J. E. (1986) Anomalous effects of denaturants on sulfitolysis of protein disulfide bonds. Int. J. Peptide Prot. Res. 28:586-592.
Maertens, G. and Stuyver, L. (1997) Genotypes and genetic variation of hepatitis C virus. In The molecular medicine of viral hepatitis, T. J. Harrison and A. J. Zuckerman, eds John Wiley & Sons), pp. 183-233.
Major, M. E. and Feinstone, S. M. (1997) The molecular virology of hepatitis C. Hepatology 25:1527-1538.
Mustilli, A. C., Izzo, E., Houghton, M., and Galeotti, C. L. (1999a) Comparison of secretion of a hepatitis C virus glycoprotein in Saccharomyces cerevisiae and Kluyveromyces lactis. Res. Microbiol. 150:179-187.
Mustilli, A. C., Izzo, E., Houghton, M., and Galeotti, C. L. (1999b) Comparison of secretion of a hepatitis C virus glycoprotein in Saccharomyces cerevisiae and Kluyveromyces lactis. Res. Microbiol. 150:179-187.
Mustilli, A. C., Izzo, E., Houghton, M., and Galeotti, C. L. (1999c) Comparison of secretion of a hepatitis C virus glycoprotein in Saccharomyces cerevisiae and Kluyveromyces lactis. Res. Microbiol. 150:179-187.
Mustilli, A. C., Izzo, E., Houghton, M., and Galeotti, C. L. (1999d) Comparison of secretion of a hepatitis C virus glycoprotein in Saccharomyces cerevisiae and Kluyveromyces lactis. Res. Microbiol. 150:179-187.
Nagai, K. and Thogersen, H. C. (1984) Generation of beta-globin by sequence-specific proteolysis of a hybrid protein produced in Escherichia coli. Nature 309:810-812.
Nielsen, P. E. (2001) Targeting double stranded DNA with peptide nucleic acid (PNA). Curr Med Chem 8:545-550.
Okabayashi, K., Nakagawa, Y., Hayasuke, N., Ohi, H., Miura, M., Ishida, Y., Shimizu, M., Murakami, K., Hirabayashi, K., Minamino, H., and. (1991) Secretory expression of the human serum albumin gene in the yeast, Saccharomyces cerevisiae. J. Biochem. (Tokyo) 110: 103-110.
Orum, H. and Wengel J. (2001) Locked nucleic acids: a promising molecular family for gene-function analysis and antisense drug development. Curr Opin. Mol. Ther. 3:239-243.
Padgett, K. A. and Sorge, J. A. (1996a) Creating seamless junctions independent of restriction sites in PCR cloning. Gene 168:31-35.
Padgett, K. A. and Sorge, J. A. (1996b) Creating seamless junctions independent of restriction sites in PCR cloning. Gene 168:31-35.
Pedersen, J., Lauritzen, C., Madsen, M. T., and Weis, D. S. (1999) Removal of N-terminal polyhistidine tags from recombinant proteins using engineered aminopeptidases. Protein Expr. Purif. 15:389-400.
Pomroy, N. C. and Deber, C. M. (1998) Solubilization of hydrophobic peptides by reversible cysteine PEGylation. Biochem. Biophys. Res. Commun. 245:618-621.
Raymond, C. K. (1999) Recombinant protein expression in Pichia methanolica. In Gene expression systems: using nature for the art of expression, J. M. Fernandez and J. P. Hoeffler, eds (San Diego: Academic Press), pp. 193-209.
Rein, A., Ott, D. E., Mirro, J., Arthur, L. O., Rice, W., and Henderson, L. E. (1996) Inactivation of murine leukemia virus by compounds that react with the zinc finger in the viral nucleocapsid protein. J. Virol. 70:4966-4972.
Roggenkamp, R., Hansen, H., Eckart, M., Janowicz, Z., and Hollenberg, C. P. (1986) Transformation of the methylotrophic yeast Hansenula polymorpha by autonomous replication and integration vectors. Mol Gen Genet. 202:302-308.
Rosa, D., Campagnoli, S., Moretto, C., Guenzi, E., Cousens, L., Chin, M., Dong, C., Weiner, A. J., Lau, J. Y., Choo, Q. L., Chien, D., Pileri, P., Houghton, M., and Abrignani, S. (1996) A quantitative test to estimate neutralizing antibodies to the hepatitis C virus: cytofluorimetric assessment of envelope glycoprotein 2 binding to target cells. Proc. Natl. Acad. Sci. U.S.A 93:1759-1763.
Rose, J. K. and Doms, R. W. (1988) Regulation of protein export from the endoplasmic reticulum. Annu. Rev. Cell Biol. 4:257-288.
Russell, D. W., Smith, M., Williamson, V. M., and Young, E. T. (1983) Nucleotide sequence of the yeast alcohol dehydrogenase II gene. J. Biol. Chem. 258:2674-2682.
Russell, P. R. (1983) Evolutionary divergence of the mRNA transcription initiation mechanism in yeast. Nature 301:167-169.
Russell, P. R. (1985) Transcription of the triose-phosphate-isomerase gene of Schizosaccharomyces pombe initiates from a start point different from that in Saccharomyces cerevisiae. Gene 40:125-130.
Russell, P. R. and Hall, B. D. (1983) The primary structure of the alcohol dehydrogenase gene from the fission yeast Schizosaccharomyces pombe. J. Biol. Chem. 258:143-149.
Sambrook, J., Fritsch, E. F., and Maniatis, T. (1989) Molecular Cloning: A Laboratory Manual. Cold Spring Harbor Laboratory Press.
Singh, R. and Kats, L. (1995) Catalysis of reduction of disulfide by selenol. Anal. Biochem. 232:86-91.
Sohn, J. H., Choi, E. S., Kang, H. A., Rhee, J. S., and Rhee, S. K. (1999) A family of telomere-associated autonomously replicating sequences and their functions in targeted recombination in Hansenula polymorpha DL-1. J. Bacteriol. 181:1005-1013.
Stuyver, L., van Arnhem, W., Wyseur, A., Hernandez, F., Delaporte, E., and Maertens, G. (1994) Classification of hepatitis C viruses based on phylogenetic analysis of the envelope 1 and nonstructural 5B regions and identification of five additional subtypes. Proc. Natl. Acad. Sci. U.S.A 91:10134-10138.
Sugrue, R. J., Cui, T., Xu, Q., Fu, J., and Chan, Y. C. (1997b) The production of recombinant dengue virus E protein using Escherichia coli and Pichia pastoris. J. Virol. Methods 69:159-169.
Sugrue, R. J., Cui, T., Xu, Q., Fu, J., and Chan, Y. C. (1997a) The production of recombinant dengue virus E protein using Escherichia coli and Pichia pastoris. J. Virol. Methods 69:159-169.
Thakur, M. L., DeFulvio, J., Richard, M. D., and Park, C. H. (1991) Technetium-99m labeled monoclonal antibodies: evaluation of reducing agents. Int. J. Rad. Appl. Instrum. B 18:227-233.
Vingerhoeds, M. H., Haisma, H. J., Belliot, S. O., Smit, R. H., Crommelin, D. J., and Storm, G. (1996) Immunoliposomes as enzyme-carriers (immuno-enzymosomes) for antibody-directed enzyme prodrug therapy (ADEPT): optimization of prodrug activating capacity. Pharm. Res. 13:604-610.
Wahlestedt, C., Salmi, P., Good, L., Kela, J., Johnsson, T., Hokfelt, T., Broberger, C., Porreca, F., Lai, J., Ren, K., Ossipov, M., Koshkin, A., Jakobsen, N., Skouv, J., Oerum, H., Jacobsen, M. H., and Wengel, J. (2000) Potent and nontoxic antisense oligonucleotides containing locked nucleic acids. Proc Natl Acad Sci USA 97:5633-5638.
Weydemann, U., Keup, P., Piontek, M., Strasser, A. W., Schweden, J., Gellissen, G., and Janowicz, Z. A. (1995) High-level secretion of hirudin by Hansenula polymorpha—authentic processing of three different preprohirudins. Appl. Microbiol. Biotechnol. 44:377-385.

All references, publications and patents cited and referred to herein are incorporated in their entirety herein by reference.

1. A recombinant nucleic acid comprising a nucleotide sequence encoding a protein comprising a lysozyme leader peptide or a functional equivalent thereof joined to a protein of interest or a part thereof, wherein said protein of interest or part thereof is expressed in a methylotrophic yeast cell.

2. The recombinant nucleic acid according to claim 1 wherein said lysozyme leader peptide or a functional equivalent thereof is joined to a protein of the formula
[(A1)ₐ-(PS1)_{b}-(A2)_{c}]-PROT-[(A3)_{d}-(PS2)ₑ-(A4)_{f}]
wherein:
A1, A2, A3 and A4 are adaptor peptides which can be different or the same,
PS1 and PS2 are processing sites which can be the different or the same,
PROT is a protein of interest or a part thereof,
a, b, c, d, e and f are 0 or 1, and
wherein, optionally, A1 and/or A2 are part of PS1 and/or wherein A3 and/or A4 are part of PS2.

3. The recombinant nucleic acid according to claim 1 further comprising regulatory elements for said expression of said protein of interest or a part thereof in a methylotrophic yeast cell.

4. The recombinant nucleic acid according to claim 1 wherein the lysozyme leader peptide has an amino acid sequence defined by SEQ ID NO:1 or SEQ ID NO:99 or SEQ ID NO 101.

5. The recombinant nucleic acid according to claim 2 wherein A has an amino acid sequence chosen from SEQ ID NOs:63-65, 70-72 and 74-82, wherein PS has an amino acid sequence chosen from SEQ ID NOs:66-68 and 83-84 or wherein PS is a dibasic site such as Lys-Lys, Arg-Arg, Lys-Arg and Arg-Lys or a monobasic site such as Lys, and wherein PROT is chosen from SEQ ID NOs:85-98 or SEQ ID NO:108 and fragments thereof.

6. The recombinant nucleic acid according to claim 1 wherein said methylotrophic yeast cell is a Hansenula polymorpha cell, or a Pichia pastoris cell, or a mutant cell derived from any thereof.

7. A vector comprising the recombinant nucleic acid according to claim 1.

8. The vector according to claim 7 which is an expression vector.

9. The vector according to claim 7 which is an autonomously replicating vector or an integrative vector.

10. The vector according to claim 8 which is chosen from SEQ ID NOs: 21, 32, 36, 40, 107 and 117.

11. An isolated methylotrophic yeast cell comprising the recombinant nucleic acid according to claim 1.

12. The isolated methylotrophic yeast cell according to claim 11 which is expressing the recombinant nucleic acid.

13. The isolated methylotrophic yeast cell according to claim 11 which is expressing the protein characterized by the structure L-[(A1)ₐ-(PS1)_{b}-(A2)_{c}]-PROT-[(A3)_{d}-(PS2)ₑ-(A4)_{f}]
wherein:
L is a lysozyme leader peptide or a functional equivalent thereof,
A1, A2, A3 and A4 are adaptor peptides which can be different or the same,
PS1 and PS2 are processing sites which can be the different or the same,
PROT is a protein of interest or a part thereof,
a, b, c, d, e and f are 0 or 1, and
wherein, optionally, A1 and/or A2 are part of PS1 and/or wherein A3 and/or A4 are part of PS2.

14. The isolated methylotrophic yeast cell according to claim 11 which is translocating the protein L-[(A1)ₐ-(PS1)_{b}-(A2)_{c}]-PROT-[(A3)_{d}-(PS2)ₑ-(A4)_{f}] to the endoplasmic reticulum upon removal of the L peptide wherein said protein and said L peptide are derived from the protein characterized by the structure L-[(A1)ₐ-(PS1)_{b}-(A2)_{c}]-PROT-[(A3)_{d}-(PS2)ₑ-(A4)_{f}]
wherein:
L is a lysozyme leader peptide or a functional equivalent thereof,
A1, A2, A3 and A4 are adaptor peptides which can be different or the same,
PS1 and PS2 are processing sites which can be the different or the same,
PROT is a protein of interest or a part thereof,
a, b, c, d, e and f are 0 or 1, and
wherein, optionally, A1 and/or A2 are part of PS1 and/or wherein A3 and/or A4 are part of PS2.

15. The isolated methylotrophic yeast cell according to claim 11 which is processing the processing sites PS1 and/or PS2 in said protein translocated to the endoplasmic reticulum.

16. The isolated methylotrophic yeast cell according to claim 11 which is N-glycosylating said protein translocated to the endoplasmic reticulum.

17. The isolated methylotrophic yeast cell according to claim 15 which is N-glycosylating said protein translocated to the endoplasmic reticulum and processed at said sites PS1 and/or PS2.

18. The isolated methylotrophic yeast cell according to claim 11 which is a Hansenula polymorpha cell, or a Pichia pastoris cell, or a mutant cell derived from any thereof.

19. A method for producing a protein of interest or a part thereof in a methylotrophic yeast cell, said method comprising transforming said yeast cell with the recombinant nucleic acid according to claim 1 or with the vector comprising said nucleic acid, and expressing said protein comprising the lysozyme leader peptide or a functional equivalent thereof joined to a protein of interest or a part thereof.

20. A method for producing a protein of interest or a part thereof in a methylotrophic yeast cell, said method comprising transforming said yeast cell with the recombinant nucleic acid according to claim 1 or with the vector comprising said nucleic acid, and expressing said protein characterized by the structure L-[(A1)ₐ-(PS1)_{b}-(A2)_{c}]-PROT-[(A3)_{d}-(PS2)ₑ-(A4)_{f}]
wherein:
L is a lysozyme leader peptide or a functional equivalent thereof,
A1, A2, A3 and A4 are adaptor peptides which can be different or the same,
A1 and PS2 are processing sites which can be the different or the same,
PROT is a protein of interest or a part thereof,
a, b, c, d, e and f are 0 or 1, and
wherein, optionally, A1 and/or A2 are part of PS1 and/or wherein A3 and/or A4 are part of PS2.

21. The method according to claim 19 wherein said methylotrophic yeast cell is translocating the protein L-[(A1)ₐ-(PS1)_{b}-(A2)_{c}]-PROT-[(A3)_{d}-(PS2)ₑ-(A4)_{f}] to the endoplasmic reticulum upon removal of the L peptide wherein said protein and said L peptide are derived from the protein characterized by the structure L-[(A1)ₐ-(PS1)_{b}-(A2)_{c}]-PROT-[(A3)_{d}-(PS2)ₑ-(A4)_{f}]
wherein:
L is a lysozyme leader peptide or a functional equivalent thereof,
A1, A2, A3 and A4 are adaptor peptides which can be different or the same,
PS1 and PS2 are processing sites which can be the different or the same,
PROT is a protein of interest or a part thereof,
a, b, c, d, e and f are 0 or 1, and
wherein, optionally, A1 and/or A2 are part of PS1 and/or wherein A3 and/or A4 are part of PS2.

22. The method according to claim 19 wherein said methylotrophic yeast cell is processing the processing sites PS1 and/or PS2 in said protein translocated to the endoplasmic reticulum.

23. The method according to claim 19 further comprising in vitro processing of the processing sites PS1 and/or PS2.

24. The method according to claim 19 wherein said methylotrophic yeast cell is N-glycosylating said protein translocated to the endoplasmic reticulum.

25. The method according to claim 22 wherein said methylotrophic yeast cell is N-glycosylating said protein translocated to the endoplasmic reticulum and processed at said sites PS1 and/or PS2.

26. The method according to claim 19 wherein said methylotrophic yeast cell is a Hansenula polymorpha cell, or a Pichia pastoris cell, or a mutant cell derived from any thereof.

27. The method according to claim 19 further comprising cultivation of said methylotrophic yeast cells in a suitable medium to obtain expression of said protein.

28. The method according to claim 27 further comprising isolation of the expressed protein from a culture of said methylotrophic yeast cells, or from said methylotrophic yeast cells.

29. The method according to claim 28 wherein said isolation step involves lysis of said methylotrophic yeast cells in the presence of a chaotropic agent.

30. The method according to claim 29 wherein the cysteine thiol-groups in the isolated proteins are chemically modified and wherein said chemical modification is reversible or irreversible.

31. The method according to claim 30 involving heparin affinity chromatography.


SEQUENCE LISTING (117 sequences)

SEQ ID NO: 1 - protein, 18 aa, Artificial sequence
  avian lysozyme signal peptide
  MISC_FEATURE (2)..(2): Xaa is Arg, Lys or Val
  MISC_FEATURE (3)..(3): Xaa is Ser, Ala, Val, Arg or Met
  MISC_FEATURE (4)..(4): Xaa is Leu or Phe
  MISC_FEATURE (5)..(5): Xaa is Leu or Ala
  MISC_FEATURE (6)..(6): Xaa is Ile, Thr, Phe or Val
  MISC_FEATURE (7)..(7): Xaa is Leu, Phe or Ala
  MISC_FEATURE (8)..(8): Xaa is Val, Ile, Ala, Leu or Cys
  MISC_FEATURE (9)..(9): Xaa is Leu, Phe, Ala or Ile
  MISC_FEATURE (10)..(10): Xaa is Cys, Phe, Ser or Leu
  MISC_FEATURE (11)..(11): Xaa is Phe, Leu, Ser or Pro
  MISC_FEATURE (12)..(12): Xaa is Leu, Ala or Met
  MISC_FEATURE (13)..(13): Xaa is Pro, Ala or Ile
  MISC_FEATURE (14)..(14): Xaa is Leu or Ala
  MISC_FEATURE (15)..(15): Xaa is Ala, Val, Ser or Met
  MISC_FEATURE (16)..(16): Xaa is Ala, Lys or Ser
  MISC_FEATURE (17)..(17): Xaa is Leu, Pro, Gln or Ile
MXXXXXXXXXXXXXXXXG

SEQ ID NO: 2 - protein, 135 aa, hepatitis C virus
YEVRNVSGMYHVTNDCSNSSIVYEAADMIMHTPGCVPCVRENNSSRCWVALTPTLAARNASVPTTTIRRHVDLLVGAAAFCSAMYVGDLCGSVFLVSQLFTISPRRHETVQDCNCSIYPGHITGHRMAWDMMMNW

SEQ ID NO: 3 - protein, 290 aa, hepatitis C virus
HTRVSGGAAASDTRGLVSLFSPGSAQKIQLVNTNGSWHINRTALNCNDSLQTGFFAALFYKHKFNSSGCPERLASCRSIDKFAQGWGPLTYTEPNSSDQRPYCWHYAPRPCGIVPASQVCGPVYCFTPSPVVVGTTDRFGVPTYNWGANDSDVLILNNTRPPRGNWFGCTWMNGTGFTKTCGGPPCNIGGAGNNTLTCPTDCFRKHPEATYARCGSGPWLTPRCMVHYPYRLWHYPCTVNFTIFKVRMYVGGVEHRFEAACNWTRGERCDLEDRDRSELSPLLLSTTEWQ

SEQ ID NO: 4 - protein, 141 aa, hepatitis C virus
YEVRNVSGMYHVTNDCSNSSIVYEAADMIMHTPGCVPCVRENNSSRCWVALTPTLAARNASVPTTTIRRHVDLLVGAAAFCSAMYVGDLCGSVFLVSQLFTISPRRHETVQDCNCSIYPGHITGHRMAWDMMMNWHHHHHH

SEQ ID NO: 5 - protein, 301 aa, hepatitis C virus
HTRVSGGAAASDTRGLVSLFSPGSAQKIQLVNTNGSWHINRTALNCNDSLQTGFFAALFYKHKFNSSGCPERLASCRSIDKFAQGWGPLTYTEPNSSDQRPYCWHYAPRPCGIVPASQVCGPVYCFTPSPVVVGTTDRFGVPTYNWGANDSDVLILNNTRPPRGNWFGCTWMNGTGFTKTCGGPPCNIGGAGNNTLTCPTDCFRKHPEATYARCGSGPWLTPRCMVHYPYRLWHYPCTVNFTIFKVRMYVGGVEHRFEAACNWTRGERCDLEDRDRSELSPLLLSTTEWQVIEGRHHHHHH

SEQ ID NO: 6 - DNA, 3448 bp, Artificial sequence
  vector pGEMTE1sH6
aatcactagtgcggccgcctgcaggtcgaccatatgggagagctcccaacgcgttggatgcatagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttcgataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaataccgcgcccggcgaccgagttgctcttgcccggcgtcaatacgggataatagtgtatgacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgtatgcggtgtgaaataccgcacagatgcgtaaggagaaaataccgcatcaggcgaaattgtaaacgttaatattttgttaaaattcgcgttaaatatttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcacccaaatcaagttttttgcggtcgaggtgccgtaaagctctaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggcccgacgtcgcatgctcccggccgccatggccgcgggattccaatgcatatgaggtgcgcaacgtgtccgggatgtaccatgtcacgaacgactgctccaactcaagcattgtgtatgaggcagcggacatgatcatgcacacccccgggtgcgtgccctgcgttcgggagaacaactcttcccgctgctgggtagcgctcacccccacgctcgcagctaggaacgccagcgtccccactacgacaatacgacgccacgtcgatttgctcgttggggcggctgctttctgttccgctatgtacgtgggggatctctgcggatctgtcttcctcgtctcccagctgttcaccatctcgcctcgccggcatgagacggtgcaggactgcaattgctcaatctatcccggccacataacaggtcaccgtatggcttgggatatgatgatgaactggcaccaccaccatcaccattaaggatccaag

SEQ ID NO: 7 - DNA, 37 bp, Artificial sequence
  synthetic probe or primer
agttactcttcaaggtatgaggtgcgcaacgtgtccg

SEQ ID NO: 8 - DNA, 47 bp, Artificial sequence
  synthetic probe or primer
agttactcttcacagggatcctccttaatggtgatggtggtggtgcc

SEQ ID NO: 9 - DNA, 3067 bp, Artificial sequence
  vector pCHH-Hir
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgaccctagatctctattactgcaggtattcttccgggatttcttcgaagtcgccgtcgttgtgagactgcggacgcggggtaccttcgccagtaacgcactggttacgttcgcctttagagcccaggatgcatttgttgccctggccgcaaacgttagagccttcgcacaggcacaggttctgaccggattcagtgcagtcagtgtaaacaaccctcttttccaacgggtgtgtagttccattctccaccgctagggctgcgctgggctccattggcgaggttttcaaggccgctaggatgcgatccatgcgtccgtagccttgcgtggagcgtgcgtgtgcgtgcgggagtgcgcataggtaggctacggtgatgattgctagcatggcgggaatagttttgctatacatgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 10 - DNA, 35 bp, Artificial sequence
  synthetic probe or primer
agttactcttcacctcttttccaacgggtgtgtag

SEQ ID NO: 11 - DNA, 34 bp, Artificial sequence
  synthetic probe or primer
agtcactcttcactgcaggcatgcaagcttggcg

SEQ ID NO: 12 - DNA, 6973 bp, Artificial sequence
  vector pFPMT121
ggtaccctgctcaatctccggaatggtgatctgatcgttcctgaaaacctcgacattggctccctcctgacacaggtactcgtacaggttccaggtaaacgagtcgtagttgtcgatcatgacaacgttcttagaagcggccggcattttgaaggtgactaatagcctaagaaaatatttaatttaattttcattaaattttcctatactcgctatttcagcttttcatctcatcacttcataaacgatataaaccagaaaaagaactattttcaaacacgcttctcaaaagcggtatgtccttccacgtctccttagaatctggcaagtccgcgagggggatccagatctgaattcccgatgaagcagagagcgcaggaggcggtatttatagtgccattcccctctctgagagacccggatggtagtcgagtgtatcggagacagcttgatgtagactccgtgcctgccggctcctcttattggcggacaccagtgagacaccccggaacttgctgtttttctgcaaaatccggggtgaccagtgggagcctatttgcacacacgagcgggacaccccactctggtgaagagtgccaaagtcattctttttcccgttgcggggcagccgattgcatgttttaggaaaatattacctttgctacaccctgtcagatttaccctccacacatatatattccgtcacctccagggactattattcgtcgttgcgccgccagcggaagatatccagaagctgttttccgagagactcggttggcgcctggtatatttgatggatgtcgcgctgcctcacgtcccggtacccaggaacgcggtgggatctcgggcccatcgaagactgtgctccagactgctcgcccagcaggtgtttcttgatcgccgcctctaaattgtccgcgcatcgccggtaacatttttccagctcggagtttgcgtttagatacagtttctgcgatgccaaaggagcctgcagattataacctcggatgctgtcattcagcgcttttaatttgacctccagatagttgctgtatttctgttcccattggctgctgcgcagcttcgtataactcgagttattgttgcgctctgcctcggcgtactggctcatgatctggatcttgtccgtgtcgcttttcttcgagtgtttctcgcaaacgatgtgcacggcctgcagtgtccaatcggagtcgagctggcgccgaaactggcggatctgagcctccacactgccctgtttctctatccacggcggaaccgcctcctgccgtttcagaatgttgttcaagtggtactctgtgcggtcaatgaaggcgttattgccggtgaaatctttgggaagcggttttcctcggggaagattacgaaattccccgcgtcgttgcgcttcctggatctcgaggagatcgttctccgcgtcgaggagatcgttctccgcgtcgacaccattccttgcggcggcggtgctcaacggcctcaacctactactgggctgcttcctaatgcaggagtcgcataagggagagcgtcgacaaacccgcgtttgagaacttgctcaagcttctggtaaacgttgtagtactctgaaacaaggccctagcactctgatctgtttctcttgggtagcggtgagtggtttattggagttcactggtttcagcacatctgtcatctagacaatattgttactaaatttttttgaactacaattgttcgtaattcatctattattatacatcctcgtcagcaatttctggcagacggagtttactaacgtcttgagtatgaggccgagaatccagctctgtggccatactcagtcttgacagcctgctgatgtggctgcgttcaacgcaataagcgtgtcctccgactccgagttgtgctcgttatcgtcgttctcatcctcggaaaaatcacacgaaagaacatactcaccagtaggctttctggtccctggggcacggctgtttctgacgtattccggcgttgataatagctcgaaagtgaacgccgagtcgcgggagtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatcggggggggggggggggggggggggcaaacaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaagccccccccccccccccccccccccccccccccccgcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaattcccccttacacggaggcatcaagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaa

SEQ ID NO: 13 - DNA, 7591 bp, Artificial sequence
  vector pFPMT-CHH-E1H6
ggtaccctgctcaatctccggaatggtgatctgatcgttcctgaaaacctcgacattggctccctcctgacacaggtactcgtacaggttccaggtaaacgagtcgtagttgtcgatcatgacaacgttcttagaagcggccggcattttgaaggtgactaatagcctaagaaaatatttaatttaattttcattaaattttcctatactcgctatttcagcttttcatctcatcacttcataaacgatataaaccagaaaaagaactattttcaaacacgcttctcaaaagcggtatgtccttccacgtctccttagaatctggcaagtccgcgagggggatccttaatggtgatggtggtggtgccagttcatcatcatatcccaagccatacggtgacctgttatgtggccgggatagattgagcaattgcagtcctgcaccgtctcatgccggcgaggcgagatggtgaacagctgggagacgaggaagacagatccgcagagatcccccacgtacatagcggaacagaaagcagccgccccaaccagcaaatcgacgtggcgtcgtattgtcgtagtggggacgctggcgttcctagctgcgagcgtgggggtgagcgctacccagcagcgggaagagttgttctcccgaacgcagggcacgcacccgggggtgtgcatgatcatgtccgctgcctcatacacaatgcttgagttggagcagtcgttcgtgacatggtacatcccggacacgttgcgcacctcatacctcttttccaacgggtgtgtagttccattctccaccgctagggctgcgctgggctccattggcgaggttttcaaggccgctaggatgcgatccatgcgtccgtagccttgcgtggagcgtgcgtgtgcgtgcgggagtgcgcataggtaggctacggtgatgattgctagcatggcgggaatagttttgctatacatgaattcccgatgaagcagagagcgcaggaggcggtatttatagtgccattcccctctctgagagacccggatggtagtcgagtgtatcggagacagcttgatgtagactccgtgcctgccggctcctcttattggcggacaccagtgagacaccccggaacttgctgtttttctgcaaaatccggggtgaccagtgggagcctatttgcacacacgagcgggacaccccactctggtgaagagtgccaaagtcattctttttcccgttgcggggcagccgattgcatgttttaggaaaatattacctttgctacaccctgtcagatttaccctccacacatatatattccgtcacctccagggactattattcgtcgttgcgccgccagcggaagatatccagaagctgttttccgagagactcggttggcgcctggtatatttgatggatgtcgcgctgcctcacgtcccggtacccaggaacgcggtgggatctcgggcccatcgaagactgtgctccagactgctcgcccagcaggtgtttcttgatcgccgcctctaaattgtccgcgcatcgccggtaacatttttccagctcggagtttgcgtttagatacagtttctgcgatgccaaaggagcctgcagattataacctcggatgctgtcattcagcgcttttaatttgacctccagatagttgctgtatttctgttcccattggctgctgcgcagcttcgtataactcgagttattgttgcgctctgcctcggcgtactggctcatgatctggatcttgtccgtgtcgcttttcttcgagtgtttctcgcaaacgatgtgcacggcctgcagtgtccaatcggagtcgagctggcgccgaaactggcggatctgagcctccacactgccctgtttctctatccacggcggaaccgcctcctgccgtttcagaatgttgttcaagtggtactctgtgcggtcaatgaaggcgttattgccggtgaaatctttgggaagcggttttcctcggggaagattacgaaattccccgcgtcgttgcgcttcctggatctcgaggagatcgttctccgcgtcgaggagatcgttctccgcgtcgacaccattccttgcggcggcggtgctcaacggcctcaacctactactgggctgcttcctaatgcaggagtcgcataagggagagcgtcgacaaacccgcgtttgagaacttgctcaagcttctggtaaacgttgtagtactctgaaacaaggccctagcactctgatctgtttctcttgggtagcggtgagtggtttattggagttcactggtttcagcacatctgtcatctagacaatattgttactaaatttttttgaactacaattgttcgtaattcatctattattatacatcctcgtcagcaatttctggcagacggagtttactaacgtcttgagtatgaggccgagaatccagctctgtggccatactcagtcttgacagcctgctgatgtggctgcgttcaacgcaataagcgtgtcctccgactccgagttgtgctcgttatcgtcgttctcatcctcggaaaaatcacacgaaagaacatactcaccagtaggctttctggtccctggggcacggctgtttctgacgtattccggcgttgataatagctcgaaagtgaacgccgagtcgcgggagtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatcggggggggggggggggggggggggcaaacaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaagccccccccccccccccccccccccccccccccccgcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaattcccccttacacggaggcatcaagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaa

SEQ ID NO: 14 - DNA, 50 bp, Artificial sequence
  synthetic probe or primer
aggggtaagcttggataaaaggtatgaggtgcgcaacgtgtccgggatgt

SEQ ID NO: 15 - DNA, 42 bp, Artificial sequence
  synthetic probe or primer
agttacggatccttaatggtgatggtggtggtgccagttcat

SEQ ID NO: 16 - DNA, 7648 bp, Artificial sequence
  vector pFPMT-Mfalfa-E1-H6
ggtaccctgctcaatctccggaatggtgatctgatcgttcctgaaaacctcgacattggctccctcctgacacaggtactcgtacaggttccaggtaaacgagtcgtagttgtcgatcatgacaacgttcttagaagcggccggcattttgaaggtgactaatagcctaagaaaatatttaatttaattttcattaaattttcctatactcgctatttcagcttttcatctcatcacttcataaacgatataaaccagaaaaagaactattttcaaacacgcttctcaaaagcggtatgtccttccacgtctccttagaatctggcaagtccgcgagggggatccttaatggtgatggtggtggtgccagttcatcatcatatcccaagccatacggtgacctgttatgtggccgggatagattgagcaattgcagtcctgcaccgtctcatgccggcgaggcgagatggtgaacagctgggagacgaggaagacagatccgcagagatcccccacgtacatagcggaacagaaagcagccgccccaacgagcaaatcgacgtggcgtcgtattgtcgtagtggggacgctggcgttcctagctgcgagcgtgggggtgagcgctacccagcagcgggaagagttgttctcccgaacgcagggcacgcacccgggggtgtgcatgatcatgtccgctgcctcatacacaatgcttgagttggagcagtcgttcgtgacatggtacatcccggacacgttgcgcacctcataccttttatccaagcttaccccttcttctttagcagcaatgctggcaatagtagtatttataaacaataacccgttatttgtgctgttggaaaatggcaaaacagcaacatcgaaatccccttctaaatctgagtaaccgatgacagcttcagccggaatttgtgccgtttcatcttctgttgtagtgttgactggagcagctaatgcggaggatgctgcgaataaaactgcagtaaaaattgaaggaaatctcatgaattcccgatgaagcagagagcgcaggaggcggtatttatagtgccattcccctctctgagagacccggatggtagtcgagtgtatcggagacagcttgatgtagactccgtgcctgccggctcctcttattggcggacaccagtgagacaccccggaacttgctgtttttctgcaaaatccggggtgaccagtgggagcctatttgcacacacgagcgggacaccccactctggtgaagagtgccaaagtcattctttttcccgttgcggggcagccgattgcatgttttaggaaaatattacctttgctacaccctgtcagatttaccctccacacatatatattccgtcacctccagggactattattcgtcgttgcgccgccagcggaagatatccagaagctgttttccgagagactcggttggcgcctggtatatttgatggatgtcgcgctgcctcacgtcccggtacccaggaacgcggtgggatctcgggcccatcgaagactgtgctccagactgctcgcccagcaggtgtttcttgatcgccgcctctaaattgtccgcgcatcgccggtaacatttttccagctcggagtttgcgtttagatacagtttctgcgatgccaaaggagcctgcagattataacctcggatgctgtcattcagcgcttttaatttgacctccagatagttgctgtatttctgttcccattggctgctgcgcagcttcgtataactcgagttattgttgcgctctgcctcggcgtactggctcatgatctggatcttgtccgtgtcgcttttcttcgagtgtttctcgcaaacgatgtgcacggcctgcagtgtccaatcggagtcgagctggcgccgaaactggcggatctgagcctccacactgccctgtttctctatccacggcggaaccgcctcctgccgtttcagaatgttgttcaagtggtactctgtgcggtcaatgaaggcgttattgccggtgaaatctttgggaagcggttttcctcggggaagattacgaaattccccgcgtcgttgcgcttcctggatctcgaggagatcgttctccgcgtcgaggagatcgttctccgcgtcgacaccattccttgcggcggcggtgctcaacggcctcaacctactactgggctgcttcctaatgcaggagtcgcataagggagagcgtcgacaaacccgcgtttgagaacttgctcaagcttctggtaaacgttgtagtactctgaaacaaggccctagcactctgatctgtttctcttgggtagcggtgagtggtttattggagttcactggtttcagcacatctgtcatctagacaatattgttactaaatttttttgaactacaattgttcgtaattcatctattattatacatcctcgtcagcaatttctggcagacggagtttactaacgtcttgagtatgaggccgagaatccagctctgtggccatactcagtcttgacagcctgctgatgtggctgcgttcaacgcaataagcgtgtcctccgactccgagttgtgctcgttatcgtcgttctcatcctcggaaaaatcacacgaaagaacatactcaccagtaggctttctggtccctggggcacggctgtttctgacgtattccggcgttgataatagctcgaaagtgaacgccgagtcgcgggagtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatcggggggggggggggggggggggggcaaacaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaagccccccccccccccccccccccccccccccccccgcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaattcccccttacacggaggcatcaagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaa

SEQ ID NO: 17 - DNA, 4453 bp, Artificial sequence
  vector pUC18-FMD-MFalfa-E1-H6
  misc_feature (1207)..(1208): N is any nucleotide
  misc_feature (1386)..(1387): N is any nucleotide
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatccttaatggtgatggtggtggtgccagttcatcatcatatcccaagccatacggtgacctgttatgtggccgggatagattgagcaattgcagtcctgcaccgtctcatgccggcgaggcgagatggtgaacagctgggagacgaggaagacagatccgcagagatcccccacgtacatagcggaacagaaagcagccgccccaacgagcaaatcgacgtggcgtcgtattgtcgtagtggggacgctggcgttcctagctgcgagcgtgggggtgagcgctacccagcagcgggaagagttgttctcccgaacgcagggcacgcacccgggggtgtgcatgatcatgtccgctgcctcatacacaatgcttgagttggagcagtcgttcgtgacatggtacatcccggacacgttgcgcacctcataccttttatccaagcttaccccttcttctttagcagcaatgctggcaatagtagtatttataaacaataacccgttatttgtgctgttggaaaatggcaaaacagcaacatcgaaatccccttctaaatctgagtaaccgatgacagcttcagccggaatttgtgccgtttcatcttctgttgtagtgttgactggagcagctaatgcggaggatgctgcgaataaaactgcagtaaaaattgaaggaaatctcatgaattcccgatgaaggcagagagcgcaaggaggcggtatttatagtgccattcccctctctgagagacccggatggtagtcgagtgttatcggagacagcttgatgtagactccgtgcctgccggtcctcttattggcggacaccagtgagacaccccggaacttgctgtttttctgcaaaatccggggtgaccagtgggagcctatttgcacacacgagcgggacaccccactctggtgaagagtgccaaagtcattctttttcccgtnncggggcagccgattgcatgttttaggaaaatattacctttgctacaccctgtcagatttaccctccacacatatatattccgtcacctccagggactattcttggctcgttgcgccgccgcggaagatatccagaagctgtgttttccgagagactcggttggcgcctggtatatttnnaggatgtcgcgctgcctcacgtcccggtacccaggaacgcggtgggatctcgggcccatcgaagactgtgctccagactgctcgcccagcaggtgtttcttgattgccgcctctaaatagtccgcgcatcgccggtaacatttttccagctcggagtttgcgtttagatacatttctgcgatgccaaaggagcctgcagattataacctcggatgctgtcattcagcgcttttaatttgacctccagatagttgctgtatttctgttccattggctgctggacgttcgtataactcgagttattgttgcgctctgcctcggcgtactggctcatgactgactgcggtcgcttctcgagtgttctcgcaacaggacgcctgcaggtcatcgagtcgagctggcgccgaaactggcggatctgacctccacactgccctgtatctctatccaccgggaaccgcctcctgccgttccagaatgttgttcaagtggtagctctgtgcggtcaatgaaggcgttattgccggtgaaatctttgggaagcggtttatcctcggggaagattacgaaattcccgcgcgtcgttgcgcttcctggatctcgaggaagatcgttctccgcgtcgaggagatcgttctccgcgtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 18 - DNA, 51 bp, Artificial sequence
  synthetic probe or primer
tgcttcctaccactagcagcactaggatatgaggtgcgcaacgtgtccggg

SEQ ID NO: 19 - DNA, 52 bp, Artificial sequence
  synthetic probe or primer
tagtactagtattagtaggcttcgcatgaattcccgatgaaggcagagagcg

SEQ ID NO: 20 - DNA, 4252 bp, Artificial sequence
  vector pUC18-FMD-CL-E1-H6
  misc_feature (1006)..(1007): N is any nucleotide
  misc_feature (1185)..(1186): N is any nucleotide
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatccttaatggtgatggtggtggtgccagttcatcatcatatcccaagccatacggtgacctgttatgtggccgggatagattgagcaattgcagtcctgcaccgtctcatgccggcgaggcgagatggtgaacagctgggagacgaggaagacagatccgcagagatcccccacgtacatagcggaacagaaagcagccgccccaacgagcaaatcgacgtggcgtcgtattgtcgtagtggggacgctggcgttcctagctgcgagcgtgggggtgagcgctacccagcagcgggaagagttgttctcccgaacgcagggcacgcacccgggggtgtgcatgatcatgtccgctgcctcatacacaatgcttgagttggagcagtcgttcgtgacatggtacatcccggacacgttgcgcacctcatatcctagtgctgctagtggtaggaagcatagtactagtattagtaggcttcgcatgaattcccgatgaaggcagagagcgcaaggaggcggtatttatagtgccattcccctctctgagagacccggatggtagtcgagtgttatcggagacagcttgatgtagactccgtgcctgccggtcctcttattggcggacaccagtgagacaccccggaacttgctgtttttctgcaaaatccggggtgaccagtgggagcctatttgcacacacgagcgggacaccccactctggtgaagagtgccaaagtcattctttttcccgtnncggggcagccgattgcatgttttaggaaaatattacctttgctacaccctgtcagatttaccctccacacatatatattccgtcacctccagggactattcttggctcgttgcgccgccgcggaagatatccagaagctgtgttttccgagagactcggttggcgcctggtatatttnnaggatgtcgcgctgcctcacgtcccggtacccaggaacgcggtgggatctcgggcccatcgaagactgtgctccagactgctcgcccagcaggtgtttcttgattgccgcctctaaatagtccgcgcatcgccggtaacatttttccagctcggagtttgcgtttagatacatttctgcgatgccaaaggagcctgcagattataacctcggatgctgtcattcagcgcttttaatttgacctccagatagttgctgtatttctgttccattggctgctggacgttcgtataactcgagttattgttgcgctctgcctcggcgtactggctcatgactgactgcggtcgcttctcgagtgttctcgcaacaggacgcctgcaggtcatcgagtcgagctggcgccgaaactggcggatctgacctccacactgccctgtatctctatccaccgggaaccgcctcctgccgttccagaatgttgttcaagtggtagctctgtgcggtcaatgaaggcgttattgccggtgaaatctttgggaagcggtttatcctcggggaagattacgaaattcccgcgcgtcgttgcgcttcctggatctcgaggaagatcgttctccgcgtcgaggagatcgttctccgcgtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 21 - DNA, 7447 bp, Artificial sequence
  vector pFPMT-CL-E1-H6
ggtaccctgctcaatctccggaatggtgatctgatcgttcctgaaaacctcgacattggctccctcctgacacaggtactcgtacaggttccaggtaaacgagtcgtagttgtcgatcatgacaacgttcttagaagcggccggcattttgaaggtgactaatagcctaagaaaatatttaatttaattttcattaaattttcctatactcgctatttcagcttttcatctcatcacttcataaacgatataaaccagaaaaagaactattttcaaacacgcttctcaaaagcggtatgtccttccacgtctccttagaatctggcaagtccgcgagggggatccttaatggtgatggtggtggtgccagttcatcatcatatcccaagccatacggtgacctgttatgtggccgggatagattgagcaattgcagtcctgcaccgtctcatgccggcgaggcgagatggtgaacagctgggagacgaggaagacagatccgcagagatcccccacgtacatagcggaacagaaagcagccgccccaacgagcaaatcgacgtggcgtcgtattgtcgtagtggggacgctggcgttcctagctgcgagcgtgggggtgagcgctacccagcagcgggaagagttgttctcccgaacgcagggcacgcacccgggggtgtgcatgatcatgtccgctgcctcatacacaatgcttgagttggagcagtcgttcgtgacatggtacatcccggacacgttgcgcacctcatatcctagtgctgctagtggtaggaagcatagtactagtattagtaggcttcgcatgaattcccgatgaagcagagagcgcaggaggcggtatttatagtgccattcccctctctgagagacccggatggtagtcgagtgtatcggagacagcttgatgtagactccgtgcctgccggctcctcttattggcggacaccagtgagacaccccggaacttgctgtttttctgcaaaatccggggtgaccagtgggagcctatttgcacacacgagcgggacaccccactctggtgaagagtgccaaagtcattctttttcccgttgcggggcagccgattgcatgttttaggaaaatattacctttgctacaccctgtcagatttaccctccacacatatatattccgtcacctccagggactattattcgtcgttgcgccgccagcggaagatatccagaagctgttttccgagagactcggttggcgcctggtatatttgatggatgtcgcgctgcctcacgtcccggtacccaggaacgcggtgggatctcgggcccatcgaagactgtgctccagactgctcgcccagcaggtgtttcttgatcgccgcctctaaattgtccgcgcatcgccggtaacatttttccagctcggagtttgcgtttagatacagtttctgcgatgccaaaggagcctgcagattataacctcggatgctgtcattcagcgcttttaatttgacctccagatagttgctgtatttctgttcccattggctgctgcgcagcttcgtataactcgagttattgttgcgctctgcctcggcgtactggctcatgatctggatcttgtccgtgtcgcttttcttcgagtgtttctcgcaaacgatgtgcacggcctgcagtgtccaatcggagtcgagctggcgccgaaactggcggatctgagcctccacactgccctgtttctctatccacggcggaaccgcctcctgccgtttcagaatgttgttcaagtggtactctgtgcggtcaatgaaggcgttattgccggtgaaatctttgggaagcggttttcctcggggaagattacgaaattccccgcgtcgttgcgcttcctggatctcgaggagatcgttctccgcgtcgaggagatcgttctccgcgtcgacaccattccttgcggcggcggtgctcaacggcctcaacctactactgggctgcttcctaatgcaggagtcgcataagggagagcgtcgacaaacccgcgtttgagaacttgctcaagcttctggtaaacgttgtagtactctgaaacaaggccctagcactctgatctgtttctcttgggtagcggtgagtggtttattggagttcactggtttcagcacatctgtcatctagacaatattgttactaaatttttttgaactacaattgttcgtaattcatctattattatacatcctcgtcagcaatttctggcagacggagtttactaacgtcttgagtatgaggccgagaatccagctctgtggccatactcagtcttgacagcctgctgatgtggctgcgttcaacgcaataagcgtgtcctccgactccgagttgtgctcgttatcgtcgttctcatcctcggaaaaatcacacgaaagaacatactcaccagtaggctttctggtccctggggcacggctgtttctgacgtattccggcgttgataatagctcgaaagtgaacgccgagtcgcgggagtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatcggggggggggggggggggggggggcaaacaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaagccccccccccccccccccccccccccccccccccgcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaattcccccttacacggaggcatcaagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaa

SEQ ID NO: 22 - DNA, 3730 bp, Artificial sequence
  vector pSP72E2H6
gaactcgagcagctgaagcttgaattcatgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctagataaaaggcatacccgcgtgtcaggaggggcagcagcctccgataccaggggccttgtgtccctctttagccccgggtcggctcagaaaatccagctcgtaaacaccaacggcagttggcacatcaacaggactgccctgaactgcaacgactccctccaaacagggttctttgccgcactattctacaaacacaaattcaactcgtctggatgcccagagcgcttggccagctgtcgctccatcgacaagttcgctcaggggtggggtcccctcacttacactgagcctaacagctcggaccagaggccctactgctggcactacgcgcctcgaccgtgtggtattgtacccgcgtctcaggtgtgcggtccagtgtattgcttcaccccgagccctgttgtggtggggacgaccgatcggtttggtgtccccacgtataactggggggcgaacgactcggatgtgctgattctcaacaacacgcggccgccgcgaggcaactggttcggctgtacatggatgaatggcactgggttcaccaagacgtgtgggggccccccgtgcaacatcgggggggccggcaacaacaccttgacctgccccactgactgttttcggaagcaccccgaggccacttacgccagatgcggttctgggccctggctgacacctaggtgtatggttcattacccatataggctctggcactacccctgcactgtcaacttcaccatcttcaaggttaggatgtacgtggggggcgtggagcacaggttcgaagccgcatgcaattggactcgaggagagcgttgtgacttggaggacagggatagatcagagcttagctcgctgctgctgtctacaacagagtggcaggtgatcgagggcagacaccatcaccaccatcactaatagttaattaacgatctcgacttggttgaacacgttgccaaggcttaagtgaatttactttaaagtcttgcatttaaataaattttctttttatagctttatgacttagtttcaatttatatactattttaatgacattttcgattcattgattgaaagctatcagatctgccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactata

SEQ ID NO: 23 - DNA, 7370 bp, Artificial sequence
  vector pMPT121
  misc_feature (778)..(778): N is any nucleotide
ggtaccctgctcaatctccggaatggtgatctgatcgttcctgaaaacctcgacattggctccctcctgacacaggtactcgtacaggttccaggtaaacgagtcgtagttgtcgatcatgacaacgttcttagaagcggccggcattttgaaggtgactaatagcctaagaaaatatttaatttaattttcattaaattttcctatactcgctatttcagcttttcatctcatcacttcataaacgatataaaccagaaaaagaactattttcaaacacgcttctcaaaagcggtatgtccttccacgtctccttagaatctggcaagtccgcgagggggatccagatctgaattcgtttttgtactttagattgatgtcaccaccgtgcactggcagcagtatttatagatggaccgtgtggggacggttgggtacacttagcggcagcgctgaccccatctgtgatcaagtagggcaaaaactggggatgtcggagtcgctgcacggtagcataagaatttactttctggccggttcacccgcatttgcactgtggagaaacagcctgtccgacaccccaccagttgccacatcggccctctgctgctctggtgattttctggtagcaggcacagacagcagtgggtagcgccgtccggttaggcaaggtcacgttgtaggctaccccagcaaacagagcctcacatgacaccatccagctgcgtcctcgaagcgaaaagttcggttgcggctgcagaaccccctcagttgccanattcacaagttttacgcgacggctaaagcgagtgggttttaaaaacttgcggtgcaaggatgcatgcggcaacaattaattggtgcatccagcacagcaagcccagtctcgagatgtccagtcgctacagagtggagtacgcactcaaggaacaccgtcgagatggcctcatagaatggatcaagggcctgctggccacgccgttcgtcctgtacgcggtgaagagcaacggcatctctgcagtggacgacctcatggtaaactctgaggcaaaacgccgctacgcggaaatcttccacgacctcgaactcctcatcgacgacaacattgaaatgaccaaagccggcacccccgaattgtctcggctcgtgcagctggttccgagcgttggcagcttcttcacgagactgcctctggaaaaggccttctacatcgaggacgagcgccgcgccatcagcaaacgccggcttgtggccccctcgttcaacgacgtccggctcattctcaacacggcccagctgttggagatgtcgcggttcttccattccaaaaccatccgagatcgcaagctgcagctcattacattcgatggtgacatcacactgtacgacgacggcaaaaatttcgatgccgagtcgcccatcctgccccacctcatcaaactaatggccaaggacctctatgtgggtatcgtcaccgcggccggctacagcgacggaacaagtactacgagcgcctcaagggcctcatcgacgccgtccagacgtccccgctgctcacaggccaccagaaagagaacctgttcattatgggcggcgaggcaaactacctcttccggtacagtaacgaggagcagagattacgcttctactccaaagacagatggctgctcgagaacatgctgaattggtccgaggaggacattcatctgacactggactttgcgcaggacgttctaaacgacctcgttcacaaactgggctcgccagccaccgtggtccgcaaggagcgtcgcgtcggcctggttccattaccgggccacaagctgatccgcgagcagctcgaggagatcgttctccgcgtcgacaccattccttgcggcggcggtgctcaacggcctcaacctactactgggctgcttcctaatgcaggagtcgcataagggagagcgtcgactcccgcgactcggcgttcactttcgagctattatcaacgccggaatacgtcagaaacagccgtgccccagggaccagaaagcctactggtgagtatgttctttcgtgtgatttttccgaggatgagaacgacgataacgagcacaactcggagtcggaggacacgcttattgcgttgaacgcagccacatcagcaggctgtcaagactgagtatggccacagagctggattctcggcctcatactcaagacgttagtaaactccgtctgccagaaattgctgacgaggatgtataataatagatgaattacgaacaattgtagttcaaaaaaatttagtaacaatattgtctagatgacagatgtgctgaaaccagtgaactccaataaaccactcaccgctacccaagagaaacagatcagagtgctagggccttgtttcagagtactacaacgtttaccagaagcttgagcaagttctcaaacgcgggtttgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatcggggggggggggggggggggggggcaaacaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaagccccccccccccccccccccccccccccccccccgcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaattcccccttacacggaggcatcaagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaa

SEQ ID NO: 24 - DNA, 8298 bp, Artificial sequence
  vector pFMPT-MFalfa-E2-H6
ggtaccctgctcaatctccggaatggtgatctgatcgttcctgaaaacctcgacattggctccctcctgacacaggtactcgtacaggttccaggtaaacgagtcgtagttgtcgatcatgacaacgttcttagaagcggccggcattttgaaggtgactaatagcctaagaaaatatttaatttaattttcattaaattttcctatactcgctatttcagcttttcatctcatcacttcataaacgatataaaccagaaaaagaactattttcaaacacgcttctcaaaagcggtatgtccttccacgtctccttagaatctggcaagtccgcgagggggatccagatctgatagctttcaatcaatgaatcgaaaatgtcattaaaatagtatataaattgaaactaagtcataaagctataaaaagaaaatttatttaaatgcaagactttaaagtaaattcacttaagccttggcaacgtgttcaaccaagtcgagatcgttaattaactattagtgatggtggtgatggtgtctgccctcgatcacctgccactctgttgtagacagcagcagcgagctaagctctgatctatccctgtcctccaagtcacaacgctctcctcgagtccaattgcatgcggcttcgaacctgtgctccacgccccccacgtacatcctaaccttgaagatggtgaagttgacagtgcaggggtagtgccagagcctatatgggtaatgaaccatacacctaggtgtcagccagggcccagaaccgcatctggcgtaagtggcctcggggtgcttccgaaaacagtcagtggggcaggtcaaggtgttgttgccggcccccccgatgttgcacggggggcccccacacgtcttggtgaacccagtgccattcatccatgtacagccgaaccagttgcctcgcggcggccgcgtgttgttgagaatcagcacatccgagtcgttcgccccccagttatacgtggggacaccaaaccgatcggtcgtccccaccacaacagggctcggggtgaagcaatacactggaccgcacacctgagacgcgggtacaataccacacggtcgaggcgcgtagtgccagcagtagggcctctggtccgagctgttaggctcagtgtaagtgaggggaccccacccctgagcgaacttgtcgatggagcgacagctggccaagcgctctgggcatccagacgagttgaatttgtgtttgtagaatagtgcggcaaagaaccctgtttggagggagtcgttgcagttcagggcagtcctgttgatgtgccaactgccgttggtgtttacgagctggattttctgagccgacccggggctaaagagggacacaaggcccctggtatcggaggctgctgcccctcctgacacgcgggtatgccttttatctagagataccccttcttctttagcagcaatgctggcaatagtagtatttataaacaataacccgttatttgtgctgttggaaaatggcaaaacagcaacatcgaaatccccttctaaatctgagtaaccgatgacagcttcagccggaatttgtgccgtttcatcttctgttgtagtgttgactggagcagctaatgcggaggatgctgcgaataaaactgcagtaaaaattgaaggaaatctcatgaattcccgatgaagcagagagcgcaggaggcggtatttatagtgccattcccctctctgagagacccggatggtagtcgagtgtatcggagacagcttgatgtagactccgtgcctgccggctcctcttattggcggacaccagtgagacaccccggaacttgctgtttttctgcaaaatccggggtgaccagtgggagcctatttgcacacacgagcgggacaccccactctggtgaagagtgccaaagtcattctttttcccgttgcggggcagccgattgcatgttttaggaaaatattacctttgctacaccctgtcagatttaccctccacacatatatattccgtcacctccagggactattattcgtcgttgcgccgccagcggaagatatccagaagctgttttccgagagactcggttggcgcctggtatatttgatggatgtcgcgctgcctcacgtcccggtacccaggaacgcggtgggatctcgggcccatcgaagactgtgctccagactgctcgcccagcaggtgtttcttgatcgccgcctctaaattgtccgcgcatcgccggtaacatttttccagctcggagtttgcgtttagatacagtttctgcgatgccaaaggagcctgcagattataacctcggatgctgtcattcagcgcttttaatttgacctccagatagttgctgtatttctgttcccattggctgctgcgcagcttcgtataactcgagttattgttgcgctctgcctcggcgtactggctcatgatctggatcttgtccgtgtcgcttttcttcgagtgtttctcgcaaacgatgtgcacggcctgcagtgtccaatcggagtcgagctggcgccgaaactggcggatctgagcctccacactgccctgtttctctatccacggcggaaccgcctcctgccgtttcagaatgttgttcaagtggtactctgtgcggtcaatgaaggcgttattgccggtgaaatctttgggaagcggttttcctcggggaagattacgaaattccccgcgtcgttgcgcttcctggatctcgaggagatcgttctccgcgtcgaggagatcgttctccgcgtcgacaccattccttgcggcggcggtgctcaacggcctcaacctactactgggctgcttcctaatgcaggagtcgcataagggagagcgtcgacaaacccgcgtttgagaacttgctcaagcttctggtaaacgttgtagtactctgaaacaaggccctagcactctgatctgtttctcttgggtagcggtgagtggtttattggagttcactggtttcagcacatctgtcatctagacaatattgttactaaatttttttgaactacaattgttcgtaattcatctattattatacatcctcgtcagcaatttctggcagacggagtttactaacgtcttgagtatgaggccgagaatccagctctgtggccatactcagtcttgacagcctgctgatgtggctgcgttcaacgcaataagcgtgtcctccgactccgagttgtgctcgttatcgtcgttctcatcctcggaaaaatcacacgaaagaacatactcaccagtaggctttctggtccctggggcacggctgtttctgacgtattccggcgttgataatagctcgaaagtgaacgccgagtcgcgggagtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatcggggggggggggggggggggggggcaaacaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaagccccccccccccccccccccccccccccccccccgcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaattcccccttacacggaggcatcaagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaa

SEQ ID NO: 25 - DNA, 8695 bp, Artificial sequence
  vector pMPT-Mfalfa-E2-H6
  misc_feature (2103)..(2103): N is any nucleotide
ggtaccctgctcaatctccggaatggtgatctgatcgttcctgaaaacctcgacattggctccctcctgacacaggtactcgtacaggttccaggtaaacgagtcgtagttgtcgatcatgacaacgttcttagaagcggccggcattttgaaggtgactaatagcctaagaaaatatttaatttaattttcattaaattttcctatactcgctatttcagcttttcatctcatcacttcataaacgatataaaccagaaaaagaactattttcaaacacgcttctcaaaagcggtatgtccttccacgtctccttagaatctggcaagtccgcgagggggatccagatctgatagctttcaatcaatgaatcgaaaatgtcattaaaatagtatataaattgaaactaagtcataaagctataaaaagaaaatttatttaaatgcaagactttaaagtaaattcacttaagccttggcaacgtgttcaaccaagtcgagatcgttaattaactattagtgatggtggtgatggtgtctgccctcgatcacctgccactctgttgtagacagcagcagcgagctaagctctgatctatccctgtcctccaagtcacaacgctctcctcgagtccaattgcatgcggcttcgaacctgtgctccacgccccccacgtacatcctaaccttgaagatggtgaagttgacagtgcaggggtagtgccagagcctatatgggtaatgaaccatacacctaggtgtcagccagggcccagaaccgcatctggcgtaagtggcctcggggtgcttccgaaaacagtcagtggggcaggtcaaggtgttgttgccggcccccccgatgttgcacggggggcccccacacgtcttggtgaacccagtgccattcatccatgtacagccgaaccagttgcctcgcggcggccgcgtgttgttgagaatcagcacatccgagtcgttcgccccccagttatacgtggggacaccaaaccgatcggtcgtccccaccacaacagggctcggggtgaagcaatacactggaccgcacacctgagacgcgggtacaataccacacggtcgaggcgcgtagtgccagcagtagggcctctggtccgagctgttaggctcagtgtaagtgaggggaccccacccctgagcgaacttgtcgatggagcgacagctggccaagcgctctgggcatccagacgagttgaatttgtgtttgtagaatagtgcggcaaagaaccctgtttggagggagtcgttgcagttcagggcagtcctgttgatgtgccaactgccgttggtgtttacgagctggattttctgagccgacccggggctaaagagggacacaaggcccctggtatcggaggctgctgcccctcctgacacgcgggtatgccttttatctagagataccccttcttctttagcagcaatgctggcaatagtagtatttataaacaataacccgttatttgtgctgttggaaaatggcaaaacagcaacatcgaaatccccttctaaatctgagtaaccgatgacagcttcagccggaatttgtgccgtttcatcttctgttgtagtgttgactggagcagctaatgcggaggatgctgcgaataaaactgcagtaaaaattgaaggaaatctcatgaattcgtttttgtactttagattgatgtcaccaccgtgcactggcagcagtatttatagatggaccgtgtggggacggttgggtacacttagcggcagcgctgaccccatctgtgatcaagtagggcaaaaactggggatgtcggagtcgctgcacggtagcataagaatttactttctggccggttcacccgcatttgcactgtggagaaacagcctgtccgacaccccaccagttgccacatcggccctctgctgctctggtgattttctggtagcaggcacagacagcagtgggtagcgccgtccggttaggcaaggtcacgttgtaggctaccccagcaaacagagcctcacatgacaccatccagctgcgtcctcgaagcgaaaagttcggttgcggctgcagaaccccctcagttgccanattcacaagttttacgcgacggctaaagcgagtgggttttaaaaacttgcggtgcaaggatgcatgcggcaacaattaattggtgcatccagcacagcaagcccagtctcgagatgtccagtcgctacagagtggagtacgcactcaaggaacaccgtcgagatggcctcatagaatggatcaagggcctgctggccacgccgttcgtcctgtacgcggtgaagagcaacggcatctctgcagtggacgacctcatggtaaactctgaggcaaaacgccgctacgcggaaatcttccacgacctcgaactcctcatcgacgacaacattgaaatgaccaaagccggcacccccgaattgtctcggctcgtgcagctggttccgagcgttggcagcttcttcacgagactgcctctggaaaaggccttctacatcgaggacgagcgccgcgccatcagcaaacgccggcttgtggccccctcgttcaacgacgtccggctcattctcaacacggcccagctgttggagatgtcgcggttcttccattccaaaaccatccgagatcgcaagctgcagctcattacattcgatggtgacatcacactgtacgacgacggcaaaaatttcgatgccgagtcgcccatcctgccccacctcatcaaactaatggccaaggacctctatgtgggtatcgtcaccgcggccggctacagcgacggaacaagtactacgagcgcctcaagggcctcatcgacgccgtccagacgtccccgctgctcacaggccaccagaaagagaacctgttcattatgggcggcgaggcaaactacctcttccggtacagtaacgaggagcagagattacgcttctactccaaagacagatggctgctcgagaacatgctgaattggtccgaggaggacattcatctgacactggactttgcgcaggacgttctaaacgacctcgttcacaaactgggctcgccagccaccgtggtccgcaaggagcgtcgcgtcggcctggttccattaccgggccacaagctgatccgcgagcagctcgaggagatcgttctccgcgtcgacaccattccttgcggcggcggtgctcaacggcctcaacctactactgggctgcttcctaatgcaggagtcgcataagggagagcgtcgactcccgcgactcggcgttcactttcgagctattatcaacgccggaatacgtcagaaacagccgtgccccagggaccagaaagcctactggtgagtatgttctttcgtgtgatttttccgaggatgagaacgacgataacgagcacaactcggagtcggaggacacgcttattgcgttgaacgcagccacatcagcaggctgtcaagactgagtatggccacagagctggattctcggcctcatactcaagacgttagtaaactccgtctgccagaaattgctgacgaggatgtataataatagatgaattacgaacaattgtagttcaaaaaaatttagtaacaatattgtctagatgacagatgtgctgaaaccagtgaactccaataaaccactcaccgctacccaagagaaacagatcagagtgctagggccttgtttcagagtactacaacgtttaccagaagcttgagcaagttctcaaacgcgggtttgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatcggggggggggggggggggggggggcaaacaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaagccccccccccccccccccccccccccccccccccgcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaattcccccttacacggaggcatcaagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaa

SEQ ID NO: 26 - DNA, 36 bp, Artificial sequence
  synthetic probe or primer
agtcactcttcaaggcatacccgcgtgtcaggaggg

SEQ ID NO: 27 - DNA, 39 bp, Artificial sequence
  synthetic probe or primer
agtcactcttcacagggatccttagtgatggtggtgatg

SEQ ID NO: 28 - DNA, 4190 bp, Artificial sequence
  vector pMF30
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcagttgattgcagatgccagatcccgaaagaacagaggacggagcgtaaacttgtggcattccaccagaaattgatacagataagcttccggagtcaccagctaaaacggaattgcaagaaataatatcgataactttatcaccactagaatagccggtgttgctgacagtaatatcctgtgacccgtttgaacctaaattattaaaaatggaaatcaattgattagcatcgctacccttcctagtggctatatagtggtctgaagaagaaacaactgaggatttgtaagttgaataggcagaatccttcttaatagcttgatttcttatttgatttagtttactgattagctcgtagtattctgaatcggtattatatccacttaaccataaagcttctctattggcaggatcggaaccaccattgagaccttgttcttggccataataaataattgggataccatcacccaaaattataaaagccatgtcattcttaatcaaggatgtgtctgaggtaactgatggaaatctaacttggtcatggttttcaataaagtttcccaacaaagagacgtccgaacaagatgactgtaacgtggagatcattgaagttaactcactggaagtcgccgaagtatcactgaagaatctatatactggatagtataatggatagttggtaactcctttcatataattctgatatggacaagtataagttggatctccttgataaacttcacctaagttataaacaccagaagcgtcctcaaacttcgttaatgaagcggtatctacgtgctttgcactatcaattcttaaaccatcgattgaatagttttgaacaaaatctgacacccaagtttgaaatactcctataacttcattatcctcggtacttaaatctggaagggagacttcagtatcaccttcccaacaatcttcaacattggtttgatcattataatttgtaatcaaacaataatcgtggaagtaagattgttgattgaatggagtgaaactagaataatctacgcttgaaccatctccgttccaagcataatggttgtaaacaacgtcgaccatcaataacatgcttctggaatgcaattcgctagctaattgtttcaattcatcagcggtaccaaaattagtgttcaattcatcaatatttttcatccaataaccatggtaagcataaccataagcagtattgtcaggaatttgctcaacaactggggagatccagatcgcagtgaaacccataccttgaatataatccaacttgtcgataatccctttataagatccaccacagtacttgcgatcactcactaaacagtcagctgtggtcgagccatcagatctggcaaacctatcagtaacgatttgataaatcgattggtctttccatttatcagctgacgagctaacatccctcttgtcaaaaataatcggttgagcagataccaatcttgagaatgctaaaattgctgcaacaactttacttgtaaatccttcagttgaaaatctcattgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 29 - DNA, 38 bp, Artificial sequence
  synthetic probe or primer
agtcactcttcacctcttgtcaaaaataatcggttgag

SEQ ID NO: 30 - DNA, 52 bp, Artificial sequence
  synthetic probe or primer
tgcttcctaccactagcagcactaggacatacccgcgtgtcaggaggggcag

SEQ ID NO: 31 - DNA, 57 bp, Artificial sequence
  synthetic probe or primer
tagtactagtattagtaggcttcgcatggaattcactggccgtcgttttacaacgtc

SEQ ID NO: 32 - DNA, 7927 bp, Artificial sequence
  vector pFMPT-CL-E2-H6
ggtaccctgctcaatctccggaatggtgatctgatcgttcctgaaaacctcgacattggctccctcctgacacaggtactcgtacaggttccaggtaaacgagtcgtagttgtcgatcatgacaacgttcttagaagcggccggcattttgaaggtgactaatagcctaagaaaatatttaatttaattttcattaaattttcctatactcgctatttcagcttttcatctcatcacttcataaacgatataaaccagaaaaagaactattttcaaacacgcttctcaaaagcggtatgtccttccacgtctccttagaatctggcaagtccgcgagggggatccttagtgatggtggtgatggtgtctgccctcgatcacctgccactctgttgtagacagcagcagcgagctaagctctgatctatccctgtcctccaagtcacaacgctctcctcgagtccaattgcatgcggcttcgaacctgtgctccacgccccccacgtacatcctaaccttgaagatggtgaagttgacagtgcaggggtagtgccagagcctatatgggtaatgaaccatacacctaggtgtcagccagggcccagaaccgcatctggcgtaagtggcctcggggtgcttccgaaaacagtcagtggggcaggtcaaggtgttgttgccggcccccccgatgttgcacggggggcccccacacgtcttggtgaacccagtgccattcatccatgtacagccgaaccagttgcctcgcggcggccgcgtgttgttgagaatcagcacatccgagtcgttcgccccccagttatacgtggggacaccaaaccgatcggtcgtccccaccacaacagggctcggggtgaagcaatacactggaccgcacacctgagacgcgggtacaataccacacggtcgaggcgcgtagtgccagcagtagggcctctggtccgagctgttaggctcagtgtaagtgaggggaccccacccctgagcgaacttgtcgatggagcgacagctggccaagcgctctgggcatccagacgagttgaatttgtgtttgtagaatagtgcggcaaagaaccctgtttggagggagtcgttgcagttcagggcagtcctgttgatgtgccaactgccgttggtgtttacgagctggattttctgagccgacccggggctaaagagggacacaaggcccctggtatcggaggctgctgcccctcctgacacgcgggtatgtcctagtgctgctagtggtaggaagcatagtactagtattagtaggctgcgcatgaattcccgatgaagcagagagcgcaggaggcggtatttatagtgccattcccctctctgagagacccggatggtagtcgagtgtatcggagacagcttgatgtagactccgtgcctgccggctcctcttattggcggacaccagtgagacaccccggaacttgctgtttttctgcaaaatccggggtgaccagtgggagcctatttgcacacacgagcgggacaccccactctggtgaagagtgccaaagtcattctttttcccgttgcggggcagccgattgcatgttttaggaaaatattacctttgctacaccctgtcagatttaccctccacacatatatattccgtcacctccagggactattattcgtcgttgcgccgccagcggaagatatccagaagctgttttccgagagactcggttggcgcctggtatatttgatggatgtcgcgctgcctcacgtcccggtacccaggaacgcggtgggatctcgggcccatcgaagactgtgctccagactgctcgcccagcaggtgtttcttgatcgccgcctctaaattgtccgcgcatcgccggtaacatttttccagctcggagtttgcgtttagatacagtttctgcgatgccaaaggagcctgcagattataacctcggatgctgtcattcagcgcttttaatttgacctccagatagttgctgtatttctgttcccattggctgctgcgcagcttcgtataactcgagttattgttgcgctctgcctcggcgtactggctcatgatctggatcttgtccgtgtcgcttttcttcgagtgtttctcgcaaacgatgtgcacggcctgcagtgtccaatcggagtcgagctggcgccgaaactggcggatctgagcctccacactgccctgtttctctatccacggcggaaccgcctcctgccgtttcagaatgttgttcaagtggtactctgtgcggtcaatgaaggcgttattgccggtgaaatctttgggaagcggttttcctcggggaagattacgaaattccccgcgtcgttgcgcttcctggatctcgaggagatcgttctccgcgtcgaggagatcgttctccgcgtcgacaccattccttgcggcggcggtgctcaacggcctcaacctactactgggctgcttcctaatgcaggagtcgcataagggagagcgtcgacaaacccgcgtttgagaacttgctcaagcttctggtaaacgttgtagtactctgaaacaaggccctagcactctgatctgtttctcttgggtagcggtgagtggtttattggagttcactggtttcagcacatctgtcatctagacaatattgttactaaatttttttgaactacaattgttcgtaattcatctattattatacatcctcgtcagcaatttctggcagacggagtttactaacgtcttgagtatgaggccgagaatccagctctgtggccatactcagtcttgacagcctgctgatgtggctgcgttcaacgcaataagcgtgtcctccgactccgagttgtgctcgttatcgtcgttctcatcctcggaaaaatcacacgaaagaacatactcaccagtaggctttctggtccctggggcacggctgtttctgacgtattccggcgttgataatagctcgaaagtgaacgccgagtcgcgggagtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatcggggggggggggggggggggggggcaaacaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaagccccccccccccccccccccccccccccccccccgcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaattcccccttacacggaggcatcaagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaa

SEQ ID NO: 33 - DNA, 24 bp, Artificial sequence
  synthetic probe or primer
taaggatccccgggtaccgagctc

SEQ ID NO: 34 - DNA, 25 bp, Artificial sequence
  synthetic probe or primer
ccagttcatcatcatatcccaagcc

SEQ ID NO: 35 - DNA, 4234 bp, Artificial sequence
  vector pUC18-FMD-CL-E1
  misc_feature (988)..(989): N is any nucleotide
  misc_feature (1167)..(1168): N is any nucleotide
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatccttaccagttcatcatcatatcccaagccatacggtgacctgttatgtggccgggatagattgagcaattgcagtcctgcaccgtctcatgccggcgaggcgagatggtgaacagctgggagacgaggaagacagatccgcagagatcccccacgtacatagcggaacagaaagcagccgccccaacgagcaaatcgacgtggcgtcgtattgtcgtagtggggacgctggcgttcctagctgcgagcgtgggggtgagcgctacccagcagcgggaagagttgttctcccgaacgcagggcacgcacccgggggtgtgcatgatcatgtccgctgcctcatacacaatgcttgagttggagcagtcgttcgtgacatggtacatcccggacacgttgcgcacctcatatcctagtgctgctagtggtaggaagcatagtactagtattagtaggcttcgcatgaattcccgatgaaggcagagagcgcaaggaggcggtatttatagtgccattcccctctctgagagacccggatggtagtcgagtgttatcggagacagcttgatgtagactccgtgcctgccggtcctcttattggcggacaccagtgagacaccccggaacttgctgtttttctgcaaaatccggggtgaccagtgggagcctatttgcacacacgagcgggacaccccactctggtgaagagtgccaaagtcattctttttcccgtnncggggcagccgattgcatgttttaggaaaatattacctttgctacaccctgtcagatttaccctccacacatatatattccgtcacctccagggactattcttggctcgttgcgccgccgcggaagatatccagaagctgtgttttccgagagactcggttggcgcctggtatatttnnaggatgtcgcgctgcctcacgtcccggtacccaggaacgcggtgggatctcgggcccatcgaagactgtgctccagactgctcgcccagcaggtgtttcttgattgccgcctctaaatagtccgcgcatcgccggtaacatttttccagctcggagtttgcgtttagatacatttctgcgatgccaaaggagcctgcagattataacctcggatgctgtcattcagcgcttttaatttgacctccagatagttgctgtatttctgttccattggctgctggacgttcgtataactcgagttattgttgcgctctgcctcggcgtactggctcatgactgactgcggtcgcttctcgagtgttctcgcaacaggacgcctgcaggtcatcgagtcgagctggcgccgaaactggcggatctgacctccacactgccctgtatctctatccaccgggaaccgcctcctgccgttccagaatgttgttcaagtggtagctctgtgcggtcaatgaaggcgttattgccggtgaaatctttgggaagcggtttatcctcggggaagattacgaaattcccgcgcgtcgttgcgcttcctggatctcgaggaagatcgttctccgcgtcgaggagatcgttctccgcgtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 36 - DNA, 7429 bp, Artificial sequence
  vector pFPMT-CL-E1
ggtaccctgctcaatctccggaatggtgatctgatcgttcctgaaaacctcgacattggctccctcctgacacaggtactcgtacaggttccaggtaaacgagtcgtagttgtcgatcatgacaacgttcttagaagcggccggcattttgaaggtgactaatagcctaagaaaatatttaatttaattttcattaaattttcctatactcgctatttcagcttttcatctcatcacttcataaacgatataaaccagaaaaagaactattttcaaacacgcttctcaaaagcggtatgtccttccacgtctccttagaatctggcaagtccgcgagggggatccttaccagttcatcatcatatcccaagccatacggtgacctgttatgtggccgggatagattgagcaattgcagtcctgcaccgtctcatgccggcgaggcgagatggtgaacagctgggagacgaggaagacagatccgcagagatcccccacgtacatagcggaacagaaagcagccgccccaacgagcaaatcgacgtggcgtcgtattgtcgtagtggggacgctggcgttcctagctgcgagcgtgggggtgagcgctacccagcagcgggaagagttgttctcccgaacgcagggcacgcacccgggggtgtgcatgatcatgtccgctgcctcatacacaatgcttgagttggagcagtcgttcgtgacatggtacatcccggacacgttgcgcacctcatatcctagtgctgctagtggtaggaagcatagtactagtattagtaggcttcgcatgaattcccgatgaagcagagagcgcaggaggcggtatttatagtgccattcccctctctgagagacccggatggtagtcgagtgtatcggagacagcttgatgtagactccgtgcctgccggctcctcttattggcggacaccagtgagacaccccggaacttgctgtttttctgcaaaatccggggtgaccagtgggagcctatttgcacacacgagcgggacaccccactctggtgaagagtgccaaagtcattctttttcccgttgcggggcagccgattgcatgttttaggaaaatattacctttgctacaccctgtcagatttaccctccacacatatatattccgtcacctccagggactattattcgtcgttgcgccgccagcggaagatatccagaagctgttttccgagagactcggttggcgcctggtatatttgatggatgtcgcgctgcctcacgtcccggtacccaggaacgcggtgggatctcgggcccatcgaagactgtgctccagactgctcgcccagcaggtgtttcttgatcgccgcctctaaattgtccgcgcatcgccggtaacatttttccagctcggagtttgcgtttagatacagtttctgcgatgccaaaggagcctgcagattataacctcggatgctgtcattcagcgcttttaatttgacctccagatagttgctgtatttctgttcccattggctgctgcgcagcttcgtataactcgagttattgttgcgctctgcctcggcgtactggctcatgatctggatcttgtccgtgtcgcttttcttcgagtgtttctcgcaaacgatgtgcacggcctgcagtgtccaatcggagtcgagctggcgccgaaactggcggatctgagcctccacactgccctgtttctctatccacggcggaaccgcctcctgccgtttcagaatgttgttcaagtggtactctgtgcggtcaatgaaggcgttattgccggtgaaatctttgggaagcggttttcctcggggaagattacgaaattccccgcgtcgttgcgcttcctggatctcgaggagatcgttctccgcgtcgaggagatcgttctccgcgtcgacaccattccttgcggcggcggtgctcaacggcctcaacctactactgggctgcttcctaatgcaggagtcgcataagggagagcgtcgacaaacccgcgtttgagaacttgctcaagcttctggtaaacgttgtagtactctgaaacaaggccctagcactctgatctgtttctcttgggtagcggtgagtggtttattggagttcactggtttcagcacatctgtcatctagacaatattgttactaaatttttttgaactacaattgttcgtaattcatctattattatacatcctcgtcagcaatttctggcagacggagtttactaacgtcttgagtatgaggccgagaatccagctctgtggccatactcagtcttgacagcctgctgatgtggctgcgttcaacgcaataagcgtgtcctccgactccgagttgtgctcgttatcgtcgttctcatcctcggaaaaatcacacgaaagaacatactcaccagtaggctttctggtccctggggcacggctgtttctgacgtattccggcgttgataatagctcgaaagtgaacgccgagtcgcgggagtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatcggggggggggggggggggggggggcaaacaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaagccccccccccccccccccccccccccccccccccgcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaattcccccttacacggaggcatcaagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaa

SEQ ID NO: 37 - DNA, 39 bp, Artificial sequence
  synthetic probe or primer
catcacaaatatgaggtgcgcaacgtgtccgggatgtac

SEQ ID NO: 38 - DNA, 42 bp, Artificial sequence
  synthetic probe or primer
gtgatggtggtgtcctagtgctgctagtggtaggaagcatag

SEQ ID NO: 39 - DNA, 4273 bp, Artificial sequence
  vector pUC18-FMD-CL-E1-H-K6
  misc_feature (1027)..(1028): N is any nucleotide
  misc_feature (1206)..(1207): N is any nucleotide
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatccttaatggtgatggtggtggtgccagttcatcatcatatcccaagccatacggtgacctgttatgtggccgggatagattgagcaattgcagtcctgcaccgtctcatgccggcgaggcgagatggtgaacagctgggagacgaggaagacagatccgcagagatcccccacgtacatagcggaacagaaagcagccgccccaacgagcaaatcgacgtggcgtcgtattgtcgtagtggggacgctggcgttcctagctgcgagcgtgggggtgagcgctacccagcagcgggaagagttgttctcccgaacgcagggcacgcacccgggggtgtgcatgatcatgtccgctgcctcatacacaatgcttgagttggagcagtcgttcgtgacatggtacatcccggacacgttgcgcacctcatatttgtgatggtgatggtggtgtcctagtgctgctagtggtaggaagcatagtactagtattagtaggcttcgcatgaattcccgatgaaggcagagagcgcaaggaggcggtatttatagtgccattcccctctctgagagacccggatggtagtcgagtgttatcggagacagcttgatgtagactccgtgcctgccggtcctcttattggcggacaccagtgagacaccccggaacttgctgtttttctgcaaaatccggggtgaccagtgggagcctatttgcacacacgagcgggacaccccactctggtgaagagtgccaaagtcattctttttcccgtnncggggcagccgattgcatgttttaggaaaatattacctttgctacaccctgtcagatttaccctccacacatatatattccgtcacctccagggactattcttggctcgttgcgccgccgcggaagatatccagaagctgtgttttccgagagactcggttggcgcctggtatatttnnaggatgtcgcgctgcctcacgtcccggtacccaggaacgcggtgggatctcgggcccatcgaagactgtgctccagactgctcgcccagcaggtgtttcttgattgccgcctctaaatagtccgcgcatcgccggtaacatttttccagctcggagtttgcgtttagatacatttctgcgatgccaaaggagcctgcagattataacctcggatgctgtcattcagcgcttttaatttgacctccagatagttgctgtatttctgttccattggctgctggacgttcgtataactcgagttattgttgcgctctgcctcggcgtactggctcatgactgactgcggtcgcttctcgagtgttctcgcaacaggacgcctgcaggtcatcgagtcgagctggcgccgaaactggcggatctgacctccacactgccctgtatctctatccaccgggaaccgcctcctgccgttccagaatgttgttcaagtggtagctctgtgcggtcaatgaaggcgttattgccggtgaaatctttgggaagcggtttatcctcggggaagattacgaaattcccgcgcgtcgttgcgcttcctggatctcgaggaagatcgttctccgcgtcgaggagatcgttctccgcgtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 40 - DNA, 7330 bp, Artificial sequence
  vector pFPMT-CL-H6-K-E1
  misc_feature (1098)..(1099): N is any nucleotide
  misc_feature (1277)..(1278): N is any nucleotide
ggtaccctgctcaatctccggaatggtgatctgatcgttcctgaaaacctcgacattggctccctcctgacacaggtactcgtacaggttccaggtaaacgagtcgtagttgtcgatcatgacaacgttcttagaagcggccggcattttgaaggtgactaatagcctaagaaaatatttaatttaattttcattaaattttcctatactcgctatttcagcttttcatctcatcacttcataaacgatataaaccagaaaaagaactattttcaaacacgcttctcaaaagcggtatgtccttccacgtctccttagaatctggcaagtccgcgagggggatccttaccagttcatcatcatatcccaagccatacggtgacctgttatgtggccgggatagattgagcaattgcagtcctgcaccgtctcatgccggcgaggcgagatggtgaacagctgggagacgaggaagacagatccgcagagatcccccacgtacatagcggaacagaaagcagccgccccaacgagcaaatcgacgtggcgtcgtattgtcgtagtggggacgctggcgttcctagctgcgagcgtgggggtgagcgctacccagcagcgggaagagttgttctcccgaacgcagggcacgcacccgggggtgtgcatgatcatgtccgctgcctcatacacaatgcttgagttggagcagtcgttcgtgacatggtacatcccggacacgttgcgcacctcatatttgtgatggtgatggtggtgtcctagtgctgctagtggtaggaagcatagtactagtattagtaggcttcgcatgaattcccgatgaaggcagagagcgcaaggaggcggtatttatagtgccattcccctctctgagagacccggatggtagtcgagtgttatcggagacagcttgatgtagactccgtgcctgccggtcctcttattggcggacaccagtgagacaccccggaacttgctgtttttctgcaaaatccggggtgaccagtgggagcctatttgcacacacgagcgggacaccccactctggtgaagagtgccaaagtcattctttttcccgtnncggggcagccgattgcatgttttaggaaaatattacctttgctacaccctgtcagatttaccctccacacatatatattccgtcacctccagggactattcttggctcgttgcgccgccgcggaagatatccagaagctgtgttttccgagagactcggttggcgcctggtatatttnnaggatgtcgcgctgcctcacgtcccggtacccaggaacgcggtgggatctcgggcccatcgaagactgtgctccagactgctcgcccagcaggtgtttcttgattgccgcctctaaatagtccgcgcatcgccggtaacatttttccagctcggagtttgcgtttagatacatttctgcgatgccaaaggagcctgcagattataacctcggatgctgtcattcagcgcttttaatttgacctccagatagttgctgtatttctgttccattggctgctggacgttcgtataactcgagttattgttgcgctctgcctcggcgtactggctcatgactgactgcggtcgcttctcgagtgttctcgcaacaggacgcctgcaggtcatcgagtcgagctggcgccgaaactggcggatctgacctccacactgccctgtatctctatccaccgggaaccgcctcctgccgttccagaatgttgttcaagtggtagctctgtgcggtcaatgaaggcgttattgccggtgaaatctttgggaagcggtttatcctcggggaagattacgaaattcccgcgcgtcgttgcgcttcctggatctcgaggaagatcgttctccgcgtcgaggagatcgttctccgcgtcgacctgcaggcatgcaagcttctggtaaacgttgtagtactctgaaacaaggccctagcactctgatctgtttctcttgggtagcggtgagtggtttattggagttcactggtttcagcacatctgtcatctagacaatattgttactaaatttttttgaactacaattgttcgtaattcatctattattatacatcctcgtcagcaatttctggcagacggagtttactaacgtcttgagtatgaggccgagaatccagctctgtggccatactcagtcttgacagcctgctgatgtggctgcgttcaacgcaataagcgtgtcctccgactccgagttgtgctcgttatcgtcgttctcatcctcggaaaaatcacacgaaagaacatactcaccagtaggctttctggtccctggggcacggctgtttctgacgtattccggcgttgataatagctcgaaagtgaacgccgagtcgcgggagtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatcggggggggggggggggggggggggcaaacaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaagccccccccccccccccccccccccccccccccccgcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaattcccccttacacggaggcatcaagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaa

SEQ ID NO: 41 - DNA, 5202 bp, Artificial sequence
  vector pYIG5
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaatttaatacgactcactatagggaattcgaggatccttcaatatgcgcacatacgctgttatgttcaaggtcccttcgtttaagaacgaaagcggtcttccttttgagggatgtttcaagttgttcaaatctatcaaatttgcaaatccccagtctgtatctagagcgttgaatcggtgatgcgatttgttaattaaattgatggtgtcaccattaccaggtctagatataccaatggcaaactgagcacaacaataccagtccggatcaactggcaccatctctcccgtagtctcatctaatttttcttccggatgaggttccagatataccgcaacacctttattatggtttccctgagggaataatagaatgtcccattcgaaatcaccaattctaaacctgggcgaattgtatttcgggtttgttaactcgttccagtcaggaatgttccacgtgaagctatcttccagcaaagtctccacttcttcatcaaattgtggagaatactcccaatgctcttatctatgggacttccgggaaacacagtaccgatacttcccaattcgtcttcagagctcattgtttgtttgaagagactaatcaaagaatcgttttctcaaaaaaattaatatcttaactgatagtttgatcaaaggggcaaaacgtaggggcaaacaaacggaaaaatcgtttctcaaattttctgatgccaagaactctaaccagtcttatctaaaaattgccttatgatccgtctctccggttacagcctgtgtaactgattaatcctgcctttctaatcaccattctaatgttttaattaagggattttgtcttcattaacggctttcgctcataaaaatgttatgacgttttgcccgcaggcgggaaaccatccacttcacgagactgatctcctctgccggaacaccgggcatctccaacttataagttggagaaataagagaatttcagattgagagaatgaaaaaaaaaaaccctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaccatgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctagataaaaggcctgtcgacggtaccagatctcgacttggttgaacacgttgccaaggcttaagtgaatttactttaaagtcttgcatttaaataaattttctttttatagctttatgacttagtttcaatttatatactattttaatgacattttcgattcattgattgaaagctttgtgttttttcttgatgcgctattgcattgttcttgtctttttcgccacatgtaatatctgtagtagatacctgatacattgtggatgctgagtgaaattttagttaataatggaggcgctcttaataattttggggatattggcttttttttttaaagtttacaaatgaattttttccgccaggataacgattctgaagttactcttagcgttcctatcggtacagccatcaaatcatgcctataaatcatgcctatatttgcgtgcagtcagtatcatctacatgaaaaaaactcccgcaatttcttatagaatacgttgaaaattaaatgtacgcgccaagataagataacatatatctagctagatgcagtaatatacacagattcccgcggacgtgggaaggaaaaaattagataacaaaatctgagtgatatggaaattccgctgtatagctcatatctttcccttcaacaccagaaatgtaaaaatcttgttacgaaggatctttttgctaatgtttctcgctcaatcctcatttcttccctacgaagagtcaaatctacttgttttctgccggtatcaagatccatatcttctagtttcaccatcaaagtccaatttctagtatacagtttatgtcccaacgtaacagacaatcaaaattggaaaggataagtatccttcaaagaatgattctgcgctggctcctgaaccgcctaatgggaacagagaagtccaaaacgatgctataagaaccagaaataaaacgataaaaccataccaggatccaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 42 - DNA, 5613 bp, Artificial sequence
  vector pYIG5E1H6
ggatccttcaatatgcgcacatacgctgttatgttcaaggtcccttcgtttaagaacgaaagcggtcttccttttgagggatgtttcaagttgttcaaatctatcaaatttgcaaatccccagtctgtatctagagcgttgaatcggtgatgcgatttgttaattaaattgatggtgtcaccattaccaggtctagatataccaatggcaaactgagcacaacaataccagtccggatcaactggcaccatctctcccgtagtctcatctaatttttcttccggatgaggttccagatataccgcaacacctttattatggtttccctgagggaataatagaatgtcccattcgaaatcaccaattctaaacctgggcgaattgtatttcgggtttgttaactcgttccagtcaggaatgttccacgtgaagctatcttccagcaaagtctccacttcttcatcaaattgtggagaatactcccaatgctcttatctatgggacttccgggaaacacagtaccgatacttcccaattcgtcttcagagctcattgtttgtttgaagagactaatcaaagaatcgttttctcaaaaaaattaatatcttaactgatagtttgatcaaaggggcaaaacgtaggggcaaacaaacggaaaaatcgtttctcaaattttctgatgccaagaactctaaccagtcttatctaaaaattgccttatgatccgtctctccggttacagcctgtgtaactgattaatcctgcctttctaatcaccattctaatgttttaattaagggattttgtcttcattaacggctttcgctcataaaaatgttatgacgttttgcccgcaggcgggaaaccatccacttcacgagactgatctcctctgccggaacaccgggcatctccaacttataagttggagaaataagagaatttcagattgagagaatgaaaaaaaaaaaccctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaccatgagatttccttcaatttttactgcagttttattcgcagcatcctccgcattagctgctccagtcaacactacaacagaagatgaaacggcacaaattccggctgaagctgtcatcggttacttagatttagaaggggatttcgatgttgctgttttgccattttccaacagcacaaataacgggttattgtttataaatactactattgccagcattgctgctaaagaagaaggggtatctctagataaaaggtatgaggtgcgcaacgtgtccgggatgtaccatgtcacgaacgactgctccaactcaagcattgtgtatgaggcagcggacatgatcatgcacacccccgggtgcgtgccctgcgttcgggagaacaactcttcccgctgctgggtagcgctcacccccacgctcgcagctaggaacgccagcgtccccactacgacaatacgacgccacgtcgatttgctcgttggggcggctgctttctgttccgctatgtacgtgggggatctctgcggatctgtcttcctcgtctcccagctgttcaccatctcgcctcgccggcatgagacggtgcaggactgcaattgctcaatctatcccggccacataacaggtcaccgtatggcttgggatatgatgatgaactggcaccaccaccatcaccattaaagatctcgacttggttgaacacgttgccaaggcttaagtgaatttactttaaagtcttgcatttaaataaattttctttttatagctttatgacttagtttcaatttatatactattttaatgacattttcgattcattgattgaaagctttgtgttttttcttgatgcgctattgcattgttcttgtctttttcgccacatgtaatatctgtagtagatacctgatacattgtggatgctgagtgaaattttagttaataatggaggcgctcttaataattttggggatattggcttttttttttaaagtttacaaatgaattttttccgccaggataacgattctgaagttactcttagcgttcctatcggtacagccatcaaatcatgcctataaatcatgcctatatttgcgtgcagtcagtatcatctacatgaaaaaaactcccgcaatttcttatagaatacgttgaaaattaaatgtacgcgccaagataagataacatatatctagctagatgcagtaatatacacagattcccgcggacgtgggaaggaaaaaattagataacaaaatctgagtgatatggaaattccgctgtatagctcatatctttcccttcaacaccagaaatgtaaaaatcttgttacgaaggatctttttgctaatgtttctcgctcaatcctcatttcttccctacgaagagtcaaatctacttgttttctgccggtatcaagatccatatcttctagtttcaccatcaaagtccaatttctagtatacagtttatgtcccaacgtaacagacaatcaaaattggaaaggataagtatccttcaaagaatgattctgcgctggctcctgaaccgcctaatgggaacagagaagtccaaaacgatgctataagaaccagaaataaaacgataaaaccataccaggatccaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaatttaatacgactcactatagggaattcga

SEQ ID NO: 43 - DNA, 13020 bp, Artificial sequence
  vector pSY1
atcgataagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaaagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcccctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaattcccccttacacggaggcatcaagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggtgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaaataggcgtatcacgaggccctttcgtcttcaagaattctcatgtttgacagcttatcatcgatccacttgtatatttggatgaatttttgaggaattctgaaccagtcctaaaacgagtaaataggaccggcaattcttcaagcaataaacaggaataccaattattaaaagataacttagtcagatcgtacaataaagctttgaagaaaaatgcgccttattcaatctttgcataaaaaaatggcccaaaatctcacattggaagacatttgatgacctcatttctttcaatgaagggcctaacggagttgactaatgttgtgggaaattggaccgataagcgtgcttctgccgtggccaggacaacgtatactcatcagataacagcaatacctgatcactacttcgcactagtttctcggtactatgcatatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataaccatcccatccacctttcgctccttgaacttgcatctaaactcgacctctacattttttatgtttatctctagtattacctcttagacaaaaaaattgtagtaagaactattcatagagttaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattcttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttaacaaagatatgctattgaagtgcaagatggaaacgcagaaaatgaaccggggatgcgacgtgcaagattacctatgcaatagatgcaatagtttctccaggaaccgaaatacatacattgtcttccgtaaagcgctagactatatattattatacaggttcaaatatactatctgtttcagggaaaactcccaggttcggatgttcaaaattcaatgatgggtaacaagtacgatcgtaaatctgtaaaacagtttgtcggatattaggctgtatctcctcaaagcgtattcgaatatcattgagaagctgcattttttttttttttttttttttttttttttttatatatatttcaaggatataccattgtaatgtctgcccctaagaagatcgtcgttttgccaggtgaccacgttggtcaagaaatcacagccgaagccattaaggttcttaaagctatttctgatgttcgttccaatgtcaagttcgatttcgaaaatcatttaattggtggtgctgctatcgatgctacaggtgtcccacttccagatgaggcgctggaagcctccaagaaggttgatgccgttttgttaggtgctgtgggtggtcctaaatggggtaccggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatgttttggcctcttcaagattatggagaaaaactgtggaggaaaccatcaagaacgaattccctacattgaaggttcaacatcaattgattgattctgccgccatgatcctagttaagaacccaacccacctaaatggtattataatcaccagcaacatgtttggtgatatcatctccgatgaagcctccgttatcccaggttccttgggtttgttgccatctgcgtccttggcctctttgccagacaagaacaccgcatttggtttgtacgaaccatgccacggttctgctccagatttgccaaagaataaggttgaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccaccgaagtcggtgatgctgtcgccgaagaagttaagaaaatccttgcttaaaaagattctctttttttatgatatttgtacaaaaaaaaaaaaaaaaaaaaaaaaaaaaaaaaaaaaaaaaaaaaaatgcagcgtcacatcggataataatgatggcagccattgtagaagtgccttttgcatttctagtctctttctcggtctagctagttttactacatcgcgaagatagaatcttagatcacactgcctttgctgagctggatcatatgagtaacaaaagagtggtaaggcctcgttaaaggacaaggacctgagcggaagtgtatcgtaaagtagacggagtatactagtatagtctatagtccgtggaattctaagtgccagctttataatgtcattctccttactacagacccgcctgaaagtagacacatcatcatcagtaagctttgacaaaaagcattgagtagctaactcttctatgcaatctatagctgttttataaggcattcaatggacagattgaggtttttgaaacatactagtgaaattagccttaatcccttctcgaagttaatcatgcattatggtgtaaaaaatgcaactcgcgttgctctactttttcccgaatttccaaatacgcagctggggtgattgctcgatttcgtaacgaaagttttgtttataaaaaccgcgaaaaccttctgtaacagatagatttttacagcgctgatatacaatgacatcagctgtaatggaaaataactgaaatatgaatggcgagagactgcttgcttgtattaagcaatgtattatgcagcacttccaacctatggtgtacgatgaaagtaggtgtgtaatcgagacgacaagggggacttttccagttcctgatcattataagaaatacaaaacgttagcatttgcatttgttggacatgtactgaatacagacgacacaccggtaattgaaaaagaactggattggcctgatcctgcactagtgtacaatacaattgtcgatcgaatcataaatcacccagaattatcacagtttatatcggttgcatttattagtcagttaaaggccaccatcggagagggtttagatattaatgtaaaaggcacgctaaaccgcaggggaaagggtatcagaaggcctaaaggcgtattttttagatacatggaatctccatttgtcaatacaaaggtcactgcattcttctcttatcttcgagattataataaaattgcctcagaatatcacaataatactaaattcattctcacgttttcatgtcaagcatattgggcatctggcccaaacttctccgccttgaagaatgttatttggtgctccataattcatgaatacatttctaagtttgtggaaagagaacaggataaaggtcatataggagatcaggagctaccgcctgaagaggacccttctcgtgaactaaacaatgtacaacatgaagtcaatagtttaacggaacaagatgcggaggcggatgaaggattgtggggtgaaatagattcattatgtgaaaaatggcagtctgaagcggagagtcaaactgaggcggagataatagccgacaggataattggaaatagccagaggatggcgaacctcaaaattcgtcgtacaaagttcaaaagtgtcttgtatcatatactaaaggaactaattcaatctcagggaaccgtaaaggtttatcgcggtagtagtttttcacacgattcgataaagataagcttacattatgaagagcagcatattacagccgtatgggtctacttgatagtaaaatttgaagagcattggaagcctgttgatgtagaggtcgagtttagatgcaagttcaaggagcgaaaggtggatgggtaggttatatagggatatagcacagagatatatagcaaagagatacttttgaggcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgcattcatttcctttgatattggatcataccctagaagtattacgtgattttctgccccttaccctcgttgctactctcctttttttcgtgggaaccgctttagggccctcagtgatggtgttttgtaatttatatgctcctcttgcatttgtgtctctacttcttgttcgcctggagggaacttcttcatttgtattagcatggttcacttcagtccttccttccaactcactctttttttgctgtaaacgattctctgccgccagttcattgaaactattgaatatatcctttagagattccgggatgaataaatcacctattaaagcagcttgacgatctggtggaactaaagtaagcaattgggtaacgacgcttacgagcttcataacatcttcttccgttggagctggtgggactaataactgtgtacaatccatttttctcatgagcatttcggtagctctcttcttgtctttctcgggcaatcttcctattattatagcaatagatttgtatagttgctttctattgtctaacagcttgttattctgtagcatcaaatctatggcagcctgacttgcttcttgtgaagagagcataccatttccaatcgaagatacgctggaatcttctgcgctagaatcaagaccatacggcctaccggttgtgagagattccatgggccttatgacatatcctggaaagagtagctcatcagacttacgtttactctctatatcaatatctacatcaggagcaatcatttcaataaacagccgacatacatcccagacgctataagctgtacgtgcttttaccgtcagattcttggctgtttcaatgtcgtccattttggttttcttttaccagtattgttcgtttgataatgtattcttgcttattacattataaaatctgtgcagatcacatgtcaaaacaactttttatcacaagatagtaccgcaaaacgaacctgcgggccgtctaaaaattaaggaaaagcagcaaaggtgcatttttaaaatatgaaatgaagataccgcagtaccaattattttcgcagtacaaataatgcgcggccggtgcatttttcgaaagaacgcgagacaaacaggacaattaaagttagtttttcgagttagcgtgtttgaatactgcaagatacaagataaatagagtagttgaaactagatatcaattgcacacaagatcggcgctaagcatgccacaatttggtatattatgtaaaacaccacctaaggtgcttgttcgtcagtttgtggaaaggtttgaaagaccttcaggtgagaaaatagcattatgtgctgctgaactaacctatttatgttggatgattacacataacggaacagcaatcaagagagccacattcatgagctataatactatcataagcaattcgctgagtttcgatattgtcaataaatcactccagtttaaatacaagacgcaaaaagcaacaattctggaagcctcattaaagaaattgattcctgcttgggaatttacaattattccttactatggacaaaaacatcaatctgatatcactgatattgtaagtagtttgcaattacagttcgaatcatcggaagaagcagataagggaaatagccacagtaaaaaaatgctaaagcacttctaagtgagggtgaaagcatctgggagatcactgagaaaatactaaattcgtttgagtatacttcgagatttacaaaaacaaaaactttataccaattcctcttcctagctactttcatcaattgtggaagattcagcgatattaagaacgttgatccgaaatcatttaaattagtccaaaataagtatctgggagtaataatccagtgtttagtgacagagacaaagacaagcgttagtaggcacatatacttctttagcgcaaggggtag

SEQ ID NO: 44 - DNA, 15810 bp, Artificial sequence
  vector pSY1AMFE1sH6a
atcgataagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaaagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatccttcaatatgcgcacatacgctgttatgttcaaggtcccttcgtttaagaacgaaagcggtcttccttttgagggatgtttcaagttgttcaaatctatcaaatttgcaaatccccagtctgtatctagagcgttgaatcggtgatgcgatttgttaattaaattgatggtgtcaccattaccaggtctagatataccaatggcaaactgagcacaacaataccagtccggatcaactggcaccatctctcccgtagtctcatctaatttttcttccggatgaggttccagatataccgcaacacctttattatggtttccctgagggaataatagaatgtcccattcgaaatcaccaattctaaacctgggcgaattgtatttcgggtttgttaactcgttccagtcaggaatgttccacgtgaagctatcttccagcaaagtctccacttcttcatcaaattgtggagaatactcccaatgctcttatctatgggacttccgggaaacacagtaccgatacttcccaattcgtcttcagagctcattgtttgtttgaagagactaatcaaagaatcgttttctcaaaaaaattaatatcttaactgatagtttgatcaaaggggcaaaacgtaggggcaaacaaacggaaaaatcgtttctcaaattttctgatgccaagaactctaaccagtcttatctaaaaattgccttatgatccgtctctccggttacagcctgtgtaactgattaatcctgcctttctaatcaccattctaatgttttaattaagggattttgtcttcattaacggctttcgctcataaaaatgttatgacgttttgcccgcaggcgggaaaccatccacttcacgagactgatctcctctgccggaacaccgggcatctccaacttataagttggagaaataagagaatttcagattgagagaatgaaaaaaaaaaaccctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaccatgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctagataaaaggtatgaggtgcgcaacgtgtccgggatgtaccatgtcacgaacgactgctccaactcaagcattgtgtatgaggcagcggacatgatcatgcacacccccgggtgcgtgccctgcgttcgggagaacaactcttcccgctgctgggtagcgctcacccccacgctcgcagctaggaacgccagcgtccccactacgacaatacgacgccacgtcgatttgctcgttggggcggctgctttctgttccgctatgtacgtgggggatctctgcggatctgtcttcctcgtctcccagctgttcaccatctcgcctcgccggcatgagacggtgcaggactgcaattgctcaatctatcccggccacataacgggtcaccgtatggcttgggatatgatgatgaactggcaccaccaccatcaccattaaagatctcgacttggttgaacacgttgccaaggcttaagtgaatttactttaaagtcttgcatttaaataaattttctttttatagctttatgacttagtttcaatttatatactattttaatgacattttcgattcattgattgaaagctttgtgttttttcttgatgcgctattgcattgttcttgtctttttcgccacatgtaatatctgtagtagatacctgatacattgtggatgctgagtgaaattttagttaataatggaggcgctcttaataattttggggatattggcttttttttttaaagtttacaaatgaattttttccgccaggataacgattctgaagttactcttagcgttcctatcggtacagccatcaaatcatgcctataaatcatgcctatatttgcgtgcagtcagtatcatctacatgaaaaaaactcccgcaatttcttatagaatacgttgaaaattaaatgtacgcgccaagataagataacatatatctagctagatgcagtaatatacacagattcccgcggacgtgggaaggaaaaaattagataacaaaatctgagtgatatggaaattccgctgtatagctcatatctttcccttcaacaccagaaatgtaaaaatcttgttacgaaggatctttttgctaatgtttctcgctcaatcctcatttcttccctacgaagagtcaaatctacttgttttctgccggtatcaagatccatatcttctagtttcaccatcaaagtccaatttctagtatacagtttatgtcccaacgtaacagacaatcaaaattggaaaggataagtatccttcaaagaatgattctgcgctggctcctgaaccgcctaatgggaacagagaagtccaaaacgatgctataagaaccagaaataaaacgataaaaccataccaggatcctctacgccggacgcatcgtggccggcatcaccggcgccacaggtgcggttgctggcccctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaattcccccttacacggaggcatcaagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggtgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaaataggcgtatcacgaggccctttcgtcttcaagaattctcatgtttgacagcttatcatcgatccacttgtatatttggatgaatttttgaggaattctgaaccagtcctaaaacgagtaaataggaccggcaattcttcaagcaataaacaggaataccaattattaaaagataacttagtcagatcgtacaataaagctttgaagaaaaatgcgccttattcaatctttgcataaaaaaatggcccaaaatctcacattggaagacatttgatgacctcatttctttcaatgaagggcctaacggagttgactaatgttgtgggaaattggaccgataagcgtgcttctgccgtggccaggacaacgtatactcatcagataacagcaatacctgatcactacttcgcactagtttctcggtactatgcatatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataaccatcccatccacctttcgctccttgaacttgcatctaaactcgacctctacattttttatgtttatctctagtattacctcttagacaaaaaaattgtagtaagaactattcatagagttaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattcttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttaacaaagatatgctattgaagtgcaagatggaaacgcagaaaatgaaccggggatgcgacgtgcaagattacctatgcaatagatgcaatagtttctccaggaaccgaaatacatacattgtcttccgtaaagcgctagactatatattattatacaggttcaaatatactatctgtttcagggaaaactcccaggttcggatgttcaaaattcaatgatgggtaacaagtacgatcgtaaatctgtaaaacagtttgtcggatattaggctgtatctcctcaaagcgtattcgaatatcattgagaagctgcattttttttttttttttttttttttttttttttatatatatttcaaggatataccattgtaatgtctgcccctaagaagatcgtcgttttgccaggtgaccacgttggtcaagaaatcacagccgaagccattaaggttcttaaagctatttctgatgttcgttccaatgtcaagttcgatttcgaaaatcatttaattggtggtgctgctatcgatgctacaggtgtcccacttccagatgaggcgctggaagcctccaagaaggttgatgccgttttgttaggtgctgtgggtggtcctaaatggggtaccggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatgttttggcctcttcaagattatggagaaaaactgtggaggaaaccatcaagaacgaattccctacattgaaggttcaacatcaattgattgattctgccgccatgatcctagttaagaacccaacccacctaaatggtattataatcaccagcaacatgtttggtgatatcatctccgatgaagcctccgttatcccaggttccttgggtttgttgccatctgcgtccttggcctctttgccagacaagaacaccgcatttggtttgtacgaaccatgccacggttctgctccagatttgccaaagaataaggttgaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccaccgaagtcggtgatgctgtcgccgaagaagttaagaaaatccttgcttaaaaagattctctttttttatgatatttgtacaaaaaaaaaaaaaaaaaaaaaaaaaaaaaaaaaaaaaaaaaaaaaatgcagcgtcacatcggataataatgatggcagccattgtagaagtgccttttgcatttctagtctctttctcggtctagctagttttactacatcgcgaagatagaatcttagatcacactgcctttgctgagctggatcatatgagtaacaaaagagtggtaaggcctcgttaaaggacaaggacctgagcggaagtgtatcgtaaagtagacggagtatactagtatagtctatagtccgtggaattctaagtgccagctttataatgtcattctccttactacagacccgcctgaaagtagacacatcatcatcagtaagctttgacaaaaagcattgagtagctaactcttctatgcaatctatagctgttttataaggcattcaatggacagattgaggtttttgaaacatactagtgaaattagccttaatcccttctcgaagttaatcatgcattatggtgtaaaaaatgcaactcgcgttgctctactttttcccgaatttccaaatacgcagctggggtgattgctcgatttcgtaacgaaagttttgtttataaaaaccgcgaaaaccttctgtaacagatagatttttacagcgctgatatacaatgacatcagctgtaatggaaaataactgaaatatgaatggcgagagactgcttgcttgtattaagcaatgtattatgcagcacttccaacctatggtgtacgatgaaagtaggtgtgtaatcgagacgacaagggggacttttccagttcctgatcattataagaaatacaaaacgttagcatttgcatttgttggacatgtactgaatacagacgacacaccggtaattgaaaaagaactggattggcctgatcctgcactagtgtacaatacaattgtcgatcgaatcataaatcacccagaattatcacagtttatatcggttgcatttattagtcagttaaaggccaccatcggagagggtttagatattaatgtaaaaggcacgctaaaccgcaggggaaagggtatcagaaggcctaaaggcgtattttttagatacatggaatctccatttgtcaatacaaaggtcactgcattcttctcttatcttcgagattataataaaattgcctcagaatatcacaataatactaaattcattctcacgttttcatgtcaagcatattgggcatctggcccaaacttctccgccttgaagaatgttatttggtgctccataattcatgaatacatttctaagtttgtggaaagagaacaggataaaggtcatataggagatcaggagctaccgcctgaagaggacccttctcgtgaactaaacaatgtacaacatgaagtcaatagtttaacggaacaagatgcggaggcggatgaaggattgtggggtgaaatagattcattatgtgaaaaatggcagtctgaagcggagagtcaaactgaggcggagataatagccgacaggataattggaaatagccagaggatggcgaacctcaaaattcgtcgtacaaagttcaaaagtgtcttgtatcatatactaaaggaactaattcaatctcagggaaccgtaaaggtttatcgcggtagtagtttttcacacgattcgataaagataagcttacattatgaagagcagcatattacagccgtatgggtctacttgatagtaaaatttgaagagcattggaagcctgttgatgtagaggtcgagtttagatgcaagttcaaggagcgaaaggtggatgggtaggttatatagggatatagcacagagatatatagcaaagagatacttttgaggcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgcattcatttcctttgatattggatcataccctagaagtattacgtgattttctgccccttaccctcgttgctactctcctttttttcgtgggaaccgctttagggccctcagtgatggtgttttgtaatttatatgctcctcttgcatttgtgtctctacttcttgttcgcctggagggaacttcttcatttgtattagcatggttcacttcagtccttccttccaactcactctttttttgctgtaaacgattctctgccgccagttcattgaaactattgaatatatcctttagagattccgggatgaataaatcacctattaaagcagcttgacgatctggtggaactaaagtaagcaattgggtaacgacgcttacgagcttcataacatcttcttccgttggagctggtgggactaataactgtgtacaatccatttttctcatgagcatttcggtagctctcttcttgtctttctcgggcaatcttcctattattatagcaatagatttgtatagttgctttctattgtctaacagcttgttattctgtagcatcaaatctatggcagcctgacttgcttcttgtgaagagagcataccatttccaatcgaagatacgctggaatcttctgcgctagaatcaagaccatacggcctaccggttgtgagagattccatgggccttatgacatatcctggaaagagtagctcatcagacttacgtttactctctatatcaatatctacatcaggagcaatcatttcaataaacagccgacatacatcccagacgctataagctgtacgtgcttttaccgtcagattcttggctgtttcaatgtcgtccattttggttttcttttaccagtattgttcgtttgataatgtattcttgcttattacattataaaatctgtgcagatcacatgtcaaaacaactttttatcacaagatagtaccgcaaaacgaacctgcgggccgtctaaaaattaaggaaaagcagcaaaggtgcatttttaaaatatgaaatgaagataccgcagtaccaattattttcgcagtacaaataatgcgcggccggtgcatttttcgaaagaacgcgagacaaacaggacaattaaagttagtttttcgagttagcgtgtttgaatactgcaagatacaagataaatagagtagttgaaactagatatcaattgcacacaagatcggcgctaagcatgccacaatttggtatattatgtaaaacaccacctaaggtgcttgttcgtcagtttgtggaaaggtttgaaagaccttcaggtgagaaaatagcattatgtgctgctgaactaacctatttatgttggatgattacacataacggaacagcaatcaagagagccacattcatgagctataatactatcataagcaattcgctgagtttcgatattgtcaataaatcactccagtttaaatacaagacgcaaaaagcaacaattctggaagcctcattaaagaaattgattcctgcttgggaatttacaattattccttactatggacaaaaacatcaatctgatatcactgatattgtaagtagtttgcaattacagttcgaatcatcggaagaagcagataagggaaatagccacagtaaaaaaatgctaaagcacttctaagtgagggtgaaagcatctgggagatcactgagaaaatactaaattcgtttgagtatacttcgagatttacaaaaacaaaaactttataccaattcctcttcctagctactttcatcaattgtggaagattcagcgatattaagaacgttgatccgaaatcatttaaattagtccaaaataagtatctgggagtaataatccagtgtttagtgacagagacaaagacaagcgttagtaggcacatatacttctttagcgcaaggggtag

SEQ ID NO: 45 - DNA, 3928 bp, Artificial sequence
  vector pBKS-E2sH6
cacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtaccgggccccccctcgaggtcgacggtatcgataagcttgcatgcctgcagttaattaactattagtgatggtggtgatggtgtctgccctcgatcacctgccactctgttgtagacagcagcagcgggctaagctctgatctatccctgtcctccaagtcacaacgctctcctcgagtccaattgcatgcggcttcgaacctgtgctccacgccccccacgtacatcctaaccttgaagatggtgaagttgacagtgcaggggtagtgccagagcctatatgggtaatgaaccatacacctaggtgtcagccagggcccagaaccgcatctggcgtaggtggcctcggggtgcttccgaaaacagtcagtggggcaggtcaaggtgttgttgccggcccccccgatgttgcacggggggcccccacacgtcttggtgaacccagtgccattcatccatgtacagccgaaccagttgcctcgcggcggccgcgtgttgttgagaatcagcacatccgagtcgttcgccccccagttatacgtggggacaccaaaccgatcggtcgtccccaccacaacagggctcggggtgaagcaatacactggaccgcacacctgagacgcgggtacaataccacacggtcgaggcgcgtagtgccagcagtagggcctctggtccgagctgttaggctcagtgtaagtgaggggaccccacccctgagcgaacttgtcgatggagcgacagctggccaagcgctctgggcatccagacgagttgaatttgtgtttgtagaatagtgcggcaaagaaccctgtttggagggagtcgttgcagttcagggcagtcctgttgatgtgccaactgccgttggtgtttacgagctggattttctgagccgacccggggctaaagagggacacaaggcccctggtatcggaggctgctgcccctcctgacacgcgggtatggtaccgggccccccctcgaggtcgacggtatcgataagcttgatatcgaattcctgcagcccgggggatccactagttctagagcggccgccaccgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgc

SEQ ID NO: 46 - DNA, 6104 bp, Artificial sequence
  vector pYIG5HCCL-22aH6
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaatttaatacgactcactatagggaattcgaggatccttcaatatgcgcacatacgctgttatgttcaaggtcccttcgtttaagaacgaaagcggtcttccttttgagggatgtttcaagttgttcaaatctatcaaatttgcaaatccccagtctgtatctagagcgttgaatcggtgatgcgatttgttaattaaattgatggtgtcaccattaccaggtctagatataccaatggcaaactgagcacaacaataccagtccggatcaactggcaccatctctcccgtagtctcatctaatttttcttccggatgaggttccagatataccgcaacacctttattatggtttccctgagggaataatagaatgtcccattcgaaatcaccaattctaaacctgggcgaattgtatttcgggtttgttaactcgttccagtcaggaatgttccacgtgaagctatcttccagcaaagtctccacttcttcatcaaattgtggagaatactcccaatgctcttatctatgggacttccgggaaacacagtaccgatacttcccaattcgtcttcagagctcattgtttgtttgaagagactaatcaaagaatcgttttctcaaaaaaattaatatcttaactgatagtttgatcaaaggggcaaaacgtaggggcaaacaaacggaaaaatcgtttctcaaattttctgatgccaagaactctaaccagtcttatctaaaaattgccttatgatccgtctctccggttacagcctgtgtaactgattaatcctgcctttctaatcaccattctaatgttttaattaagggattttgtcttcattaacggctttcgctcataaaaatgttatgacgttttgcccgcaggcgggaaaccatccacttcacgagactgatctcctctgccggaacaccgggcatctccaacttataagttggagaaataagagaatttcagattgagagaatgaaaaaaaaaaaccctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaccatgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctagataaaaggcatacccgcgtgtcaggaggggcagcagcctccgataccaggggccttgtgtccctctttagccccgggtcggctcagaaaatccagctcgtaaacaccaacggcagttggcacatcaacaggactgccctgaactgcaacgactccctccaaacagggttctttgccgcactattctacaaacacaaattcaactcgtctggatgcccagagcgcttggccagctgtcgctccatcgacaagttcgctcaggggtggggtcccctcacttacactgagcctaacagctcggaccagaggccctactgctggcactacgcgcctcgaccgtgtggtattgtacccgcgtctcaggtgtgcggtccagtgtattgcttcaccccgagccctgttgtggtggggacgaccgatcggtttggtgtccccacgtataactggggggcgaacgactcggatgtgctgattctcaacaacacgcggccgccgcgaggcaactggttcggctgtacatggatgaatggcactgggttcaccaagacgtgtgggggccccccgtgcaacatcgggggggccggcaacaacaccttgacctgccccactgactgttttcggaagcaccccgaggccacttacgccagatgcggttctgggccctggctgacacctaggtgtatggttcattacccatataggctctggcactacccctgcactgtcaacttcaccatcttcaaggttaggatgtacgtggggggcgtggagcacaggttcgaagccgcatgcaattggactcgaggagagcgttgtgacttggaggacagggatagatcagagcttagctcgctgctgctgtctacaacagagtggcaggtgatcgagggcagacaccatcaccaccatcactaatagttaattaacgatctcgacttggttgaacacgttgccaaggcttaagtgaatttactttaaagtcttgcatttaaataaattttctttttatagctttatgacttagtttcaatttatatactattttaatgacattttcgattcattgattgaaagctttgtgttttttcttgatgcgctattgcattgttcttgtctttttcgccacatgtaatatctgtagtagatacctgatacattgtggatgctgagtgaaattttagttaataatggaggcgctcttaataattttggggatattggcttttttttttaaagtttacaaatgaattttttccgccaggataacgattctgaagttactcttagcgttcctatcggtacagccatcaaatcatgcctataaatcatgcctatatttgcgtgcagtcagtatcatctacatgaaaaaaactcccgcaatttcttatagaatacgttgaaaattaaatgtacgcgccaagataagataacatatatctagctagatgcagtaatatacacagattcccgcggacgtgggaaggaaaaaattagataacaaaatctgagtgatatggaaattccgctgtatagctcatatctttcccttcaacaccagaaatgtaaaaatcttgttacgaaggatctttttgctaatgtttctcgctcaatcctcatttcttccctacgaagagtcaaatctacttgttttctgccggtatcaagatccatatcttctagtttcaccatcaaagtccaatttctagtatacagtttatgtcccaacgtaacagacaatcaaaattggaaaggataagtatccttcaaagaatgattctgcgctggctcctgaaccgcctaatgggaacagagaagtccaaaacgatgctataagaaccagaaataaaacgataaaaccataccaggatccaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 47 - DNA, 16301 bp, Artificial sequence
  vector pYYIGSE2H6
atcgataagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaaagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatccttcaatatgcgcacatacgctgttatgttcaaggtcccttcgtttaagaacgaaagcggtcttccttttgagggatgtttcaagttgttcaaatctatcaaatttgcaaatccccagtctgtatctagagcgttgaatcggtgatgcgatttgttaattaaattgatggtgtcaccattaccaggtctagatataccaatggcaaactgagcacaacaataccagtccggatcaactggcaccatctctcccgtagtctcatctaatttttcttccggatgaggttccagatataccgcaacacctttattatggtttccctgagggaataatagaatgtcccattcgaaatcaccaattctaaacctgggcgaattgtatttcgggtttgttaactcgttccagtcaggaatgttccacgtgaagctatcttccagcaaagtctccacttcttcatcaaattgtggagaatactcccaatgctcttatctatgggacttccgggaaacacagtaccgatacttcccaattcgtcttcagagctcattgtttgtttgaagagactaatcaaagaatcgttttctcaaaaaaattaatatcttaactgatagtttgatcaaaggggcaaaacgtaggggcaaacaaacggaaaaatcgtttctcaaattttctgatgccaagaactctaaccagtcttatctaaaaattgccttatgatccgtctctccggttacagcctgtgtaactgattaatcctgcctttctaatcaccattctaatgttttaattaagggattttgtcttcattaacggctttcgctcataaaaatgttatgacgttttgcccgcaggcgggaaaccatccacttcacgagactgatctcctctgccggaacaccgggcatctccaacttataagttggagaaataagagaatttcagattgagagaatgaaaaaaaaaaaccctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaccatgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctagataaaaggcatacccgcgtgtcaggaggggcagcagcctccgataccaggggccttgtgtccctctttagccccgggtcggctcagaaaatccagctcgtaaacaccaacggcagttggcacatcaacaggactgccctgaactgcaacgactccctccaaacagggttctttgccgcactattctacaaacacaaattcaactcgtctggatgcccagagcgcttggccagctgtcgctccatcgacaagttcgctcaggggtggggtcccctcacttacactgagcctaacagctcggaccagaggccctactgctggcactacgcgcctcgaccgtgtggtattgtacccgcgtctcaggtgtgcggtccagtgtattgcttcaccccgagccctgttgtggtggggacgaccgatcggtttggtgtccccacgtataactggggggcgaacgactcggatgtgctgattctcaacaacacgcggccgccgcgaggcaactggttcggctgtacatggatgaatggcactgggttcaccaagacgtgtgggggccccccgtgcaacatcgggggggccggcaacaacaccttgacctgccccactgactgttttcggaagcaccccgaggccacttacgccagatgcggttctgggccctggctgacacctaggtgtatggttcattacccatataggctctggcactacccctgcactgtcaacttcaccatcttcaaggttaggatgtacgtggggggcgtggagcacaggttcgaagccgcatgcaattggactcgaggagagcgttgtgacttggaggacagggatagatcagagcttagctcgctgctgctgtctacaacagagtggcaggtgatcgagggcagacaccatcaccaccatcactaatagttaattaacgatctcgacttggttgaacacgttgccaaggcttaagtgaatttactttaaagtcttgcatttaaataaattttctttttatagctttatgacttagtttcaatttatatactattttaatgacattttcgattcattgattgaaagctttgtgttttttcttgatgcgctattgcattgttcttgtctttttcgccacatgtaatatctgtagtagatacctgatacattgtggatgctgagtgaaattttagttaataatggaggcgctcttaataattttggggatattggcttttttttttaaagtttacaaatgaattttttccgccaggataacgattctgaagttactcttagcgttcctatcggtacagccatcaaatcatgcctataaatcatgcctatatttgcgtgcagtcagtatcatctacatgaaaaaaactcccgcaatttcttatagaatacgttgaaaattaaatgtacgcgccaagataagataacatatatctagctagatgcagtaatatacacagattcccgcggacgtgggaaggaaaaaattagataacaaaatctgagtgatatggaaattccgctgtatagctcatatctttcccttcaacaccagaaatgtaaaaatcttgttacgaaggatctttttgctaatgtttctcgctcaatcctcatttcttccctacgaagagtcaaatctacttgttttctgccggtatcaagatccatatcttctagtttcaccatcaaagtccaatttctagtatacagtttatgtcccaacgtaacagacaatcaaaattggaaaggataagtatccttcaaagaatgattctgcgctggctcctgaaccgcctaatgggaacagagaagtccaaaacgatgctataagaaccagaaataaaacgataaaaccataccaggatcctctacgccggacgcatcgtggccggcatcaccggcgccacaggtgcggttgctggcccctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaattcccccttacacggaggcatcaagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggtgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaaataggcgtatcacgaggccctttcgtcttcaagaattctcatgtttgacagcttatcatcgatccacttgtatatttggatgaatttttgaggaattctgaaccagtcctaaaacgagtaaataggaccggcaattcttcaagcaataaacaggaataccaattattaaaagataacttagtcagatcgtacaataaagctttgaagaaaaatgcgccttattcaatctttgcataaaaaaatggcccaaaatctcacattggaagacatttgatgacctcatttctttcaatgaagggcctaacggagttgactaatgttgtgggaaattggaccgataagcgtgcttctgccgtggccaggacaacgtatactcatcagataacagcaatacctgatcactacttcgcactagtttctcggtactatgcatatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataaccatcccatccacctttcgctccttgaacttgcatctaaactcgacctctacattttttatgtttatctctagtattacctcttagacaaaaaaattgtagtaagaactattcatagagttaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattcttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttaacaaagatatgctattgaagtgcaagatggaaacgcagaaaatgaaccggggatgcgacgtgcaagattacctatgcaatagatgcaatagtttctccaggaaccgaaatacatacattgtcttccgtaaagcgctagactatatattattatacaggttcaaatatactatctgtttcagggaaaactcccaggttcggatgttcaaaattcaatgatgggtaacaagtacgatcgtaaatctgtaaaacagtttgtcggatattaggctgtatctcctcaaagcgtattcgaatatcattgagaagctgcattttttttttttttttttttttttttttttttatatatatttcaaggatataccattgtaatgtctgcccctaagaagatcgtcgttttgccaggtgaccacgttggtcaagaaatcacagccgaagccattaaggttcttaaagctatttctgatgttcgttccaatgtcaagttcgatttcgaaaatcatttaattggtggtgctgctatcgatgctacaggtgtcccacttccagatgaggcgctggaagcctccaagaaggttgatgccgttttgttaggtgctgtgggtggtcctaaatggggtaccggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatgttttggcctcttcaagattatggagaaaaactgtggaggaaaccatcaagaacgaattccctacattgaaggttcaacatcaattgattgattctgccgccatgatcctagttaagaacccaacccacctaaatggtattataatcaccagcaacatgtttggtgatatcatctccgatgaagcctccgttatcccaggttccttgggtttgttgccatctgcgtccttggcctctttgccagacaagaacaccgcatttggtttgtacgaaccatgccacggttctgctccagatttgccaaagaataaggttgaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccaccgaagtcggtgatgctgtcgccgaagaagttaagaaaatccttgcttaaaaagattctctttttttatgatatttgtacaaaaaaaaaaaaaaaaaaaaaaaaaaaaaaaaaaaaaaaaaaaaaatgcagcgtcacatcggataataatgatggcagccattgtagaagtgccttttgcatttctagtctctttctcggtctagctagttttactacatcgcgaagatagaatcttagatcacactgcctttgctgagctggatcatatgagtaacaaaagagtggtaaggcctcgttaaaggacaaggacctgagcggaagtgtatcgtaaagtagacggagtatactagtatagtctatagtccgtggaattctaagtgccagctttataatgtcattctccttactacagacccgcctgaaagtagacacatcatcatcagtaagctttgacaaaaagcattgagtagctaactcttctatgcaatctatagctgttttataaggcattcaatggacagattgaggtttttgaaacatactagtgaaattagccttaatcccttctcgaagttaatcatgcattatggtgtaaaaaatgcaactcgcgttgctctactttttcccgaatttccaaatacgcagctggggtgattgctcgatttcgtaacgaaagttttgtttataaaaaccgcgaaaaccttctgtaacagatagatttttacagcgctgatatacaatgacatcagctgtaatggaaaataactgaaatatgaatggcgagagactgcttgcttgtattaagcaatgtattatgcagcacttccaacctatggtgtacgatgaaagtaggtgtgtaatcgagacgacaagggggacttttccagttcctgatcattataagaaatacaaaacgttagcatttgcatttgttggacatgtactgaatacagacgacacaccggtaattgaaaaagaactggattggcctgatcctgcactagtgtacaatacaattgtcgatcgaatcataaatcacccagaattatcacagtttatatcggttgcatttattagtcagttaaaggccaccatcggagagggtttagatattaatgtaaaaggcacgctaaaccgcaggggaaagggtatcagaaggcctaaaggcgtattttttagatacatggaatctccatttgtcaatacaaaggtcactgcattcttctcttatcttcgagattataataaaattgcctcagaatatcacaataatactaaattcattctcacgttttcatgtcaagcatattgggcatctggcccaaacttctccgccttgaagaatgttatttggtgctccataattcatgaatacatttctaagtttgtggaaagagaacaggataaaggtcatataggagatcaggagctaccgcctgaagaggacccttctcgtgaactaaacaatgtacaacatgaagtcaatagtttaacggaacaagatgcggaggcggatgaaggattgtggggtgaaatagattcattatgtgaaaaatggcagtctgaagcggagagtcaaactgaggcggagataatagccgacaggataattggaaatagccagaggatggcgaacctcaaaattcgtcgtacaaagttcaaaagtgtcttgtatcatatactaaaggaactaattcaatctcagggaaccgtaaaggtttatcgcggtagtagtttttcacacgattcgataaagataagcttacattatgaagagcagcatattacagccgtatgggtctacttgatagtaaaatttgaagagcattggaagcctgttgatgtagaggtcgagtttagatgcaagttcaaggagcgaaaggtggatgggtaggttatatagggatatagcacagagatatatagcaaagagatacttttgaggcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgcattcatttcctttgatattggatcataccctagaagtattacgtgattttctgccccttaccctcgttgctactctcctttttttcgtgggaaccgctttagggccctcagtgatggtgttttgtaatttatatgctcctcttgcatttgtgtctctacttcttgttcgcctggagggaacttcttcatttgtattagcatggttcacttcagtccttccttccaactcactctttttttgctgtaaacgattctctgccgccagttcattgaaactattgaatatatcctttagagattccgggatgaataaatcacctattaaagcagcttgacgatctggtggaactaaagtaagcaattgggtaacgacgcttacgagcttcataacatcttcttccgttggagctggtgggactaataactgtgtacaatccatttttctcatgagcatttcggtagctctcttcttgtctttctcgggcaatcttcctattattatagcaatagatttgtatagttgctttctattgtctaacagcttgttattctgtagcatcaaatctatggcagcctgacttgcttcttgtgaagagagcataccatttccaatcgaagatacgctggaatcttctgcgctagaatcaagaccatacggcctaccggttgtgagagattccatgggccttatgacatatcctggaaagagtagctcatcagacttacgtttactctctatatcaatatctacatcaggagcaatcatttcaataaacagccgacatacatcccagacgctataagctgtacgtgcttttaccgtcagattcttggctgtttcaatgtcgtccattttggttttcttttaccagtattgttcgtttgataatgtattcttgcttattacattataaaatctgtgcagatcacatgtcaaaacaactttttatcacaagatagtaccgcaaaacgaacctgcgggccgtctaaaaattaaggaaaagcagcaaaggtgcatttttaaaatatgaaatgaagataccgcagtaccaattattttcgcagtacaaataatgcgcggccggtgcatttttcgaaagaacgcgagacaaacaggacaattaaagttagtttttcgagttagcgtgtttgaatactgcaagatacaagataaatagagtagttgaaactagatatcaattgcacacaagatcggcgctaagcatgccacaatttggtatattatgtaaaacaccacctaaggtgcttgttcgtcagtttgtggaaaggtttgaaagaccttcaggtgagaaaatagcattatgtgctgctgaactaacctatttatgttggatgattacacataacggaacagcaatcaagagagccacattcatgagctataatactatcataagcaattcgctgagtttcgatattgtcaataaatcactccagtttaaatacaagacgcaaaaagcaacaattctggaagcctcattaaagaaattgattcctgcttgggaatttacaattattccttactatggacaaaaacatcaatctgatatcactgatattgtaagtagtttgcaattacagttcgaatcatcggaagaagcagataagggaaatagccacagtaaaaaaatgctaaagcacttctaagtgagggtgaaagcatctgggagatcactgagaaaatactaaattcgtttgagtatacttcgagatttacaaaaacaaaaactttataccaattcctcttcctagctactttcatcaattgtggaagattcagcgatattaagaacgttgatccgaaatcatttaaattagtccaaaataagtatctgggagtaataatccagtgtttagtgacagagacaaagacaagcgttagtaggcacatatacttctttagcgcaaggggtag

SEQ ID NO: 48 - DNA, 4989 bp, Artificial sequence
  vector pYIG7
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaatttaatacgactcactatagggaattcggatccttcaatatgcgcacatacgctgttatgttcaaggtcccttcgtttaagaacgaaagcggtcttccttttgagggatgtttcaagttgttcaaatctatcaaatttgcaaatccccagtctgtatctagagcgttgaatcggtgatgcgatttgttaattaaattgatggtgtcaccattaccaggtctagatataccaatggcaaactgagcacaacaataccagtccggatcaactggcaccatctctcccgtagtctcatctaatttttcttccggatgaggttccagatataccgcaacacctttattatggtttccctgagggaataatagaatgtcccattcgaaatcaccaattctaaacctgggcgaattgtatttcgggtttgttaactcgttccagtcaggaatgttccacgtgaagctatcttccagcaaagtctccacttcttcatcaaattgtggagaatactcccaatgctcttatctatgggacttccgggaaacacagtaccgatacttcccaattcgtcttcagagctcattgtttgtttgaagagactaatcaaagaatcgttttctcaaaaaaattaatatcttaactgatagtttgatcaaaggggcaaaacgtaggggcaaacaaacggaaaaatcgtttctcaaattttctgatgccaagaactctaaccagtcttatctaaaaattgccttatgatccgtctctccggttacagcctgtgtaactgattaatcctgcctttctaatcaccattctaatgttttaattaagggattttgtcttcattaacggctttcgctcataaaaatgttatgacgttttgcccgcaggcgggaaaccatccacttcacgagactgatctcctctgccggaacaccgggcatctccaacttataagttggagaaataagagaatttcagattgagagaatgaaaaaaaaaaaccctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaccatgaggtctttgctaatactagtgctttgcttcctgcccctggctgctctgggggtaccagatctcgacttggttgaacacgttgccaaggcttaagtgaatttactttaaagtcttgcatttaaataaattttctttttatagctttatgacttagtttcaatttatatactattttaatgacattttcgattcattgattgaaagctttgtgttttttcttgatgcgctattgcattgttcttgtctttttcgccacatgtaatatctgtagtagatacctgatacattgtggatgctgagtgaaattttagttaataatggaggcgctcttaataattttggggatattggcttttttttttaaagtttacaaatgaattttttccgccaggataacgattctgaagttactcttagcgttcctatcggtacagccatcaaatcatgcctataaatcatgcctatatttgcgtgcagtcagtatcatctacatgaaaaaaactcccgcaatttcttatagaatacgttgaaaattaaatgtacgcgccaagataagataacatatatctagctagatgcagtaatatacacagattcccgcggacgtgggaaggaaaaaattagataacaaaatctgagtgatatggaaattccgctgtatagctcatatctttcccttcaacaccagaaatgtaaaaatcttgttacgaaggatctttttgctaatgtttctcgctcaatcctcatttcttccctacgaagagtcaaatctacttgttttctgccggtatcaagatccatatcttctagtttcaccatcaaagtccaatttctagtatacagtttatgtcccaacgtaacagacaatcaaaattggaaaggataagtatccttcaaagaatgattctgcgctggctcctgaaccgcctaatgggaacagagaagtccaaaacgatgctataagaaccagaaataaaacgataaaaccataccaggatccaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 49 - DNA, 5422 bp, Artificial sequence
  vector pYIG7E1
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaatttaatacgactcactatagggaattcggatccttcaatatgcgcacatacgctgttatgttcaaggtcccttcgtttaagaacgaaagcggtcttccttttgagggatgtttcaagttgttcaaatctatcaaatttgcaaatccccagtctgtatctagagcgttgaatcggtgatgcgatttgttaattaaattgatggtgtcaccattaccaggtctagatataccaatggcaaactgagcacaacaataccagtccggatcaactggcaccatctctcccgtagtctcatctaatttttcttccggatgaggttccagatataccgcaacacctttattatggtttccctgagggaataatagaatgtcccattcgaaatcaccaattctaaacctgggcgaattgtatttcgggtttgttaactcgttccagtcaggaatgttccacgtgaagctatcttccagcaaagtctccacttcttcatcaaattgtggagaatactcccaatgctcttatctatgggacttccgggaaacacagtaccgatacttcccaattcgtcttcagagctcattgtttgtttgaagagactaatcaaagaatcgttttctcaaaaaaattaatatcttaactgatagtttgatcaaaggggcaaaacgtaggggcaaacaaacggaaaaatcgtttctcaaattttctgatgccaagaactctaaccagtcttatctaaaaattgccttatgatccgtctctccggttacagcctgtgtaactgattaatcctgcctttctaatcaccattctaatgttttaattaagggattttgtcttcattaacggctttcgctcataaaaatgttatgacgttttgcccgcaggcgggaaaccatccacttcacgagactgatctcctctgccggaacaccgggcatctccaacttataagttggagaaataagagaatttcagattgagagaatgaaaaaaaaaaaccctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaccatgaggtctttgctaatactagtgctttgcttcctgcccctggctgctctggggtatgaggtgcgcaacgtgtccgggatgtaccatgtcacgaacgactgctccaactcaagcattgtgtatgaggcagcggacatgatcatgcacacccccgggtgcgtgccctgcgttcgggagaacaactcttcccgctgctgggtagcgctcacccccacgctcgcagctaggaacgccagcgtccccaccacgacaatacgacgccacgtcgatttgctcgttggggcggctgctttctgttccgctatgtacgtgggggacctctgcggatctgtcttcctcgtctcccagctgttcaccatctcgcctcgccggcatgagacggtgcaggactgcaattgctcaatctatcccggccacataacgggtcaccgtatggcttgggatatgatgatgaactggtaatagacccttctcacctcggccgataagctcagatctcgacttggttgaacacgttgccaaggcttaagtgaatttactttaaagtcttgcatttaaataaattttctttttatagctttatgacttagtttcaatttatatactattttaatgacattttcgattcattgattgaaagctttgtgttttttcttgatgcgctattgcattgttcttgtctttttcgccacatgtaatatctgtagtagatacctgatacattgtggatgctgagtgaaattttagttaataatggaggcgctcttaataattttggggatattggcttttttttttaaagtttacaaatgaattttttccgccaggataacgattctgaagttactcttagcgttcctatcggtacagccatcaaatcatgcctataaatcatgcctatatttgcgtgcagtcagtatcatctacatgaaaaaaactcccgcaatttcttatagaatacgttgaaaattaaatgtacgcgccaagataagataacatatatctagctagatgcagtaatatacacagattcccgcggacgtgggaaggaaaaaattagataacaaaatctgagtgatatggaaattccgctgtatagctcatatctttcccttcaacaccagaaatgtaaaaatcttgttacgaaggatctttttgctaatgtttctcgctcaatcctcatttcttccctacgaagagtcaaatctacttgttttctgccggtatcaagatccatatcttctagtttcaccatcaaagtccaatttctagtatacagtttatgtcccaacgtaacagacaatcaaaattggaaaggataagtatccttcaaagaatgattctgcgctggctcctgaaccgcctaatgggaacagagaagtccaaaacgatgctataagaaccagaaataaaacgataaaaccataccaggatccaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 50 - DNA, 15621 bp, Artificial sequence
  vector pSY1YIG7E1s
atcgataagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaaagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctggtatggttttatcgttttatttctggttcttatagcatcgttttggacttctctgttcccattaggcggttcaggagccagcgcagaatcattctttgaaggatacttatcctttccaattttgattgtctgttacgttgggacataaactgtatactagaaattggactttgatggtgaaactagaagatatggatcttgataccggcagaaaacaagtagatttgactcttcgtagggaagaaatgaggattgagcgagaaacattagcaaaaagatccttcgtaacaagatttttacatttctggtgttgaagggaaagatatgagctatacagcggaatttccatatcactcagattttgttatctaattttttccttcccacgtccgcgggaatctgtgtatattactgcatctagctagatatatgttatcttatcttggcgcgtacatttaattttcaacgtattctataagaaattgcgggagtttttttcatgtagatgatactgactgcacgcaaatataggcatgatttataggcatgatttgatggctgtaccgataggaacgctaagagtaacttcagaatcgttatcctggcggaaaaaattcatttgtaaactttaaaaaaaaaagccaatatccccaaaattattaagagcgcctccattattaactaaaatttcactcagcatccacaatgtatcaggtatctactacagatattacatgtggcgaaaaagacaagaacaatgcaatagcgcatcaagaaaaaacacaaagctttcaatcaatgaatcgaaaatgtcattaaaatagtatataaattgaaactaagtcataaagctataaaaagaaaatttatttaaatgcaagactttaaagtaaattcacttaagccttggcaacgtgttcaaccaagtcgagatctgagcttatcggccgaggtgagaagggtctattaccagttcatcatcatatcccaagccatacggtgacccgttatgtggccgggatagattgagcaattgcagtcctgcaccgtctcatgccggcgaggcgagatggtgaacagctgggagacgaggaagacagatccgcagaggtcccccacgtacatagcggaacagaaagcagccgccccaacgagcaaatcgacgtggcgtcgtattgtcgtggtggggacgctggcgttcctagctgcgagcgtgggggtgagcgctacccagcagcgggaagagttgttctcccgaacgcagggcacgcacccgggggtgtgcatgatcatgtccgctgcctcatacacaatgcttgagttggagcagtcgttcgtgacatggtacatcccggacacgttgcgcacctcataccccagagcagccaggggcaggaagcaaagcactagtattagcaaagacctcatggtgtttgtttatgtgtgtttattcgaaactaagttcttggtgttttaaaactaaaaaaaagactaactataaaagtagaatttaagaagtttaagaaatagatttacagaattacaatcaatacctaccgtctttatatacttattagtcaagtaggggaataatttcagggaactggtttcaaccttttttttcagggtttttttttttcattctctcaatctgaaattctcttatttctccaacttataagttggagatgcccggtgttccggcagaggagatcagtctcgtgaagtggatggtttcccgcctgcgggcaaaacgtcataacatttttatgagcgaaagccgttaatgaagacaaaatcccttaattaaaacattagaatggtgattagaaaggcaggattaatcagttacacaggctgtaaccggagagacggatcataaggcaatttttagataagactggttagagttcttggcatcagaaaatttgagaaacgatttttccgtttgtttgcccctacgttttgcccctttgatcaaactatcagttaagatattaatttttttgagaaaacgattctttgattagtctcttcaaacaaacaatgagctctgaagacgaattgggaagtatcggtactgtgtttcccggaagtcccatagataagagcattgggagtattctccacaatttgatgaagaagtggagactttgctggaagatagcttcacgtggaacattcctgactggaacgagttaacaaacccgaaatacaattcgcccaggtttagaattggtgatttcgaatgggacattctattattccctcagggaaaccataataaaggtgttgcggtatatctggaacctcatccggaagaaaaattagatgagactacgggagagatggtgccagttgatccggactggtattgttgtgctcagtttgccattggtatatctagacctggtaatggtgacaccatcaatttaattaacaaatcgcatcaccgattcaacgctctagatacagactggggatttgcaaatttgatagatttgaacaacttgaaacatccctcaaaaggaagaccgctttcgttcttaaacgaagggaccttgaacataacagcgtatgtgcgcatattgaaggatcctctacgccggacgcatcgtggccggcatcaccggcgccacaggtgcggttgctggcccctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaattcccccttacacggaggcatcaagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggtgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaaataggcgtatcacgaggccctttcgtcttcaagaattctcatgtttgacagcttatcatcgatccacttgtatatttggatgaatttttgaggaattctgaaccagtcctaaaacgagtaaataggaccggcaattcttcaagcaataaacaggaataccaattattaaaagataacttagtcagatcgtacaataaagctttgaagaaaaatgcgccttattcaatctttgcataaaaaaatggcccaaaatctcacattggaagacatttgatgacctcatttctttcaatgaagggcctaacggagttgactaatgttgtgggaaattggaccgataagcgtgcttctgccgtggccaggacaacgtatactcatcagataacagcaatacctgatcactacttcgcactagtttctcggtactatgcatatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataaccatcccatccacctttcgctccttgaacttgcatctaaactcgacctctacattttttatgtttatctctagtattacctcttagacaaaaaaattgtagtaagaactattcatagagttaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattcttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttaacaaagatatgctattgaagtgcaagatggaaacgcagaaaatgaaccggggatgcgacgtgcaagattacctatgcaatagatgcaatagtttctccaggaaccgaaatacatacattgtcttccgtaaagcgctagactatatattattatacaggttcaaatatactatctgtttcagggaaaactcccaggttcggatgttcaaaattcaatgatgggtaacaagtacgatcgtaaatctgtaaaacagtttgtcggatattaggctgtatctcctcaaagcgtattcgaatatcattgagaagctgcattttttttttttttttttttttttttttttttatatatatttcaaggatataccattgtaatgtctgcccctaagaagatcgtcgttttgccaggtgaccacgttggtcaagaaatcacagccgaagccattaaggttcttaaagctatttctgatgttcgttccaatgtcaagttcgatttcgaaaatcatttaattggtggtgctgctatcgatgctacaggtgtcccacttccagatgaggcgctggaagcctccaagaaggttgatgccgttttgttaggtgctgtgggtggtcctaaatggggtaccggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatgttttggcctcttcaagattatggagaaaaactgtggaggaaaccatcaagaacgaattccctacattgaaggttcaacatcaattgattgattctgccgccatgatcctagttaagaacccaacccacctaaatggtattataatcaccagcaacatgtttggtgatatcatctccgatgaagcctccgttatcccaggttccttgggtttgttgccatctgcgtccttggcctctttgccagacaagaacaccgcatttggtttgtacgaaccatgccacggttctgctccagatttgccaaagaataaggttgaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccaccgaagtcggtgatgctgtcgccgaagaagttaagaaaatccttgcttaaaaagattctctttttttatgatatttgtacaaaaaaaaaaaaaaaaaaaaaaaaaaaaaaaaaaaaaaaaaaaaaatgcagcgtcacatcggataataatgatggcagccattgtagaagtgccttttgcatttctagtctctttctcggtctagctagttttactacatcgcgaagatagaatcttagatcacactgcctttgctgagctggatcatatgagtaacaaaagagtggtaaggcctcgttaaaggacaaggacctgagcggaagtgtatcgtaaagtagacggagtatactagtatagtctatagtccgtggaattctaagtgccagctttataatgtcattctccttactacagacccgcctgaaagtagacacatcatcatcagtaagctttgacaaaaagcattgagtagctaactcttctatgcaatctatagctgttttataaggcattcaatggacagattgaggtttttgaaacatactagtgaaattagccttaatcccttctcgaagttaatcatgcattatggtgtaaaaaatgcaactcgcgttgctctactttttcccgaatttccaaatacgcagctggggtgattgctcgatttcgtaacgaaagttttgtttataaaaaccgcgaaaaccttctgtaacagatagatttttacagcgctgatatacaatgacatcagctgtaatggaaaataactgaaatatgaatggcgagagactgcttgcttgtattaagcaatgtattatgcagcacttccaacctatggtgtacgatgaaagtaggtgtgtaatcgagacgacaagggggacttttccagttcctgatcattataagaaatacaaaacgttagcatttgcatttgttggacatgtactgaatacagacgacacaccggtaattgaaaaagaactggattggcctgatcctgcactagtgtacaatacaattgtcgatcgaatcataaatcacccagaattatcacagtttatatcggttgcatttattagtcagttaaaggccaccatcggagagggtttagatattaatgtaaaaggcacgctaaaccgcaggggaaagggtatcagaaggcctaaaggcgtattttttagatacatggaatctccatttgtcaatacaaaggtcactgcattcttctcttatcttcgagattataataaaattgcctcagaatatcacaataatactaaattcattctcacgttttcatgtcaagcatattgggcatctggcccaaacttctccgccttgaagaatgttatttggtgctccataattcatgaatacatttctaagtttgtggaaagagaacaggataaaggtcatataggagatcaggagctaccgcctgaagaggacccttctcgtgaactaaacaatgtacaacatgaagtcaatagtttaacggaacaagatgcggaggcggatgaaggattgtggggtgaaatagattcattatgtgaaaaatggcagtctgaagcggagagtcaaactgaggcggagataatagccgacaggataattggaaatagccagaggatggcgaacctcaaaattcgtcgtacaaagttcaaaagtgtcttgtatcatatactaaaggaactaattcaatctcagggaaccgtaaaggtttatcgcggtagtagtttttcacacgattcgataaagataagcttacattatgaagagcagcatattacagccgtatgggtctacttgatagtaaaatttgaagagcattggaagcctgttgatgtagaggtcgagtttagatgcaagttcaaggagcgaaaggtggatgggtaggttatatagggatatagcacagagatatatagcaaagagatacttttgaggcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgcattcatttcctttgatattggatcataccctagaagtattacgtgattttctgccccttaccctcgttgctactctcctttttttcgtgggaaccgctttagggccctcagtgatggtgttttgtaatttatatgctcctcttgcatttgtgtctctacttcttgttcgcctggagggaacttcttcatttgtattagcatggttcacttcagtccttccttccaactcactctttttttgctgtaaacgattctctgccgccagttcattgaaactattgaatatatcctttagagattccgggatgaataaatcacctattaaagcagcttgacgatctggtggaactaaagtaagcaattgggtaacgacgcttacgagcttcataacatcttcttccgttggagctggtgggactaataactgtgtacaatccatttttctcatgagcatttcggtagctctcttcttgtctttctcgggcaatcttcctattattatagcaatagatttgtatagttgctttctattgtctaacagcttgttattctgtagcatcaaatctatggcagcctgacttgcttcttgtgaagagagcataccatttccaatcgaagatacgctggaatcttctgcgctagaatcaagaccatacggcctaccggttgtgagagattccatgggccttatgacatatcctggaaagagtagctcatcagacttacgtttactctctatatcaatatctacatcaggagcaatcatttcaataaacagccgacatacatcccagacgctataagctgtacgtgcttttaccgtcagattcttggctgtttcaatgtcgtccattttggttttcttttaccagtattgttcgtttgataatgtattcttgcttattacattataaaatctgtgcagatcacatgtcaaaacaactttttatcacaagatagtaccgcaaaacgaacctgcgggccgtctaaaaattaaggaaaagcagcaaaggtgcatttttaaaatatgaaatgaagataccgcagtaccaattattttcgcagtacaaataatgcgcggccggtgcatttttcgaaagaacgcgagacaaacaggacaattaaagttagtttttcgagttagcgtgtttgaatactgcaagatacaagataaatagagtagttgaaactagatatcaattgcacacaagatcggcgctaagcatgccacaatttggtatattatgtaaaacaccacctaaggtgcttgttcgtcagtttgtggaaaggtttgaaagaccttcaggtgagaaaatagcattatgtgctgctgaactaacctatttatgttggatgattacacataacggaacagcaatcaagagagccacattcatgagctataatactatcataagcaattcgctgagtttcgatattgtcaataaatcactccagtttaaatacaagacgcaaaaagcaacaattctggaagcctcattaaagaaattgattcctgcttgggaatttacaattattccttactatggacaaaaacatcaatctgatatcactgatattgtaagtagtttgcaattacagttcgaatcatcggaagaagcagataagggaaatagccacagtaaaaaaatgctaaagcacttctaagtgagggtgaaagcatctgggagatcactgagaaaatactaaattcgtttgagtatacttcgagatttacaaaaacaaaaactttataccaattcctcttcctagctactttcatcaattgtggaagattcagcgatattaagaacgttgatccgaaatcatttaaattagtccaaaataagtatctgggagtaataatccagtgtttagtgacagagacaaagacaagcgttagtaggcacatatacttctttagcgcaaggggtag

SEQ ID NO: 51 - DNA, 3593 bp, Artificial sequence
  vector pPICZalphaA
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctgaattcacgtggcccagccggccgtctcggatcggtacctcgagccgcggcggccgccagctttctagaacaaaaactcatctcagaagaggatctgaatagcgccgtcgaccatcatcatcatcatcattgagtttgtagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 52 - DNA, 3547 bp, Artificial sequence
  vector pPICZalphaD′
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaaggggcccgaattcgcatgcggccgccagctttctagaacaaaaactcatctcagaagaggatctgaatagcgccgtcgaccatcatcatcatcatcattgagtttgtagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 53 - DNA, 3558 bp, Artificial sequence
  vector pPICZalphaE′
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagcctgcagcatatgctcgaggccgccagctttctagaacaaaaactcatctcagaagaggatctgaatagcgccgtcgaccatcatcatcatcatcattgagtttgtagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 54 - DNA, 28 bp, Artificial sequence
  synthetic probe or primer
tcgagaaaaggggcccgaattcgcatgc

SEQ ID NO: 55 - DNA, 28 bp, Artificial sequence
  synthetic probe or primer
ggccgcatgcgaattcgggccccttttc

SEQ ID NO: 56 - DNA, 35 bp, Artificial sequence
  synthetic probe or primer
tcgagaaaagagaggctgaagcctgcagcatatgc

SEQ ID NO: 57 - DNA, 35 bp, Artificial sequence
  synthetic probe or primer
ggccgcatatgctgcaggcttcagcctctcttttc

SEQ ID NO: 58 - DNA, 3997 bp, Artificial sequence
  vector pPICZalphaD′ E1sH6
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaaggtatgaggtgcgcaacgtgtccgggatgtaccatgtcacgaacgactgctccaactcaagcattgtgtatgaggcagcggacatgatcatgcacacccccgggtgcgtgccctgcgttcgggagaacaactcttcccgctgctgggtagcgctcacccccacgctcgcagctaggaacgccagcgtccccactacgacaatacgacgccacgtcgatttgctcgttggggcggctgctttctgttccgctatgtacgtgggggatctctgcggatctgtcttcctcgtctcccagctgttcaccatctcgcctcgccggcatgagacggtgcaggactgcaattgctcaatctatcccggccacataacaggtcaccgtatggcttgggatatgatgatgaactggcaccaccaccatcaccattaaagatctaagcttgaatcccgcggccatgcgaattcgcatgcggccgccagctttctagaacaaaaactcatctcagaagaggatctgaatagcgccgtcgaccatcatcatcatcatcattgagtttgtagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 59 - DNA, 4004 bp, Artificial sequence
  vector pPICZalphaE′ E1sH6
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagcctatgaggtgcgcaacgtgtccgggatgtaccatgtcacgaacgactgctccaactcaagcattgtgtatgaggcagcggacatgatcatgcacacccccgggtgcgtgccctgcgttcgggagaacaactcttcccgctgctgggtagcgctcacccccacgctcgcagctaggaacgccagcgtccccactacgacaatacgacgccacgtcgatttgctcgttggggcggctgctttctgttccgctatgtacgtgggggatctctgcggatctgtcttcctcgtctcccagctgttcaccatctcgcctcgccggcatgagacggtgcaggactgcaattgctcaatctatcccggccacataacgggtcaccgtatggcttgggatatgatgatgaactggcaccaccaccatcaccattaaagatctaagcttgaatcccgcggccatggcatatgcggccgccagctttctagaacaaaaactcatctcagaagaggatctgaatagcgccgtcgaccatcatcatcatcatcattgagtttgtagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 60 - DNA, 4492 bp, Artificial sequence
  vector pPICZalphaD′ E2sH6
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaaggcatacccgcgtgtcaggaggggcagcagcctccgataccaggggccttgtgtccctctttagccccgggtcggctcagaaaatccagctcgtaaacaccaacggcagttggcacatcaacaggactgccctgaactgcaacgactccctccaaacagggttctttgccgcactattctacaaacacaaattcaactcgtctggatgcccagagcgcttggccagctgtcgctccatcgacaagttcgctcaggggtggggtcccctcacttacactgagcctaacagctcggaccagaggccctactgctggcactacgcgcctcgaccgtgtggtattgtacccgcgtctcaggtgtgcggtccagtgtattgcttcaccccgagccctgttgtggtggggacgaccgatcggtttggtgtccccacgtataactggggggcgaacgactcggatgtgctgattctcaacaacacgcggccgccgcgaggcaactggttcggctgtacatggatgaatggcactgggttcaccaagacgtgtgggggccccccgtgcaacatcgggggggccggcaacaacaccttgacctgccccactgactgttttcggaagcaccccgaggccacctacgccagatgcggttctgggccctggctgacacctaggtgtatggttcattacccatataggctctggcactacccctgcactgtcaacttcaccatcttcaaggttaggatgtacgtggggggcgtggagcacaggttcgaagccgcatgcaattggactcgaggagagcgttgtgacttggaggacagggatagatcagagcttagcccgctgctgctgtctacaacagagtggcaggtgatcgagggcagacaccatcaccaccatcactaatagttaattaactgcaggcatgcaagcttatcgataccgtcgacgaattcgcatgcggccgccagctttctagaacaaaaactcatctcagaagaggatctgaatagcgccgtcgaccatcatcatcatcatcattgagtttgtagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 61 - DNA, 4431 bp, Artificial sequence
  vector pPICZalphaE′ E2sH6
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagcccatacccgcgtgtcaggaggggcagcagcctccgataccaggggccttgtgtccctctttagccccgggtcggctcagaaaatccagctcgtaaacaccaacggcagttggcacatcaacaggactgccctgaactgcaacgactccctccaaacagggttctttgccgcactattctacaaacacaaattcaactcgtctggatgcccagagcgcttggccagctgtcgctccatcgacaagttcgctcaggggtggggtcccctcacttacactgagcctaacagctcggaccagaggccctactgctggcactacgcgcctcgaccgtgtggtattgtacccgcgtctcaggtgtgcggtccagtgtattgcttcaccccgagccctgttgtggtggggacgaccgatcggtttggtgtccccacgtataactggggggcgaacgactcggatgtgctgattctcaacaacacgcggccgccgcgaggcaactggttcggctgtacatggatgaatggcactgggttcaccaagacgtgtgggggccccccgtgcaacatcgggggggccggcaacaacaccttgacctgccccactgactgttttcggaagcaccccgaggccacctacgccagatgcggttctgggccctggctgacacctaggtgtatggttcattacccatataggctctggcactacccctgcactgtcaacttcaccatcttcaaggttaggatgtacgtggggggcgtggagcacaggttcgaagccgcatgcaattggactcgaggagagcgttgtgacttggaggacagggatagatcagagcttagcccgctgctgctgtctacaacagagtggcaggtgatcgagggcagacaccatcaccaccatcactaatagttaattaactgcaggcatgcaagcttatcgataccgtcgaccatcatcatcatcatcattgagtttgtagccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaccttcgtttgtgcggatcccccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctggagaccaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagatc

SEQ ID NO: 62 - DNA, 2880 bp, Artificial sequence
  vector pUC18MFa
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttaccccttcttctttagcagcaatgctggcaatagtagtatttataaacaataacccgttatttgtgctgttggaaaatggcaaaacagcaacatcgaaatccccttctaaatctgagtaaccgatgacagcttcagccggaatttgtgccgtttcatcttctgttgtagtgttgactggagcagctaatgcggaggatgctgcgaataaaactgcagtaaaaattgaaggaaatctcatgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 63 - protein, 6 aa, Artificial sequence
  adaptor peptide
HHHHHH

SEQ ID NO: 64 - protein, 6 aa, Artificial sequence
  adaptor peptide
EEGEPK

SEQ ID NO: 65 - protein, 6 aa, Artificial sequence
  adaptor peptide
EEAEPK

SEQ ID NO: 66 - protein, 5 aa, Artificial sequence
  processing site
  MISC_FEATURE (5)..(5): X is any amino acid
IEGRX

SEQ ID NO: 67 - protein, 5 aa, Artificial sequence
  processing site
  MISC_FEATURE (5)..(5): X is any amino acid
IDGRX

SEQ ID NO: 68 - protein, 5 aa, Artificial sequence
  processing site
  MISC_FEATURE (5)..(5): X is any amino acid
AEGRX

SEQ ID NO: 69 - protein, 5 aa, Artificial sequence
  adaptor peptide
VIEGR

SEQ ID NO: 70 - protein, 4 aa, Artificial sequence
  adaptor peptide
IEGR

SEQ ID NO: 71 - protein, 4 aa, Artificial sequence
  adaptor peptide
IDGR

SEQ ID NO: 72 - protein, 4 aa, Artificial sequence
  adaptor peptide
AEGR

SEQ ID NO: 73 - protein, 4 aa, Artificial sequence
  HCV E1
NNSS

SEQ ID NO: 74 - protein, 8 aa, Artificial sequence
  FLAG epitope
DYKDDDDK

SEQ ID NO: 75 - protein, 12 aa, Artificial sequence
  Protein C epitope
EDQVDPRLIDGK

SEQ ID NO: 76 - protein, 11 aa, Artificial sequence
  VSV epitope
YTDIEMNRLGK

SEQ ID NO: 77 - protein, 9 aa, Artificial sequence
  streptag
AWRHPQFGG

SEQ ID NO: 78 - protein, 12 aa, Artificial sequence
  Tag100 epitope
EETARFQPGYRS

SEQ ID NO: 79 - protein, 10 aa, Artificial sequence
  c-myc epitope
EQKLISEEDL

SEQ ID NO: 80 - protein, 11 aa, Artificial sequence
  HA epitope
YPYDVPDYASL

SEQ ID NO: 81 - protein, 9 aa, Artificial sequence
  HA epitope
YPYDVPDYA

SEQ ID NO: 82 - protein, 12 aa, Artificial sequence
  HA epitope
CYPYDVPDYASL

SEQ ID NO: 83 - protein, 6 aa, Artificial sequence
  thrombin cleavage site
LVPRGS

SEQ ID NO: 84 - protein, 4 aa, Artificial sequence
  collagenase recognition site
  MISC_FEATURE (2)..(2): Xaa is any amino acid but most frequently a neutral amino acid
PXGP

SEQ ID NO: 85 - protein, 192 aa, hepatitis C virus
YQVRNSSGLYHVTNDCPNSSVVYEAADAILHTPGCVPCVREGNASRCWVAVTPTVATRDGKLPTTQLRRHIDLLVGSATLCSALYVGDLCGSVFLVGQLFTFSPRHHWTTQDCNCSIYPGHITGHRMAWDMMMNWSPTAALVVAQLLRIPQAIMDMIAGAHWGVLAGIAYFSMVGNWAEVLVVLLLFAGVDA

SEQ ID NO: 86 - protein, 209 aa, hepatitis C virus
MLGKLIDTLTCGFADLMGYIPLVGAPLGGAARALAHGARVLEDGVIYATGNLPGCSFSIFLLALLSCLTVPASAYQVRNSSGLYHVTNDCPNSSVVYEAADAILHTPGCVPCVREGNASRCWVAVTPTVATRDGKLPTTQLRRHIDLLVGSATLCSALYVGDLCGSVFLVGQLFTFSPRHHWTTQDCNCSIYPGHITGHRMAWDMMMNW

SEQ ID NO: 87 - protein, 192 aa, hepatitis C virus
YEVRNVSGMYHVTNDCSNSSIVYEAADMIMHTPGCVPCVRENNSSRCWVALTPTLAARNASVPTTTIRRHVDLLVGAAAFCSAMYVGDLCGSVFLVSQLFTISPRRHETVQDCNCSIYPGHITGHRMAWDMMMNWSPTTALVVSQLLRIPQAVVDMVAGAHWGVLAGLAYYSMVGNWAKVLVVMLLFAGVDG

SEQ ID NO: 88 - protein, 209 aa, hepatitis C virus
MLGKVIDTLTCGFADLMGYIPLVGAPLGGAARALAHGVRVLEDGVNYATGNLPGCSFSIFLLALLSCLTIPASAYEVRNVSGMYHVTNDCSNSSIVYEAADMIMHTPGCVPCVRENNSSRCWVALTPTLAARNASVPTTTIRRHVDLLVGAAAFCSAMYVGDLCGSVFLVSQLFTISPRRHETVQDCNCSIYPGHITGHRMAWDMMMNW

SEQ ID NO: 89 - protein, 209 aa, hepatitis C virus
MLGKVIDTLTCGFADLMGYIPVVGAPVGGVARALAHGVRVLEDGINYATGNLPGCSFSIFLLALLSCITTPVSAVEVKNNSNSYMATNDCSNSSIIWQLEGAVLHTPGCVPCELADNTSRCWVPVTPNMAIRQPGELTKGLRAHVDVIVMSATLCSALYVGDVCGALMIAAQVVVVSPQHHHFVQECNCSIYPGHITGHRMAWDMMMNW

SEQ ID NO: 90 - protein, 209 aa, hepatitis C virus
MLGKVIDTLTCGFADLMGYIPVVGAPVGGVARALAHGVRVLEDGINYATGNLPGCSFSIFLLALLSCVTAPVSAVEVKNTSQAYMATNDCSNNSIVWQLEDAVLHVPGCVPCENSSGRFHCWIPISPNIAVSKPGALTKGLRARIDAVVMSATLCSALYVGDVCGAVMIAAQAFIVAPKRHYFVQECNCSIYPGHITGHRMAWDMMMNW

SEQ ID NO: 91 - protein, 209 aa, hepatitis C virus
MLGKVIDTLTCGFADLMGYIPLVGAPVGGVARALAHGVRALEDGINFATGNLPGCSFSIFLLALFSCLIHPAASLEWRNTSGLYVLTNDCSNSSIVYEADDVILHTPGCIPCVQDGNTSTCWTPVTPTVAVKYVGATTASIRSHVDLLVGAATMCSALYVGDMCGAVFLVGQAFTFRPRRHQTVQTCNCSLYPGHLSGHRMAWDMMMNW

SEQ ID NO: 92 - protein, 209 aa, hepatitis C virus
MSGKVIDTLTCGFADLMGYIPLVGAPVGGVARALAHGVRAVEDGINYATGNLPGCSFSIFLLALLSCLTVPTSAVNYRNASGVYHITNDCPNSSIVYETEHHILHLPGCLPCVRVGNQSRCWVALTPTVAAPYIGAPLESLRSHVDLMVGAATACSALYIGDLCGGVFLVGQMFSFQPRRHWTTQDCNCSIYAGHVTGHRMAWDMMMNW

SEQ ID NO: 93 - protein, 209 aa, hepatitis C virus
MLGKVIDTLTCGFADLMGYIPLVGGPIGGVARALAHGVRVLEDGVNYATGNLPGCSFSIFILALLSCLTVPASAVPYRNASGIYHVTNDCPNSSIVYEADNLILHAPGCVPCVMTGNVSRCWVQITPTLSAPSLGAVTAPLRRAVDYLAGGAALCSALYVGDACGALFLVGQMFTYRPRQHATVQNCNCSIYSGHVTGHRMAWDMMMNW

SEQ ID NO: 94 - protein, 209 aa, hepatitis C virus
MLGKVIDTLTCGFADLMGYIPVVGAPLGGVAAAFAHGVRALEDGINYATGNLPGCSFSIFLLALLSCLTTPASALTYGNSSGLYHLTNDCPNSSIVLEADAMILHLPGCLPCVRVNNQSTCWHAVSPTLAIPNASTPATGFRRHVDLLAGAAVVCSSLYIGDLCGSLFLAGQLFTFQPRRHWTVQDCNCSIYTGHVTGHRMAWDMMMNW

SEQ ID NO: 95 - protein, 209 aa, hepatitis C virus
MLGKVIDTLTCGFADLMGYIPVVGAPLGGIAAALAHGVRAVEDGINYATGNLPGCSFSIFLLALLSCLTTPASAVHYANKSGLYHLTNDCPNSSIVYEAPAVIMHLPGCVPCVKVGNQSTCWLPASPTLAVPNASTPLTRFRKHVDLMVGAAAFCSAMYVGDICGGLFLLGQVVTIRPRLHQTVQECNCSIYTGKITGHRMAWDIMMNW

SEQ ID NO: 96 - protein, 209 aa, hepatitis C virus
MLGKVIDTLTCGLADLMGYIPVLGGPLGGVAAALAHGVRAIEDGVNYATGNLPGCSFSILLLALLSCLTIPASAIQVKNASGIYHLTNDCSNNSIVFEAETMILHLPGCVPCIKAGNESRCWLPVSPTLAVPNSSVPIHGFRRHVDLLVGAAAFCSAMYIGDLCGSIFLVGQLFTFRPKYHQVTQDCNCSIYAGHITGHRMAWDMMMNW

SEQ ID NO: 97 - protein, 363 aa, hepatitis C virus
ETHVTGGNAGRTTAGPVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAIKWEYVVLLFLLLADARVCSEFWMMLLISQAEA

SEQ ID NO: 98 - protein, 363 aa, hepatitis C virus
HTRVSGGAAASDTRGLVSLFSPGSAQKIQLVNTNGSWHINRTALNCNDSLQTGFFAALFYKHKFNSSGCPERLASCRSIDKFAQGWGPLTYTEPNSSDQRPYCWHYAPRPCGIVPASQVCGPVYCFTPSPVVVGTTDRFGVPTYNWGANDSDVLILNNTRPPRGNWFGCTWMNGTGFTKTCGGPPCNIGGAGNNTLTCPTDCFRKHPEATYARCGSGPWLTPRCMVHYPYRLWHYPCTVNFTIFKVRMYVGGVEHRFEAACNWTRGERCDLEDRDRSELSPLLLSTTEWQILPCSFTTLPALSTGLIHLHQNIVDVQYLYGVGSAVVSLVIKWEYVLLLFLLLADARICACLWMMLLIAQAEA

SEQ ID NO: 99 - protein, 18 aa, chicken lysozyme leader peptide
MRSLLILVLCFLPLAALG

SEQ ID NO: 100 - DNA, 480 bp, human lysozyme leader peptide + Hepatitis C Virus + adaptor peptide
atgaaagcactaatagtcctaggcctggtactcctatcagtgactgtacaaggctatgaggtgcgcaacgtgtccgggatgtaccatgtcacgaacgactgctccaactcaagcattgtgtatgaggcagcggacatgatcatgcacacccccgggtgcgtgccctgcgttcgggagaacaactcttcccgctgctgggtagcgctcacccccacgctcgcagctaggaacgccagcgtccccactacgacaatacgacgccacgtcgatttgctcgttggggcggctgctttctgttccgctatgtacgtgggggatctctgcggatctgtcttcctcgtctcccagctgttcaccatctcgcctcgccggcatgagacggtgcaggactgcaattgctcaatctatcccggccacataacaggtcaccgtatggcttgggatatgatgatgaactggcaccaccaccatcaccattaa

SEQ ID NO: 101 - protein, 18 aa, human lysozyme leader peptide
MKALIVLGLVLLSVTVQG

SEQ ID NO: 102 - DNA, 56 bp, synthetic probe or primer
tggtactcctatcagtgactgtacaaggctatgaggtgcgcaacgtgtccgggatg

SEQ ID NO: 103 - DNA, 50 bp, synthetic probe or primer
ggcctaggactattagtgctttcatgagttcccgatgaaggcagagagcg

SEQ ID NO: 104 - DNA, 4201 bp, vector pUC18-FMD-M-E1-H6
  misc_feature (1134)..(1134): N is any nucleotide
  misc_feature (955)..(955): N is any nucleotide
  misc_feature (956)..(956): N is any nucleotide
  misc_feature (1135)..(1135): N is any nucleotide
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatccttaatggtgatggtggtggtgccagttcatcatcatatcccaagccatacggtgacctgttatgtggccgggatagattgagcaattgcagtcctgcaccgtctcatgccggcgaggcgagatggtgaacagctgggagacgaggaagacagatccgcagagatcccccacgtacatagcggaacagaaagcagccgccccaacgagcaaatcgacgtggcgtcgtattgtcgtagtggggacgctggcgttcctagctgcgagcgtgggggtgagcgctacccagcagcgggaagagttgttctcccgaacgcagggcacgcacccgggggtgtgcatgatcatgtccgctgcctcatacacaatgcttgagttggagcagtcgttcgtgacatggtacatcccggacacgttgcgcacctcatacatgaattcccgatgaaggcagagagcgcaaggaggcggtatttatagtgccattcccctctctgagagacccggatggtagtcgagtgttatcggagacagcttgatgtagactccgtgcctgccggtcctcttattggcggacaccagtgagacaccccggaacttgctgtttttctgcaaaatccggggtgaccagtgggagcctatttgcacacacgagcgggacaccccactctggtgaagagtgccaaagtcattctttttcccgtnncggggcagccgattgcatgttttaggaaaatattacctttgctacaccctgtcagatttaccctccacacatatatattccgtcacctccagggactattcttggctcgttgcgccgccgcggaagatatccagaagctgtgttttccgagagactcggttggcgcctggtatatttnnaggatgtcgcgctgcctcacgtcccggtacccaggaacgcggtgggatctcgggcccatcgaagactgtgctccagactgctcgcccagcaggtgtttcttgattgccgcctctaaatagtccgcgcatcgccggtaacatttttccagctcggagtttgcgtttagatacatttctgcgatgccaaaggagcctgcagattataacctcggatgctgtcattcagcgcttttaatttgacctccagatagttgctgtatttctgttccattggctgctggacgttcgtataactcgagttattgttgcgctctgcctcggcgtactggctcatgactgactgcggtcgcttctcgagtgttctcgcaacaggacgcctgcaggtcatcgagtcgagctggcgccgaaactggcggatctgacctccacactgccctgtatctctatccaccgggaaccgcctcctgccgttccagaatgttgttcaagtggtagctctgtgcggtcaatgaaggcgttattgccggtgaaatctttgggaagcggtttatcctcggggaagattacgaaattcccgcgcgtcgttgcgcttcctggatctcgaggaagatcgttctccgcgtcgaggagatcgttctccgcgtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 105 - DNA, 4252 bp, vector pUC18-FMD-HL-E1-H6
  misc_feature (1006)..(1006): N is any nucleotide
  misc_feature (1007)..(1007): N is any nucleotide
  misc_feature (1185)..(1185): N is any nucleotide
  misc_feature (1186)..(1186): N is any nucleotide
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatccttaatggtgatggtggtggtgccagttcatcatcatatcccaagccatacggtgacctgttatgtggccgggatagattgagcaattgcagtcctgcaccgtctcatgccggcgaggcgagatggtgaacagctgggagacgaggaagacagatccgcagagatcccccacgtacatagcggaacagaaagcagccgccccaacgagcaaatcgacgtggcgtcgtattgtcgtagtggggacgctggcgttcctagctgcgagcgtgggggtgagcgctacccagcagcgggaagagttgttctcccgaacgcagggcacgcacccgggggtgtgcatgatcatgtccgctgcctcatacacaatgcttgagttggagcagtcgttcgtgacatggtacatcccggacacgttgcgcacctcatagccttgtacagtcactgataggagtaccaggcctaggactattagtgctttcatgaattcccgatgaaggcagagagcgcaaggaggcggtatttatagtgccattcccctctctgagagacccggatggtagtcgagtgttatcggagacagcttgatgtagactccgtgcctgccggtcctcttattggcggacaccagtgagacaccccggaacttgctgtttttctgcaaaatccggggtgaccagtgggagcctatttgcacacacgagcgggacaccccactctggtgaagagtgccaaagtcattctttttcccgtnncggggcagccgattgcatgttttaggaaaatattacctttgctacaccctgtcagatttaccctccacacatatatattccgtcacctccagggactattcttggctcgttgcgccgccgcggaagatatccagaagctgtgttttccgagagactcggttggcgcctggtatatttnnaggatgtcgcgctgcctcacgtcccggtacccaggaacgcggtgggatctcgggcccatcgaagactgtgctccagactgctcgcccagcaggtgtttcttgattgccgcctctaaatagtccgcgcatcgccggtaacatttttccagctcggagtttgcgtttagatacatttctgcgatgccaaaggagcctgcagattataacctcggatgctgtcattcagcgcttttaatttgacctccagatagttgctgtatttctgttccattggctgctggacgttcgtataactcgagttattgttgcgctctgcctcggcgtactggctcatgactgactgcggtcgcttctcgagtgttctcgcaacaggacgcctgcaggtcatcgagtcgagctggcgccgaaactggcggatctgacctccacactgccctgtatctctatccaccgggaaccgcctcctgccgttccagaatgttgttcaagtggtagctctgtgcggtcaatgaaggcgttattgccggtgaaatctttgggaagcggtttatcctcggggaagattacgaaattcccgcgcgtcgttgcgcttcctggatctcgaggaagatcgttctccgcgtcgaggagatcgttctccgcgtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 106 - DNA, 6973 bp, vector pFPMT121; identical to SEQ ID NO: 12

SEQ ID NO: 107 - DNA, 7428 bp, vector pFPMT-HL-E1-H6
  misc_feature (1095)..(1095): N is any nucleotide
  misc_feature (1096)..(1096): N is any nucleotide
  misc_feature (1274)..(1274): N is any nucleotide
  misc_feature (1275)..(1275): N is any nucleotide
ggtaccctgctcaatctccggaatggtgatctgatcgttcctgaaaacctcgacattggctccctcctgacacaggtactcgtacaggttccaggtaaacgagtcgtagttgtcgatcatgacaacgttcttagaagcggccggcattttgaaggtgactaatagcctaagaaaatatttaatttaattttcattaaattttcctatactcgctatttcagcttttcatctcatcacttcataaacgatataaaccagaaaaagaactattttcaaacacgcttctcaaaagcggtatgtccttccacgtctccttagaatctggcaagtccgcgagggggatccttaatggtgatggtggtggtgccagttcatcatcatatcccaagccatacggtgacctgttatgtggccgggatagattgagcaattgcagtcctgcaccgtctcatgccggcgaggcgagatggtgaacagctgggagacgaggaagacagatccgcagagatcccccacgtacatagcggaacagaaagcagccgccccaacgagcaaatcgacgtggcgtcgtattgtcgtagtggggacgctggcgttcctagctgcgagcgtgggggtgagcgctacccagcagcgggaagagttgttctcccgaacgcagggcacgcacccgggggtgtgcatgatcatgtccgctgcctcatacacaatgcttgagttggagcagtcgttcgtgacatggtacatcccggacacgttgcgcacctcatagccttgtacagtcactgataggagtaccaggcctaggactattagtgctttcatgaattcccgatgaaggcagagagcgcaaggaggcggtatttatagtgccattcccctctctgagagacccggatggtagtcgagtgttatcggagacagcttgatgtagactccgtgcctgccggtcctcttattggcggacaccagtgagacaccccggaacttgctgtttttctgcaaaatccggggtgaccagtgggagcctatttgcacacacgagcgggacaccccactctggtgaagagtgccaaagtcattctttttcccgtnncggggcagccgattgcatgttttaggaaaatattacctttgctacaccctgtcagatttaccctccacacatatatattccgtcacctccagggactattcttggctcgttgcgccgccgcggaagatatccagaagctgtgttttccgagagactcggttggcgcctggtatatttnnaggatgtcgcgctgcctcacgtcccggtacccaggaacgcggtgggatctcgggcccatcgaagactgtgctccagactgctcgcccagcaggtgtttcttgattgccgcctctaaatagtccgcgcatcgccggtaacatttttccagctcggagtttgcgtttagatacatttctgcgatgccaaaggagcctgcagattataacctcggatgctgtcattcagcgcttttaatttgacctccagatagttgctgtatttctgttccattggctgctggacgttcgtataactcgagttattgttgcgctctgcctcggcgtactggctcatgactgactgcggtcgcttctcgagtgttctcgcaacaggacgcctgcaggtcatcgagtcgagctggcgccgaaactggcggatctgacctccacactgccctgtatctctatccaccgggaaccgcctcctgccgttccagaatgttgttcaagtggtagctctgtgcggtcaatgaaggcgttattgccggtgaaatctttgggaagcggtttatcctcggggaagattacgaaattcccgcgcgtcgttgcgcttcctggatctcgaggaagatcgttctccgcgtcgaggagatcgttctccgcgtcgacaccattccttgcggcggcggtgctcaacggcctcaacctactactgggctgcttcctaatgcaggagtcgcataagggagagcgtcgacaaacccgcgtttgagaacttgctcaagcttctggtaaacgttgtagtactctgaaacaaggccctagcactctgatctgtttctcttgggtagcggtgagtggtttattggagttcactggtttcagcacatctgtcatctagacaatattgttactaaatttttttgaactacaattgttcgtaattcatctattattatacatcctcgtcagcaatttctggcagacggagtttactaacgtcttgagtatgaggccgagaatccagctctgtggccatactcagtcttgacagcctgctgatgtggctgcgttcaacgcaataagcgtgtcctccgactccgagttgtgctcgttatcgtcgttctcatcctcggaaaaatcacacgaaagaacatactcaccagtaggctttctggtccctggggcacggctgtttctgacgtattccggcgttgataatagctcgaaagtgaacgccgagtcgcgggagtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatcggggggggggggggggggggggggcaaacaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaagccccccccccccccccccccccccccccccccccgcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaattcccccttacacggaggcatcaagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaa

SEQ ID NO: 108 - protein, 129 aa, homo sapiens [sequence omitted]

SEQ ID NO: 109 - DNA, 7597 bp, vector pFPMT-MFa-IL-4
  misc_feature (1260)..(1260): N is any nucleotide
  misc_feature (1261)..(1261): N is any nucleotide
  misc_feature (1439)..(1439): N is any nucleotide
  misc_feature (1440)..(1440): N is any nucleotide
ggtaccctgctcaatctccggaatggtgatctgatcgttcctgaaaacctcgacattggctccctcctgacacaggtactcgtacaggttccaggtaaacgagtcgtagttgtcgatcatgacaacgttcttagaagcggccggcattttgaaggtgactaatagcctaagaaaatatttaatttaattttcattaaattttcctatactcgctatttcagcttttcatctcatcacttcataaacgatataaaccagaaaaagaactattttcaaacacgcttctcaaaagcggtatgtccttccacgtctccttagaatctggcaagtccgcgagggggatcctcagctcgaacactttgaatatttctctctcatgatcgtctttagcctttccaagaagttttccaacgtactctggttggcttccttcacaggacaggaattcaagcccgccaggccccagaggttcctgtcgagccgtttcaggaatcggatcagctgcttgtgcctgtggaactgctgtgcagtcgcacccaggcagcgagtgtccttctcatggtggctgtagaactgccggagcacagtcgcagccctgcagaaggtttccttctcagttgtgttcttggaggcagcaaagatgtctgttacggtcaactcggtgcacagagtcttctgctctgtgaggctgttcaaagttttgatgatctcctgtaaggtgatatcgcacttgtgtcttttatccaagcttaccccttcttctttagcagcaatgctggcaatagtagtatttataaacaataacccgttatttgtgctgttggaaaatggcaaaacagcaacatcgaaatccccttctaaatctgagtaaccgatgacagcttcagccggaatttgtgccgtttcatcttctgttgtagtgttgactggagcagctaatgcggaggatgctgcgaataaaactgcagtaaaaattgaaggaaatctcatgaattcccgatgaaggcagagagcgcaaggaggcggtatttatagtgccattcccctctctgagagacccggatggtagtcgagtgttatcggagacagcttgatgtagactccgtgcctgccggtcctcttattggcggacaccagtgagacaccccggaacttgctgtttttctgcaaaatccggggtgaccagtgggagcctatttgcacacacgagcgggacaccccactctggtgaagagtgccaaagtcattctttttcccgtnncggggcagccgattgcatgttttaggaaaatattacctttgctacaccctgtcagatttaccctccacacatatatattccgtcacctccagggactattcttggctcgttgcgccgccgcggaagatatccagaagctgtgttttccgagagactcggttggcgcctggtatatttnnaggatgtcgcgctgcctcacgtcccggtacccaggaacgcggtgggatctcgggcccatcgaagactgtgctccagactgctcgcccagcaggtgtttcttgattgccgcctctaaatagtccgcgcatcgccggtaacatttttccagctcggagtttgcgtttagatacatttctgcgatgccaaaggagcctgcagattataacctcggatgctgtcattcagcgcttttaatttgacctccagatagttgctgtatttctgttccattggctgctggacgttcgtataactcgagttattgttgcgctctgcctcggcgtactggctcatgactgactgcggtcgcttctcgagtgttctcgcaacaggacgcctgcaggtcatcgagtcgagctggcgccgaaactggcggatctgacctccacactgccctgtatctctatccaccgggaaccgcctcctgccgttccagaatgttgttcaagtggtagctctgtgcggtcaatgaaggcgttattgccggtgaaatctttgggaagcggtttatcctcggggaagattacgaaattcccgcgcgtcgttgcgcttcctggatctcgaggaagatcgttctccgcgtcgaggagatcgttctccgcgtcgacaccattccttgcggcggcggtgctcaacggcctcaacctactactgggctgcttcctaatgcaggagtcgcataagggagagcgtcgacaaacccgcgtttgagaacttgctcaagctagcttctggtaaacgttgtagtactctgaaacaaggccctagcactctgatctgtttctcttgggtagcggtgagtggtttattggagttcactggtttcagcacatctgtcatctagacaatattgttactaaatttttttgaactacaattgttcgtaattcatctattattatacatcctcgtcagcaatttctggcagacggagtttactaacgtcttgagtatgaggccgagaatccagctctgtggccatactcagtcttgacagcctgctgatgtggctgcgttcaacgcaataagcgtgtcctccgactccgagttgtgctcgttatcgtcgttctcatcctcggaaaaatcacacgaaagaacatactcaccagtaggctttctggtccctggggcacggctgtttctgacgtattccggcgttgataatagctcgaaagtgaacgccgagtcgcgggagtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatcggggggggggggggggggggggggcaaacaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaagccccccccccccccccccccccccccccccccccgcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaattcccccttacacggaggcatcaagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaa

SEQ ID NO: 110 - DNA, 3304 bp, vector pUC18-MFa-IL-4
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcagggatcctcagctcgaacactttgaatatttctctctcatgatcgtctttagcctttccaagaagttttccaacgtactctggttggcttccttcacaggacaggaattcaagcccgccaggccccagaggttcctgtcgagccgtttcaggaatcggatcagctgcttgtgcctgtggaactgctgtgcagtcgcacccaggcagcgagtgtccttctcatggtggctgtagaactgccggagcacagtcgcagccctgcagaaggtttccttctcagttgtgttcttggaggcagcaaagatgtctgttacggtcaactcggtgcacagagtcttctgctctgtgaggctgttcaaagttttgatgatctcctgtaaggtgatatcgcacttgtgtcttttatccaagcttaccccttcttctttagcagcaatgctggcaatagtagtatttataaacaataacccgttatttgtgctgttggaaaatggcaaaacagcaacatcgaaatccccttctaaatctgagtaaccgatgacagcttcagccggaatttgtgccgtttcatcttctgttgtagtgttgactggagcagctaatgcggaggatgctgcgaataaaactgcagtaaaaattgaaggaaatctcatgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 111 - DNA, 57 bp, synthetic probe or primer
tgcttcctaccactagcagcactaggacacaagtgcgatatcaccttacaggagatc

SEQ ID NO: 112 - DNA, 54 bp, synthetic probe or primer
tagtactagtattagtaggctgcgcatgaattcactggccgtcgttttacaacg

SEQ ID NO: 113 - DNA, 3103 bp, vector pUC18-CL-IL-4
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcagggatcctcagctcgaacactttgaatatttctctctcatgatcgtctttagcctttccaagaagttttccaacgtactctggttggcttccttcacaggacaggaattcaagcccgccaggccccagaggttcctgtcgagccgtttcaggaatcggatcagctgcttgtgcctgtggaactgctgtgcagtcgcacccaggcagcgagtgtccttctcatggtggctgtagaactgccggagcacagtcgcagccctgcagaaggtttccttctcagttgtgttcttggaggcagcaaagatgtctgttacggtcaactcggtgcacagagtcttctgctctgtgaggctgttcaaagttttgatgatctcctgtaaggtgatatcgcacttgtgtcctagtgctgctagtggtaggaagcatagtactagtattagtaggctgcgcatgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 114 - DNA, 3121 bp, vector pUC18-CL-IL-4-H6
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcagggatcctcaatggtgatggtggtggtggctcgaacactttgaatatttctctctcatgatcgtctttagcctttccaagaagttttccaacgtactctggttggcttccttcacaggacaggaattcaagcccgccaggccccagaggttcctgtcgagccgtttcaggaatcggatcagctgcttgtgcctgtggaactgctgtgcagtcgcacccaggcagcgagtgtccttctcatggtggctgtagaactgccggagcacagtcgcagccctgcagaaggtttccttctcagttgtgttcttggaggcagcaaagatgtctgttacggtcaactcggtgcacagagtcttctgctctgtgaggctgttcaaagttttgatgatctcctgtaaggtgatatcgcacttgtgtcctagtgctgctagtggtaggaagcatagtactagtattagtaggctgcgcatgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 115 - DNA, 39 bp, synthetic probe or primer
catcaccattaaggatccctgcaggcatgcaagcttggc

SEQ ID NO: 116 - DNA, 40 bp, synthetic probe or primer
gtggtggtggctcgaacactttgaatatttctctctcatg

SEQ ID NO: 117 - DNA, 7410 bp, vector pFPMT-CL-IL-4-H6
  misc_feature (1077)..(1077): N is any nucleotide
  misc_feature (1078)..(1078): N is any nucleotide
  misc_feature (1256)..(1256): N is any nucleotide
  misc_feature (1257)..(1257): N is any nucleotide
ggtaccctgctcaatctccggaatggtgatctgatcgttcctgaaaacctcgacattggctccctcctgacacaggtactcgtacaggttccaggtaaacgagtcgtagttgtcgatcatgacaacgttcttagaagcggccggcattttgaaggtgactaatagcctaagaaaatatttaatttaattttcattaaattttcctatactcgctatttcagcttttcatctcatcacttcataaacgatataaaccagaaaaagaactattttcaaacacgcttctcaaaagcggtatgtccttccacgtctccttagaatctggcaagtccgcgagggggatcctcaatggtgatggtggtggtggctcgaacactttgaatatttctctctcatgatcgtctttagcctttccaagaagttttccaacgtactctggttggcttccttcacaggacaggaattcaagcccgccaggccccagaggttcctgtcgagccgtttcaggaatcggatcagctgcttgtgcctgtggaactgctgtgcagtcgcacccaggcagcgagtgtccttctcatggtggctgtagaactgccggagcacagtcgcagccctgcagaaggtttccttctcagttgtgttcttggaggcagcaaagatgtctgttacggtcaactcggtgcacagagtcttctgctctgtgaggctgttcaaagttttgatgatctcctgtaaggtgatatcgcacttgtgtcctagtgctgctagtggtaggaagcatagtactagtattagtaggcttcgcatgaattcccgatgaaggcagagagcgcaaggaggcggtatttatagtgccattcccctctctgagagacccggatggtagtcgagtgttatcggagacagcttgatgtagactccgtgcctgccggtcctcttattggcggacaccagtgagacaccccggaacttgctgtttttctgcaaaatccggggtgaccagtgggagcctatttgcacacacgagcgggacaccccactctggtgaagagtgccaaagtcattctttttcccgtnncggggcagccgattgcatgttttaggaaaatattacctttgctacaccctgtcagatttaccctccacacatatatattccgtcacctccagggactattcttggctcgttgcgccgccgcggaagatatccagaagctgtgttttccgagagactcggttggcgcctggtatatttnnaggatgtcgcgctgcctcacgtcccggtacccaggaacgcggtgggatctcgggcccatcgaagactgtgctccagactgctcgcccagcaggtgtttcttgattgccgcctctaaatagtccgcgcatcgccggtaacatttttccagctcggagtttgcgtttagatacatttctgcgatgccaaaggagcctgcagattataacctcggatgctgtcattcagcgcttttaatttgacctccagatagttgctgtatttctgttccattggctgctggacgttcgtataactcgagttattgttgcgctctgcctcggcgtactggctcatgactgactgcggtcgcttctcgagtgttctcgcaacaggacgcctgcaggtcatcgagtcgagctggcgccgaaactggcggatctgacctccacactgccctgtatctctatccaccgggaaccgcctcctgccgttccagaatgttgttcaagtggtagctctgtgcggtcaatgaaggcgttattgccggtgaaatctttgggaagcggtttatcctcggggaagattacgaaattcccgcgcgtcgttgcgcttcctggatctcgaggaagatcgttctccgcgtcgaggagatcgttctccgcgtcgacaccattccttgcggcggcggtgctcaacggcctcaacctactactgggctgcttcctaatgcaggagtcgcataagggagagcgtcgacaaacccgcgtttgagaacttgctcaagcttctggtaaacgttgtagtactctgaaacaaggccctagcactctgatctgtttctcttgggtagcggtgagtggtttattggagttcactggtttcagcacatctgtcatctagacaatattgttactaaatttttttgaactacaattgttcgtaattcatctattattatacatcctcgtcagcaatttctggcagacggagtttactaacgtcttgagtatgaggccgagaatccagctctgtggccatactcagtcttgacagcctgctgatgtggctgcgttcaacgcaataagcgtgtcctccgactccgagttgtgctcgttatcgtcgttctcatcctcggaaaaatcacacgaaagaacatactcaccagtaggctttctggtccctggggcacggctgtttctgacgtattccggcgttgataatagctcgaaagtgaacgccgagtcgcgggagtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatcggggggggggggggggggggggggcaaacaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaagccccccccccccccccccccccccccccccccccgcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaattcccccttacacggaggcatcaagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaa